(19) United States
(12) Patent Application Publication
(10) Pub. No.: US 2015/0166528 A1
(43) Pub. Date: Jun. 18, 2015

(54) Pesticidal methods using substituted 3-pyridyl thiazole compounds and derivatives for combating animal pests

(71) Applicant: BASF SE, Ludwigshafen (DE)

(72) Inventors: Florian Kaiser, Mannheim (DE); Arun Narine, Mannheim (DE); Joachim Dickhaut, Heidelberg (DE); Karsten Körber, Eppelheim (DE); Nina Gertrud Bandur, Mannheim (DE)

(21) Appl. No.: 14/407,731

(22) PCT Filed: Jun. 4, 2013
(86) PCT No.: PCT/EP2013/061508

§ 371 Date: Dec. 12, 2014

Related U.S. Application Data
(60) Provisional application No. 61/659,461, filed on Jun. 14, 2012.

(51) Int. Cl. C07D 417/14; A01N 43/78

(52) U.S. Cl. CPC C07D 417/14; A01N 43/78

(57) ABSTRACT

The present invention relates to pesticidal methods for the use and application of substituted 3-pyridyl thiazole compounds and the stereoisomers, salts, tautomers and N-oxides thereof and to compositions comprising the same. The invention also relates to insecticidal substituted 3-pyridyl thiazole compounds or of the compositions comprising such compounds for combating invertebrate pests and uses thereof.

The substituted 3-pyridyl thiazole compounds of the present invention are defined by the following general formula (I):

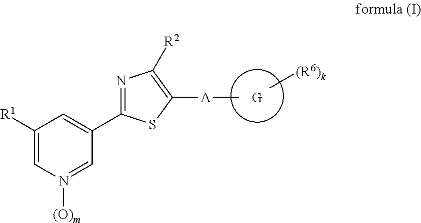

wherein m, k, R¹, R², R⁶, G, and A are defined as in the description.

The present invention relates to pesticidal methods for the use and application of substituted 3-pyridyl thiazole compounds, and the stereoisomers, salts, tautomers and N-oxides thereof and to compositions comprising the same. The invention also relates to insecticidal substituted 3-pyridyl thiazole compounds, or to the compositions comprising such compounds for combating invertebrate pests and uses thereof.

Invertebrate pests and in particular insects, arthropods and nematodes destroy growing and harvested crops and attack wooden dwelling and commercial structures, thereby causing large economic loss to the food supply and to property. While a large number of pesticidal agents are known, due to the ability of target pests to develop resistance to said agents, there is an ongoing need for new agents for combating invertebrate pests such as insects, arachnids and nematodes. It is therefore an object of the present invention to provide compounds having a good pesticidal activity and showing a broad activity spectrum against a large number of different invertebrate pests, especially against difficult to control insects, arachnids and nematodes.

It has been found that these objectives can be achieved by substituted 3-pyridyl thiazole compounds of the general formula (I), as defined below, including their stereoisomers, their salts, in particular their agriculturally or veterinarily acceptable salts, their tautomers and their N-oxides.

Therefore, in a first aspect the present invention relates to pesticidal methods for using substituted 3-pyridyl thiazole compounds of formula (I):

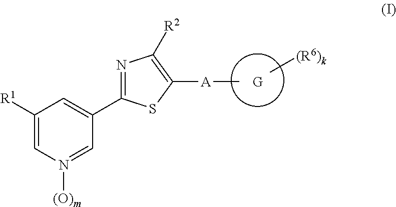

wherein
m is an integer selected from 0 or 1;
k is an integer selected from 0, 1, 2, 3, 4, 5 or 6;
A is selected from O, S(O)ₙ, NR³ or CR^{4a}R^{4b};
G is a molecular group representing a ring system selected from
   a 3, 4-, 5-, 6- or 7-membered heterocyclic or heteroaromatic monocyclic ring system, or
   a 7-11-membered heterobiaromatic or heterobicyclic or heterospirobicyclic ring system, and
   wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized, and
   wherein the ring system may be optionally substituted with k substituents R⁶, wherein said substituents R⁶ are selected independently from one another, if k is more than one;
R¹ is selected from the group consisting of hydrogen, cyano and halogen;
R² is selected from the group consisting of hydrogen, halogen, cyano, C₁-C₆-alkyl and C₁-C₆-cycloalkyl, wherein the latter two may be partially or fully halogenated and/or may be substituted by 1, 2, 3 or 4 radicals R⁵;
R³ is selected from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷, said substituents R⁷ being identical or different from one another if more than one substituent R⁷ is present,
   OR⁸, NR^{9a}R^{9b}, S(O)ₙR⁸, S(O)ₙNR^{9a}R^{9b}, Si(R¹¹)₂R¹²;
   phenyl which may be substituted 1, 2, 3, 4 or 5 substituents R¹⁰, said substituents R¹⁰ being identical or different from one another if more than one substituent R¹⁰ is present, and
   a 3-, 4-, 5-, 6- or 7-membered saturated, partially unsaturated or fully unsaturated heterocyclic ring, wherein said heterocyclic ring comprises 1, 2 or 3 heteroatoms selected from the group consisting of oxygen, nitrogen and sulfur atoms, wherein said nitrogen and sulfur atom(s), independently of one another, may be oxidized, and wherein the heterocyclic ring may be substituted with 1, 2, 3, 4 or 5 substituents R¹⁰, said substituents R¹⁰ being identical or different from one another if more than one substituent R¹⁰ is present;
R^{4a}, R^{4b} are selected independently of one another from the group consisting of hydrogen, cyano, C₁-C₁₀-alkyl, C₃-C₈-cycloalkyl, C₂-C₁₀-alkenyl, C₂-C₁₀-alkynyl,
   wherein the aforementioned aliphatic and cycloaliphatic radicals may be substituted with 1 to 10 substituents R⁷ and wherein said substituents R⁷ are selected independently from one another,
   OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶, C(═S)R¹⁵, C(═S)NR^{17a}R^{17b}, C(═S)OR¹⁶, C(═S)SR¹⁶, C(═NR^{17a})R¹⁵, C(═NR^{17a})NR^{17a}R^{17b}, Si(R¹¹)₂R¹²,
   or
   R^{4a} and R^{4b} together may form a ═CHR¹³, ═CR⁷R¹³, ═NR^{9a} or ═NOR⁸ radical;
R⁵ is each independently from one another selected from the group consisting of cyano, SCN, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
   phenyl, optionally substituted with 1, 2, 3, 4 or 5 substituents R¹⁸, which are independently selected from one another,
   a 3-, 4-, 5-, or 6-membered saturated, partly saturated or unsaturated heterocyclic ring comprising 1, 2 or 3 heteroatoms selected from oxygen, nitrogen and/or sulfur, wherein said nitrogen and sulfur atoms, independently of one another, may be oxidized, and wherein the heterocyclic ring may be substituted with 1, 2, 3, 4 or 5 substituents R¹⁸, said substituents R¹⁸ being identical or different from one another if more than one substituent R¹⁸ is present;
R⁶ is each independently from one another selected from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, and wherein the carbon atoms of the aforementioned aliphatic and cyclo-aliphatic radicals may optionally be further substituted independently from one another with one or more R⁷,
   OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
      phenyl, optionally substituted with 1, 2, 3, 4 or 5 substituents R¹⁰ selected independently from one another from,
   a 3-, 4-, 5-, 6- or 7-membered saturated, partly saturated or unsaturated aromatic heterocyclic ring comprising 1, 2, or 3 heteroatoms selected from oxygen, nitrogen and/or sulfur, optionally substituted with 1, 2, 3, 4 or 5 substituents R¹⁰, selected independently from one another, and wherein the nitrogen and/or the sulfur atom(s) of the heterocyclic ring may optionally be oxidized;
R⁷ is each independently from one another selected from the group consisting of hydrogen, halogen, cyano, azido, nitro, —SCN, SF₅, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁸,
   phenyl, optionally substituted with 1, 2, 3, 4 or 5 sub-stituents R¹⁸, selected independently from one another,
   a 3-, 4-, 5-, 6- or 7-membered saturated, partly saturated or unsaturated aromatic heterocyclic ring comprising 1, 2 or 3 heteroatoms selected from oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized,
   or
   two R⁷ present on one carbon atom may together form ═O, ═CR¹³R¹⁴; ═S, ═NR^{17a}, ═NOR¹⁸; ═NNR^{17a};
   or
   two R⁷ may form a 3-, 4-, 5-, 6-, 7- or 8-membered saturated or partly unsaturated carbocyclic or heterocyclic ring together with the carbon atoms to which the two R⁷ are bonded to;
R⁸ is each independently from one another selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₄-C₈-alkylcycloalkyl, C₃-C₈-halocycloalkyl, C₂-C₆-alkenyl, C₂-C₆-haloalkenyl, C₂-C₆-alkinyl, C₂-C₆ haloalkinyl, —Si(R¹¹)₂R¹², S(O)ₙR¹⁶, S(O)ₙNR^{17a}R^{17b}, NR^{17a}R^{17b}, —N═CR¹³R¹⁴, —C(═O)R¹⁵, C(═O)NR^{17a}R^{17b}, C(═S)NR^{17a}R^{17b}, C(═O)OR¹⁸,
   phenyl, optionally substituted with one or more substituents R¹⁸; which are selected independently from one another,
   a 3-, 4-, 5-, 6- or 7-membered saturated, partly saturated or unsaturated aromatic heterocyclic ring comprising 1, 2 or 3 heteroatoms selected from oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized;
R^{9a}, R^{9b} are each independently from one another selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
   S(O)ₙR¹⁶, —S(O)ₙNR^{17a}R^{17b}, C(═O)R¹⁵, C(═O)OR¹⁶, C(═O)NR^{17a}R^{17b}, C(═S)R¹⁵, C(═S)SR¹⁶, C(═S)NR^{17a}R^{17b}, C(═NR^{17a})R¹⁵;
   phenyl, optionally substituted with 1, 2, 3 or 4 substituents R¹⁸, selected independently from one another;
   a 3-, 4-, 5-, 6- or 7-membered saturated, partly saturated or unsaturated aromatic heterocyclic ring comprising 1, 2 or 3 heteroatoms selected from oxygen, nitrogen and/or sulfur, optionally substituted with 1, 2, 3 or 4, substituents R¹⁸, selected independently from one another, and wherein the nitrogen and/or the sulfur atom(s) of the heterocyclic ring may optionally be oxidized;
   or,
   R^{9a} and R^{9b} are together a C₂-C₇ alkylene chain and form a 3-, 4-, 5-, 6-, 7- or 8-membered saturated, partly saturated or unsaturated aromatic ring together with the nitrogen atom they are bonded to, wherein the alkylene chain may contain one or two heteratoms selected from oxygen, sulfur or nitrogen, and may optionally be substituted with halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
      phenyl, optionally substituted with one or more substituents R¹⁸; which are selected independently from one another, a 3-, 4-, 5-, 6-, or 7-membered saturated, partly saturated or unsaturated aromatic heterocyclic ring comprising 1, 2 or 3 heteroatoms selected from oxygen, nitrogen and/or sulfur, optionally substituted with one or more substituents R¹⁸, selected independently from one another, and wherein the nitrogen and/or the sulfur atom(s) of the heterocyclic ring may optionally be oxidized;
   or
   R^{9a} and R^{9b} together may form a ═CR¹³R¹⁴, ═NR¹⁷ or ═NOR¹⁸ radical;
R¹⁰ is each independently from one another selected from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, wherein the carbon atoms of the aforementioned aliphatic and cyclo-aliphatic radicals may optionally be substituted with one or more R¹⁵, which are selected independently from one another,
   Si(R¹¹)₂R¹², OR¹⁶, OS(O)ₙR¹⁸, —S(O)ₙR¹⁸, S(O)ₙNR^{17a}R^{17b}, NR^{17a}R^{17b}, C(═O)R¹⁵, C(═O)OR¹⁶, —C(═NR^{17a})R¹⁵, C(═O)NR^{17a}R^{17b}, C(═S)NR^{17a}R^{17b},
   phenyl, optionally substituted with halogen, cyano, nitro, C₁-C₆-alkyl, C₁-C₆-haloalkyl, C₁-C₆-alkoxy or C₁-C₆-haloalkoxy,
   a 3-, 4-, 5-, 6- or 7-membered saturated, partly saturated or unsaturated aromatic heterocyclic ring comprising 1, 2 or 3 heteroatoms selected from oxygen, nitrogen and/or sulfur, optionally substituted with one or more substituents selected independently from one another from halogen, cyano, NO₂, C₁-C₆-alkyl, C₁-C₆-haloalkyl, C₁-C₆-alkoxy or C₁-C₆-haloalkoxy, and wherein the nitrogen and/or the sulfur atom(s) of the heterocyclic ring may optionally be oxidized;
   or
   two R¹⁰ present together on one atom of a partly saturated heterocyclic may be ═O, ═CR¹³R¹⁴; ═S, ═NR^{17a}, ═NOR¹⁶ or ═NNR^{17a};
   or,
   two R¹⁰ on adjacent carbon atoms may be a bridge selected from CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17a}, CH₂CH═N, CH═CH—NR^{17a}, OCH═N, SCH═N and form together with the carbon atoms to which the two R¹⁰ are bonded to a 5-membered or 6-membered partly saturated or unsaturated, aromatic carbocyclic or heteocyclic ring, wherein the ring may optionally be substituted with one or two substituents selected from ═O, OH, CH₃, OCH₃, halogen, cyano, halomethyl or halomethoxy;
R¹¹, R¹² are each independently from one another selected from the group consisting of hydrogen, halogen, C₁-C₆ alkyl, C₁-C₆ haloalkyl, C₁-C₆ alkoxy, C₁-C₆ alkoxyalkyl, C₂-C₆ alkenyl, C₂-C₆ haloalkenyl, C₂-C₆ alkinyl, C₂-C₆ haloalkinyl, C₃-C₈ cycloalkyl, C₃-C₈ halocycloalkyl, C₁-C₆ alkoxyalkyl, C₁-C₆ haloalkoxyalkyl and phenyl, optionally substituted with one or more substituents R¹⁸; which are selected independently from one another;
R¹³, R¹⁴ are each independently from one another selected from the group consisting of hydrogen, C₁-C₄ alkyl, C₁-C₆ cycloalkyl, C₁-C₄ alkoxyalkyl, phenyl and benzyl;
R¹⁵ is each independently from one another selected from the group consisting of hydrogen, halogen, cyano, nitro, OH, SH, SCN, SF₅, C₁-C₆-alkoxy, C₁-C₆-haloalkoxy, C₁-C₆-alkylthio, C₁-C₆-alkylsulfonyl, C₁-C₆-haloalkylthio, trimethylsilyl, triethylsilyl, tenbutyldimethylsilyl,
   C₁-C₆-alkyl, C₂-C₆-alkenyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄ alkoxy;
   phenyl, benzyl, pyridyl, phenoxy, wherein the last four radicals may be unsubstituted, partially or fully halogenated and/or to carry 1, 2 or 3 substituents selected from C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino or di-(C₁-C₆-alkyl)amino,
   or
   two R¹⁵ present on the same carbon atom may together be ═O, ═CH(C₁-C₄), ═C(C₁-C₄-alkyl)C₁-C₄-alkyl, ═N(C₁-C₆-alkyl) or ═NO(C₁-C₆-alkyl);
R¹⁶ is each independently from one another selected from the group consisting of hydrogen, cyano, C₁-C₆-alkoxy, C₁-C₆-haloalkoxy, C₁-C₆-alkylthio, C₁-C₆-alkylsulfonyl, C₁-C₆-haloalkylthio, trimethylsilyl, triethylsilyl, tertbutyldimethylsilyl,
   C₁-C₆-alkyl, C₂-C₆-alkenyl, C₃-C₈-cycloalkyl, wherein the four last mentioned radicals may be unsubstituted, partially or fully halogenated and/or oxygenated and/or may carry 1 or 2 radicals selected from C₁-C₄ alkoxy, phenyl, benzyl, pyridyl, phenoxy, wherein the last four radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy or (C₁-C₆-alkoxy)carbonyl;
R^{17a}, R^{17b} are each independently from one another selected from the group consisting of hydrogen, cyano, C₁-C₆-alkoxy, C₁-C₆-haloalkoxy, C₁-C₆-alkylthio, C₁-C₆-alkylsulfonyl, C₁-C₆-haloalkylthio, trimethylsilyl, triethylsilyl, tertbutyldimethylsilyl,
   C₁-C₆-alkyl, C₂-C₆-alkenyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
   phenyl, benzyl, pyridyl, phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy or (C₁-C₆-alkoxy)carbonyl,
   or,
   R^{17a} and R^{17b} may together be a C₂-C₆ alkylene chain forming a 3- to 7-membered saturated, partly saturated or unsaturated ring together with the nitrogen atom R^{17a} and R^{17b} are bonded to, wherein the alkylene chain may contain 1 or 2 heteroatoms selected from oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
R¹⁸ is each independently from one another selected from the group consisting of hydrogen, halogen, nitro, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
   C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
   phenyl, benzyl, pyridyl, phenoxy, wherein the four last mentioned radicals may be unsubstituted, partially or fully halogenated and/or carry 1, 2 or 3 substituents selected from C₁-C₆-alkyl, C₁-C₆-haloalkyl, C₁-C₆-alkoxy, C₁-C₆ haloalkoxy), (C₁-C₆-alkoxy)carbonyl;
   or
   two R¹⁸ present together on one atom of a partly saturated atom may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl; or,
   two R¹⁸ on two adjacent carbon atoms may be together a C₂-C₆ alkylene chain, which form together with the carbon atom they are bonded to a 3-, 4-, 5-, 6- or 7-membered saturated, partly saturated or unsaturated aromatic, wherein the alkylene chain may contain 1 or 2 heteroatoms selected from oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
n is an integer selected from 0, 1 or 2;
   and/or an enantiomer, diastereomer or agriculturally or veterinarily acceptable salts thereof.

One embodiment of the present invention is a method for combating or controlling invertebrate pests comprising contacting the invertebrate pests, or their food supply, habitat or breeding grounds with a substituted 3-pyridyl thiazole compound of the general formula (I) as defined above or an agricultural composition comprising at least one compound of formula (I) as defined above.

One embodiment of the present invention is a method for protecting crops, plants, plant proparagation material, e.g. such as seeds, and/or growing plants from attack or infestation by invertebrate pests comprising contacting or treating the crops, plants, plant proparagation material and growing plants, or soil, material, surface, space, area or water in which the crops, plants, plant proparagation material is stored or the plant is growing, with a substituted 3-pyridyl thiazole compound of the general formula (I) as defined above or an agricultural composition comprising at least one compound of formula (I) as defined above.

One embodiment of the present invention is a method for treating, controlling, preventing or protecting animals against infestation or infection by parasites by administering or applying orally, topically or parenterally to the animals a substituted 3-pyridyl thiazole compound of the general formula (I) as defined above or a veterinary composition comprising at least one compound of formula (I) as defined above.

In another aspect, the present inventions relates also to substituted 3-pyridyl thiazole compounds of formulae (I)-1, (I)-2 and (I)-3 as defined herein below.

One embodiment of the present invention are substituted 3-pyridyl thiazole compounds of the general formula (I)-1:

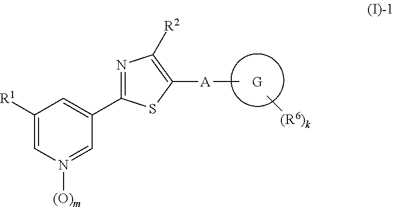

wherein
A is O or NR³, and wherein
m, k, G, R¹, R², R³ and R⁶ are defined as herein above;
   and/or an enantiomer, diastereomer or agriculturally or veterinarily acceptable salts thereof.

One further embodiment of the present invention are substituted 3-pyridyl thiazole compounds of the general formula (I)-2:

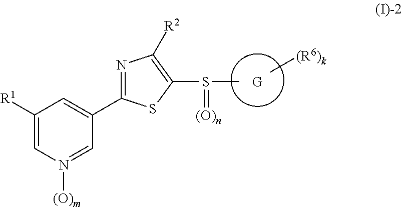

wherein
G is a molecular group representing a ring system selected from
a 3, 4-, 5-, 6- or 7-membered heterocyclic or heteroaromatic monocyclic ring system, or
a 7-11-membered heterobiaromatic or heterobicyclic or heterospirobicyclic ring system, wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized,
wherein the ring system is attached via a carbon ring atom to the remainder of the molecule, and
wherein the ring system may be optionally substituted with k substituents R⁶, wherein said substituents R⁶ are selected independently from one another, if k is more than one,
   and wherein
   n, m, k, R¹, R² and R⁶ are defined as herein above;
   and/or an enantiomer, diastereomer or agriculturally or veterinarily acceptable salts thereof.

Another embodiment of the present invention are substituted 3-pyridyl thiazole compound of the general formula (I)-3:

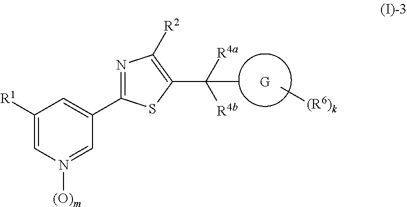

wherein m, k, R¹, R^{4a}, R^{4b} and R⁶ are defined as herein above, and
the combination of G and R² denotes either, that
R² is selected from the group consisting of hydrogen, halogen, cyano, C₃-C₆-alkyl, and C₁-C₆-cycloalkyl, the latter two may be partially or fully halogenated and/or may be substituted by one or more, e.g. 1, 2, 3 or 4, radicals R⁵;
   and
G is a molecular group representing a ring system selected from
   a 3, 4-, 5-, 6- or 7-membered heterocyclic or heteroaromatic monocyclic ring system, or
   a 7-11-membered heterobiaromatic or heterobicyclic or heterospirobicyclic ring system, wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized, and
   wherein the ring system may be optionally substituted with k substituents R⁶, wherein said substituents R⁶ are selected independently from one another, if k is more than one;
      or the combination of G and R² denotes that
R² is selected from the group consisting of methyl, ethyl, difluoromethyl, trifluoromethyl, pentafluoroethyl, 2,2-difluoroethyl and 2,2,2-trifluoroethyl.
G is a molecular group representing a ring system selected from
   a 3, 4-, 5-, 6- or 7-membered heterocyclic or heteroaromatic monocyclic ring system, or
   a 7-11-membered heterobiaromatic or heterobicyclic or heterospirobicyclic ring system, wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized,
   wherein the ring system is attached via a carbon ring atom to the remainder of the molecule, and
   wherein the ring system may be optionally substituted with k substituents R⁶, wherein said substituents R⁶ are selected independently from one another, if k is more than one,
      and/or an enantiomer, diastereomer or agriculturally or veterinarily acceptable salts thereof.

Furthermore, the invention relates to processes for the synthesis of compounds of formula (I) according to the present invention and to intermediate compounds for the synthesis of compounds of formula (I).

One embodiment of the present invention is therefore an intermediate compound of the formula (I-4)

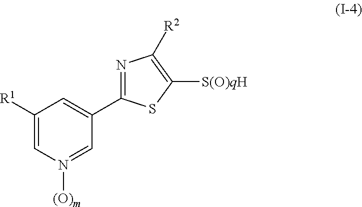

wherein
R¹ is hydrogen or fluoro;
R² is selected from the group consisting of hydrogen, halogen, C₁-C₆-alkyl, C₁-C₆-haloalkyl, C₁-C₆-cycloalkyl and C₁-C₆-cyclohaloalkyl;
q is 0 or 3;
and
m is 0 or 1;
   for the preparation of a compound of formula (I).

Another embodiment of the present invention is a process for the preparation of compounds of formula (I), wherein an intermediate compound of formula (I-4) is used.

One embodiment of the present invention is also an intermediate compound of the formula (I-5)

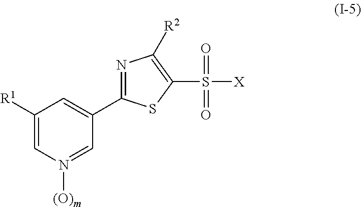

wherein
R¹ is hydrogen or fluoro;
R² is selected from the group consisting of hydrogen, halogen, C₂-C₆-alkyl, C₁-C₆-haloalkyl, C₁-C₆ cycloalkyl and C₁-C₆ cyclohaloalkyl;
X is halogen;
and
m is 0 or 1;
   for the preparation of a compound of formula (I).

Another embodiment of the present invention is a process for the preparation of compounds of formula (I), wherein an intermediate compound of formula (I-5) is used.

One embodiment of the present invention is also an intermediate compound of the formula (I-6)

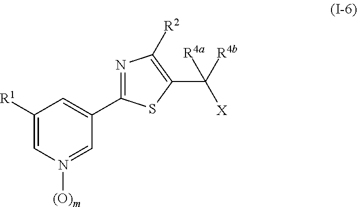

wherein
m is 0 or 1
R¹ is hydrogen or fluoro;
R² is selected from the group consisting of hydrogen, halogen, C₂-C₆-alkyl, C₁-C₆-haloalkyl, C₁-C₆ cycloalkyl and C₁-C₆ cyclohaloalkyl;
R^{4a}, R^{4b} are selected independently from one another from group the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷ and wherein said substituents R⁷ are selected independently from one another,
   C(═O)R⁷, C(═O)NR^{9a}R^{9b}, C(═O)OR⁹, C(═S)R⁷, C(═S)NR^{9a}R^{9b}, C(═S)OR⁹, C(═S)SR⁹, C(═NR^{9a})R⁷, C(═NR^{9a})NR^{9a}R^{9b},
X is halogen;
   for the preparation of compound of formula (I).

Another embodiment of the present invention is a process for the preparation of compounds of formula (I), wherein an intermediate compound of formula (I-6) is used.

One embodiment of the present invention is also an intermediate compound of the formula (I-7)

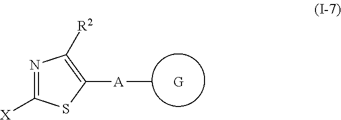

wherein
A is O or NR³;
R² is selected from the group consisting of halogen, C₁-C₆-alkyl, C₁-C₈ haloalkyl, C₁-C₆ cycloalkyl and C₁-C₆ halocycloalkyl;
X is halogen;
and wherein G and R³ are defined as hereinabove.
   for the preparation of compound of formula (I).

Another embodiment of the present invention is a process for the preparation of compounds of formula (I), wherein an intermediate compound of formula (I-7) is used.

One embodiment of the present invention is also an intermediate compound of the formula (I-8)

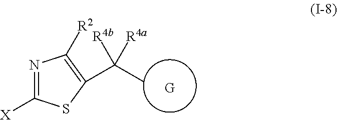

wherein
R² is selected from the group consisting of halogen, C₂-C₆-alkyl, C₁-C₆ haloalkyl, C₁-C₆ cycloalkyl and C₁-C₆ halocycloalkyl;
X is halogen;
R^{4a}, R^{4b} are selected independently from one another from group the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷ and wherein said substituents R⁷ are selected independently from one another,
   C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b},
and wherein G is defined as hereinabove
   for the preparation of compound of formula (I).

Another embodiment of the present invention is a process for the preparation of compounds of formula (I), wherein an intermediate compound of formula (I-8) is used.

The compounds of the present invention, i.e. the compounds of formula (I), their stereoisomers, their salts or their N-oxides, are particularly useful for controlling invertebrate pests, in particular for controlling arthropods and nematodes and especially insects. Therefore, the invention relates to the use of a compound of the present invention, for combating or controlling invertebrate pests, in particular invertebrate pests of the group of insects, arachnids or nematodes.

The term “compound(s) according to the invention” or “compound(s) of formula (I)” comprises the compound(s) as defined herein as well as a stereoisomer, salt, tautomer or N-oxide thereof. The term “compound(s) of the present invention” is to be understood as equivalent to the term “compound(s) according to the invention”, therefore also comprising a stereoisomer, salt, tautomer or N-oxide thereof.

The term “composition(s) according to the invention” or “composition(s) of the present invention” encompasses composition(s) for agricultural or veterinary uses comprising at least one compound of formula (I) according to the invention as defined above.

The present invention relates to a composition comprising at least one compound according to the invention, including a stereoisomer, salt, tautomer or N-oxide thereof, and at least one inert liquid and/or solid carrier. In particular, the invention relates to an agricultural or veterinary composition comprising at least one compound according to the invention including a stereoisomer, an agriculturally or veterinarily acceptable salt, tautomer or an N-oxide thereof, and at least one liquid and/or solid carrier.

The present invention relates to a method for combating or controlling invertebrate pests of the group of insects, arachnids or nematodes, which method comprises contacting said pest or its food supply, habitat or breeding grounds with a pesticidally effective amount of at least one compound according to the invention including a stereoisomer, an agriculturally or veterinarily acceptable salt, tautomer or N-oxide thereof or a composition according to the invention.

The present invention also relates to a method for protecting growing plants from attack or infestation by invertebrate pests of the group of insects, arachnids or nematodes, which method comprises contacting a plant, or soil or water in which the plant is growing or may grow, with a pesticidally effective amount of at least one compound according to the invention including a stereoisomer, an agriculturally acceptable salt, tautomer or N-oxide thereof or a composition according to the invention.

The present invention also relates to a method for the protection of plant propagation material, preferably seeds, from soil insects and of the seedlings' roots and shoots from soil and foliar insects comprising contacting the seeds before sowing and/or after pregermination with at least one compound according to the invention including a stereoisomer, an agriculturally acceptable salt, tautomer or N-oxide thereof or a composition according to the invention.

The present invention also relates to plant propagation material, preferably seed, comprising a compound according to the invention including a stereoisomer, salt, tautomer or N-oxide thereof.

The present invention also relates to the use of a compound according to the invention including a stereoisomer, an agriculturally or veterinarily acceptable salt, tautomer or N-oxide thereof or a composition according to the invention for combating or controlling invertebrate pests of the group of insects, arachnids or nematodes.

The present invention also relates to the use of a compound according to the invention including a stereoisomer, an agriculturally acceptable salt, tautomer or N-oxide thereof or a composition according to the invention for protecting growing plants from attack or infestation by invertebrate pests of the group of insects, arachnids or nematodes.

The present invention also relates to the use of a compound according to the invention including a stereoisomer, veterinarily acceptable salt, tautomer or N-oxide thereof or a composition according to the invention for combating or controlling invertebrate parasites in and on animals.

The present invention also relates to a method for treating an animal infested or infected by parasites or for preventing animals from getting infested or infected by parasites or for protecting an animal against infestation or infection by parasites which comprises orally, topically or parenterally administering or applying to the animal a parasiticidally effective amount of a compound according to the invention including a stereoisomer, veterinarily acceptable salt, tautomer or N-oxide thereof or a composition according to the invention.

The present invention also relates to the use of a compound according to the invention including a stereoisomer, veterinarily acceptable salt or N-oxide thereof or a composition according to the invention for the manufacture of a medicament for protecting an animal against infestation or infection by parasites or treating an animal infested or infected by parasites.

The present invention also relates to a process for the preparation of a composition for treating animals infested or infected by parasites, for preventing animals of getting infected or infested by parasites or protecting animals against infestation or infection by parasites which comprises a compound according to the invention including a stereoisomer, veterinarily acceptable salt, tautomer or N-oxide thereof.

The present invention also relates to a compound according to the invention including a stereoisomer, veterinarily acceptable salt, tautomer or N-oxide thereof for use as a veterinary medicament.

The present invention also relates to a compound according to the invention including a stereoisomer, veterinarily acceptable salt, tautomer or N-oxide thereof for use in the treatment, control, prevention or protection of animals against infestation or infection by parasites.

Certain 3-pyridyl thiazole compounds have been described previously.

Pesticidal 3-pyridyl thiazole carboxamides have been described in the U.S. Pat. No. 4,260,765. Similar compounds are described in U.S. Pat. No. 4,153,703.

The WO 2009149858 describes pyridyl thiazole carboxamide derivatives and their applications as pesticide.

The WO 2010006713 describes pyridyl thiazole-substituted heterocycle derivatives and their use as pesticides. Similar thiazole-substituted heterocycles are likewise disclosed in WO 2011134964, WO 2011138285 and WO 2012000896. Also WO 2012168361, WO 2013000931 and JP 2013075871 describe 2-(3-pyridyl)-thiazole derivatives and their use as pesticides.

The WO 2010129497 describes pyridyl thiazole amines and their applications as pesticides.

Similar pesticidal compounds are likewise disclosed in WO 2011128304. The WO 2012030681 describes corresponding thiadiazole amines as pesticides.

Certain insecticidal heterocyclyl substituted 3-pyridines are also disclosed in WO 2012061949, WO 2012052412, WO 2012061290 and WO 2012061228.

4-haloalkyl-3-heterocyclylpyridines as pesticides are disclosed in WO 9857969. Similar compounds are likewise disclosed in WO 2000035285 and US 20030162812.

Heterocyclyl-substituted thiazole derivatives and their use as fungicides have been described in WO 2007033780.

Substituted haloalkyl thiazole derivatives and their use as insecticides are disclosed in WO 2004056177

Substituted 4-haloalkyl-3-thiazolyl-pyridines are described as pesticides in U.S. Pat. No. 6,521,610. Similar compounds are disclosed in WO 000035285.

Certain substituted mesoionic 2H-pyrido[1,2-a]pyrimidinium-1-[[2-(4-methyl-3-pyridinyl)-5-thiazolyl]methyl]-2,4-dioxo-compounds are described as pesticides in WO 2011017342.

Certain heterocyclyl-substituted pyridyl thiazoles and their use for transcription promotion activity of TFF2 are disclosed in WO 2006035954.

Certain substituted pyridyl thiazole carboxamides are disclosed in WO 2009012482 and WO 2004060281 as specific receptor activity modulators or KCNQ modulators.

However, substituted 3-pyridyl thiazole compounds with the characteristic substitution pattern as in this present invention have not yet been described.

Depending on the substitution pattern, the compounds of the formula (I) may have one or more centers of chirality, in which case they are present as mixtures of enantiomers or diastereomers. The invention provides both the single pure enantiomers or pure diastereomers of the compounds of formula (I), and their mixtures and the use according to the invention of the pure enantiomers or pure diastereomers of the compound of formula (I) or its mixtures. Suitable compounds of the formula (I) also include all possible geometrical stereoisomers (cis/trans isomers) and mixtures thereof. Cis/trans isomers may be present with respect to an alkene, carbonnitrogen double-bond or amide group. The term “stereoisomer(s)” encompasses both optical isomers, such as enantiomers or diastereomers, the latter existing due to more than one center of chirality in the molecule, as well as geometrical isomers (cis/trans isomers). The present invention relates to every possible stereoisomer of the compounds of formula I, i.e. to single enantiomers or diastereomers, as well as to mixtures thereof.

Depending on the substitution pattern, the compounds of the formulae (I) may be present in the form of their tautomers. Hence the invention also relates to the tautomers of the formula (I) and the stereoisomers, salts, tautomers and N-oxides of said tautomers.

The compounds of the present invention may be amorphous or may exist in one or more different crystalline states (polymorphs) or modifications which may have a different macroscopic properties such as stability or show different biological properties such as activities. The present invention includes both amorphous and crystalline compounds of the formula I, mixtures of different crystalline states or modifications of the respective compound I, as well as amorphous or crystalline salts thereof.

Salts of the compounds of the formula I are preferably agriculturally and/or veterinary acceptable salts. They can be formed in a customary method, e.g. by reacting the compound with an acid of the anion in question if the compound of formula I has a basic functionality or by reacting an acidic compound of formula I with a suitable base.

Suitable agriculturally or veterinary useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formulae I with an acid of the corresponding anion, preferably of hydrochloric acid, hydrobromic acid, sulfuric acid, phosphoric acid or nitric acid.

The term “N-oxide” includes any compound of the present invention which has at least one tertiary nitrogen atom that is oxidized to an N-oxide moiety.

The organic moieties mentioned in the above definitions of the variables are—like the term halogen—collective terms for individual listings of the individual group members. The prefix Cₙ-Cₘ indicates in each case the possible number of carbon atoms in the group.

“Halogen” will be taken to mean fluoro, chloro, bromo and iodo.

The term “partially or fully halogenated” will be taken to mean that 1 or more, e.g. 1, 2, 3, 4 or 5 or all of the hydrogen atoms of a given radical have been replaced by a halogen atom, in particular by fluorine or chlorine.

The term “Cₙ-Cₘ-alkyl” as used herein (and also in Cₙ-Cₘ-alkylamino, di-Cₙ-Cₘ-alkylamino, Cₙ-Cₘ-alkylaminocarbonyl, di-(Cₙ-Cₘ-alkylamino)carbonyl, Cₙ-Cₘ-alkylthio, Cₙ-Cₘ-alkylsulfinyl and Cₙ-Cₘ-alkylsulfonyl) refers to a branched or unbranched saturated hydrocarbon group having n to m, e.g. 1 to 10 carbon atoms, preferably 1 to 6 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

The term “Cₙ-Cₘ-haloalkyl” as used herein (and also in Cₙ-Cₘ-haloalkylsulfinyl and Cₙ-Cₘ-haloalkylsulfonyl) refers to a straight-chain or branched alkyl group having n to m carbon atoms, e.g. 1 to 10 in particular 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₁₀-haloalkyl in particular comprises C₁-C₂-fluoroalkyl, which is synonym with methyl or ethyl, wherein 1, 2, 3, 4 or 5 hydrogen atoms are substituted by fluorine atoms, such as fluoromethyl, difluoromethyl, trifluoromethyl, 1-fluoroethyl, 2-fluoroethyl, 2,2-difluoroethyl, 2,2,2-trifluoroethyl and pentafluoromethyl.

Similarly, “Cₙ-Cₘ-alkoxy” and “Cₙ-Cₘ-alkylthio” (or Cₙ-Cₘ-alkylsulfenyl, respectively) refer to straight-chain or branched alkyl groups having n to m carbon atoms, e.g. 1 to 10, in particular 1 to 6 or 1 to 4 carbon atoms (as mentioned above) bonded through oxygen or sulfur linkages, respectively, at any bond in the alkyl group. Examples include C₁-C₄-alkoxy such as methoxy, ethoxy, propoxy, isopropoxy, butoxy, sec-butoxy, isobutoxy and tert-butoxy, further C₁-C₄-alkylthio such as methylthio, ethylthio, propylthio, isopropylthio, and n-butylthio.

Accordingly, the terms “Cₙ-Cₘ-haloalkoxy” and “Cₙ-Cₘ-haloalkylthio” (or Cₙ-Cₘ-haloalkylsulfenyl, respectively) refer to straight-chain or branched alkyl groups having n to m carbon atoms, e.g. 1 to 10, in particular 1 to 6 or 1 to 4 carbon atoms (as mentioned above) bonded through oxygen or sulfur linkages, respectively, at any bond in the alkyl group, where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy, further C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like. Similarly the terms C₁-C₂-fluoroalkoxy and C₁-C₂-fluoroalkylthio refer to C₁-C₂-fluoroalkyl which is bound to the remainder of the molecule via an oxygen atom or a sulfur atom, respectively.

The term “C₂-Cₘ-alkenyl” as used herein intends a branched or unbranched unsaturated hydrocarbon group having 2 to m, e.g. 2 to 10 or 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “C₂-Cₘ-alkynyl” as used herein refers to a branched or unbranched unsaturated hydrocarbon group having 2 to m, e.g. 2 to 10 or 2 to 6 carbon atoms and containing at least one triple bond, such as ethynyl, propynyl, 1-butynyl, 2-butynyl, and the like.

The term “C₁-C₄-alkoxy-C₁-C₄-alkyl” as used herein refers to alkyl having 1 to 4 carbon atoms, e.g. like specific examples mentioned above, wherein one hydrogen atom of the alkyl radical is replaced by an C₁-C₄-alkoxy group.

The term “C₃-Cₘ-cycloalkyl” as used herein refers to a monocyclic 3- to m-membered saturated cycloaliphatic radicals, e.g. cyclopropyl, cyclobutyl, cyclopentyl, cyclohexyl, cycloheptyl, cyclooctyl and cyclodecyl.

The term “aryl” as used herein refers to an aromatic hydrocarbon radical such as naphthyl or in particular phenyl.

The term “3- to 6-membered carbocyclic ring” as used herein refers to cyclopropane, cyclobutane, cyclopentane and cyclohexane rings.

The term “3-, 4-, 5-, 6- or 7-membered saturated, partially unsaturated or aromatic heterocyclic ring containing 1, 2 or 3 heteroatoms” or “containing heteroatom groups”, wherein those heteroatom(s) (group(s)) are selected from N, O, S, NO, SO and SO₂ and are ring members, as used herein refers to monocyclic radicals, the monocyclic radicals being saturated, partially unsaturated or aromatic. The heterocyclic radical may be attached to the remainder of the molecule via a carbon ring member or via a nitrogen ring member.

Examples of 3-, 4-, 5-, 6- or 7-membered saturated heterocyclyl or heterocyclic rings include: Oxiranyl, aziridinyl, azetidinyl, 2 tetrahydrofuranyl, 3-tetrahydrofuranyl, 2 tetrahydrothienyl, 3 tetrahydrothienyl, 2-pyrrolidinyl, 3-pyrrolidinyl, 3 pyrazolidinyl, 4 pyrazolidinyl, 5-pyrazolidinyl, 2 imidazolidinyl, 4 imidazolidinyl, 2-oxazolidinyl, 4-oxazolidinyl, 5 oxazolidinyl, 3-isoxazolidinyl, 4 isoxazolidinyl, 5 isoxazolidinyl, 2 thiazolidinyl, 4-thiazolidinyl, 5-thiazolidinyl, 3 isothiazolidinyl, 4-isothiazolidinyl, 5 isothiazolidinyl, 1,2,4-oxadiazolidin-3-yl, 1,2,4 oxadiazolidin 5 yl, 1,2,4-thiadiazolidin-3-yl, 1,2,4 thiadiazolidin-5-yl, 1,2,4 triazolidin-3-yl, 1,3,4-oxadiazolidin-2-yl, 1,3,4 thiadiazolidin-2-yl, 1,3,4 triazolidin-2-yl, 2-tetrahydropyranyl, 4 tetrahydropyranyl, 1,3-dioxan-5-yl, 1,4-dioxan-2-yl, 2-piperidinyl, 3-piperidinyl, 4-piperidinyl, 3-hexahydropyridazinyl, 4 hexahydropyridazinyl, 2-hexahydropyrimidinyl, 4-hexahydropyrimidinyl, 5 hexahydropyrimidinyl, 2-piperazinyl, 1,3,5-hexahydrotriazin-2-yl and 1,2,4 hexahydrotriazin-3-yl, 2-morpholinyl, 3-morpholinyl, 2-thiomorpholinyl, 3-thiomorpholinyl, 1-oxothiomorpholin-2-yl, 1-oxothiomorpholin-3-yl, 1,1-dioxothiomorpholin-2-yl, 1,1-dioxothiomorpholin-3-yl, hexahydroazepin-1-, -2-, -3- or -4-yl, hexahydrooxepinyl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of 3-, 4-, 5-, 6- or 7-membered partially unsaturated heterocyclyl or heterocyclic rings include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3 dihydrothien-3-yl, 2,4 dihydrothien-2-yl, 2,4-dihydrothien-3-yl, 2-pyrrolin-2-yl, 2-pyrrolin-3-yl, 3 pyrrolin-2-yl, 3-pyrrolin-3-yl, 2-isoxazolin-3-yl, 3-isoxazolin-3-yl, 4 isoxazolin 3 yl, 2-isoxazolin-4-yl, 3-isoxazolin-4-yl, 4-isoxazolin-4-yl, 2 isoxazolin-5-yl, 3-isoxazolin-5-yl, 4-isoxazolin-5-yl, 2-isothiazolin-3-yl, 3 isothiazolin-3-yl, 4-isothiazolin-3-yl, 2-isothiazolin-4-yl, 3-isothiazolin-4-yl, 4 isothiazolin-4-yl, 2-isothiazolin-5-yl, 3-isothiazolin-5-yl, 4-isothiazolin-5-yl, 2,3 dihydropyrazol-1-yl, 2,3-dihydropyrazol-2-yl, 2,3-dihydropyrazol-3-yl, 2,3 dihydropyrazol-4-yl, 2,3-dihydropyrazol-5-yl, 3,4-dihydropyrazol-1-yl, 3,4 dihydropyrazol-3-yl, 3,4-dihydropyrazol-4-yl, 3,4-dihydropyrazol-5-yl, 4,5 dihydropyrazol-1-yl, 4,5-dihydropyrazol-3-yl, 4,5-dihydropyrazol-4-yl, 4,5 dihydropyrazol-5-yl, 2,3-dihydrooxazol-2-yl, 2,3-dihydrooxazol-3-yl, 2,3 dihydrooxazol-4-yl, 2,3-dihydrooxazol-5-yl, 3,4-dihydrooxazol-2-yl, 3,4 dihydrooxazol-3-yl, 3,4-dihydrooxazol-4-yl, 3,4-dihydrooxazol-5-yl, 3,4 dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4 di- or tetrahydropyridazinyl, 2-di- or tetrahydropyrimidinyl, 4-di- or tetrahydropyrimidinyl, 5 di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7 tetrahydro[1H]azepin-1-, -2-, -3-, -4-, -5-, -6- or -7-yl, 2,3,6,7 tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7 tetrahydro[1H]oxepin-2-, -3-, -4-, -5-, -6- or -7-yl, 2,3,6,7 tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

Examples of 5- or 6-membered aromatic heterocyclyl (hetaryl) or heteroaromatic rings are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4 thiazolyl, 5-thiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

A “C₂-Cₘ-alkylene” is divalent branched or preferably unbranched saturated aliphatic chain having 2 to m, e.g. 2 to 7 carbon atoms, for example CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂, CH₂CH₂CH₂CH₂CH₂CH₂, and CH₂CH₂CH₂CH₂CH₂CH₂CH₂.

### PREFERENCES AND EMBODIMENTS

Embodiments and preferred compounds of the present invention for use in pesticidal methods and for insecticidal application purposes are outlined in the following paragraphs.

The remarks made below concerning preferred embodiments of the variables of the compounds of formula I, especially with respect to their substituents A, G, R¹, R² and R⁶ as well as to the meaning of m and k, are valid both, on their own and, in particular, in every possible combination with each other.

Preferred methods of the present invention are methods using a substituted 3-pyridyl thiazole compound of the general formula (I), wherein R¹ is hydrogen or fluoro.

Preferred methods of the present invention are methods using a substituted 3-pyridyl thiazole compound of the general formula (I), wherein R² is selected from the group consisting of hydrogen, fluoro, chloro, bromo, cyano, methyl, ethyl, propyl, isopropyl, tertbutyl, cyclopropyl, difluoromethyl, trifluoromethyl, pentafluoroethyl, 2,2-difluoroethyl or 2,2,2-trifluoroethyl.

Preferred methods of the present invention are methods using a substituted 3-pyridyl thiazole compound of the general formula (I), wherein A is O.

Preferred methods of the present invention are methods using a substituted 3-pyridyl thiazole compound of the general formula (I), wherein A is S(O)ₙ and n is 0, 1 or 2.

Preferred methods of the present invention are methods using a substituted 3-pyridyl thiazole compound of the general formula (I), wherein A is NR³, and R³ is selected from the group consisting of hydrogen, C₁-C₆-alkyl, C₃-C₆-cycloalkyl, C₂-C₆-alkenyl, C₂-C₆-alkynyl, wherein the aforementioned aliphatic and cycloaliphatic radicals each independently may be substituted with 1 to 5 substituents R¹⁵, said substituents R¹⁵ being identical or different from one another if more than one substituent R¹⁵ is present.

Preferred methods of the present invention are methods using a substituted 3-pyridyl thiazole compound of the general formula (I), wherein A is CR^{4a}R^{4b} and R^{4a} and R^{4b} are selected independently of each other from the group consisting of hydrogen, C₁-C₆-alkyl, C₃-C₇-cycloalkyl, C₂-C₆-alkenyl, C₂-C₆-alkynyl, wherein the aforementioned aliphatic and cycloaliphatic radicals each independently may be substituted with 1 to 10 substituents R¹⁵, said substituents R¹⁵ being identical or different from one another if more than one substituent R⁷ is present, OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶, C(═S)R¹⁵, C(═S)NR^{17a}R^{17b},

or
R^{4a} and R^{4b} together may form a ═CHR¹³, ═CR⁷R¹³, ═NR^{9a} or ═NOR⁸ radical.

Preferred methods of the present invention are methods using a substituted 3-pyridyl thiazole compound of the general formula (I), wherein G is a nitrogen containing 4-, 5- or 6-membered heterocyclic or hetero-aromatic ring system selected from any of the following ring systems G-1k to G-135k:

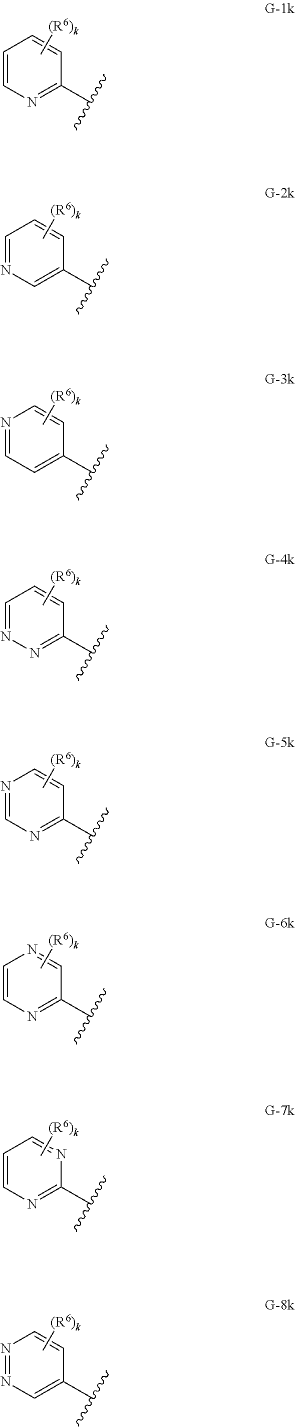

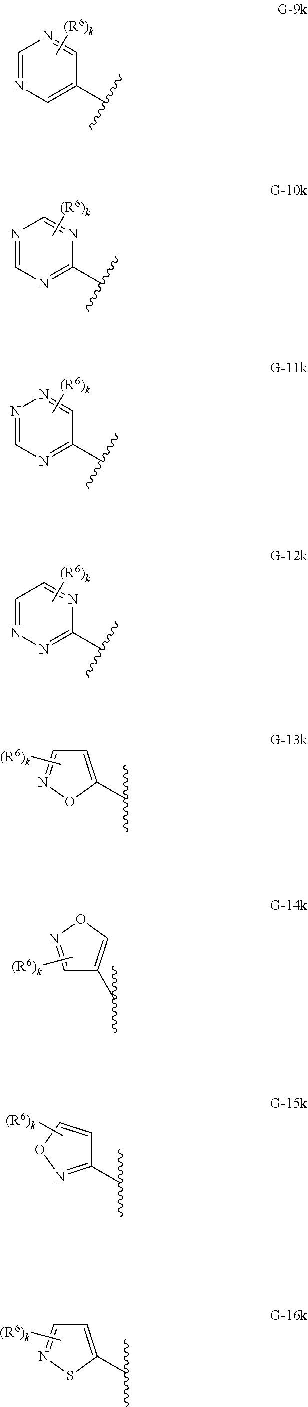

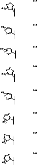

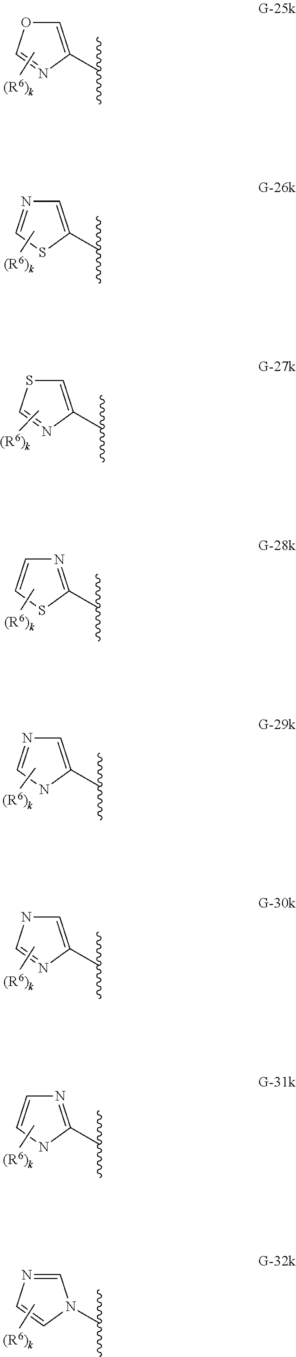

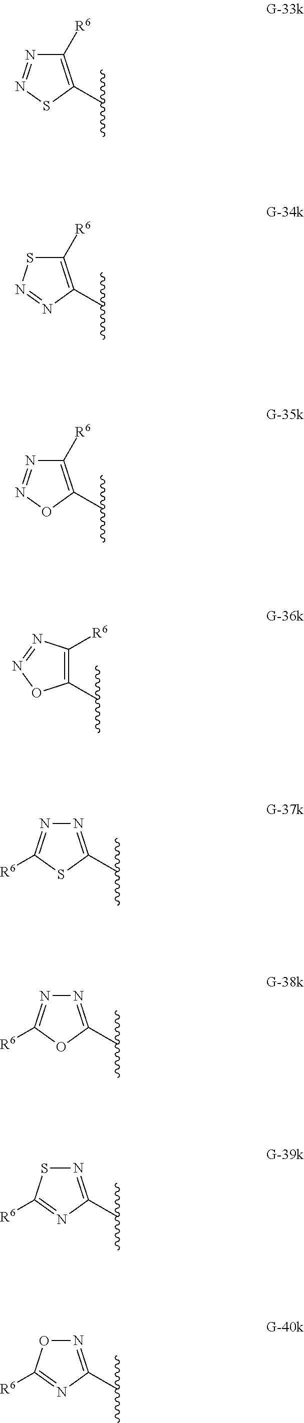

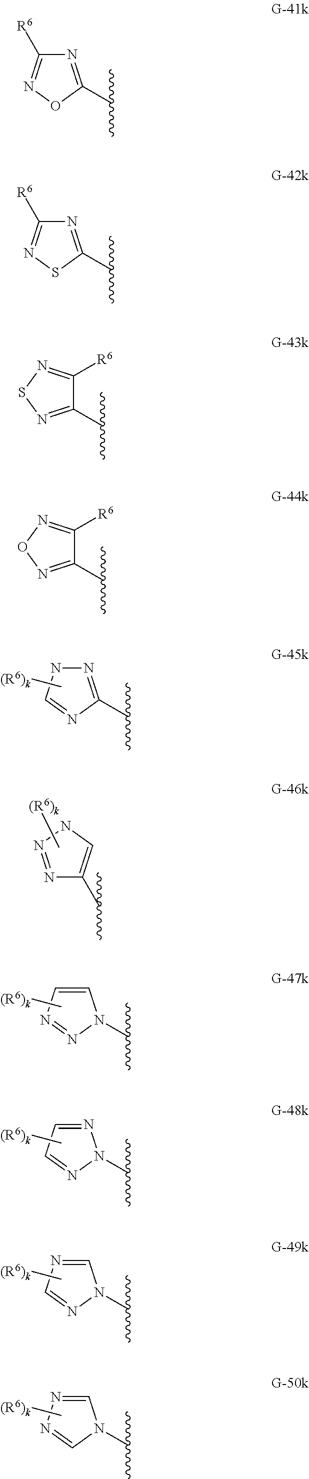

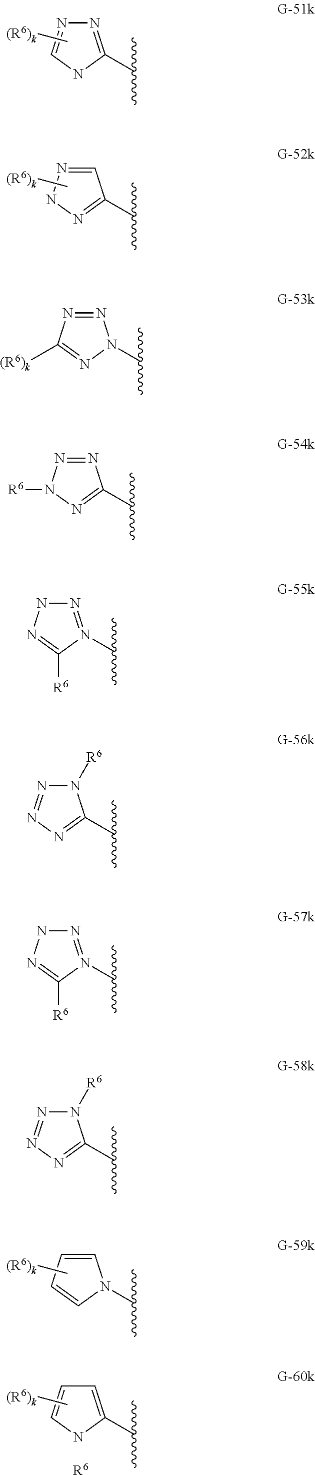

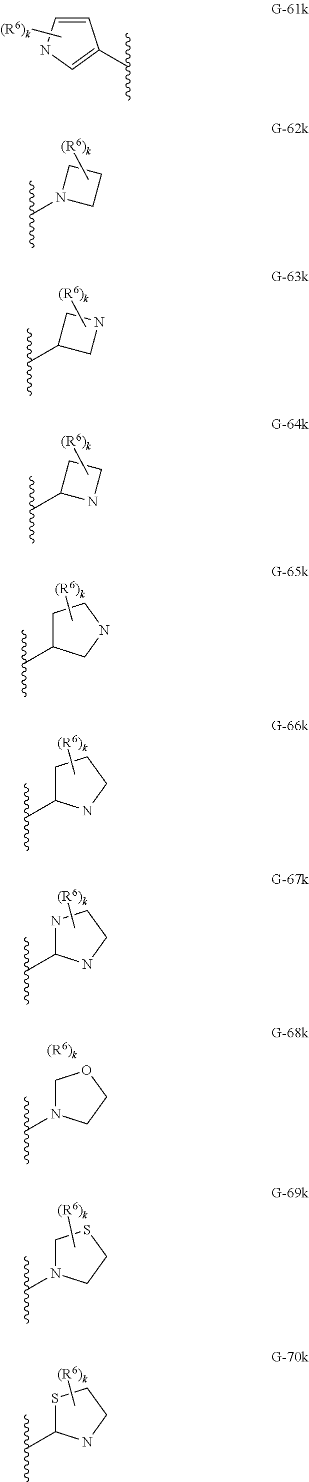

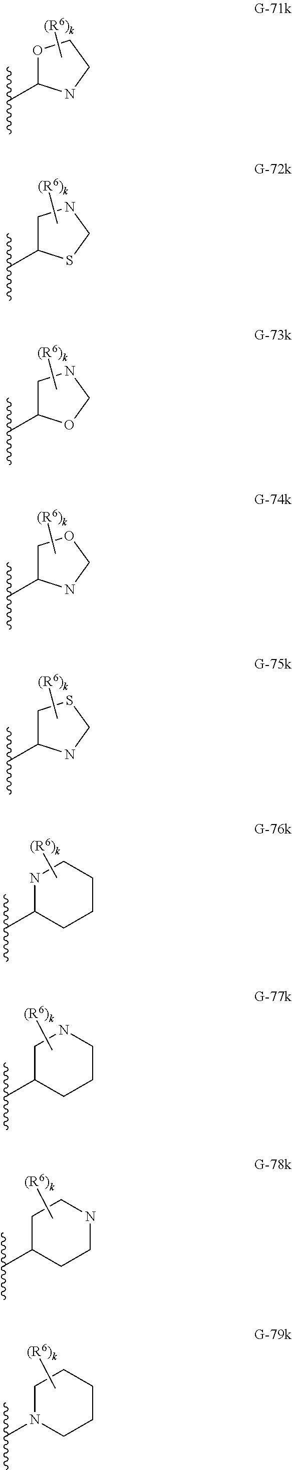

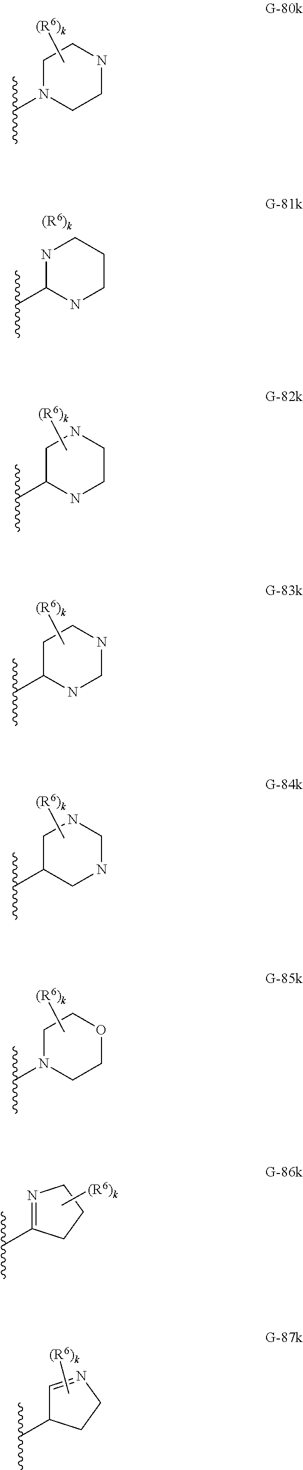

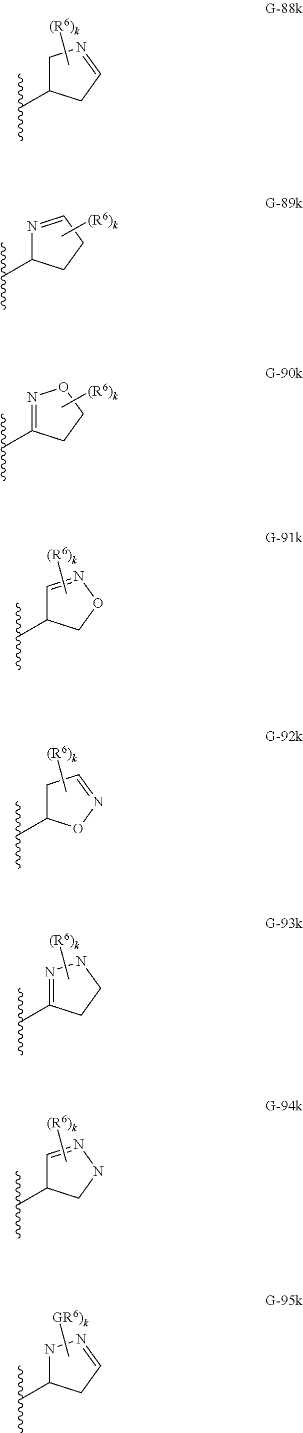

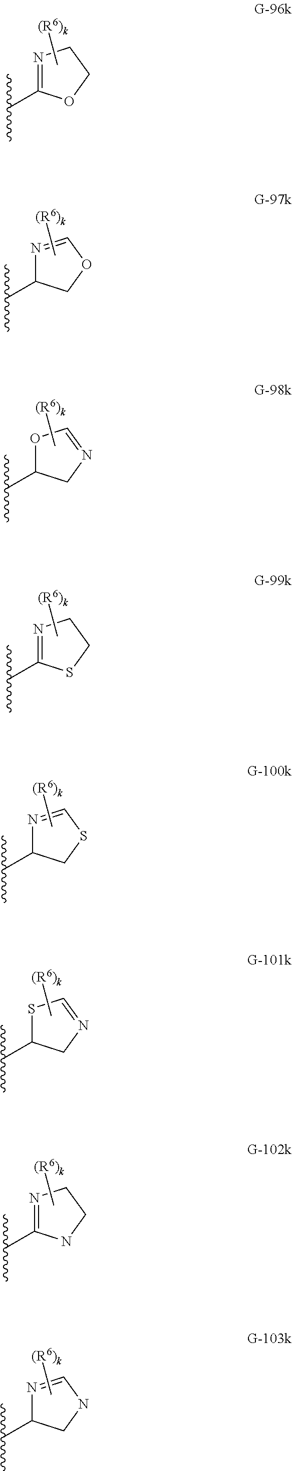

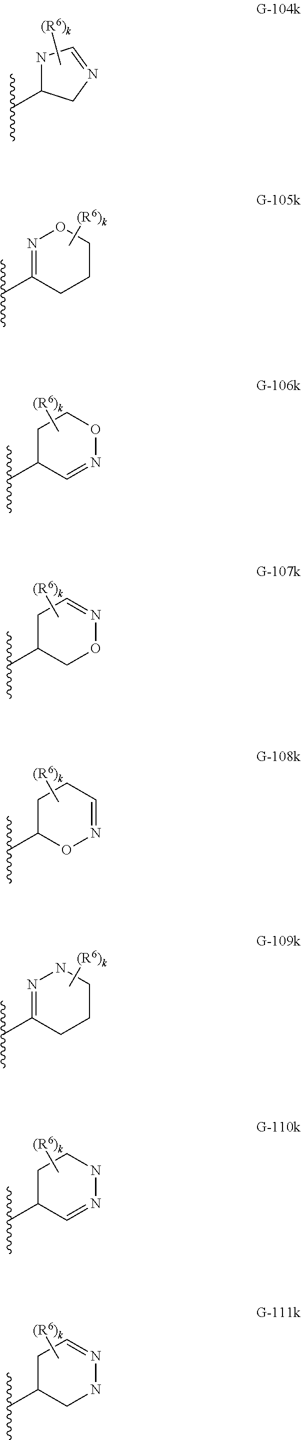

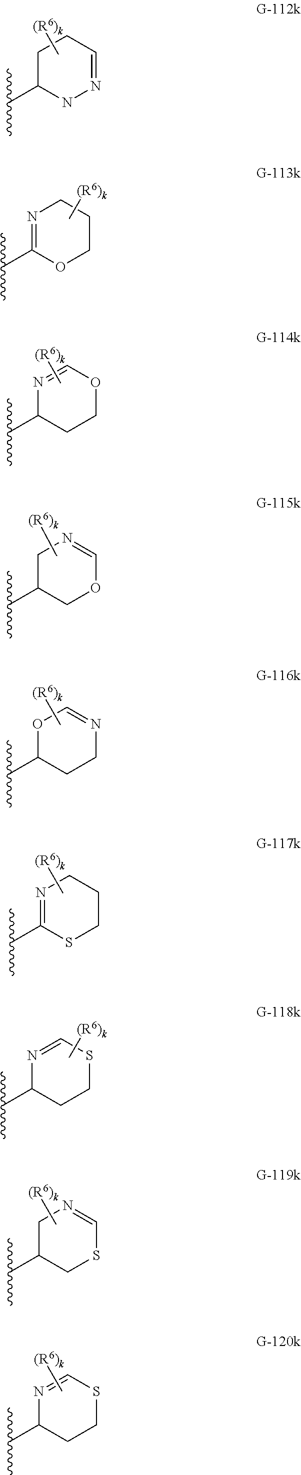

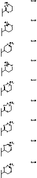

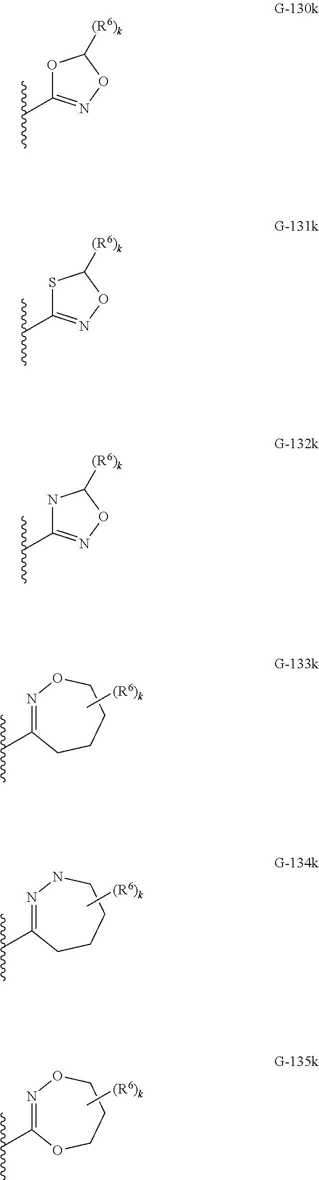

wherein the zigzag line of the moiety G denotes the bond to the group A of formula (I);
k is an integer selected from 0, 1, 2, 3, 4, 5 or 6,
and,
R⁶ is independently from one another selected from the group consisting of hydrogen, halogen, cyano, nitro, SCN, C₁-C₆-alkyl, C₃-C₆-cycloalkyl, C₂-C₆-alkenyl, C₂-C₆-alkinyl, wherein the aforementioned aliphatic and cyclo-aliphatic radicals may optionally be substituted with one or more R⁷, which are selected independently from one another,
   OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b};
   phenyl, optionally substituted with 1, 2, 3, 4, or 5 substituents R¹⁰ selected independently from one another;
   a 3-, 4-, 5-, 6- or 7-membered saturated, partly saturated or unsaturated aromatic heterocyclic ring comprising 1, 2, 3 or 4 heteroatoms selected from oxygen, nitrogen and/or sulfur, optionally substituted with one or more, e.g. 1, 2, 3, 4, or 5 substituents selected independently from R¹⁰, and wherein the nitrogen and/or the sulfur atom(s) of the heterocyclic ring may optionally be oxidized;
   or
   two R⁶ present on one ring carbon or sulfur atom may together form ═O, ═S, ═NR^{17a}.

Preferred methods of the present invention are methods using a substituted 3-pyridyl thiazole compound of the general formula (I), wherein
A is selected from O, NR³ or CR^{4a}R^{4b}, and
G is the nitrogen containing 4-, 5-, or 6-membered heterocyclic or hetero-aromatic ring system selected from any of the following ring systems:
   G-1k, G-2k, G-3k, G-5k, G-7k, G-9k, G-19k, G-21k, G-22k, G-26k, G-27k, G-28k, G-29k, G-30k, G-31k, G-32k, G-37k, G-45k, G-49k, G-50k, G-51k, G-90k, G-93k, G-96k, G-99k, G-102k, G-113k, G-117k, G-121k, G-125k, G-126k, G-127k, G-130k, G-131k, G-132k, G-135k as defined herein above, and wherein each
   R⁶ is independently from one another selected from the group consisting of hydrogen, halogen, cyano, nitro, C₁-C₆-alkyl, C₃-C₆-cycloalkyl, wherein the carbon atoms of the aforementioned aliphatic and cyclo-aliphatic radicals may optionally be partially or fully halogenated and/or may carry 1 or 2 radicals R¹⁵, which are selected independently from one another,
      OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶; or a ring system selected from phenyl, pyridyl, pyrimidyl, pyrazolyl, thiazolyl, oxazolyl or thiophenyl, thiazolinyl, oxazolinyl, isoxazolinyl, pyrazolinyl, imidazolinyl, each optionally substituted with one or more, e.g. 1, 2, 3, 4, or 5 substituents selected independently from R¹⁸;
      or two R⁶ present on one ring carbon or sulfur atom may together form ═O, ═S, ═NR^{17a};
      and wherein
      k is an integer selected from 0, 1, 2 or 3.

Preferred methods of the present invention are methods using a substituted 3-pyridyl thiazole compound of the general formula (I), wherein
A is selected from S(O)ₙ, and
G is the nitrogen containing 4-, 5-, or 6-membered heterocyclic or hetero-aromatic ring system is selected from any of the following ring systems:
   G-1k to G-21k, G-23k to G-31k, G-33k to G-46k, G-51k, G-52k, G-54k, G-56k, G-58k, G-60k, G-61k, G-63k to G-67k, G-70k to G-78k, G-81k to G-84k, G-86k to G-104k, G-113k, G-117k and G-121k,
   and
   R⁶ is independently selected from the group consisting of hydrogen, halogen, cyano, nitro, C₁-C₆-alkyl, C₃-C₆-cycloalkyl, wherein the carbon atoms of the aforementioned aliphatic and cyclo-aliphatic radicals may optionally be partially or fully halogenated and/or may carry 1 or 2 radicals R¹⁵, which are selected independently from one another,
      OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶; or a ring system selected from phenyl, pyridyl, pyrimidyl, pyrazolyl, thiazolyl, oxazolyl or thiophenyl, thiazolinyl, oxazolinyl, isoxazolinyl, pyrazolinyl, imidazolinyl, each optionally substituted with one or more, e.g. 1, 2, 3, 4, or 5 substituents selected independently from R¹⁸;
      or two R⁶ present on one ring carbon or sulfur atom may together form ═O, ═S, ═NR^{17a};
      and wherein
      k is 0, 1, 2 or 3;

One embodiment of the present invention are substituted 3-pyridyl thiazole compounds of the general formula (I)-1

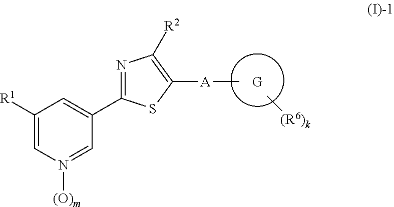

and pesticidal methods using them, wherein
A is O or NR³, and wherein m, k, G, R¹, R², R³ and R⁶ are defined as hereinabove,
and/or an enantiomer, diastereomer or agriculturally or veterinarily acceptable salts thereof.

One embodiment of the present invention are substituted 3-pyridyl thiazole compound of the general formula (I)-2

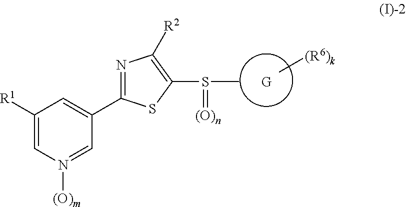

and pesticidal methods using them, wherein
G is a molecular group representing a ring system selected from
a 3, 4-, 5-, 6- or 7-membered heterocyclic or heteroaromatic monocyclic ring system or a 7-11-membered heterobiaromatic or heterobicyclic or heterospirobicyclic ring system, wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized, and
wherein the ring system is attached via a carbon ring atom to the remainder of the molecule, and
wherein the ring system may be optionally substituted with k substituents R⁶, wherein said substituents R⁶ are selected independently from one another, if k is more than one,
   and wherein n, m, k, R¹, R² and R⁶, are defined as hereinabove,
   and/or an enantiomer, diastereomer or agriculturally or veterinarily acceptable salts thereof.

With regard to substituted 3-pyridyl thiazole compound of the general formula (I)-3, wherein A is CR^{4a}R^{4b},

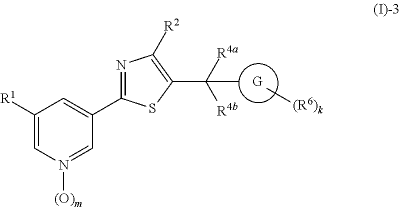

and to pesticidal methods using them, wherein
R² is selected from the group consisting of hydrogen, halogen, cyano, C₃-C₆-alkyl, C₁-C₆-cycloalkyl, the latter two may be partially or fully halogenated and/or may be substituted by one or more, e.g. 1, 2, 3 or 4, radicals R⁵;
and m, k, R¹, G, R^{4a}R^{4b}, R⁵ and R⁶ are defined as herein above;
   and/or an enantiomer, diastereomer or agriculturally or veterinarily acceptable salts thereof.

One embodiment of the present invention are such substituted 3-pyridyl thiazole compound of the general formula (I)-3, wherein A is CR^{4a}R^{4b},

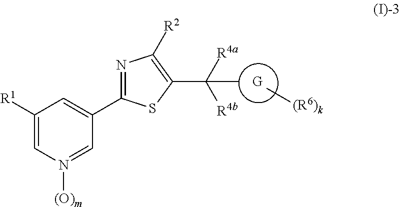

and pesticidal methods using them, wherein
R² is selected from the group consisting of methyl, ethyl, difluoromethyl, trifluoromethyl, pentafluoroethyl, 2,2-difluoroethyl or 2,2,2-trifluoroethyl,
and
G is a molecular group representing a ring system selected from
   a 3, 4-, 5-, 6- or 7-membered heterocyclic or heteroaromatic monocyclic ring system, or a 7-11-membered heterobiaromatic or heterobicyclic or heterospirobicyclic ring system, wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized, and
   wherein the ring system is attached via a carbon ring atom to the remainder of the molecule, and
   wherein the ring system may be optionally substituted with k substituents R⁶, wherein said substituents R⁶ are selected independently from one another, if k is more than one,
      and wherein m, k, R¹, R^{4a}R^{4b} and R⁶, are defined as herein above,
      and/or an enantiomer, diastereomer or agriculturally or veterinarily acceptable salts thereof.

Especially preferred are substituted 3-pyridyl thiazole compounds of formulae (I-1), (I-2) and (I-3), wherein R¹ is selected from the group consisting of hydrogen or fluoro.

Especially preferred are substituted 3-pyridyl thiazole compounds of formulae (I-1) and (I-2), wherein R¹ is selected from the group consisting of hydrogen or fluoro and R² is selected from the group consisting of fluoro, chloro, bromo, cyano, methyl, ethyl, propyl, isopropyl, tertbutyl, cyclopropyl, difluoromethyl, trifluoromethyl, 2,2-difluoroethyl or 2,2,2-trifluoroethyl.

Especially preferred are substituted 3-pyridyl thiazole compounds of formula (I-3), wherein R² is selected from the group consisting of hydrogen, fluoro, chloro, bromo, cyclopropyl, propyl, tertbutyl, and

G is a molecular group representing a ring system selected from
a 3, 4-, 5-, 6- or 7-membered heterocyclic or heteroaromatic monocyclic ring system or a 7-11-membered heterobiaromatic or heterobicyclic or heterospirobicyclic ring system, wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized, and
wherein the ring system is attached via a carbon ring atom to the remainder of the molecule, and
wherein the ring system may be optionally substituted with k substituents R⁶, wherein said substituents R⁶ are selected independently from one another, if k is more than one, and wherein
R⁶ is independently selected from the group consisting of hydrogen, halogen, cyano, nitro, C₁-C₆-alkyl, C₃-C₆-cycloalkyl, wherein the carbon atoms of the aforementioned aliphatic and cyclo-aliphatic radicals may optionally be partially or fully halogenated and/or may carry 1 or 2 radicals R¹⁵, which are selected independently from one another,
   OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶;
   or a ring system selected from phenyl, pyridyl, pyrimidyl, pyrazolyl, thiazolyl, oxazolyl or thiophenyl, thiazolinyl, oxazolinyl, isoxazolinyl, pyrazolinyl, imidazolinyl, each optionally substituted with one or more, e.g. 1, 2, 3, 4, or 5 substituents selected independently from R¹⁸;
   or two R⁶ present on one ring carbon or sulfur atom may together form ═O, ═S, ═NR^{17a}; and wherein
k is an integer of 0, 1, 2 or 3.

Especially preferred are substituted 3-pyridyl thiazole compounds of formulae (I-1) and (I-3), wherein
R¹ is selected from the group consisting of hydrogen or fluoro;
R² is selected from the group consisting of fluoro, chloro, bromo, cyano, methyl, ethyl, propyl, isopropyl, tertbutyl, cyclopropyl, difluoromethyl, trifluoromethyl, 2,2-difluoroethyl or 2,2,2-trifluoroethyl;
G is a nitrogen containing 4-, 5-, or 6-membered heterocyclic or hetero-aromatic ring system selected from any of the ring systems G-1k to G-135k defined herein above, and wherein each
   R⁶ is independently selected from the group consisting of hydrogen, halogen, cyano, nitro, C₁-C₆-alkyl, C₃-C₆-cycloalkyl, wherein the carbon atoms of the aforementioned aliphatic and cyclo-aliphatic radicals may optionally be partially or fully halogenated and/or may carry 1 or 2 radicals R¹⁵, which are selected independently from one another,
      OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶;
      or a ring system selected from phenyl, pyridyl, pyrimidyl, pyrazolyl, thiazolyl, oxazolyl or thiophenyl, thiazolinyl, oxazolinyl, isoxazolinyl, pyrazolinyl, imidazolinyl, each optionally substituted with one or more, e.g. 1, 2, 3, 4, or 5 substituents selected independently from R¹⁸;
      or two R⁶ present on one ring carbon or sulfur atom may together form ═O, ═S, ═NR^{17a};
      and wherein
   k is an integer of 0, 1, 2 or 3.

Especially preferred are substituted 3-pyridyl thiazole compounds of formulae (I-2), wherein
R¹ is selected from the group consisting of hydrogen or fluoro;
R² is selected from the group consisting of hydrogen, fluoro, chloro, bromo, cyclopropyl, propyl, tertbutyl;
G is a nitrogen containing 4-, 5-, or 6-membered heterocyclic or hetero-aromatic ring system selected from any of the following ring systems:
   G-1k to G-21k, G-23k to G-31k, G-33k to G-46k, G-51k, G-52k, G-54k, G-56k, G-58k, G-60k, G-61k, G-63k to G-67k, G-70k to G-78k, G-81k to G-84k, G-86k to G-135k as defined herein above,
   and wherein each
   R⁶ is independently selected from the group consisting of hydrogen, halogen, cyano, nitro, C₁-C₆-alkyl, C₃-C₆-cycloalkyl, wherein the carbon atoms of the aforementioned aliphatic and cyclo-aliphatic radicals may optionally be partially or fully halogenated and/or may carry 1 or 2 radicals R¹⁵, which are selected independently from one another,
      OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶;
      or a ring system selected from phenyl, pyridyl, pyrimidyl, pyrazolyl, thiazolyl, oxazolyl or thiophenyl, thiazolinyl, oxazolinyl, isoxazolinyl, pyrazolinyl, imidazolinyl, each optionally substituted with one or more, e.g. 1, 2, 3, 4, or 5 substituents selected independently from R¹⁸;
      or two R⁶ present on one ring carbon or sulfur atom may together form ═O, ═S, ═NR^{17a};
      and wherein
      k is an integer of 0, 1, 2 or 3.

Especially preferred are substituted 3-pyridyl thiazole compounds of the following 288 formulae I-aa to I-mk:

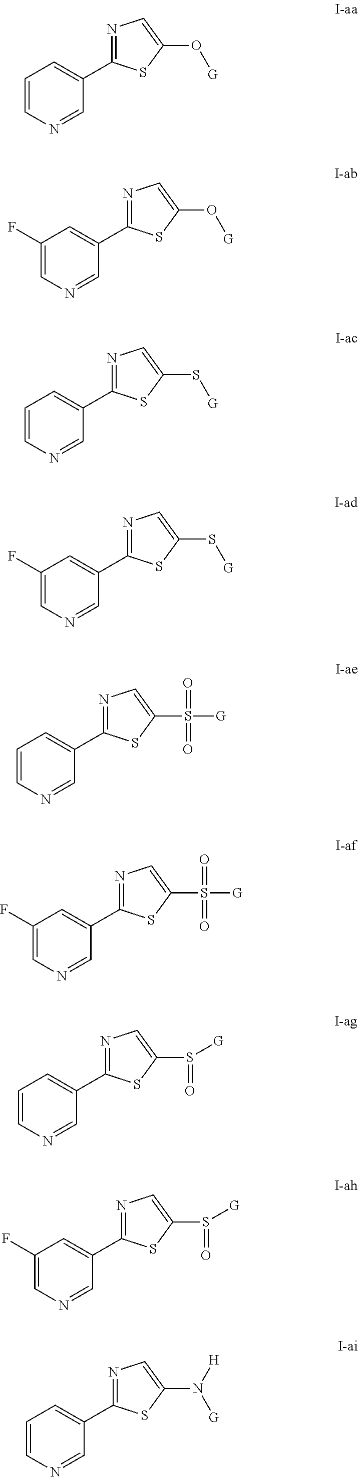

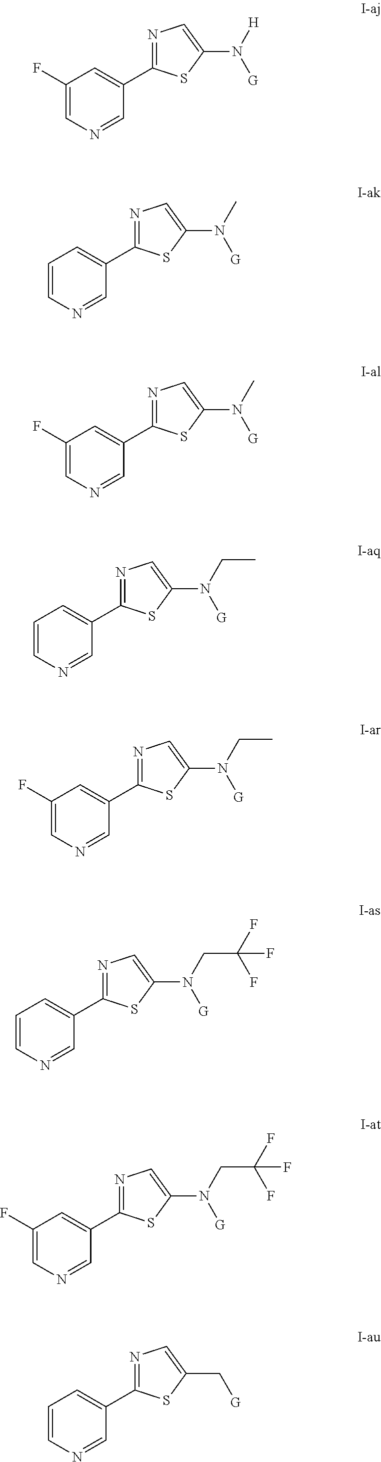

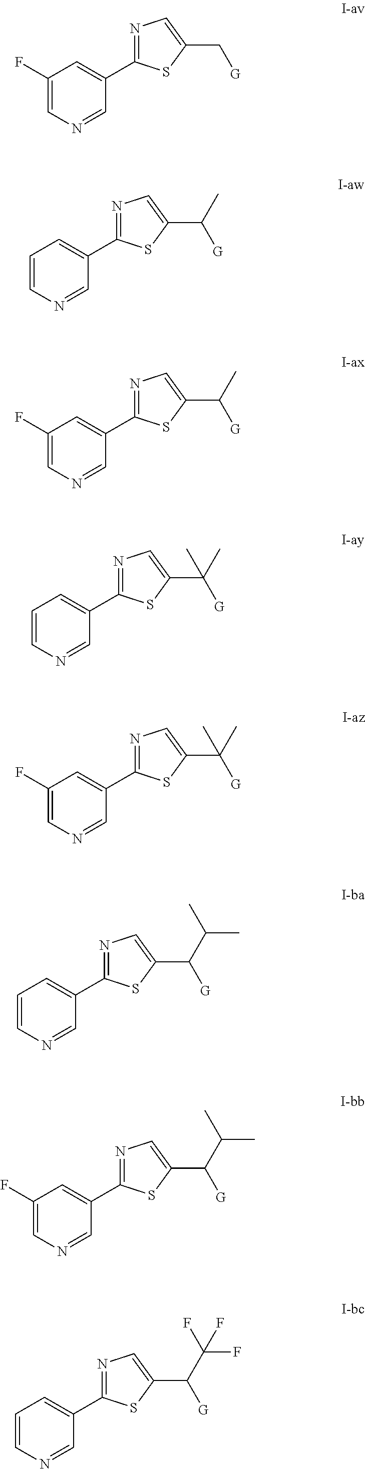

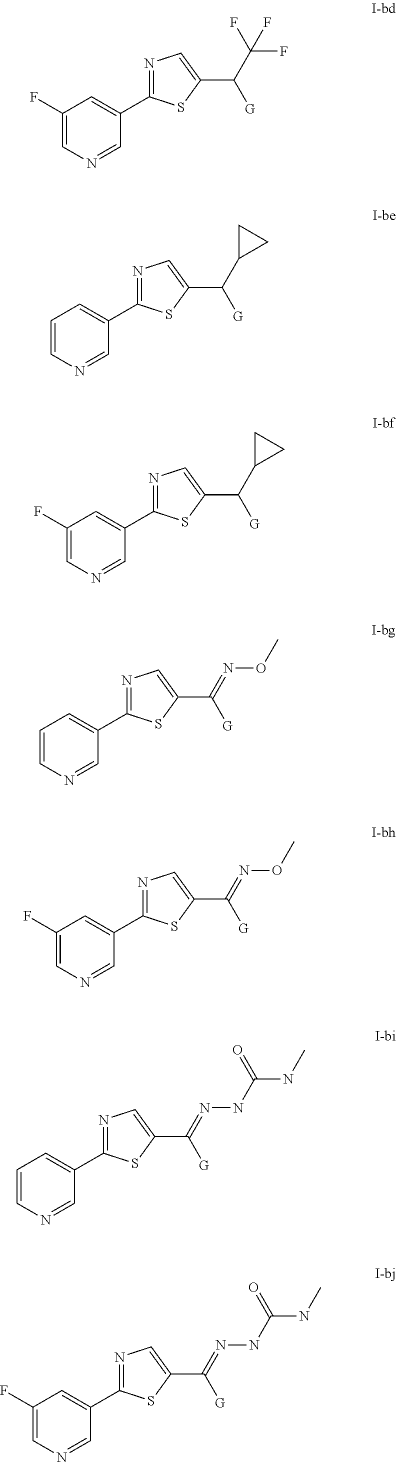

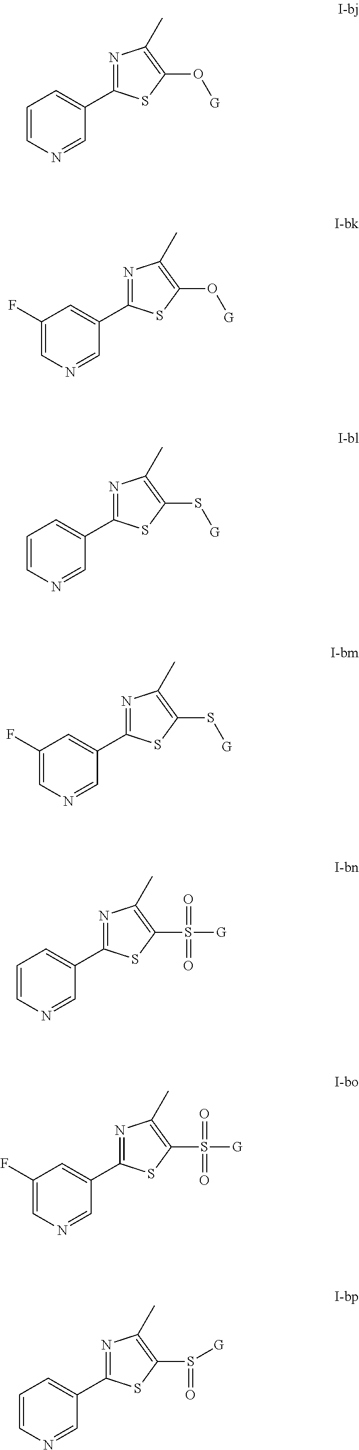

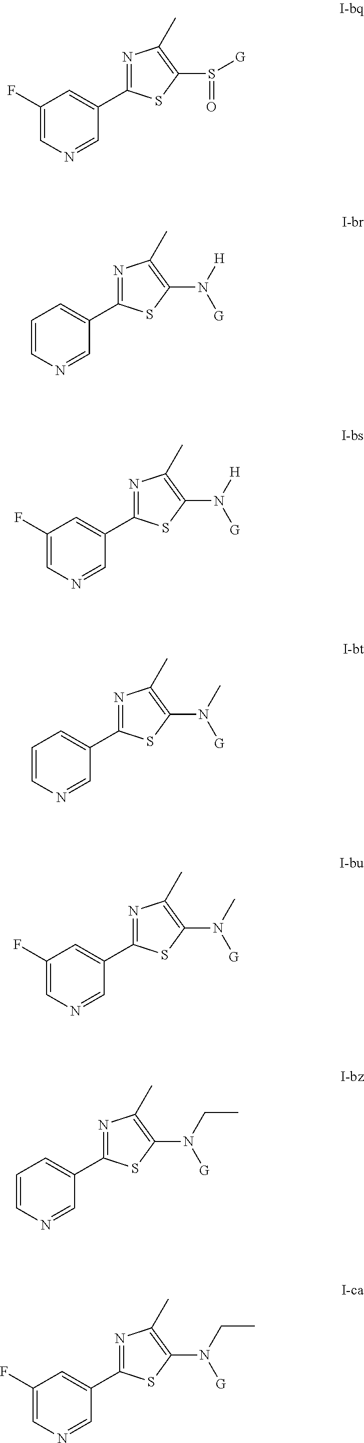

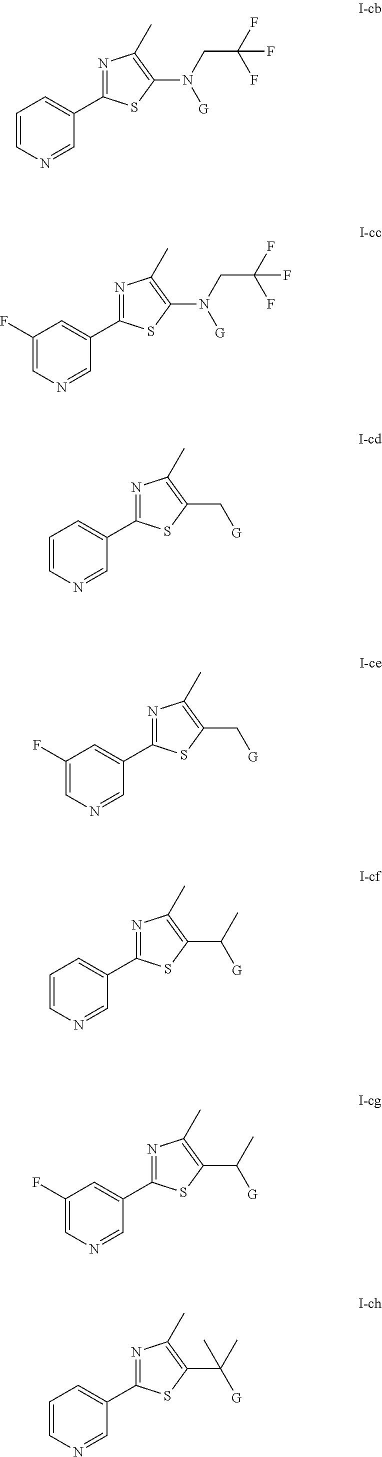

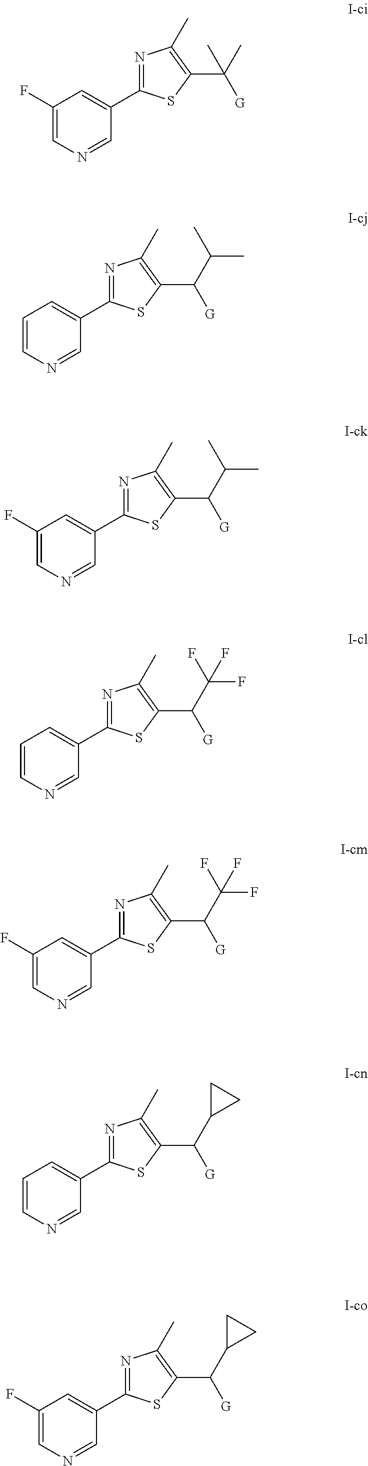

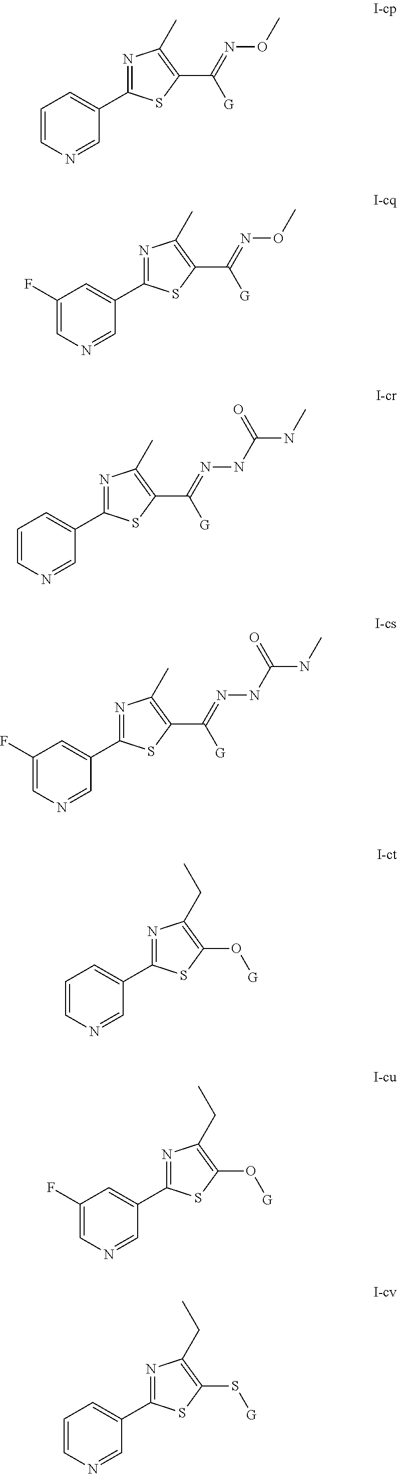

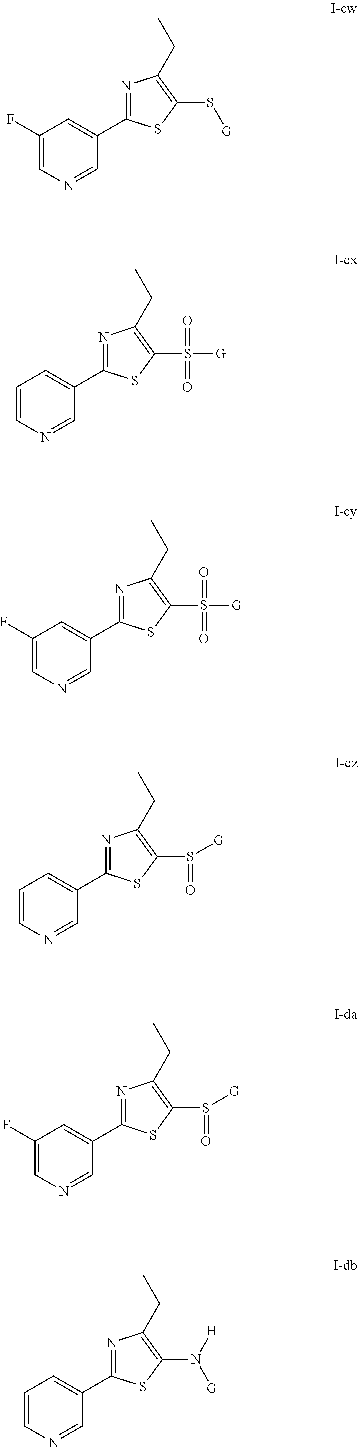

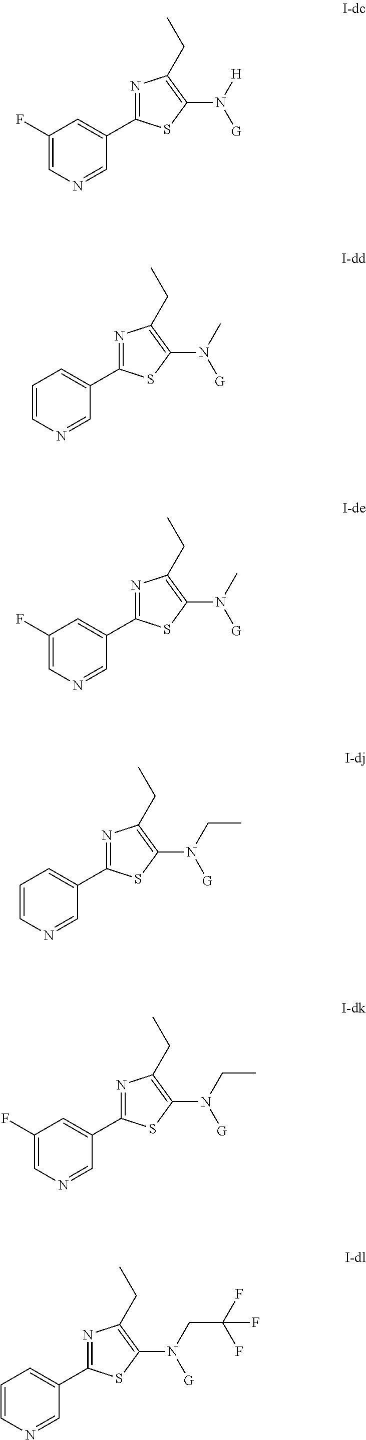

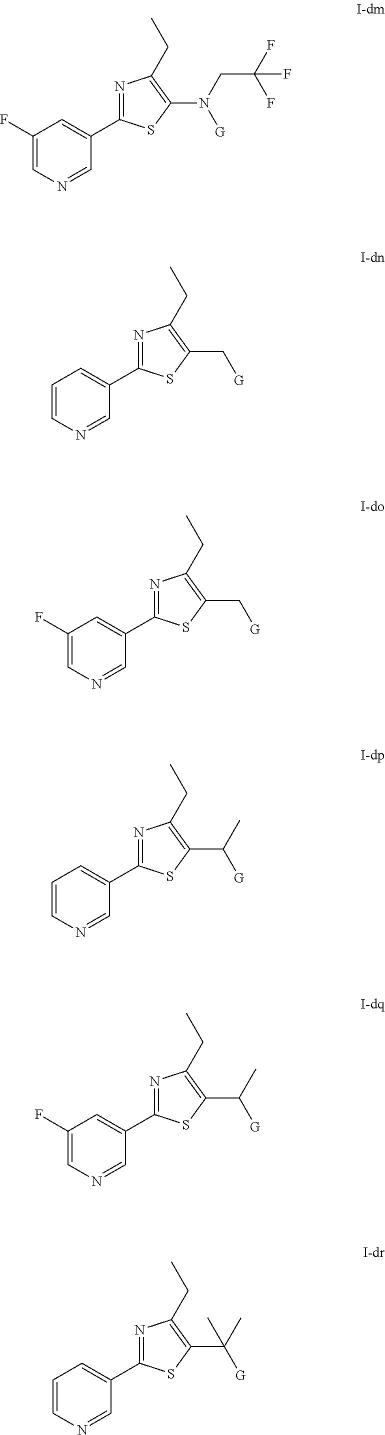

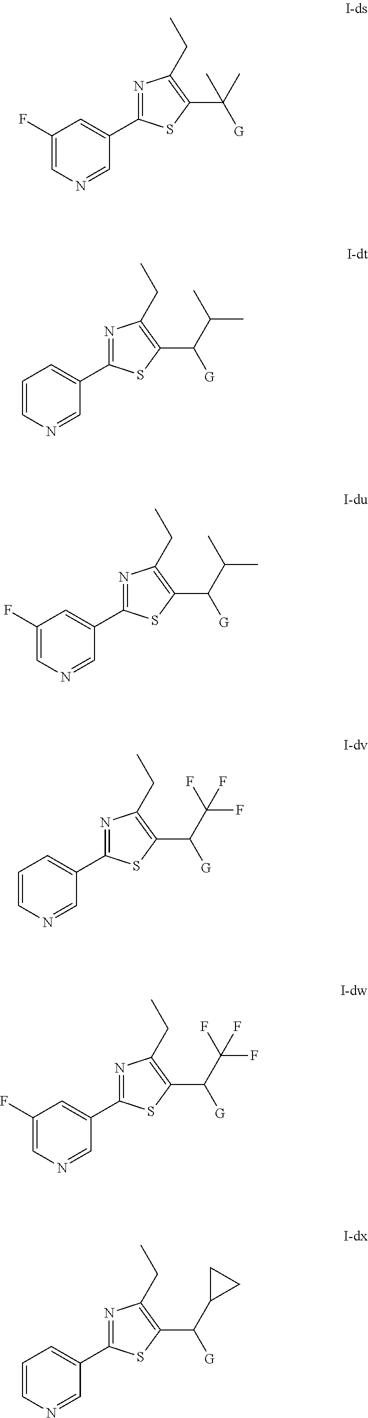

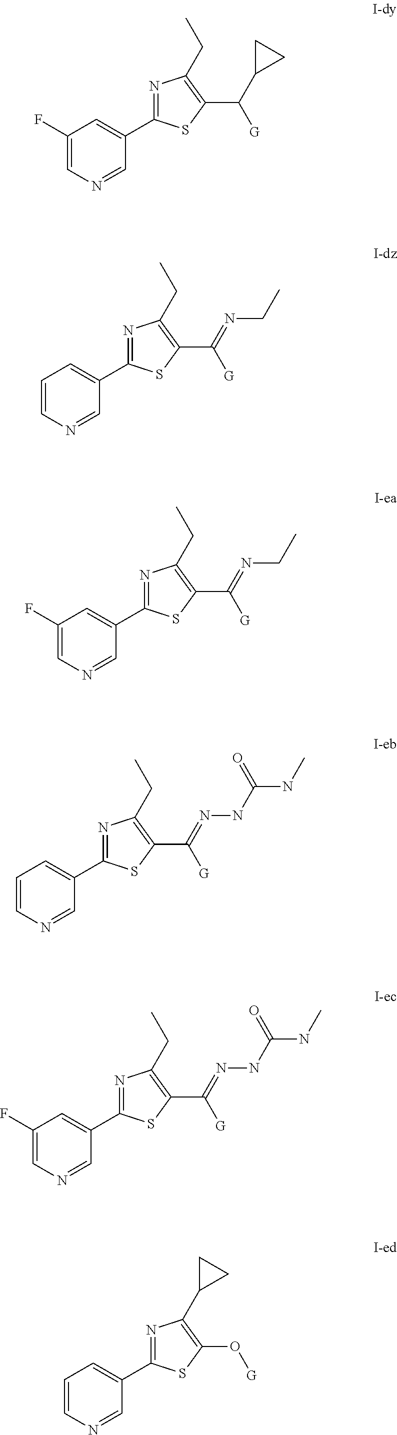

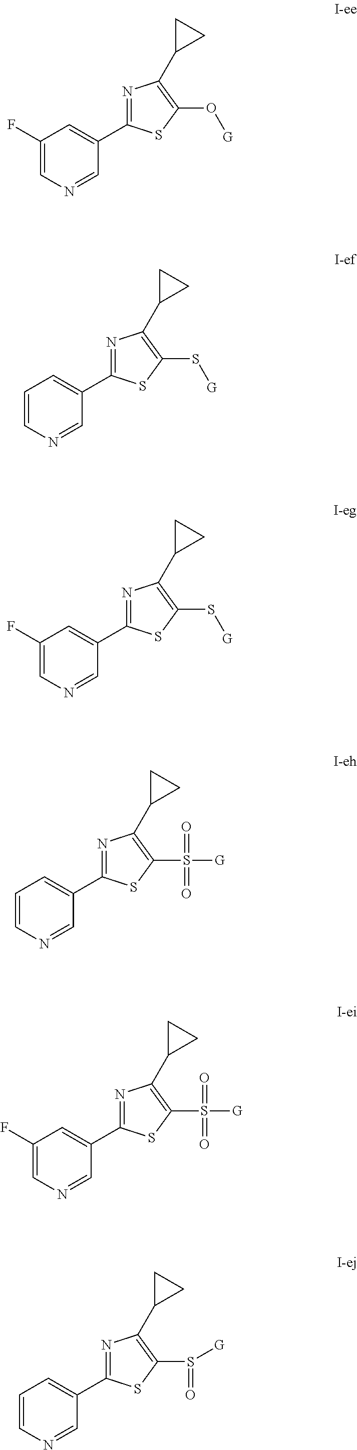

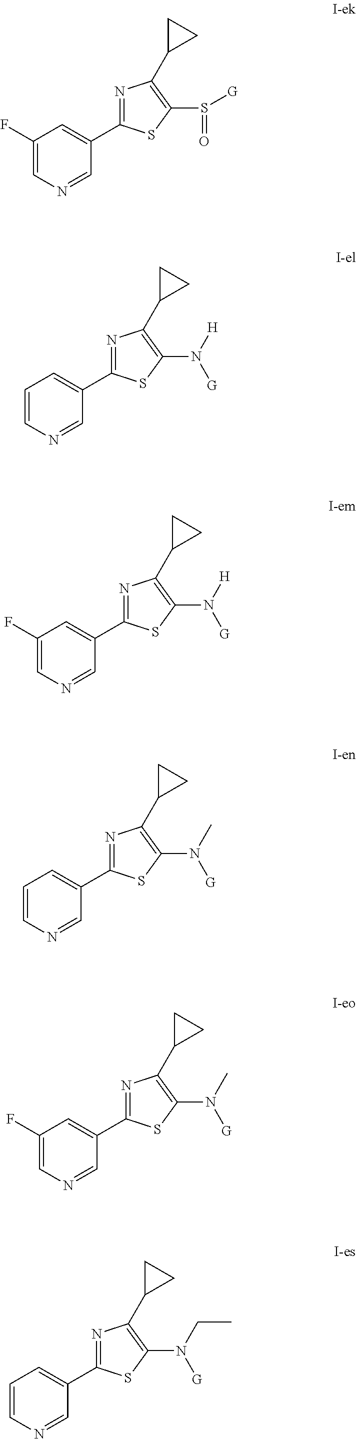

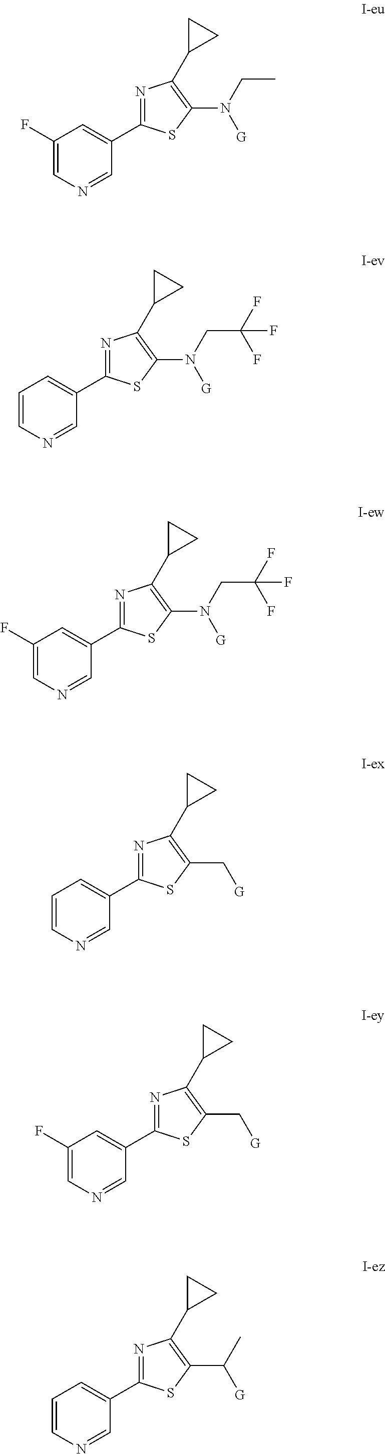

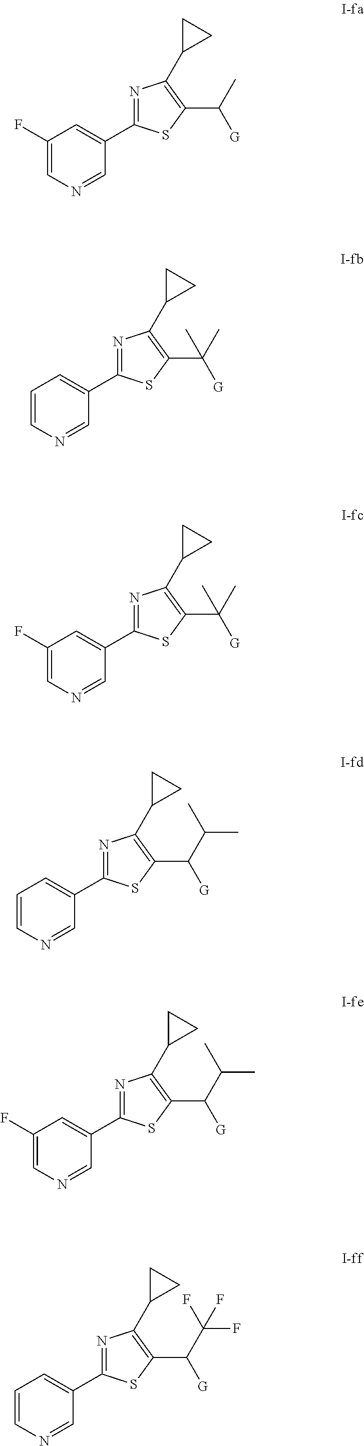

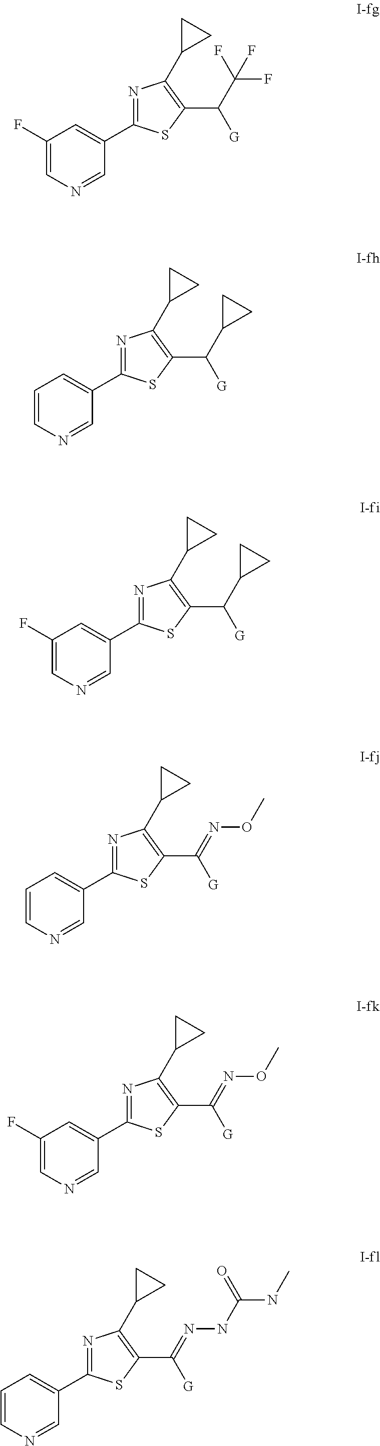

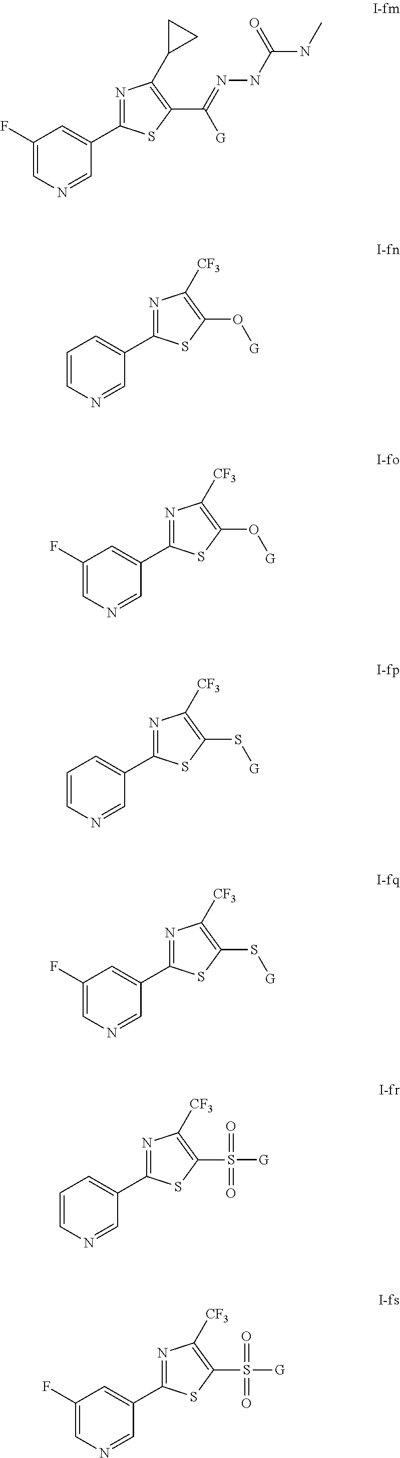

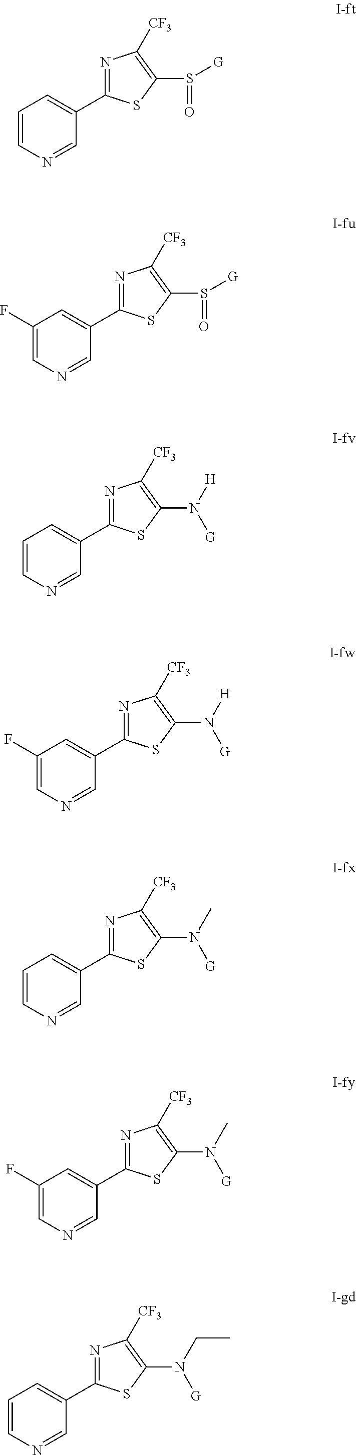

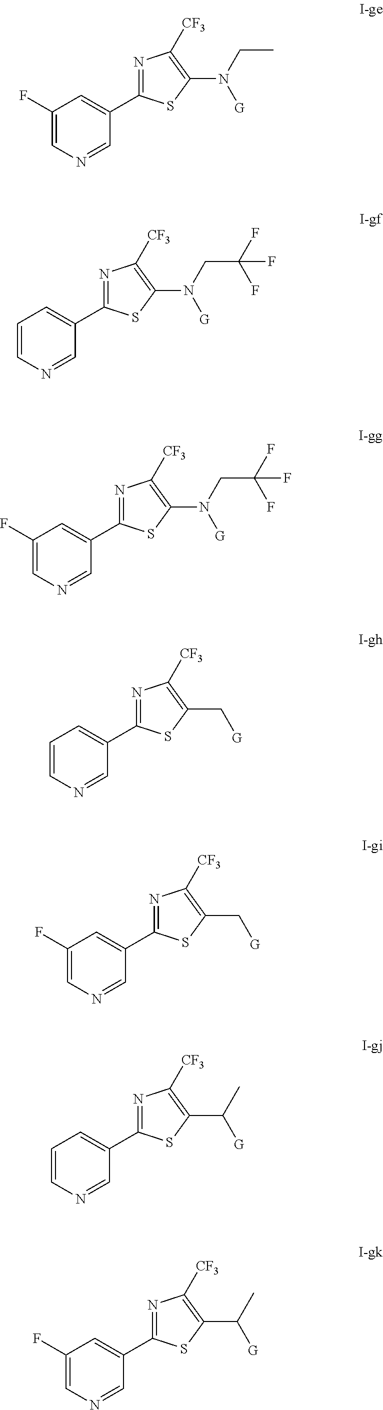

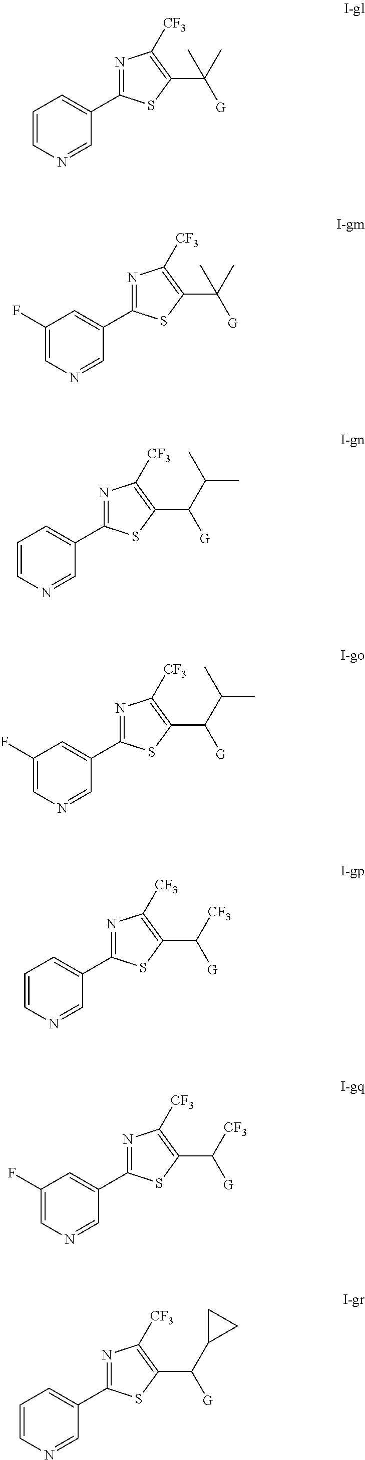

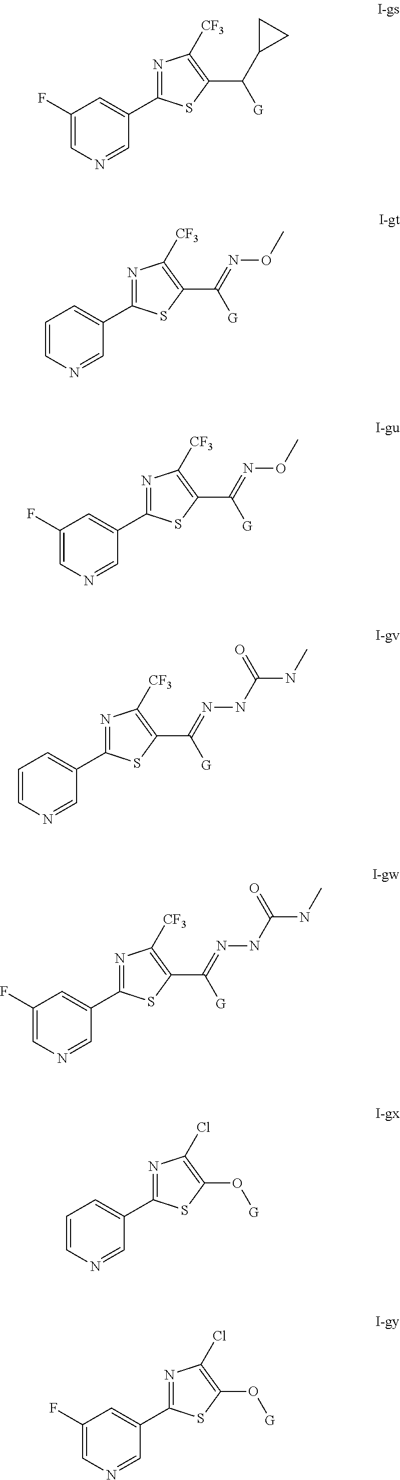

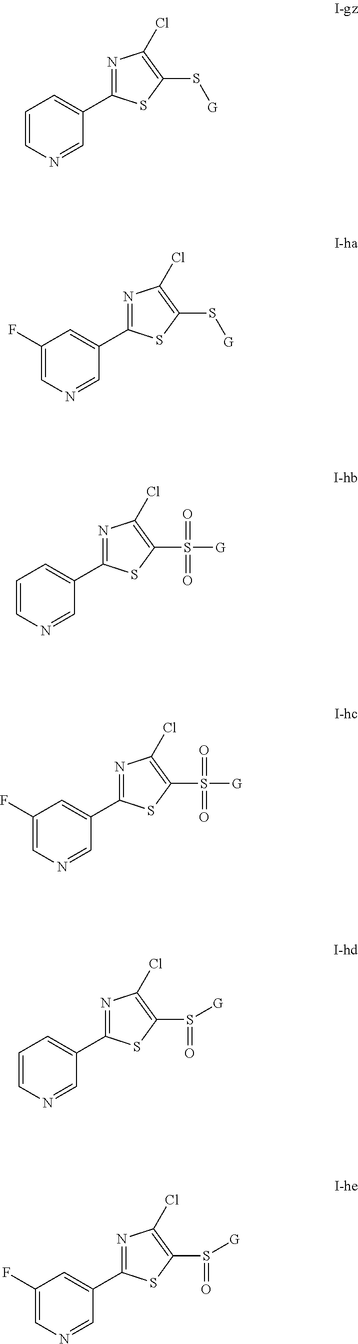

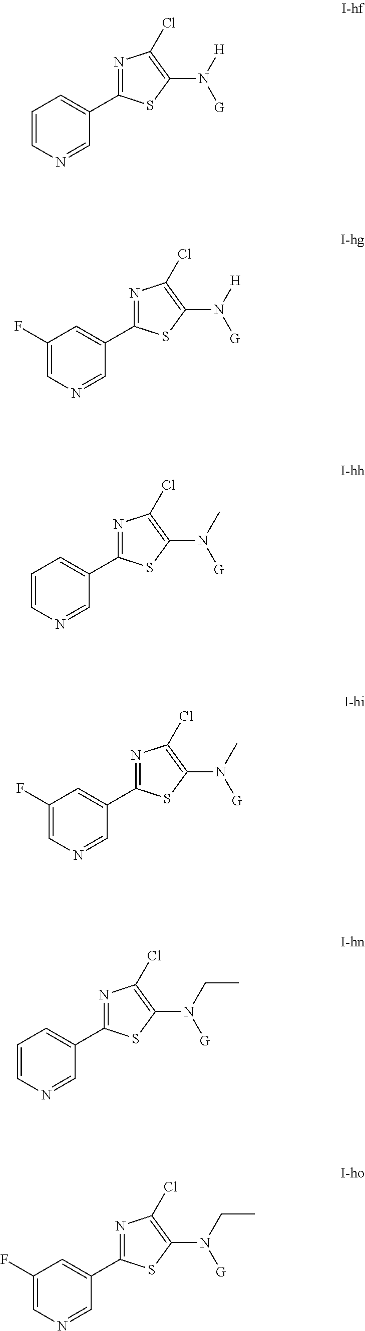

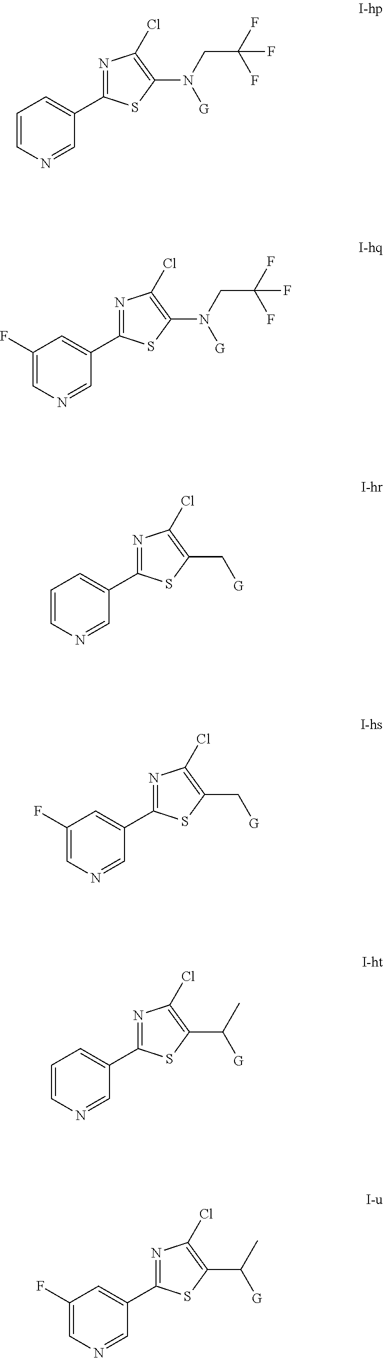

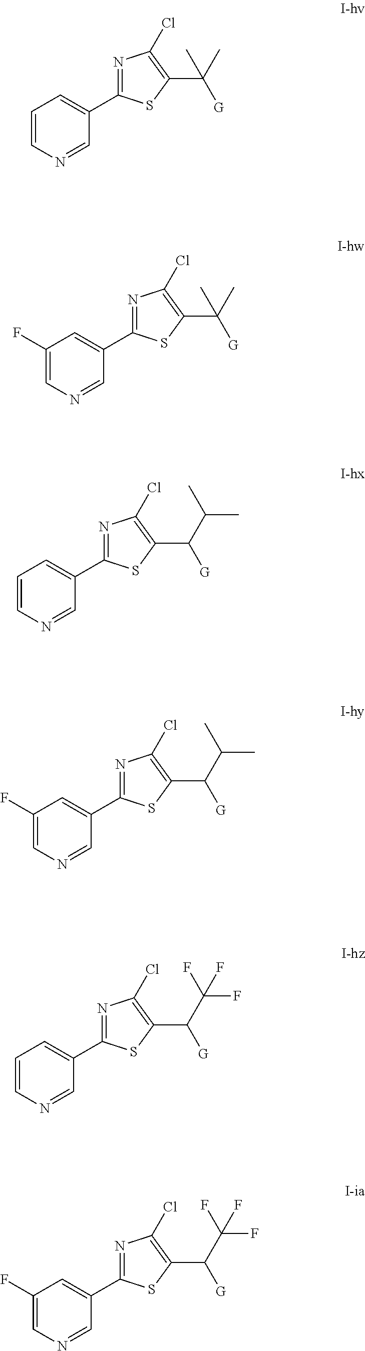

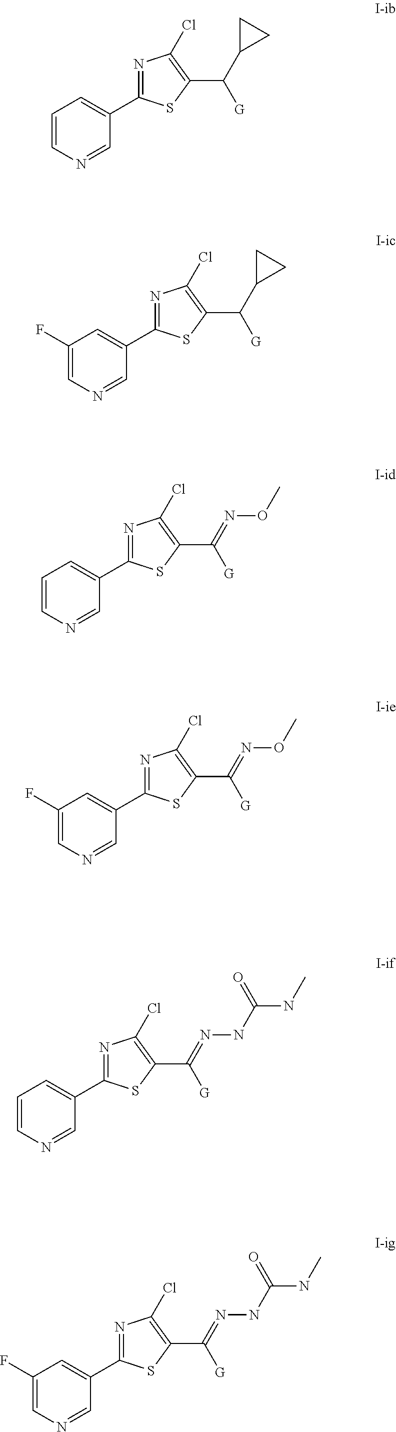

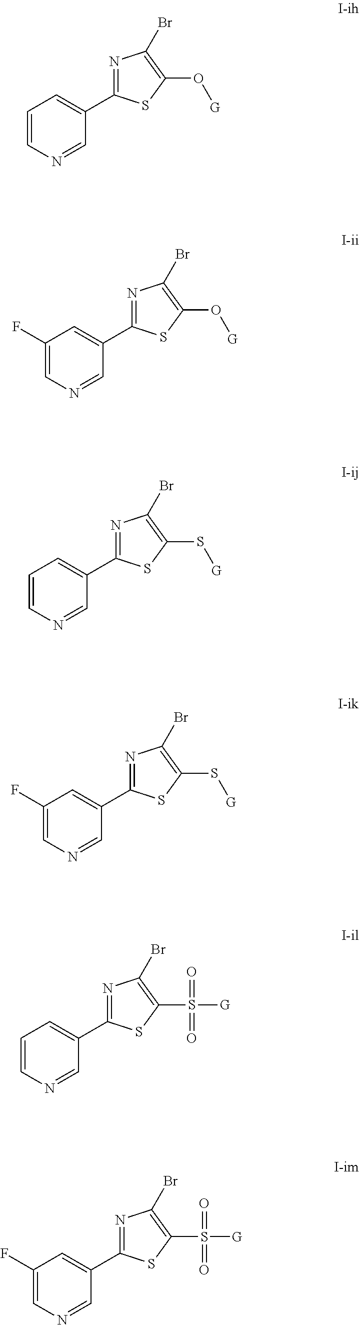

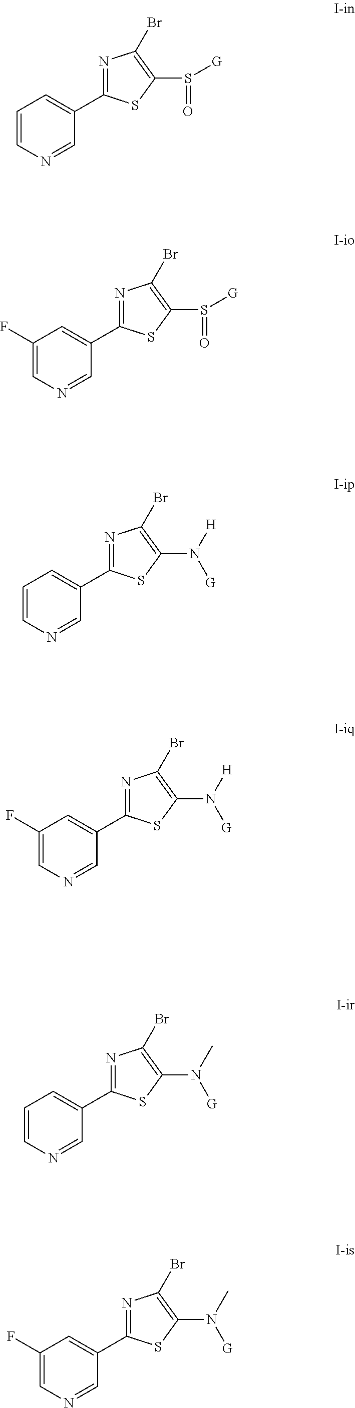

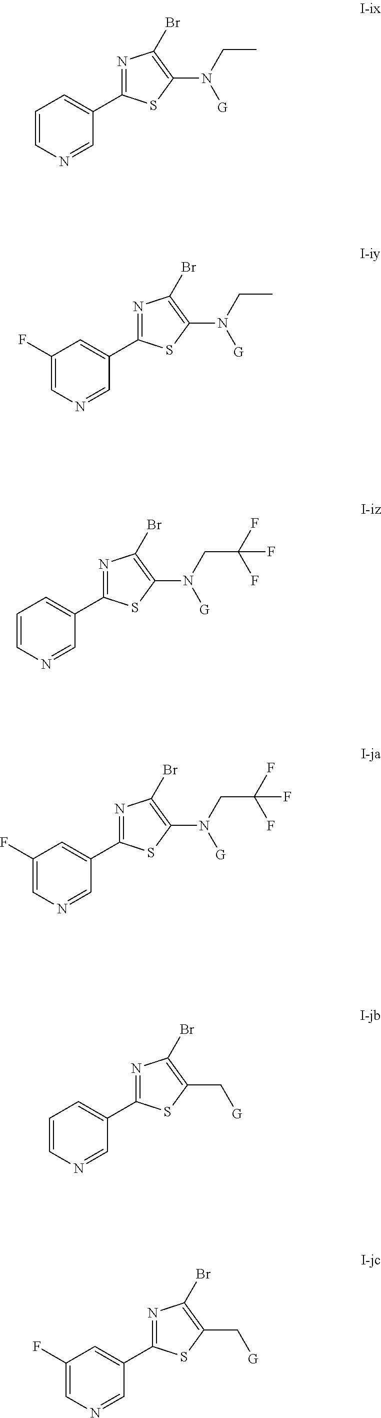

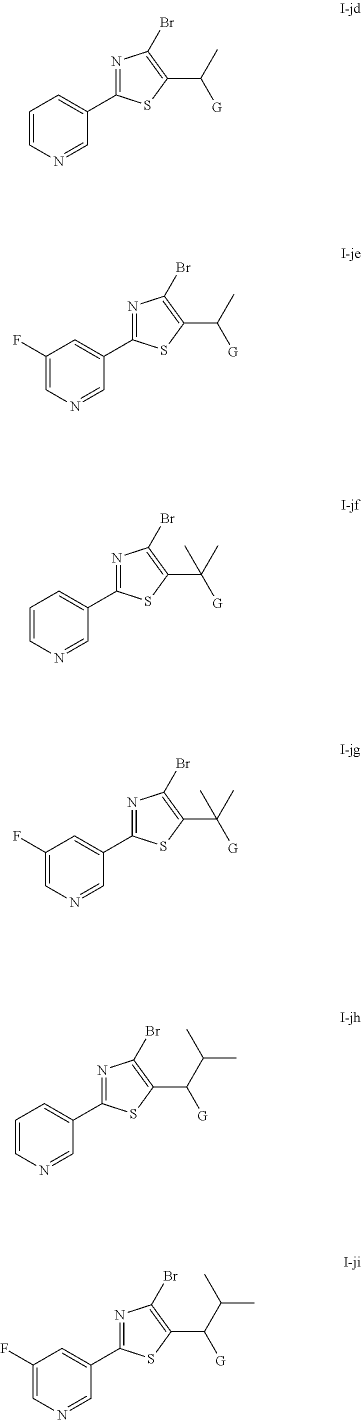

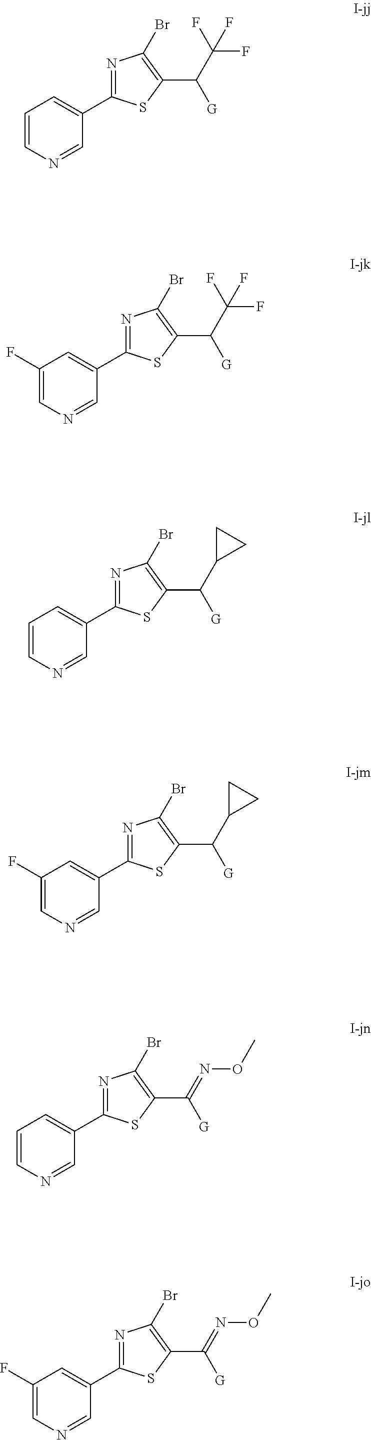

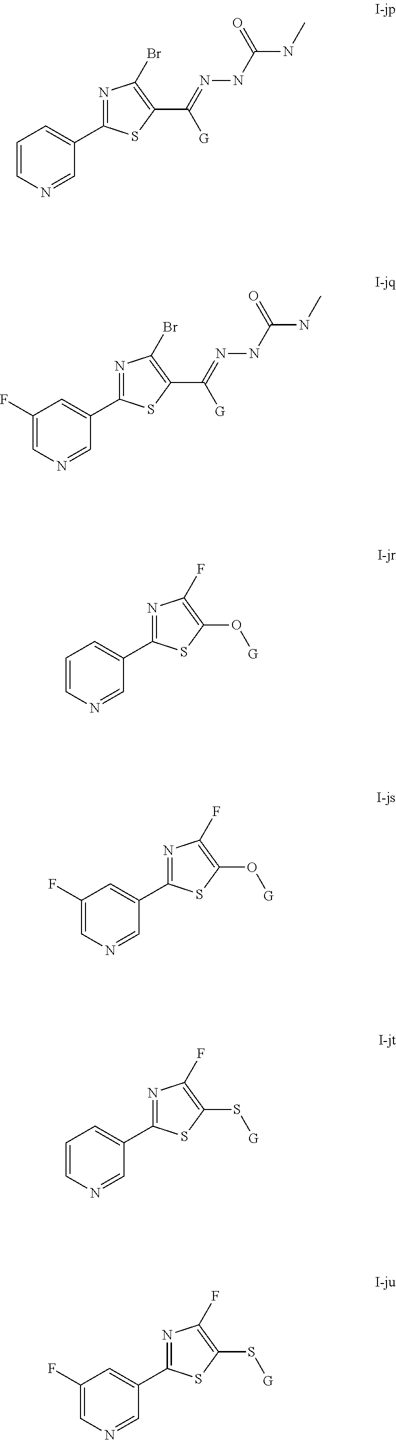

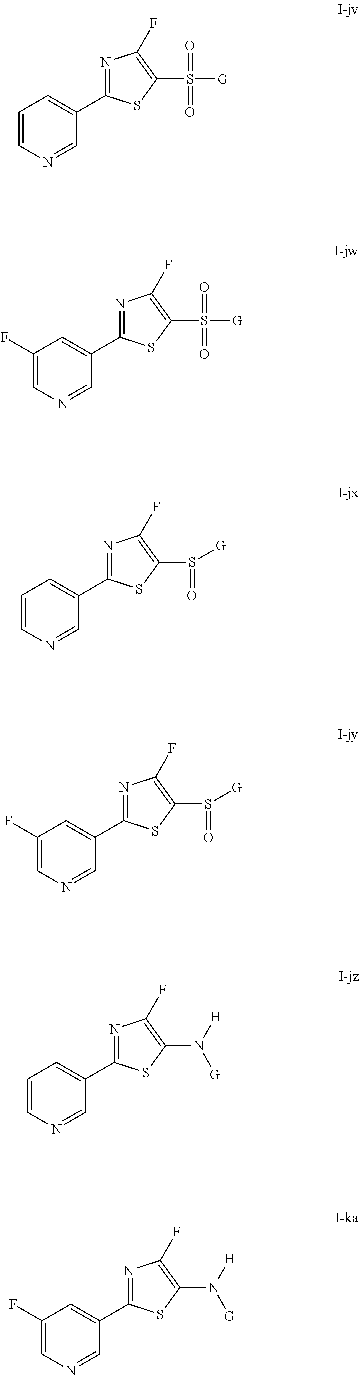

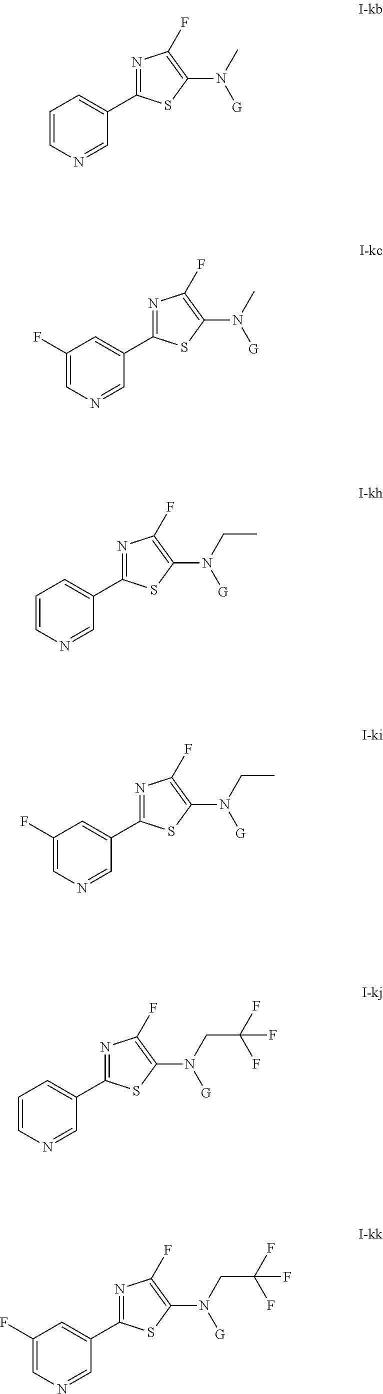

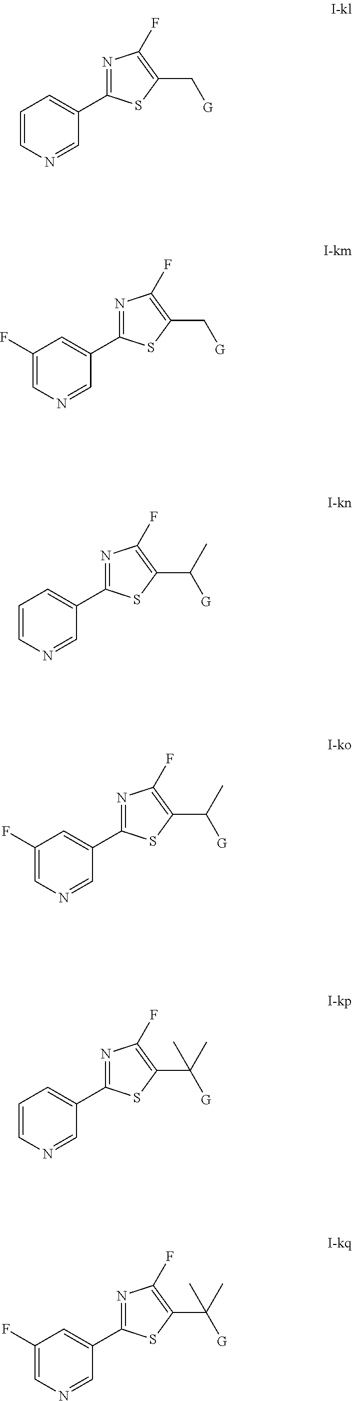

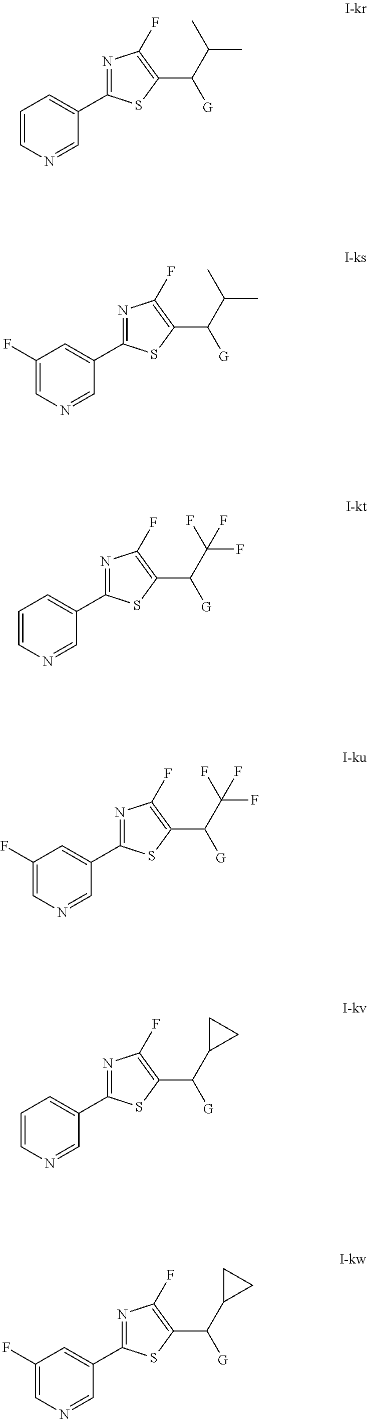

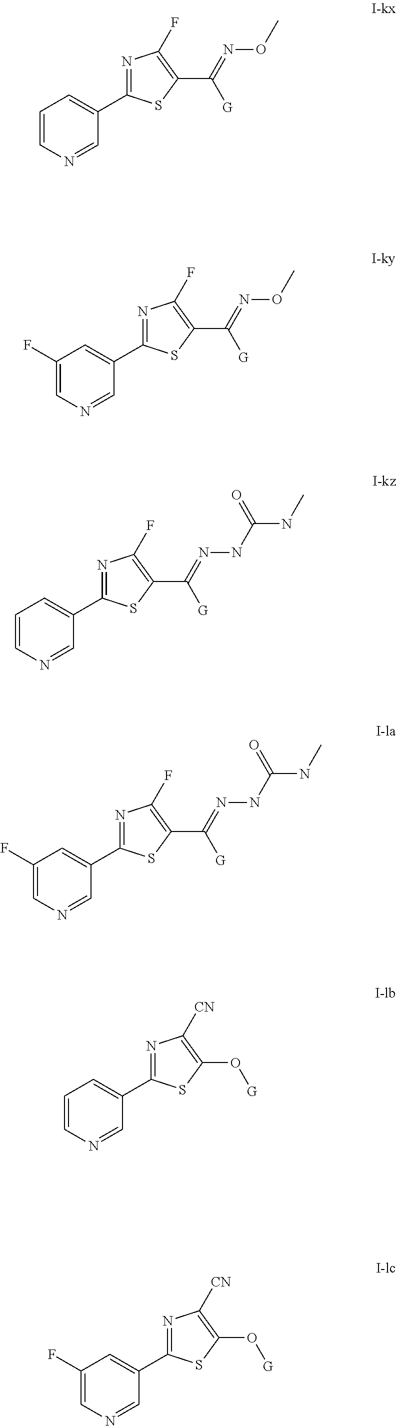

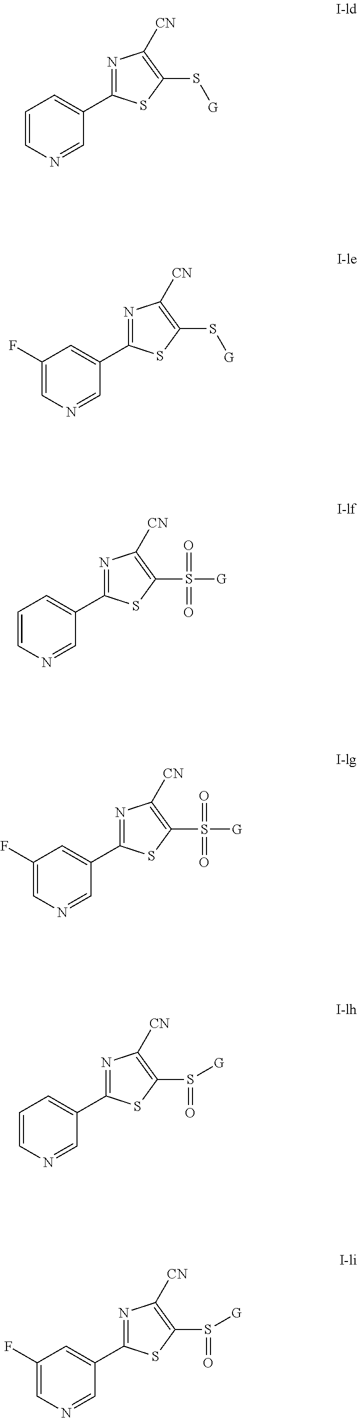

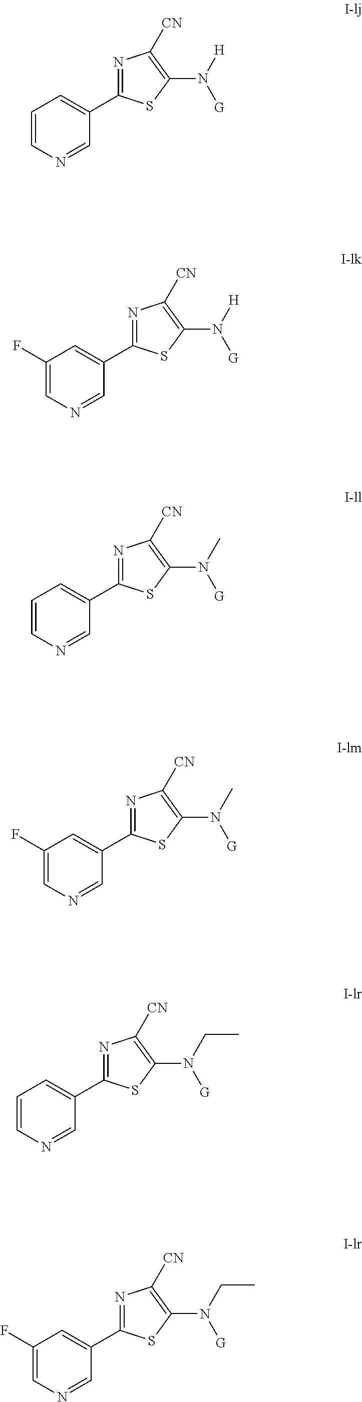

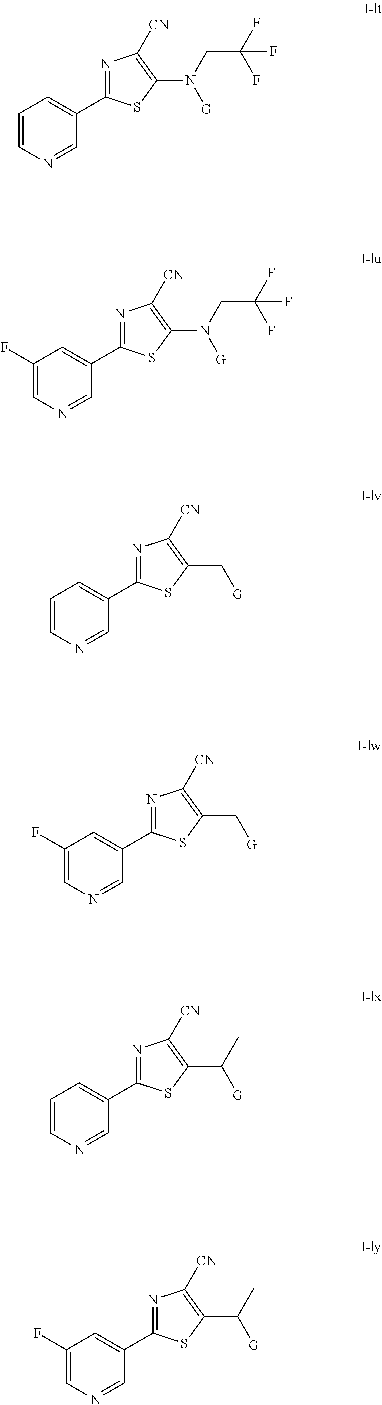

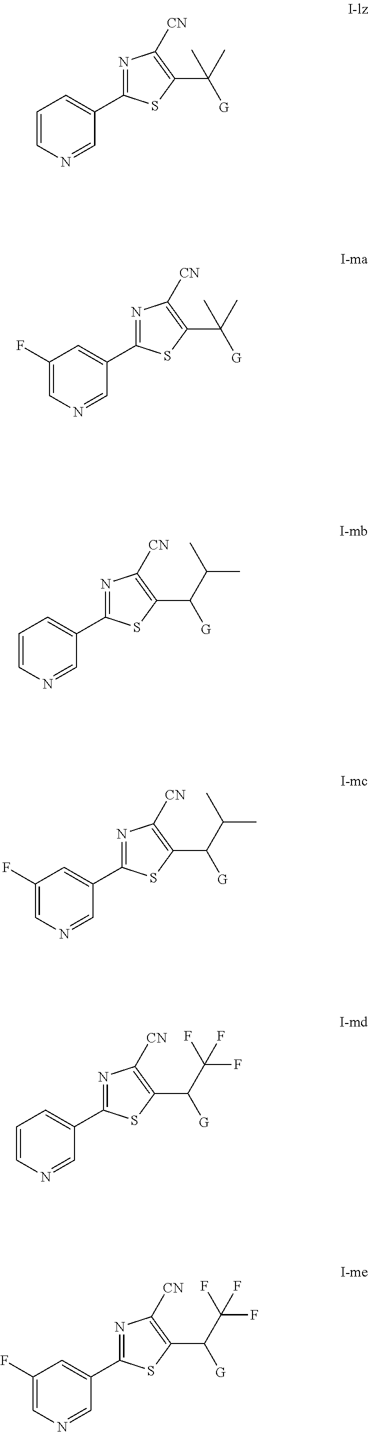

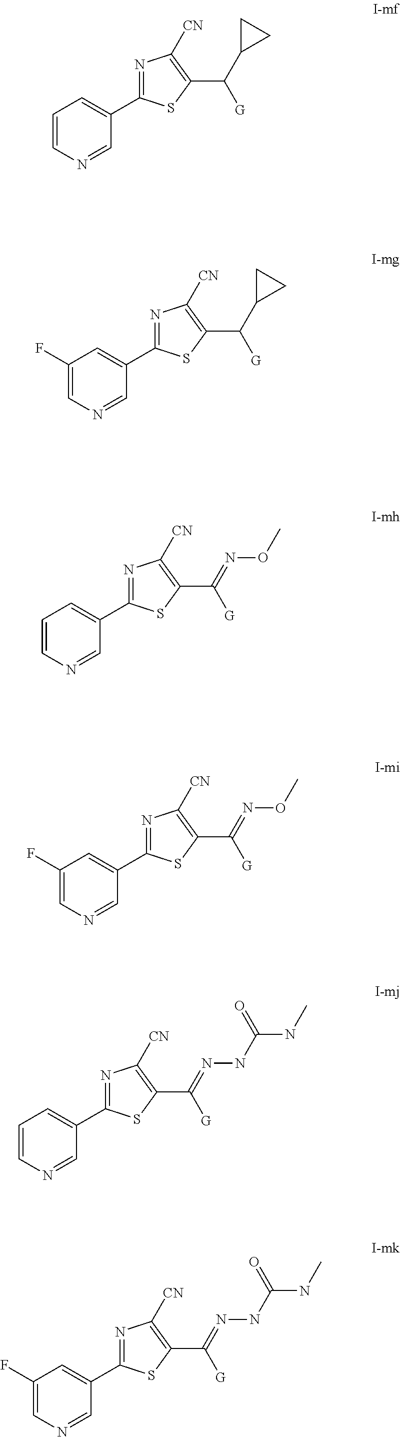

Especially preferred are such 288 substituted 3-pyridyl thiazole compounds of the above listed formulae I-aa to I-mk, wherein G has one of the preferred meanings G-1 to G-72 given herein below:

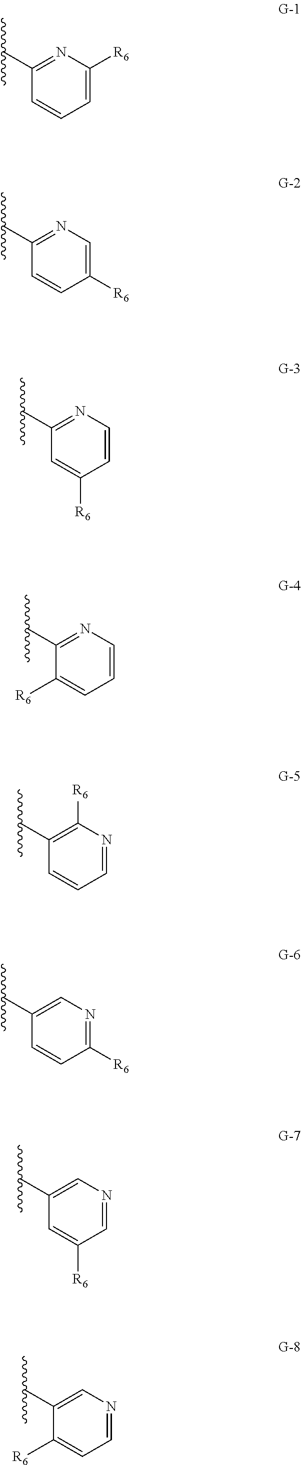

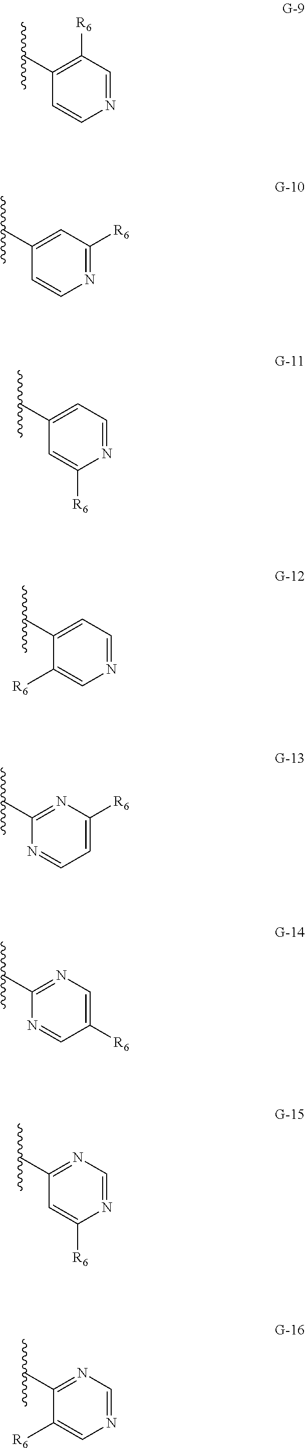

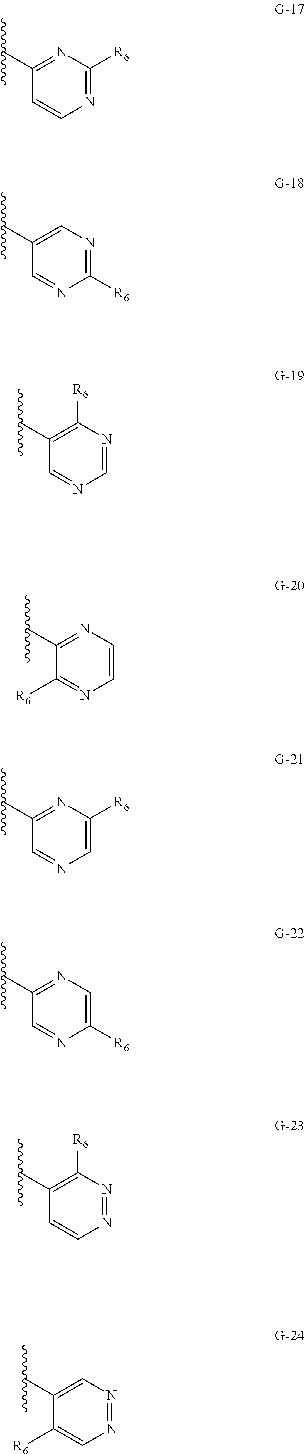

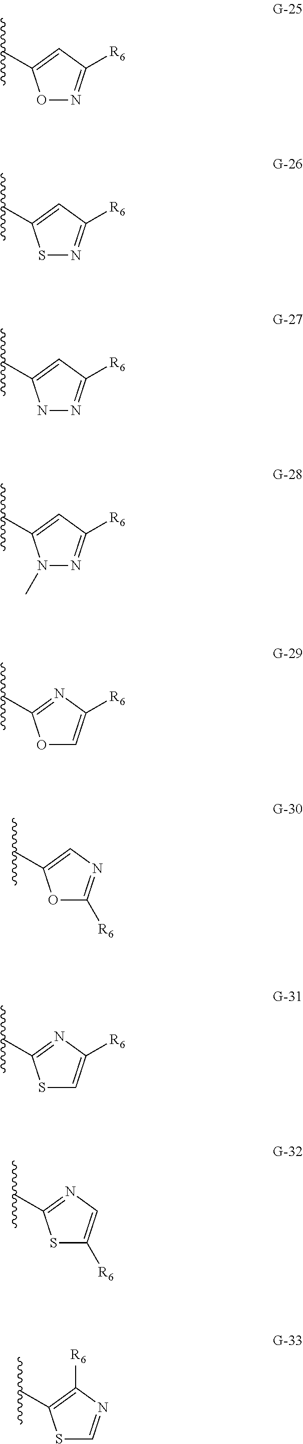

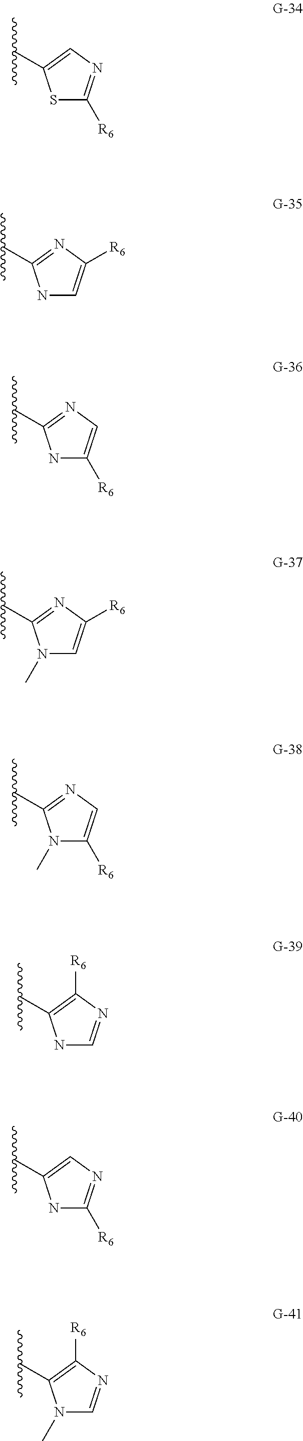

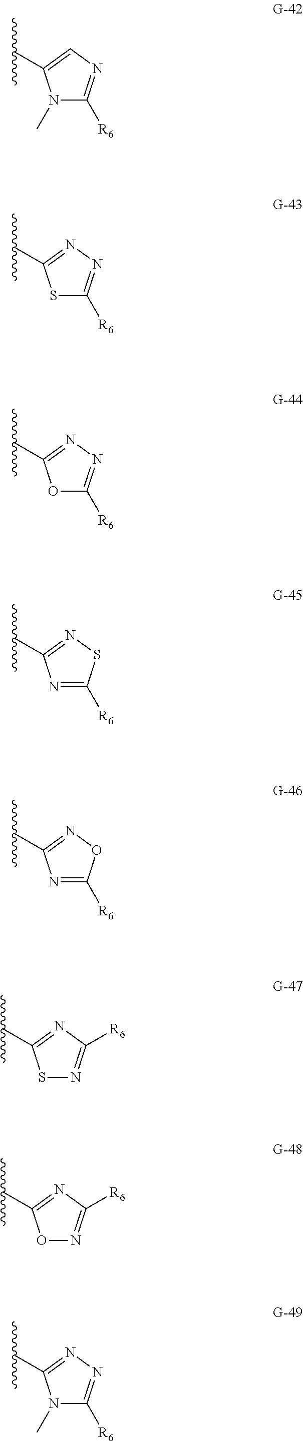

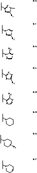

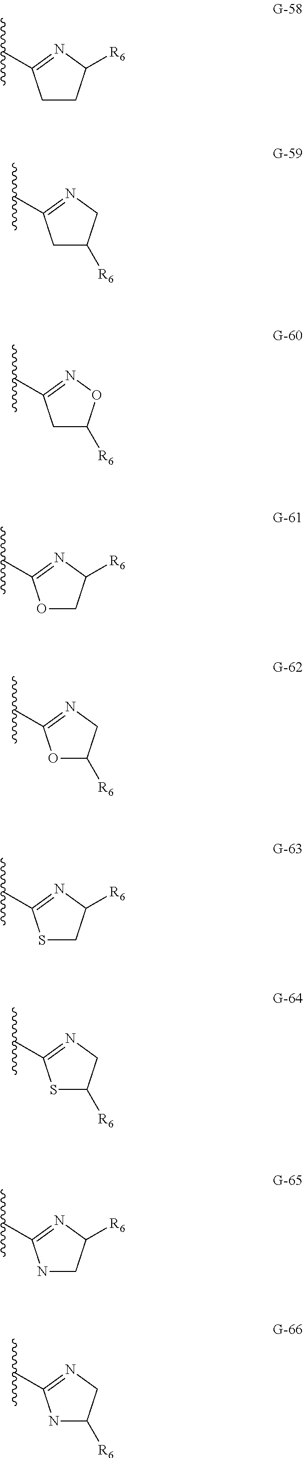

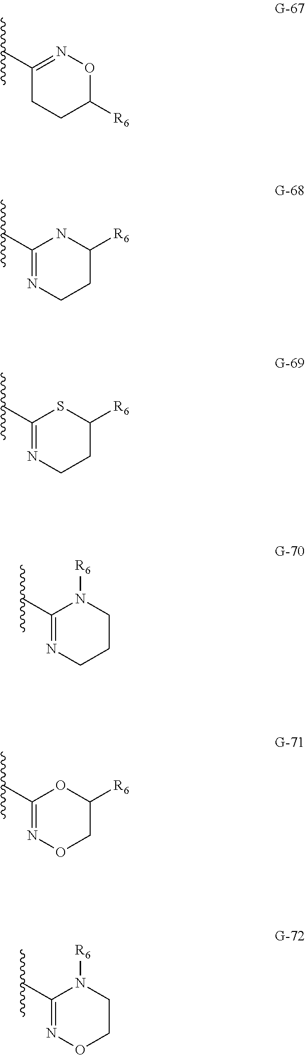

wherein the zigzag line of the moiety G denotes the bond to the remainder of the molecule.

Specific examples of especially preferred 3-pyridyl thiazole compounds of formula (I), are illustrated by formulae I-aa to I-mk in combination with the heterocyclic G-groups and the respective R⁶ substituents in table C.I.

The meaning of both substituents, G and R⁶, are defined in their combination as given in one row of table C.I, thereby showing individual preferred compounds of formulae I-aa to I-mk compiled in table C.I.

TABLE C.I

| Compound no. | G | R⁶ |
|---|---|---|
| C.I.1 | G-1 | H |
| C.I.2 | G-1 | F |
| C.I.3 | G-1 | Cl |
| C.I.4 | G-1 | Br |
| C.I.5 | G-1 | CH₃ |
| C.I.6 | G-1 | CF₃ |
| C.I.7 | G-1 | 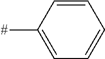 |
| C.I.8 | G-1 | 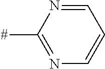 |
| C.I.9 | G-2 | H |
| C.I.10 | G-2 | F |
| C.I.11 | G-2 | Cl |
| C.I.12 | G-2 | Br |
| C.I.13 | G-2 | CH₃ |
| C.I.14 | G-2 | CF₃ |
| C.I.15 | G-2 | 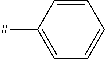 |
| C.I.16 | G-2 | 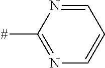 |
| C.I.17 | G-3 | H |
| C.I.18 | G-3 | F |
| C.I.19 | G-3 | Cl |
| C.I.20 | G-3 | Br |
| C.I.21 | G-3 | CH₃ |
| C.I.22 | G-3 | CF₃ |
| C.I.23 | G-3 | 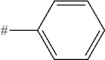 |
| C.I.24 | G-3 | 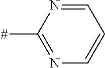 |
| C.I.25 | G-4 | H |
| C.I.26 | G-4 | F |
| C.I.27 | G-4 | Cl |
| C.I.28 | G-4 | Br |
| C.I.29 | G-4 | CH₃ |
| C.I.30 | G-4 | CF₃ |
| C.I.31 | G-4 | 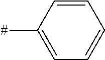 |
| C.I.32 | G-4 | 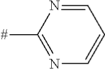 |
| C.I.33 | G-5 | H |
| C.I.34 | G-5 | F |
| C.I.35 | G-5 | Cl |
| C.I.36 | G-5 | Br |
| C.I.37 | G-5 | CH₃ |
| C.I.38 | G-5 | CF₃ |
| C.I.39 | G-5 | 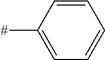 |
| C.I.40 | G-5 | 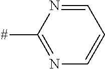 |
| C.I.41 | G-6 | H |
| C.I.42 | G-6 | F |
| C.I.43 | G-6 | Cl |
| C.I.44 | G-6 | Br |
| C.I.45 | G-6 | CH₃ |
| C.I.46 | G-6 | CF₃ |
| C.I.47 | G-6 | 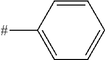 |
| C.I.48 | G-6 | 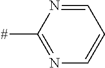 |
| C.I.49 | G-7 | H |
| C.I.50 | G-7 | F |
| C.I.51 | G-7 | Cl |
| C.I.52 | G-7 | Br |
| C.I.53 | G-7 | CH₃ |
| C.I.54 | G-7 | CF₃ |
| C.I.55 | G-7 | 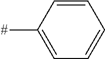 |
| C.I.56 | G-7 | 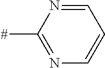 |
| C.I.57 | G-8 | H |
| C.I.58 | G-8 | F |
| C.I.59 | G-8 | Cl |
| C.I.60 | G-8 | Br |
| C.I.61 | G-8 | CH₃ |
| C.I.62 | G-8 | CF₃ |
| C.I.63 | G-8 | 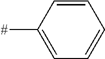 |
| C.I.64 | G-8 | 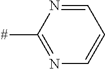 |
| C.I.65 | G-9 | H |
| C.I.66 | G-9 | F |
| C.I.67 | G-9 | Cl |
| C.I.68 | G-9 | Br |
| C.I.69 | G-9 | CH₃ |
| C.I.70 | G-9 | CF₃ |
| C.I.71 | G-9 | 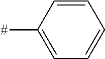 |
| C.I.72 | G-9 | 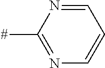 |
| C.I.73 | G-10 | H |
| C.I.74 | G-10 | F |
| C.I.75 | G-10 | Cl |
| C.I.76 | G-10 | Br |
| C.I.77 | G-10 | CH₃ |
| C.I.78 | G-10 | CF₃ |
| C.I.79 | G-10 | 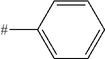 |
| C.I.80 | G-10 | 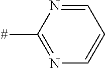 |
| C.I.81 | G-11 | H |
| C.I.82 | G-11 | F |
| C.I.83 | G-11 | Cl |
| C.I.84 | G-11 | Br |
| C.I.85 | G-11 | CH₃ |
| C.I.86 | G-11 | CF₃ |
| C.I.87 | G-11 | 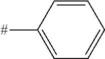 |
| C.I.88 | G-11 | 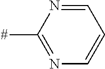 |
| C.I.89 | G-12 | H |
| C.I.90 | G-12 | F |
| C.I.91 | G-12 | Cl |
| C.I.92 | G-12 | Br |
| C.I.93 | G-12 | CH₃ |
| C.I.94 | G-12 | CF₃ |
| C.I.95 | G-12 | 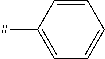 |
| C.I.96 | G-12 | 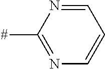 |
| C.I.97 | G-13 | H |
| C.I.98 | G-13 | F |
| C.I.99 | G-13 | Cl |
| C.I.100 | G-13 | Br |
| C.I.101 | G-13 | CH₃ |
| C.I.102 | G-13 | CF₃ |
| C.I.103 | G-13 | 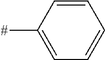 |
| C.I.104 | G-14 | H |
| C.I.105 | G-14 | F |
| C.I.106 | G-14 | Cl |
| C.I.107 | G-14 | Br |
| C.I.108 | G-14 | CH₃ |
| C.I.109 | G-14 | CF₃ |
| C.I.110 | G-14 | 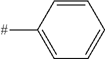 |
| C.I.111 | G-15 | H |
| C.I.112 | G-15 | F |
| C.I.113 | G-15 | Cl |
| C.I.114 | G-15 | Br |
| C.I.115 | G-15 | CH₃ |
| C.I.116 | G-15 | CF₃ |
| C.I.117 | G-15 | 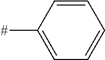 |
| C.I.118 | G-16 | H |
| C.I.119 | G-16 | F |
| C.I.120 | G-16 | Cl |
| C.I.121 | G-16 | Br |
| C.I.122 | G-16 | CH₃ |
| C.I.123 | G-16 | CF₃ |
| C.I.124 | G-16 | 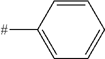 |
| C.I.125 | G-16 | H |
| C.I.126 | G-16 | F |
| C.I.127 | G-16 | Cl |
| C.I.128 | G-16 | Br |
| C.I.129 | G-16 | CH₃ |
| C.I.130 | G-16 | CF₃ |
| C.I.131 | G-16 | 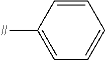 |
| C.I.132 | G-17 | H |
| C.I.133 | G-17 | F |
| C.I.134 | G-17 | Cl |
| C.I.135 | G-17 | Br |
| C.I.136 | G-17 | CH₃ |
| C.I.137 | G-17 | CF₃ |
| C.I.138 | G-17 | 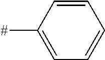 |
| C.I.139 | G-18 | H |
| C.I.140 | G-18 | F |
| C.I.141 | G-18 | Cl |
| C.I.142 | G-18 | Br |
| C.I.143 | G-18 | CH₃ |
| C.I.144 | G-18 | CF₃ |
| C.I.145 | G-18 | 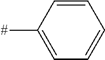 |
| C.I.146 | G-19 | H |
| C.I.147 | G-19 | F |
| C.I.148 | G-19 | Cl |
| C.I.149 | G-19 | Br |
| C.I.150 | G-19 | CH₃ |
| C.I.151 | G-19 | CF₃ |
| C.I.152 | G-19 | 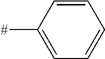 |
| C.I.153 | G-20 | H |
| C.I.154 | G-20 | F |
| C.I.155 | G-20 | Cl |
| C.I.156 | G-20 | Br |
| C.I.157 | G-20 | CH₃ |
| C.I.158 | G-20 | CF₃ |
| C.I.159 | G-20 | 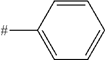 |
| C.I.160 | G-21 | H |
| C.I.161 | G-21 | F |
| C.I.162 | G-21 | Cl |
| C.I.163 | G-21 | Br |
| C.I.164 | G-21 | CH₃ |
| C.I.165 | G-21 | CF₃ |
| C.I.166 | G-21 | 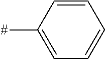 |
| C.I.167 | G-22 | H |
| C.I.168 | G-22 | F |
| C.I.169 | G-22 | Cl |
| C.I.170 | G-22 | Br |
| C.I.171 | G-22 | CH3 |
| C.I.172 | G-22 | CF3 |
| C.I.173 | G-22 | 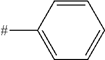 |
| C.I.174 | G-23 | H |
| C.I.175 | G-23 | F |
| C.I.176 | G-23 | Cl |
| C.I.177 | G-23 | Br |
| C.I.178 | G-23 | CH₃ |
| C.I.179 | G-23 | CF₃ |
| C.I.180 | G-24 | H |
| C.I.181 | G-24 | F |
| C.I.182 | G-24 | Cl |
| C.I.183 | G-24 | Br |
| C.I.184 | G-24 | CH₃ |
| C.I.185 | G-24 | CF₃ |
| C.I.186 | G-25 | H |
| C.I.187 | G-25 | F |
| C.I.188 | G-25 | Cl |
| C.I.189 | G-25 | Br |
| C.I.190 | G-25 | CH₃ |
| C.I.191 | G-25 | CF₃ |
| C.I.192 | G-25 | 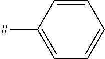 |
| C.I.193 | G-25 | 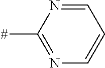 |
| C.I.194 | G-26 | H |
| C.I.195 | G-26 | F |
| C.I.196 | G-26 | Cl |
| C.I.197 | G-26 | Br |
| C.I.198 | G-26 | CH3 |
| C.I.199 | G-26 | CF3 |
| C.I.200 | G-26 | 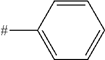 |
| C.I.201 | G-26 | 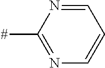 |
| C.I.202 | G-27 | H |
| C.I.203 | G-27 | F |
| C.I.204 | G-27 | Cl |
| C.I.205 | G-27 | Br |
| C.I.206 | G-27 | CH3 |
| C.I.207 | G-27 | CF3 |
| C.I.208 | G-27 | 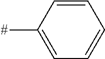 |
| C.I.209 | G-27 | 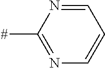 |
| C.I.210 | G-28 | H |
| C.I.211 | G-28 | F |
| C.I.212 | G-28 | Cl |
| C.I.213 | G-28 | Br |
| C.I.214 | G-28 | CH3 |
| C.I.215 | G-28 | CF3 |
| C.I.216 | G-28 | 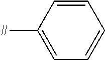 |
| C.I.217 | G-28 | 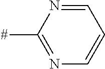 |
| C.I.218 | G-29 | H |
| C.I.219 | G-29 | F |
| C.I.220 | G-29 | Cl |
| C.I.221 | G-29 | Br |
| C.I.222 | G-29 | CH3 |
| C.I.223 | G-29 | CF3 |
| C.I.224 | G-29 | 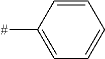 |
| C.I.225 | G-29 | 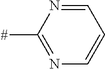 |
| C.I.226 | G-30 | H |
| C.I.227 | G-30 | F |
| C.I.228 | G-30 | Cl |
| C.I.229 | G-30 | Br |
| C.I.230 | G-30 | CH3 |
| C.I.231 | G-30 | CF3 |
| C.I.232 | G-30 | 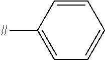 |
| C.I.233 | G-30 | 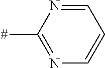 |
| C.I.234 | G-31 | H |
| C.I.235 | G-31 | F |
| C.I.236 | G-31 | Cl |
| C.I.237 | G-31 | Br |
| C.I.238 | G-31 | CH3 |
| C.I.239 | G-31 | CF3 |
| C.I.240 | G-31 | 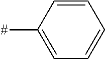 |
| C.I.241 | G-31 | 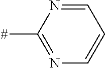 |
| C.I.242 | G-32 | H |
| C.I.243 | G-32 | F |
| C.I.244 | G-32 | Cl |
| C.I.245 | G-32 | Br |
| C.I.246 | G-32 | CH3 |
| C.I.247 | G-32 | CF3 |
| C.I.248 | G-32 | 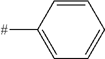 |
| C.I.249 | G-32 | 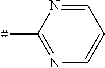 |
| C.I.250 | G-33 | H |
| C.I.251 | G-33 | F |
| C.I.252 | G-33 | Cl |
| C.I.253 | G-33 | Br |
| C.I.254 | G-33 | CH3 |
| C.I.255 | G-33 | CF3 |
| C.I.256 | G-33 | 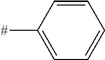 |
| C.I.257 | G-33 | 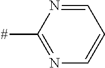 |
| C.I.258 | G-34 | H |
| C.I.259 | G-34 | F |
| C.I.260 | G-34 | Cl |
| C.I.261 | G-34 | Br |
| C.I.262 | G-34 | CH3 |
| C.I.263 | G-34 | CF3 |
| C.I.264 | G-34 | 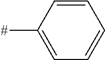 |
| C.I.265 | G-34 | 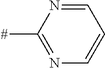 |
| C.I.266 | G-35 | H |
| C.I.267 | G-35 | F |
| C.I.268 | G-35 | Cl |
| C.I.269 | G-35 | Br |
| C.I.270 | G-35 | CH3 |
| C.I.271 | G-35 | CF3 |
| C.I.272 | G-35 | 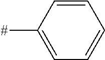 |
| C.I.273 | G-35 | 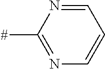 |
| C.I.274 | G-36 | H |
| C.I.275 | G-36 | F |
| C.I.276 | G-36 | Cl |
| C.I.277 | G-36 | Br |
| C.I.278 | G-36 | CH3 |
| C.I.279 | G-36 | CF3 |
| C.I.280 | G-36 | 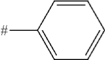 |
| C.I.281 | G-36 | 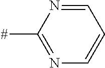 |
| C.I.282 | G-37 | H |
| C.I.283 | G-37 | F |
| C.I.284 | G-37 | Cl |
| C.I.285 | G-37 | Br |
| C.I.286 | G-37 | CH3 |
| C.I.287 | G-37 | CF3 |
| C.I.288 | G-37 | 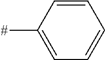 |
| C.I.289 | G-37 | 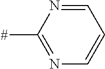 |
| C.I.290 | G-38 | H |
| C.I.291 | G-38 | F |
| C.I.292 | G-38 | Cl |
| C.I.293 | G-38 | Br |
| C.I.294 | G-38 | CH3 |
| C.I.295 | G-38 | CF3 |
| C.I.296 | G-38 | 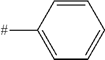 |
| C.I.297 | G-38 | 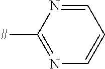 |
| C.I.298 | G-39 | H |
| C.I.299 | G-39 | F |
| C.I.300 | G-39 | Cl |
| C.I.301 | G-39 | Br |
| C.I.302 | G-39 | CH3 |
| C.I.303 | G-39 | CF3 |
| C.I.304 | G-40 | H |
| C.I.305 | G-40 | F |
| C.I.306 | G-40 | Cl |
| C.I.307 | G-40 | Br |
| C.I.308 | G-40 | CH3 |
| C.I.309 | G-40 | CF3 |
| C.I.310 | G-40 | 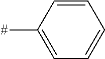 |
| C.I.311 | G-40 | 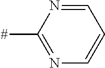 |
| C.I.312 | G-41 | H |
| C.I.313 | G-41 | F |
| C.I.314 | G-41 | Cl |
| C.I.315 | G-41 | Br |
| C.I.316 | G-41 | CH3 |
| C.I.317 | G-41 | CF3 |
| C.I.318 | G-42 | H |
| C.I.319 | G-42 | F |
| C.I.320 | G-42 | Cl |
| C.I.321 | G-42 | Br |
| C.I.322 | G-42 | CH3 |
| C.I.323 | G-42 | CF3 |
| C.I.324 | G-42 | 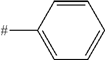 |
| C.I.325 | G-42 | 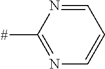 |
| C.I.326 | G-43 | H |
| C.I.327 | G-43 | F |
| C.I.328 | G-43 | Cl |
| C.I.329 | G-43 | Br |
| C.I.330 | G-43 | CH3 |
| C.I.331 | G-43 | CF3 |
| C.I.332 | G-43 | 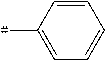 |
| C.I.333 | G-43 | 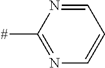 |
| C.I.334 | G-44 | H |
| C.I.335 | G-44 | F |
| C.I.336 | G-44 | Cl |
| C.I.337 | G-44 | Br |
| C.I.338 | G-44 | CH3 |
| C.I.339 | G-44 | CF3 |
| C.I.340 | G-44 | 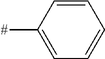 |
| C.I.341 | G-44 | 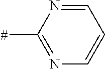 |
| C.I.342 | G-45 | H |
| C.I.343 | G-45 | F |
| C.I.344 | G-45 | Cl |
| C.I.345 | G-45 | Br |
| C.I.346 | G-45 | CH3 |
| C.I.347 | G-45 | CF3 |
| C.I.348 | G-45 | 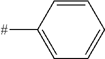 |
| C.I.349 | G-45 | 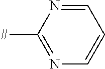 |
| C.I.350 | G-46 | H |
| C.I.351 | G-46 | F |
| C.I.352 | G-46 | Cl |
| C.I.353 | G-46 | Br |
| C.I.354 | G-46 | CH₃ |
| C.I.355 | G-46 | CF₃ |
| C.I.356 | G-46 | 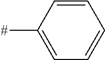 |
| C.I.357 | G-46 | 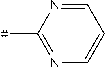 |
| C.I.358 | G-47 | H |
| C.I.359 | G-47 | F |
| C.I.360 | G-47 | Cl |
| C.I.361 | G-47 | Br |
| C.I.362 | G-47 | CH₃ |
| C.I.363 | G-47 | CF₃ |
| C.I.364 | G-47 | 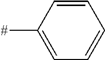 |
| C.I.365 | G-47 | 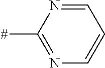 |
| C.I.366 | G-48 | H |
| C.I.367 | G-48 | F |
| C.I.368 | G-48 | Cl |
| C.I.369 | G-48 | Br |
| C.I.370 | G-48 | CH₃ |
| C.I.371 | G-48 | CF₃ |
| C.I.372 | G-48 | 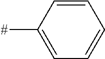 |
| C.I.373 | G-48 | 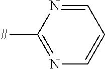 |
| C.I.374 | G-49 | H |
| C.I.375 | G-49 | CH₃ |
| C.I.376 | G-49 | CF₃ |
| C.I.377 | G-50 | H |
| C.I.378 | G-50 | CH₃ |
| C.I.379 | G-50 | CF₃ |
| C.I.380 | G-51 | H |
| C.I.381 | G-51 | CH₃ |
| C.I.382 | G-51 | CF₃ |
| C.I.383 | G-52 | H |
| C.I.384 | G-52 | CH₃ |
| C.I.385 | G-52 | CF₃ |
| C.I.386 | G-53 | H |
| C.I.387 | G-53 | CH₃ |
| C.I.388 | G-53 | CF₃ |
| C.I.389 | G-54 | H |
| C.I.390 | G-54 | CH₃ |
| C.I.391 | G-54 | CF₃ |
| C.I.392 | G-55 | — |
| C.I.393 | G-56 | H |
| C.I.394 | G-56 | CH₃ |
| C.I.395 | G-57 | — |
| C.I.396 | G-58 | H |
| C.I.397 | G-58 | CH₃ |
| C.I.398 | G-58 | CF₃ |
| C.I.399 | G-58 | 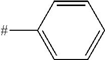 |
| C.I.400 | G-59 | H |
| C.I.401 | G-59 | CH₃ |
| C.I.402 | G-59 | CF₃ |
| C.I.403 | G-59 | 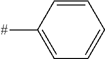 |
| C.I.404 | G-60 | H |
| C.I.405 | G-60 | CH₃ |
| C.I.406 | G-60 | CF₃ |
| C.I.407 | G-60 | 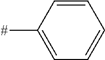 |
| C.I.408 | G-61 | H |
| C.I.409 | G-61 | CH₃ |
| C.I.410 | G-61 | CF₃ |
| C.I.411 | G-61 | 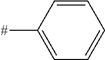 |
| C.I.412 | G-62 | H |
| C.I.413 | G-62 | CH₃ |
| C.I.414 | G-62 | CF₃ |
| C.I.415 | G-62 | 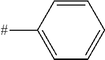 |
| C.I.416 | G-63 | H |
| C.I.417 | G-63 | CH₃ |
| C.I.418 | G-63 | CF₃ |
| C.I.419 | G-63 | 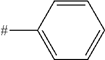 |
| C.I.420 | G-64 | H |
| C.I.421 | G-64 | CH₃ |
| C.I.422 | G-64 | CF₃ |
| C.I.423 | G-64 | 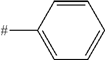 |
| C.I.424 | G-65 | H |
| C.I.425 | G-65 | CH₃ |
| C.I.426 | G-65 | CF₃ |
| C.I.427 | G-65 | 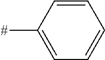 |
| C.I.428 | G-66 | H |
| C.I.429 | G-66 | CH₃ |
| C.I.430 | G-66 | CF₃ |
| C.I.431 | G-66 | 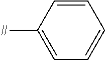 |
| C.I.432 | G-67 | H |
| C.I.433 | G-67 | CH₃ |
| C.I.434 | G-68 | H |
| C.I.435 | G-68 | CH₃ |
| C.I.436 | G-69 | H |
| C.I.437 | G-69 | CH₃ |
| C.I.438 | G-70 | H |
| C.I.439 | G-70 | CH₃ |
| C.I.440 | G-71 | H |
| C.I.441 | G-71 | CH₃ |
| C.I.442 | G-72 | H |
| C.I.443 | G-72 | CH₃ |

wherein # of R⁶ denotes the bond in the molecule

For example, the compound C.1 of structure S-6 having IUPAC name N-(4,5-dihydrothiazol-2-yl)-N-ethyl-2-(3-pyridyl)thiazol-5-amine, synthesized according to synthesis examples (see structure S-6) and characterized further below:

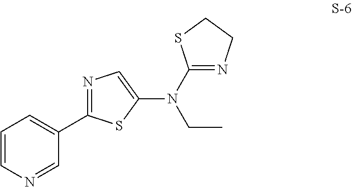 [SMILES: CCN(C1=NCCS1)c1cnc(-c2cccnc2)s1]

would correspond in table C.I. to compound example C.I.416 of formula I-aq or to compound example C.I.420 of formula I-aq, as R⁶ is hydrogen irrespective of its position.

Moreover, the meanings mentioned for those individual variables in the tables are per se, independently of the combination in which they are mentioned, a particularly preferred embodiment of the substituents in question.

### Preparation Methods

Compounds of formula (I) according to the present invention can generally be prepared by standard methods of organic chemistry e.g. by the preparation methods and preparation schemes as described below. If not otherwise specified for defined conditions, the definitions of m, A, G, R¹, R², R⁶ and k of the compounds of formula (I) and the other substituents of the molecular structures given in the schemes hereinbelow are as defined above.

Compounds of the formula (I), and any 3-pyridyl-containing synthetic precursors, can exist as in their unoxidized 3-pyridyl form (m is 0) or as their corresponding 3-pyridyl N-oxides (m is 1, Scheme 1). The N-oxide can be installed into, for example, compounds of the formula (I) or P-1 or P-3 by reaction of the corresponding pyridine compound with an oxidizing agent (e.g. meta-chloroperbenzoic acid, hydrogen peroxide, sodium periodate).

Compounds of the formula (I) can be prepared in a Suzuki coupling by reaction of a 3-pyridyl boronic acid P-1 (e.g. 3-pyridylboronic acid) in the presence of a palladium catalyst (e.g. tetrakis(triphenylphosphine)palladium), thiazole P-2, where X is for example Cl, Br, I or triflate, and base (e.g. K₂CO₃) in analogy to that described by, for example, Trullinger et al. WO 2010/129497 (see Scheme 1). Alternatively compounds of the formula (I) can be prepared in a Suzuki coupling, analogous to above, between a pyridine derivative of the formula P-3, where X is for example Cl, Br, I or trifluoromethane sulfonic acid (hereinafter triflate), and a thiazole boronic acid P-4 in analogy to that described by, for example, Duffy et al. WO 2011/146324.

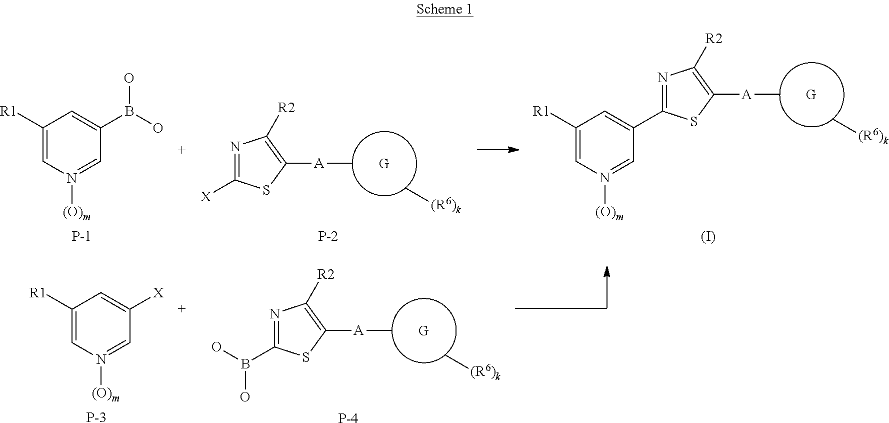

Compounds of the formula (I), where A is S, O or NR³, can also be prepared from thiazole derivatives of the formula P-5, where X is for example Cl, Br, I or triflate, and heterocycles of the formula P-6, wherein A carries additionally hydrogen such as SH, OH or NHR³ in copper or palladium-catalyzed cross-coupling reactions (Scheme 2). Representative literature precedent can be found in, for example, Nantermet et al. WO 2011/133447, Lui et al. WO 2010/101724 and Lin et al. WO 2009/152025. Alternatively compounds of the formula (I) can be prepared in copper or palladium-catalyzed cross-coupling reactions, analogous to above, between thiazoles P-7, wherein A carries additionally hydrogen such as SH, OH, NH₂ or NHR³, and heterocycles of the formula P-8, where X is Cl, Br, I or triflate.

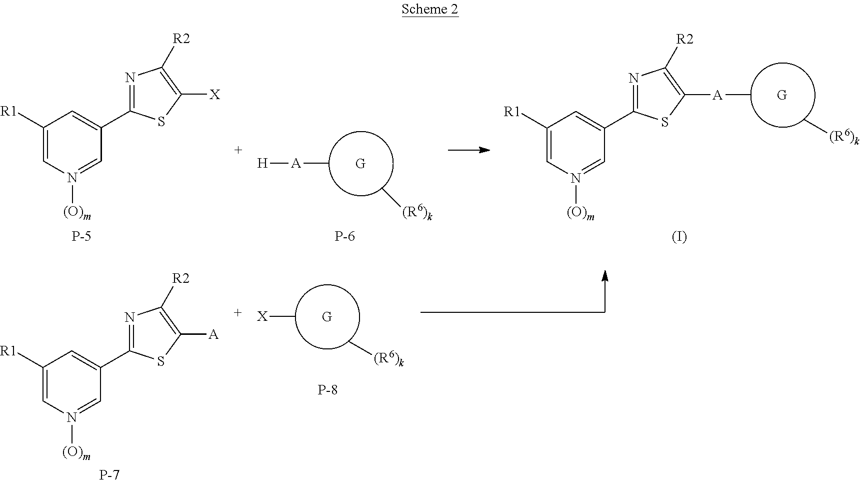

Halides of the formula P-10, where X is Cl, Br or I, can be prepared from thiazoles of the formula P-9 by reaction with a halogenating reagent (e.g. N-chloro-, N-bromo- or N-iodo-succinimide, iodine, bromine) in analogy to that described by, for example, Nantermet et al. WO 2011/133447 (Scheme 3). Alternatively halides of the formula P-10, where X is Cl, Br or I, can be prepared by reaction with a strong base (e.g. n-butyllithium) followed by an electrophilic halogen source (e.g. C₂Cl₆) in analogy to that described by, for example, Boehme et al. WO 2008/148468. Nitriles of the formula P-10 can be prepared from halides of the formula P-9 by reaction with a cyanide source (e.g. Zn(CN)₂) in the presence of a palladium catalyst (e.g. tetrakis(triphenylphosphine)palladium in analogy to that described by, for example, Chianelli et al. WO 2010/127152. Compounds of the formula (I) can be prepared from halides of the formula P-10 in Suzuki coupling reactions involving aliphatic boronic acids (e.g. ethyl boronic acid) under palladium catalysis (e.g. tetrakis(triphenylphosphine)palladium) in the absence or presence of silver oxide in analogy to that described by, for example, Zhou et al. J. Med. Chem. 2010, 53, 7251-7263.

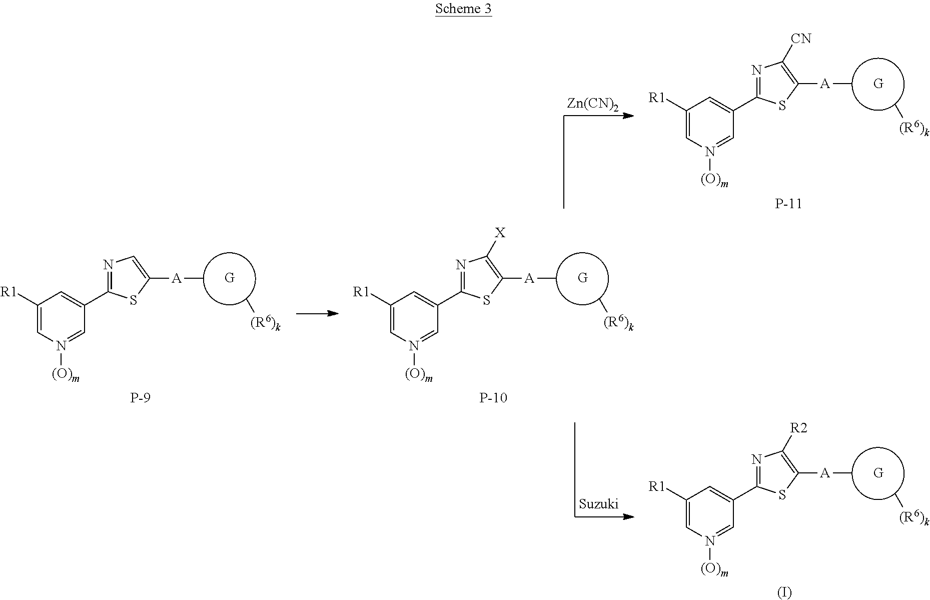

Thiazoles of the formula P-9 can be prepared from dicarbonyl compounds of the formula P-12 by reaction with Lawesson's reagent (or P₂S₅) by heating at reflux in a solvent like toluene or THF in analogy to that described by, for example, Lee et al. WO 2011/159067 (Scheme 4). Alternatively thiazoles of the formula P-9 can be prepared from thioamides of the formula P-13 and aldehydes of the formula P-14, where X is Cl, Br or I, by in the presense of a base (e.g. sodium bicarbonate) in analogy to that described by, for example, Xiang et al. Bioorg. Med. Chem. Lett. 2010 20, 4550-4554.

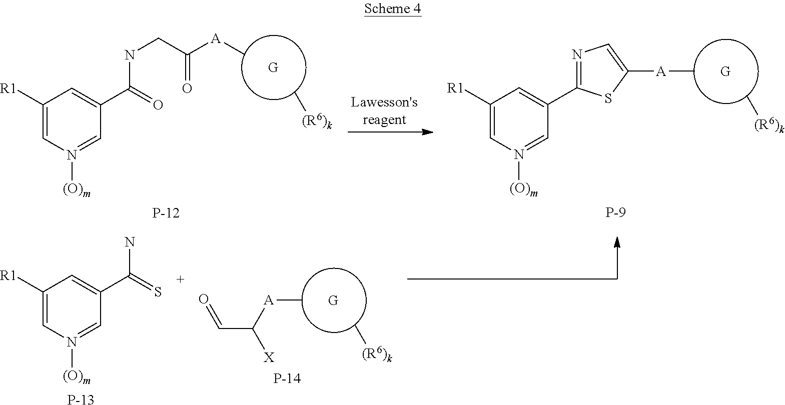

En route to N-linked compounds of the formula (I), where A is NR³, amines of the formula P-20 can be prepared in a 5-step synthesis starting from acids of the formula P-15 (Scheme 5). Firstly acids for the formula P-15 can be converted to hydrazoic acids of the formula P-16 by reaction with, for example, diphenylphosphoryl azide or thionyl chloride/sodium azide in analogy to that described by, for example, Trullinger et al. WO 2010/129497 or Allison et al. US 2006/0069286, respectively. Hydrazoic acids of the formula P-16 can then be reacted with t-butanol to form carbamates of the formula P-17 in analogy to that described by, for example, Trullinger et al. WO 2010/129497 or Binder et al. Synthesis, 1977, 4, 255-6. Carbamates of the formula P-17 can in turn be alkylated by reaction with an alkyl halide (e.g. ethyl iodide) or alkyl triflate (e.g. methyl triflate) in the presence of a base (e.g. Et₃N, NaH) in analogy to that described by, for example, Trullinger et al. WO 2010/129497. Alkyl carbamates of the formula P-18 can then be reacted under Suzuki conditions with a boronic acid (e.g. 3-pyridylboronic acid) in the presence of a palladium catalyst (e.g. tetrakis(triphenylphosphine)palladium) and a base (e.g. K₂CO₃) to form pyridyl thiazoles P-19 in analogy to that described by, for example, Trullinger et al. WO 2010/129497. Deprotection of pyridyl thiazoles of the formula P-19 under acidic conditions (e.g. TFA, aq. HCl) would afford the amines of the formula P-20 in analogy to that described by, for example, Trullinger et al. WO 2010/129497.

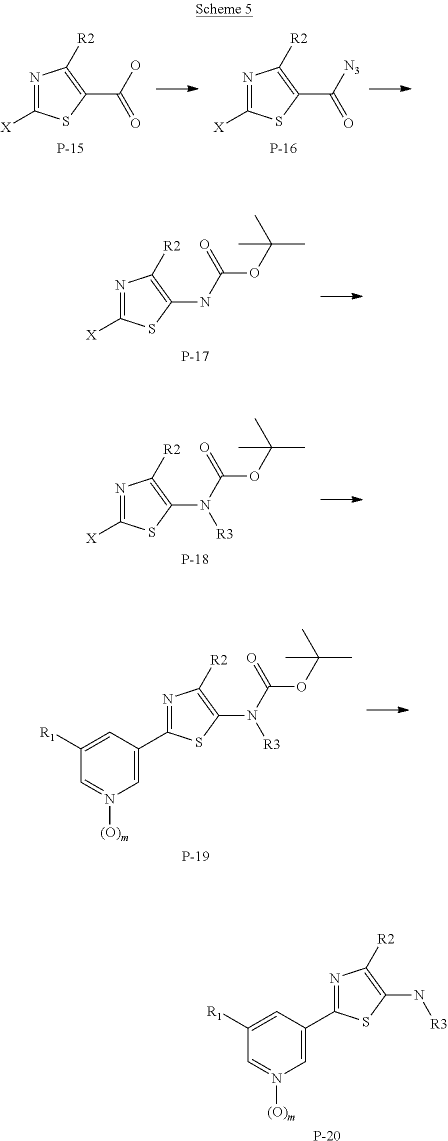

N-linked compounds of the formula (I), where A is NR³, such as amines P-21 can be prepared from amines of the formula P-20 and heterocycles of the formula P-8, where X is Cl, Br, I or triflate in copper or palladium-catalyzed cross-coupling reactions (Scheme 6).

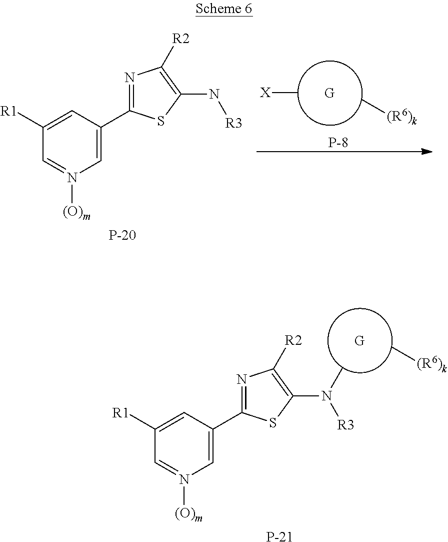

Additional N-linked compounds of the formula (I), where A is NR³, such as thiazolines of the formula P-23 and oxazolines of the formula P-26 can also be prepared from amines of the formula P-20 by reaction with isothiocyanates of the formula P-22 or isocyanates of the formula P-25, respectively, in analogy to that described by, for example, Koradin et al. WO 2008/145615 or Chow et al. WO 2009/023757, respectively (Scheme 7). Thiazoles of the formula P-24 and oxazoles of the formula P-27 can, in turn, be prepared from thiazolines of the formula P-23 and oxazolines of the formula P-26, respectively, by oxidation with, for example, manganese oxide and 2,3-dichloro-5,6-dicyano-1,4-benzoquinone in analogy to that described by, for example, Boissarie et al. Org. Lett. 2011, 13, 6256-6259.

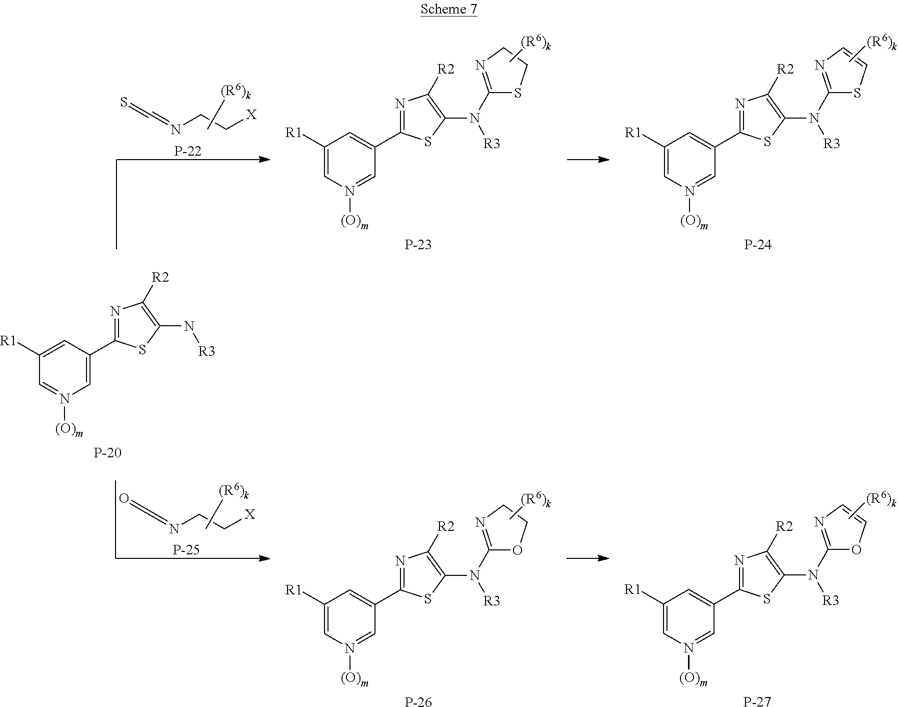

O-linked compounds of the formula (I), where A is O, such as ethers of the formula P-29 can be prepared from halides of the formula P-5, where X is F, Cl, Br or I, and heterocycles of the formula P-28 by reaction with a base (e.g. Cs₂CO₃) in a polar aprotic solvent (e.g. DMF) in analogy to that described by, for example, Yamashita et al. WO 2008/136444 (Scheme 8). O-linked compounds of the formula P-29 can also be prepared from halides of the formula P-5, where X is F, Cl, Br or I, and heterocycles of the formula P-28 in copper or palladium-catalyzed cross-coupling reactions.

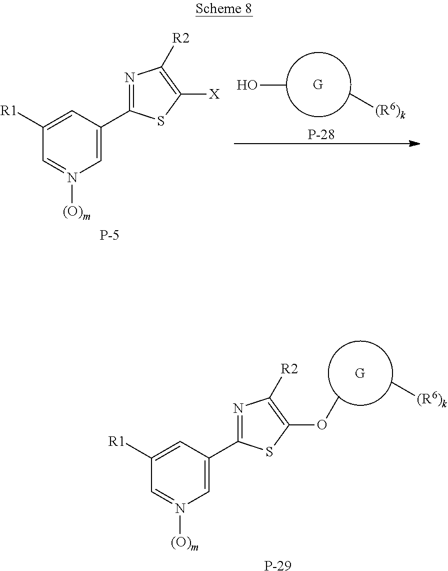

S-linked compounds of the formula (I), where A is S, such as thioethers of the formula P-31 can be prepared from halides of the formula P-5, where X is F, Cl, Br or I, and heterocycles of the formula P-30 by reaction in copper-catalyzed cross-couplings in analogy to that described by, for example, Nantermet et al. WO 2011/133447 (Scheme 9). Sulfoxides of the formula P-32 (n is 1) and sulfones of the formula P-32 (n is 2) can, in turn, be prepared from sulfides of the formula P-31 by oxidation with meta-chloroperbenzoic acid in analogy to that described by, for example, Turov et al. Russ. J. Gen. Chem. 2008, 78, 2132-2136 or Conn et al. WO 2011/143466, respectively.

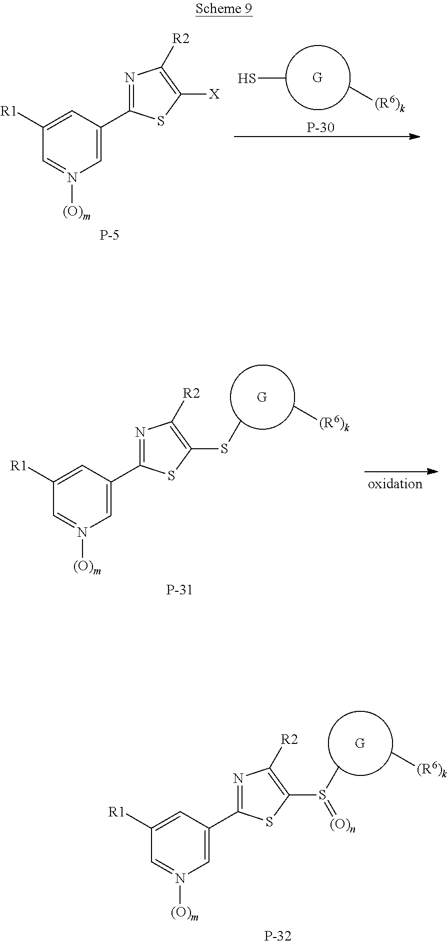

C-linked compounds of the formula (I), where A is CR^{4a}R^{4b}, such as methylene-bridged compounds of the formula P-34 can be prepared from ketoamides of the formula P-33 by reaction with Lawesson's reagent in analogy to that described by Buckman et al. WO 2011/038293 (Scheme 10). Alternatively methylene-bridged compounds P-34 can be prepared from thioamides of the formula P-13 and carbonyl compounds of the formula P-35, where X is Cl, Br or I in analogy to that described by, for example, Benjamin et al. J. Med. Chem. 1983, 26, 100-3.

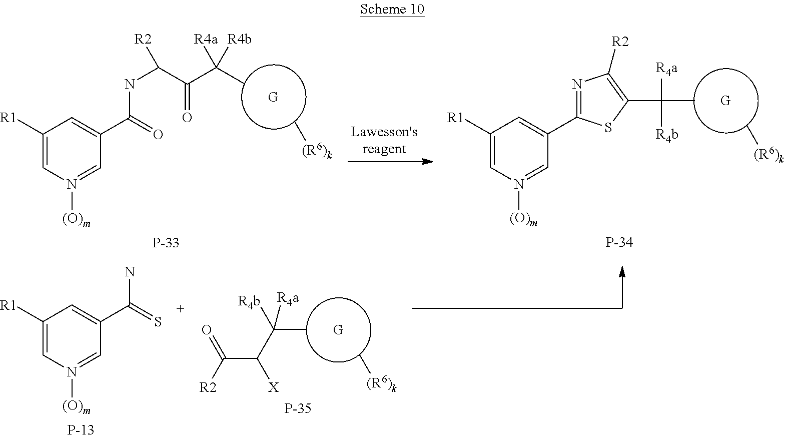

Additionally methylene-bridged compounds of the formula P-34 can be prepared from halides of the formula P-5 and zinc compounds of the formula P-36 in Negishi couplings involving a palladium catalyst (e.g. tetrakis(triphenylphosphine)palladium) in analogy to that described by, for example, Oalmann et al. WO 2010/071853 (Scheme 11).

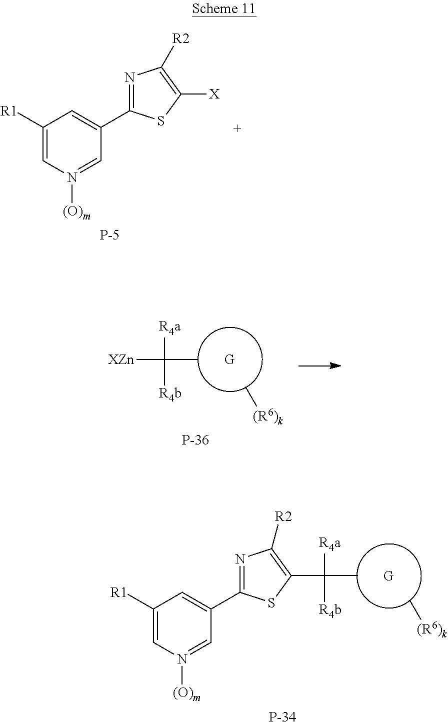

C-linked compounds of the formula (I), where A is C is NOR³ or C is NR^{9a} such as oximes of the formula P-39 or imines of the formula P-41 can be prepared from ketones of the formula P-40 by condensation with a hydroxylamine or amine derivative in analogy to that described by, for example, LaMattina et al. Tetrahedron Lett. 1984, 25, 2957-60 or Delhomel et al. WO 2008/087367 (Scheme 12). In turn ketones of the formula P-40 can be prepared in a 2-step sequence by reacting aldehydes of the formula P-37 with a Grignard reagent of the formula P-38 then oxidation with, for example, Dess-Martin periodinane in analogy to that described by, for example, Dalton et al. WO 2011/109059.

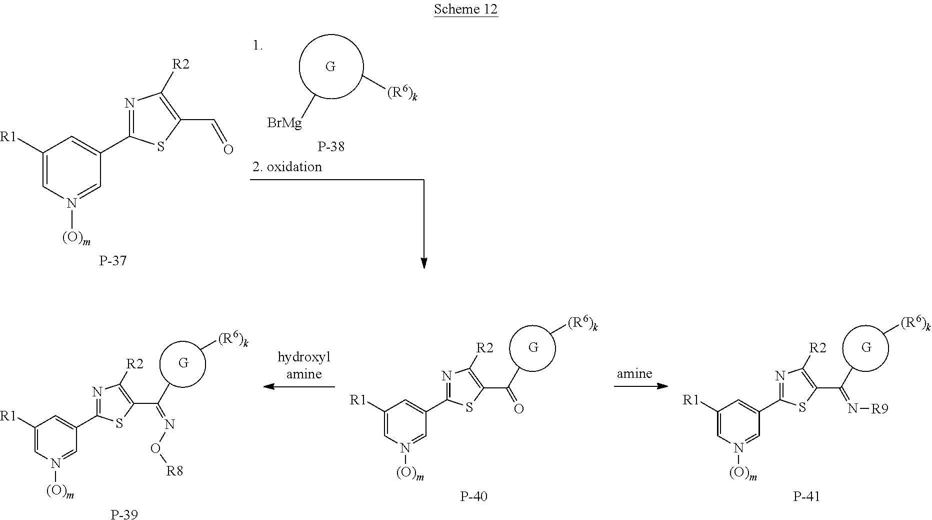

C-linked compounds of the formula (I), where A is C is CHR¹³ or C is CR⁷R¹³ such as olefins of the formula P-42 or of the formula P-43 can be prepared from ketones P-40 in olefination reactions (Scheme 13). Representative literature precedent can be found in, for example, Brown et al. WO 2008/030618 Keil et al. WO 2007/039178

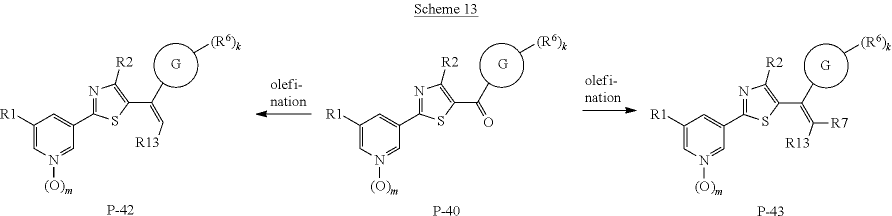

If individual compounds cannot be prepared via the above-described routes, they can be prepared by derivatization of other compounds (I) or by customary modifications of the synthesis routes described.

For example, in individual cases, certain compounds of formula (I) can advantageously be prepared from other compounds of formula (I) by derivatization, e.g. by ester hydrolysis, amidation, esterification, ether cleavage, olefination, reduction, oxidation and the like, or by customary modifications of the synthesis routes described.

The reaction mixtures are worked up in the customary manner, for example by mixing with water, separating the phases, and, if appropriate, purifying the crude products by chromatography, for example on alumina or silica gel. Some of the intermediates and end products may be obtained in the form of colorless or pale brown viscous oils, which are freed or purified from volatile components under reduced pressure and at moderately elevated temperature. If the intermediates and end products are obtained as solids, they may be purified by recrystallization, trituration or digestion. Room temperature means a temperature range between about 20 and 25° C.

### Pests

The term “invertebrate pest” as used herein encompasses animal populations, such as arthropode pests, including insects and arachnids, as well as nematodes, which may attack plants thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The compounds of the formula I, and their salts are in particular suitable for efficiently controlling arthropodal pests such as arachnids, myriapedes and insects as well as nematodes.

The compounds of the formula I are especially suitable for efficiently combating the following pests:

Insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecika ambiguella, Evetria boukana, Feltia subterranea, Gallena mellonella, Grapholitha funebrana, Grapholitha molesta, Heliothis armigera, Heliothis virescens, Heliothis zea, Hellula undalis, Hibernia defoliaria, Hyphantria cunea, Hyponomeuta Keifena lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panogs flammea, Pectinophora gossypiella, Peridroma saucia, Phalera bucephala, Phthorirnaea opercuiella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pillenana, Spodoptera frugiperda, Spodoptera littoralis, Spodoptera litura, Tuta absoluta, Thaumatopoea pityocampa, Tortrix vindana, Tnbhoplusia ni and Zeiraphera canadensis,

beetles (Coleoptera), for example Agrilus sinuatus, Agriotes lineatus, Agriotes obscurus, Amphimallus solstitialis, Anisandrus dispar, Anoplophora glabripennis, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assirndis, Ceuthorrhynchus napi, Chaetocnema Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera posfica, Ips typographus, Lema bilineata, Lema melanopus, Leptinotarsa decemlineata, Lirnonius californicus, Lissorhoptrus oryzophllus, Melanotus communis, Meligethes aeneus, Melolontha hippocastani, Melolontha melolontha, Oulema oryzae, Otiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popilli ajaponica, Sitona lineatus and Sitophilus granaria;

flies, mosquitoes (Diptera), e.g. Aedes aegypti, Aedes albopictus, Aedes vexans, Anastrepha ludens, Anopheles maculipennis, Anopheles crucians, Anopheles albirnanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliompa hominivorax, Contannia sorghicola Cordylobia anthropophaga, Culicoides furens, Culex pipiens, Culex nigripalpus, Culiex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia cambularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza Lucilla caprin, Lucilia cuprina, Lucilla sericata, Lycoria pectoralis, Mansonia titillanus, Mayetiola destructor, Musca autumnalis, Musca domesfica, Muscina stabulans, Oestrus ovis, Opomyza forum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosirnulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;

trips (Thysanoptera), e.g. Dichromothrips corbetti, Dichromothrips ssp., Frankliniella fusca, Frankliniella occidentalis, Frankliniella tritici, Scirtothrips citri, Thrips oryzae, Thrips palmi and Thrips tabaci,

termites (Isoptera), e.g. Calotermes flavicollis, Leucotermes flavipes, Heterotermes aureus, Reticulitermes flavipes, Reticulitermes virginicus, Reticulitermes lucifugus, Reticulitermes santonensis, Reticulitermes grassei, Termes natalensis, and Coptotermes formosanus;

cockroaches (Blattana-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis;

bugs, aphids, leafhoppers, whiteflies, scale insects, cicadas (Hemiptera), e.g. Acrosternum hilare, Acrosternum spp, Blissus leucopterus, Cyrtopeltis notatus, Dectes texanus texanus, Dysdercus angulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulanae, Aphis schneideri, Aphis spiraecola, Aphis sambuci, Acyrthosiphon Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypi, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis gyri, Empoasca fabae, Euschistus heros, Euschistus servus, halyomorpha halys, Hyalopterus pruni, Hyperomyzus lactucae, Macrosiphum avenae, Macrosiphum euphorbiae, Macrosiphon rosae, Megacopta cribraria, Megoura viciae, Melanaphis pyrarius, Metopolophium dirhodum, Myzus persicae, Myzus ascalonicus, Myzus cerasi, Myzus varians, Nasonovia ribis-nigri, Nezara vindula, Nilaparvata lugens, Piezodorus spp, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mala, Schizaphis graminum, Schizoneura lanuginosa, Sitobion avenae, Tnaleurodes vaporariorum, Toxoptera aurantiiand, Viteus vitifolii, Cimex lectularius, Cimex hemipterus, Reduvius senigs, Triatoma spp., and Arilus critatus;

ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Lasius niger, Monomorium pharaohis, Solenopsis geminata, Solenopsis invicta, Solenopsis richteri, Sotenopsis xyloni, Pogonomyrmex barbatus, Pogonomyrmex californicus, Pheidole megacephala, Dasymutilla occidentalis, Bombus spp., Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile;

crickets, grasshoppers, locusts (Orthoptera), e.g. Acheta domestica, Giyllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angullfera, Calllptamus italicus, Chortoicetes terminifera, and Locustana pardalina;

arachnids (Arachnida), such as acari, e.g. of the families Argasidae, Ixodidae and Sarcoptidae, such as Amblyomma americanum, Amblyomma variegatum, Amblyomma maculatum, Argas persicus, Boophilus annulatus, Boophilus decoloratus, Boophilus microplus, Dermacentor silvarum, Dermacentor andersoni, Dermacentor vanabills, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenids; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Ologonychus pratensis; Araneida, e.g. Latrodectus mactans, and Loxosceles reclusa;

fleas (Siphonaptera), e.g. Ctenocephalides fells, Ctenocephalides canis, Xenopsylla cheopis, Pulex irritans, Tunga penetrans, and Nosopsyllus fasciatus,

silverfish, firebrat (Thysanura), e.g. Lepisma saccharina and Thermobia domestica,

centipedes (Chilopoda), e.g. Scutigera coleoptrata,

millipedes (Diplopoda), e.g. Narceus spp.,

Earwigs (Dermaptera), e.g. forficula auricularia,

lice (Phthiraptera), e.g. Pedicuius humanus capitis, Pedicuius humanus corporis, Pthirus Haematopinus eurysternus, Haematopinus suis, Linognathus vituli, Bovicola bovis, Menopon gallinae, Menacanthus stramineus and Solenopotes capillatus.

Collembola (springtails), e.g. Onychiurus ssp.

They are also suitable for controlling Nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glyanes, Heterodera schachtii, Heterodera trifolii, and other Heterodera species; Seed gall nematodes, Anguina species; Stem and foliar nematodes, Aphelenchoides species; Sting nematodes, Belonolairnus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

Compounds of the formula I are particularly useful for controlling insects, preferably sucking or piercing insects such as insects from the genera Thysanoptera, Diptera and Hemiptera, in particular the following species:

Thysanoptera: Frankliniella fusca, Frankliniella occidentalis, Frankliniella tritici, Scirtothrips citri, Thrips oryzae, Thrips palmi and Thrips tabaci,

Diptera, e.g. Aedes aegypti, Aedes albopiqtus, Aedes vexans, Anastrepha ludens, Anopheles maculipennis, Anopheles crucians, Anopheles albirnanus, Anopheles gambiae, Anopheles freeborni, Anopheles leucosphyrus, Anopheles minimus, Anopheles quadrirnaculatus, Calliphora Ceratitis capitata, Chrysomya bezziana, Chrysomya hominivorax, Chrysomya macellaria, Chrysops discalis, Chrysops silacea, Chrysops atlantiqus, Cochliompa hominivorax, Contannia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza forum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosirnulium mbdum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;

Hemiptera, in particular aphids: Acyrthosiphon onobiychis, Adelges laricis, Aphidula nasturtii, Aphis fabae, Aphis forbesi, Aphis pomi, Aphis gossypii, Aphis grossulanae, Aphis schneideri, Aphis spiraecola, Aphis sambuci, Acyrthosiphon pisum, Aulacorthum solani, Brachycaudus cardui, Brachycaudus helichrysi, Brachycaudus persicae, Brachycaudus prunicola, Brevicolyne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis gyri, Empoasca fabae, Hyalopterus Pruni, Hyperomyzus lactucae, Macrosiphum avenae, Macrosiphum euphorbiae, Macrosiphon rosae, Megoura viciae, MelanaphiS pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Tnaleurodes vaporariorum, Toxoptera aurantiiand, and Viteus vitifolii.

Compounds of the formula I are particularly useful for controlling insects of the orders Hemiptera and Thysanoptera.

### Formulations

For use in a method according to the present invention, the compounds of formula I can be converted into the customary formulations, e.g. solutions, emulsions, suspensions, dusts, powders, pastes, granules and directly sprayable solutions. The use form depends on the particular purpose and application method. Formulations and application methods are chosen to ensure in each case a fine and uniform distribution of the compound of the formula I according to the present invention.

The invention therefore also relates to agrochemical compositions comprising an auxiliary and at least one compound I according to the invention.

An agrochemical composition comprises a pesticidally effective amount of a compound I. The term “effective amount” denotes an amount of the composition or of the compounds I, which is sufficient for controlling animal pests on a locus, such as crops, cultivated plants or in the protection of materials and which does not result in a substantial damage to the treated plants. Such an amount can vary in a broad range and is dependent on various factors, such as the animal pest species to be controlled, the treated cultivated plant or material, the climatic conditions and the specific compound I used.

The compounds I, their N-oxides and salts can be converted into customary types of agrochemical compositions, e.g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal artiIles (e.g. LN), as well as gel formulations for the treatment of plant propagation materials such as seeds (e.g. GF). These and further compositions types are defined in the “Catalogue of pes-ticide formulation types and international coding system”, Technical Monograph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Examples for suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharide powders, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyhnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-subsititued fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxilaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for composition types and their preparation are:

### i) Water-Soluble Concentrates (SL, LS)

10-60 wt % of a compound I according to the invention and 5-15 wt % wetting agent (e.g. alcohol alkoxylates) are dissolved in water and/or in a water-soluble solvent (e.g. alcohols) up to 100 wt %. The active substance dissolves upon dilution with water.

### ii) Dispersible Concentrates (DC)

5-25 wt % of a compound I according to the invention and 1-10 wt % dispersant (e.g. polyvinylpyrrolidone) are dissolved in up to 100 wt % organic solvent (e.g. cyclohexanone). Dilution with water gives a dispersion.
   iii) Emulsifiable Concentrates (EC)
15-70 wt % of a compound I according to the invention and 5-10 wt % emulsifiers (e.g. calcium dodecylbenzenesulfonate and castor oil ethoxylate) are dissolved in up to 100 wt % water-insoluble organic solvent (e.g. aromatic hydrocarbon). Dilution with water gives an emulsion.

### iv) Emulsions (EW, EO, ES)

5-40 wt % of a compound I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into up to 100 wt % water by means of an emulsifying machine and made into a homogeneous emulsion. Dilution with water gives an emulsion.

### v) Suspensions (SC, OD, FS)

In an agitated ball mill, 20-60 wt % of a compound I according to the invention are comminuted with addition of 2-10 wt % dispersants and wetting agents (e.g. sodium lignosulfonate and alcohol ethoxylate), 0, 1-2 wt % thickener (e.g. xanthan gum) and up to 100 wt % water to give a fine active substance suspension. Dilution with water gives a stable suspension of the active substance. For FS type composition up to 40 wt % binder (e.g. polyvinylalcohol) is added.

### vi) Water-Dispersible Granules and Water-Soluble Granules (WG, SG)

50-80 wt % of a compound I according to the invention are ground finely with addition of up to 100 wt % dispersants and wetting agents (e.g. sodium lignosulfonate and alcohol ethoxylate) and prepared as water-dispersible or water-soluble granules by means of technical appliances (e.g. extrusion, spray tower, fluidized bed). Dilution with water gives a stable dispersion or solution of the active substance.
   vii) Water-Dispersible Powders and Water-Soluble Powders (WP, SP, WS)
50-80 wt % of a compound I according to the invention are ground in a rotor-stator mill with addition of 1-5 wt % dispersants (e.g. sodium lignosulfonate), 1-3 wt % wetting agents (e.g. alcohol ethoxylate) and up to 100 wt % solid carrier, e.g. silica gel. Dilution with water gives a stable dispersion or solution of the active substance.
   viii) Gel (GW, GF)
In an agitated ball mill, 5-25 wt % of a compound I according to the invention are comminuted with addition of 3-10 wt % dispersants (e.g. sodium lignosulfonate), 1-5 wt % thickener (e.g. carboxymethylcellulose) and up to 100 wt % water to give a fine suspension of the active substance. Dilution with water gives a stable suspension of the active substance.

### iv) Microemulsion (ME)

5-20 wt % of a compound I according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water up to 100%. This mixture is stirred for 1 h to produce spontaneously a thermodynamically stable microemulsion.

### iv) Microcapsules (CS)

An oil phase comprising 5-50 wt % of a compound I according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexa-methylenediamine) results in the formation of a polyurea microcapsules. The monomers amount to 1-10 wt %. The wt % relate to the total CS composition.

### ix) Dustable Powders (DP, DS)

1-10 wt % of a compound I according to the invention are ground finely and mixed intimately with up to 100 wt % solid carrier, e.g. finely divided kaolin.

### x) Granules (GR, FG)

0.5-30 wt % of a compound I according to the invention is ground finely and associated with up to 100 wt % solid carrier (e.g. silicate). Granulation is achieved by extrusion, spraydrying or the fluidized bed.

### xi) Ultra-Low Volume Liquids (UL)

1-50 wt % of a compound I according to the invention are dissolved in up to 100 wt % organic solvent, e.g. aromatic hydrocarbon.

The compositions types i) to xi) may optionally comprise further auxiliaries, such as 0, 1-1 wt % bactericides, 5-15 wt % anti-freezing agents, 0, 1-1 wt % anti-foaming agents, and 0, 1-1 wt % colorants.

The agrochemical compositions generally comprise between 0.01 and 95%, preferably between 0.1 and 90%, and most preferably between 0.5 and 75%, by weight of active substance. The active substances are generally employed in a purity of from 90% to 100%, preferably from 95% to 100% (according to NMR spectrum).

Water-soluble concentrates (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds.

The compositions in question give, after two-to-tenfold dilution, active substance concentrations of from 0.01 to 60% by weight, preferably from 0.1 to 40% by weight, in the ready-to-use preparations.

Application can be carried out before or during sowing. Methods for applying or treating compound I and compositions thereof, respectively, on to plant propagation material, especially seeds include dressing, coating, pelleting, dusting, soaking and in-furrow application methods of the propagation material. Preferably, compound I or the compositions thereof, respectively, are applied on to the plant propagation material by a method such that germination is not induced, e.g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, in particular from 0.1 to 0.75 kg per ha. In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seed) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and other pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

### Mixtures

According to one embodiment of the present invention, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e.g. components comprising compounds I and/or active substances from the groups M.1 to M.UN.X or F.I to F.XII, may be mixed by the user in a spray tank and further auxiliaries and additives may be added, if appropriate.

In a further embodiment, either individual components of the composition according to the invention or partially premixed components, e.g. components comprising compounds I and/or active substances from the groups M.1 to M.UN.X or F.I to F.XII, can be applied jointly (e.g. after tank mix) or consecutively.

The following list M of pesticides, grouped according the Mode of Action Classification of the Insecticide Resistance Action Committee (IRAC), together with which the compounds according to the invention can be used and with which potential synergistic effects might be produced, is intended to illustrate the possible combinations, but not to impose any limitation:

M.1 Acetylcholine esterase (AChE) inhibitors from the class of
M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of
M.1B organophosphates, for example acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion, parathionmethyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;
M.2. GABA-gated chloride channel antagonists such as:
M.2A cyclodiene organochlorine compounds, as for example endosulfan or chlordane; or
M.2B fiproles (phenylpyrazoles), as for example ethiprole, fipronil, flufiprole, pyrafluprole and pyriprole;
M.3 Sodium channel modulators from the class of
M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, betacyfluthrin, cyhalothrin, lambda-cyhalothrin, gamma-cyhalothrin, cypermethrin, alphacypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, meperfluthrin, metofluthrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or
M.3B sodium channel modulators such as DDT or methoxychlor;
M.4 Nicotinic acetylcholine receptor agonists (nAChR) from the class of
M.4A neonicotinoids, for example acteamiprid, chlothianidin, dinotefuran, imidacloprid, nitenpyram, thiacloprid and thiamethoxam; or M.4B nicotine.
M.5 Nicotinic acetylcholine receptor allosteric activators from the class of spinosyns, for example spinosad or spinetoram;
M.6 Chloride channel activators from the class of avermectins and milbemycins, for example abamectin, emamectin benzoate, ivermectin, lepimectin or milbemectin;
M.7 Juvenile hormone mimics, such as
M.7A juvenile hormone analogues as hydroprene, kinoprene and methoprene; or others as
M.7B fenoxycarb or M.7C pyriproxyfen;
M.8 miscellaneous non-specific (multi-site) inhibitors, for example
M.8A alkyl halides as methyl bromide and other alkyl halides, or
M.8B chloropicrin, or M.8C sulfuryl fluoride, or M.8D borax, or M.8E tartar emetic;
M.9 Selective homopteran feeding blockers, for example
M.9B pymetrozine, or M.9C flonicamid;
M.10 Mite growth inhibitors, for example
M.10A clofentezine, hexythiazox and diflovidazin, or M.10B etoxazole;
M.11 Microbial disruptors of insect midgut membranes, for example bacillus thuringiensis or bacillus sphaericus and the insecticdal proteins they produce such as bacillus thuringiensis subsp. israelensis, bacillus sphaericus, bacillus thuringiensis subsp. aizawai, bacillus thunngiensis subsp. kurstaki and bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;
M.12 Inhibitors of mitochondrial ATP synthase, for example
M.12A diafenthiuron, or
M.12B organotin miticides such as azocyclotin, cyhexatin or fenbutatin oxide, or M.12C propargite, or M.12D tetradifon;
M.13 Uncouplers of oxidative phosphorylation via disruption of the proton gradient, for example chlorfenapyr, DNOC or sulfluramid;
M.14 Nicotinic acetylcholine receptor (nAChR) channel blockers, for example nereistoxin analogues as bensultap, cartap hydrochloride, thiocyclam or thiosultap sodium;
M.15 Inhibitors of the chitin biosynthesis type 0, such as benzoylureas as for example bistrifluron, chlorfluazuron, diflubenzuron, flucycloxuron, flufenoxuron, hexaflumuron, lufenuron, novaluron, noviflumuron, teflubenzuron or triflumuron;
M.16 Inhibitors of the chitin biosynthesis type 1, as for example buprofezin;
M.17 Moulting disruptors, Dipteran, as for example cyromazine;
M.18 Ecdyson receptor agonists such as diacylhydrazines, for example methoxyfenozide, tebufenozide, halofenozide, fufenozide or chromafenozide;
M.19 Octopamin receptor agonists, as for example amitraz;
M.20 Mitochondrial complex III electron transport inhibitors, for example
M.20A hydramethylnon, or M.20B acequinocyl, or M.20C fluacrypyrim;
M.21 Mitochondrial complex I electron transport inhibitors, for example
M.21A METI acaricides and insecticides such as fenazaquin, fenpyroximate, pyrimidifen, pyridaben, tebufenpyrad or tolfenpyrad, or M.21B rotenone;
M.22 Voltage-dependent sodium channel blockers, for example
M.22A indoxacarb, or M.22B metaflumizone;
M.23 Inhibitors of the of acetyl CoA carboxylase, such as Tetronic and Tetramic acid derivatives, for example spirodiclofen, spiromesifen or spirotetramat;
M.24 Mitochondrial complex IV electron transport inhibitors, for example
M.24A phosphine such as aluminium phosphide, calcium phosphide, phosphine or zinc phosphide, or M.24B cyanide.
M.25 Mitochondrial complex II electron transport inhibitors, such as beta-ketonitrile derivatives, for example cyenopyrafen or cyflumetofen;
M.28 Ryanodine receptor-modulators from the class of diamides, as for example flubendiamide, chlorantraniliprole (Rynaxypyr®), cyantraniliprole (Cyazypyr®), or the phthalamide compounds
M.28.1: (R)-3-Chlor-N1-{2-methyl-4-[1,2,2,2 tetrafluor-1-(trifluormethyl)ethyl]phenyl}-N2-(1-methyl-2-methylsulfonylethyl)phthalamid and
M.28.2: (S)-3-Chlor-N1-{2-methyl-4-[1,2,2,2 tetrafluor-1-(trifluormethyl)ethyl]phenyl}-N2-(1-methyl-2-methylsulfonylethyl)phthalamid, or the compound
M.28.3: 3-bromo-N-{2-bromo-4-chloro-6-[(1-cyclopropylethyl)carbamoyl]phenyl}-1-(3-chlorpyridin-2-yl)-1H-pyrazole-5-carboxamide (proposed ISO name: cyclaniliprole), or the compound
M.28.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate; or a compound selected from M.28.5a) to M.28.5h):
M.28.5a) N-[4,6-dichloro-2-Rdiethyl-lambda-4-sulfanylidene)carbamoylyphenyl]-2-(3-chloro-2-pyridyl)-5-(trifluoromethyl)pyrazole-3-carboxamide;
M.28.5b) N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide;
M.28.5c) N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide;
M.28.5d) N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide;
M.28.5e) N-[4,6-dichloro-2-Rdiethyl-lambda-4-sulfanylidene)carbamoylyphenyl]-2-(3-chloro-2-pyridyl)-5-(difluoromethyl)pyrazole-3-carboxamide;
M.28.5f) N-[4,6-d]bromo-2-[(di-2-propyl-lambda-4-sulfanylidene)carbamoylyphenyl]-2-(3-chloro-2-pyridyl)-5-(trifluoromethyl)pyrazole-3-carboxamide;
M.28.5 g) N-[4-chloro-2-[(di-2-propyl-lambda-4-sulfanylidene)carbamoyl]-6-cyano-phenyl]-2-(3-chloro-2-pyridyl)-5-(trifluoromethyl)pyrazole-3-carboxamide;
M.28.5h) N-[4,6-dibromo-2-Rdiethyl-lambda-4-sulfanylidene)carbamoylyphenyl]-2-(3-chloro-2-pyridyl)-5-(trifluoromethyl)pyrazole-3-carboxamide.
   M.UN.X insecticidal active compounds of unknown or uncertain mode of action, as for example azadirachtin, amidoflumet, benzoximate, bifenazate, bromopropylate, chinomethionat, cryolite, dicofol, flufenerim, flometoquin, fluensulfone, flupyradifurone, piperonyl butoxide, pyridalyl, pyrifluquinazon, sulfoxaflor, or the compound
M.UN.X.1: 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide, or the compound
M.UN.X.2: cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester, or the compound
M.UN.X.3: 11-(4-chloro-2,6-dimethylphenyl)-12-hydroxy-1,4-dioxa-9-azadispiro[4.2.4.2]-tetradec-11-en-10-one, or the compound
M.UN.X.4: 3-(4′-fluoro-2,4-dimethylbiphenyl-3-yl)-4-hydroxy-8-oxa-1-azaspiro[4.5]dec-3-en-2-one, or the compound
M.UN.X.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of bacillus firmus (Votivo, 1-1582); or
M.UN.X.6; a compound selected from the group of
(E/Z)—N-[1-[(6-chloro-3-pyridyl)methyl]-2-pyridylidene]-2,2,2-trifluoro-acetamide;
(E/Z)—N-[1-[(6-chloro-5-fluoro-3-pyridyl)methyl]-2-pyridylidene]-2,2,2-trifluoro-acetamide;
(E/Z)-2,2,2-trifluoro-N-[1-[(6-fluoro-3-pyridyl)methyl]-2-pyridylidene]acetamide;
(E/Z)—N-[1-[(6-bromo-3-pyridyl)methyl]-2-pyridylidene]-2,2,2-trifluoro-acetamide;
(E/Z)—N-[1-[1-(6-chloro-3-pyridypethyl]-2-pyridylidene]-2,2,2-trifluoro-acetamide;
(E/Z)—N-[1-[(6-chloro-3-pyridyl)methyl]-2-pyridylidene]-2,2-difluoro-acetamide;
(E/Z)-2-chloro-N-[1-[(6-chloro-3-pyridyl)methyl]-2-pyridylidene]-2,2-difluoro-acetamide;
(E/Z)—N-[1-[(2-chloropyrimidin-5-yOmethyl]-2-pyridylidene]-2,2,2-trifluoro-acetamide and
(E/Z)—N-[1-[(6-chloro-3-pyridyl)methyl]-2-pyridylidene]-2,2,3,3,3-pentafluoro-propanamide.).

The commercially available compounds of the group M listed above may be found in The Pesticide Manual, 15th Edition, C. D. S. Tomlin, British Crop Protection Council (2011) among other publications.

The phthalamides M.28.1 and M.28.2 are both known from WO 2007/101540. The anthranilamide M.28.3 has been described in WO2005/077934. The hydrazide compound M.28.4 has been described in WO 2007/043677. The anthranilamides M.28.5a) to M.28.5h) can be prepared as described in WO 2007/006670, WO2013/024009 and WO2013/024010.

The compounds listed in M.UN.X.6 have been described in WO2012/029672.

The quinoline derivative flometoquin is shown in WO2006/013896. The aminofuranone compounds flupyradifurone is known from WO 2007/115644. The sulfoximine compound sulfoxaflor is known from WO2007/149134. The isoxazoline compound M.X.1 has been described in WO2005/085216. The pyripyropene derivative M.X.2 has been described in WO 2006/129714. The spiroketal-substituted cyclic ketoenol derivative M.X.3 is known from WO2006/089633 and the biphenyl-substituted spirocyclic ketoenol derivative M.X.4 from WO2008/067911. Finally triazoylphenylsulfide like M.X.5 have been described in WO2006/043635 and biological control agents on basis of bacillus firmus in WO2009/124707.

The following list F of active substances, in conjunction with which the compounds according to the invention can be used, is intended to illustrate the possible combinations but does not limit them:

### F.I) Respiration Inhibitors

F.I-1) Inhibitors of complex III at Qo site:
strobilurins: azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, triclopyricarb/chlorodincarb, trifloxystrobin, 2-[2-(2,5-dimethyl-phenoxymethyl)-phenyl]-3-methoxy-acrylic acid methyl ester and 2 (2-(3-(2,6-dichlorophenyl)-1-methyl-allylideneaminooxymethyl)-phenyl)-2-methoxyimino-N methylacetamide;
oxazolidinediones and imidazolinones: famoxadone, fenamidone;
F.I-2) Inhibitors of complex II (e.g. carboxamides):
carboxanilides: benodanil, benzovindiflupyr, bixafen, boscalid, carboxin, fenfuram, fenhexamid, fluopyram, flutolanil, furametpyr, isopyrazam, isotianil, mepronil, oxycarboxin, penflufen, penthiopyrad, sedaxane, tecloftalam, thifluzamide, tiadinil, 2-amino-4 methyl-thiazole-5-carboxanilide, N-(3′,4′,5′ trifluorobiphenyl-2 yl)-3-difluoromethyl-1-methyl-1H-pyrazole-4 carboxamide (fluxapyroxad), N-(4′-trifluoromethylthiobiphenyl-2-yl)-3 difluoromethyl-1-methyl-1H pyrazole-4-carboxamide, N-(2-(1,3,3-trimethyl-butyl)-phenyl)-1,3-dimethyl-5 fluoro-1H-pyrazole-4 carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 3-(difluoromethyl)-1,5-dimethyl-N-(1,1,3-trimethylindan-4-yOpyrazole-4-carboxamide, 1,3,5-trimethyl-N-(1,1,3-trimethylindan-4-yl)pyrazole-4-carboxamide, 3-(difluoromethyl)-1-methyl-N-(1,1,3-trimethylindan-4-yOpyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 3-(difluoromethyl)-1,5-dimethyl-N-(1,1,3-trimethylindan-4-yl)pyrazole-4-carboxamide, 1,3,5-trimethyl-N-(1,1,3-trimethylindan-4-yl)pyrazole-4-carboxamide;
F.I-3) Inhibitors of complex III at Qi site: cyazofamid, amisulbrom, [(3S,6S,7R,8R)-8-benzyl-3-[(3-acetoxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl]2-methylpropanoate, [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl]2-methylpropanoate, 3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate;
F.I-4) Other respiration inhibitors (complex I, uncouplers) diflumetorim; (5,8-difluoroquinazolin-4-yl)-{2-[2-fluoro-4-(4-trifluoromethylpyridin-2-yloxy)-phenyl]-ethyl}-amine; tecnazen; ametoctradin; silthiofam; nitrophenyl derivates: binapacryl, dinobuton, dinocap, fluazinam, ferimzone, nitrthalisopropyl,
and including organometal compounds: fentin salts, such as fentin-acetate, fentin chloride or fentin hydroxide;
F.II) Sterol biosynthesis inhibitors (SBI fungicides)
F.II-1) C14 demethylase inhibitors (DMI fungicides, e.g. triazoles, imidazoles) triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole, 1-[rel-(2S; 3R)-3-(2-chlorophenyl)-2-(2,4-difluorophenyl)-oxiranylmethyl]-5-thiocyanato-1H-[1,2,4]triazole, 2-[rel-(2S; 3R)-3-(2-chlorophenyl)-2-(2,4-difluorophenyl)-oxiranylmethyl]-2H-[1,2,4]triazole-3-thiol;
imidazoles: imazalil, pefurazoate, oxpoconazole, prochloraz, triflumizole;
pyrimidines, pyridines and piperazines: fenarimol, nuarimol, pyrifenox, triforine, 1-[rel-(2S; 3R)-3-(2-chlorophenyl)-2-(2,4-difluorophenyl)-oxiranylmethyl]-5-thiocyanato-1H-[1,2,4]triazole, 2-[rel-(2S; 3R)-3-(2-chlorophenyl)-2-(2,4-difluorophenyl)-oxiranylmethyl]-2H-[1,2,4]triazole-3-thiol;
F.II-2) Deltal-4-reductase inhitors (Amines, e.g. morpholines, piperidines) morpholines: aldimorph, dodemorph, dodemorph-acetate, fenpropimorph, tridemorph;
piperidines: fenpropidin, piperalin; spiroketalamines: spiroxamine;
F.II-3) Inhibitors of 3-keto reductase: hydroxyanilides: fenhexamid;
F.III) Nucleic acid synthesis inhibitors
F.III-1) RNA, DNA synthesis
phenylamides or acyl amino acid fungicides: benalaxyl, benalaxyl-M, kiralaxyl, metalaxyl, metalaxyl-M (mefenoxam), ofurace, oxadixyl;
isoxazoles and iosothiazolones: hymexazole, octhilinone;
F.III-2) DNA topisomerase inhibitors: oxolinic acid;
F.III-3) Nucleotide metabolism (e.g. adenosin-deaminase), hydroxy (2-amino)-pyrimidines: bupirimate;
F.IV) Inhibitors of cell division and or cytoskeleton
F.IV-1) Tubulin inhibitors: benzimidazoles and thiophanates: benomyl, carbendazim, fuberidazole, thiabendazole, thiophanate-methyl;
triazolopyrimidines: 5-chloro-7 (4-methylpiperidin-1-yl)-6-(2,4,6-trifluorophenyl)-[1,2,4]triazolo[1,5 a]pyrimidine;
F.IV-2) Other cell division inhibitors
benzamides and phenyl acetamides: diethofencarb, ethaboxam, pencycuron, fluopicolide, zoxamide;
F.IV-3) Actin inhibitors: benzophenones: metrafenone, pyriofenone;
F.V) Inhibitors of amino acid and protein synthesis
F.V-1) Methionine synthesis inhibitors (anilino-pyrimidines)
anilino-pyrimidines: cyprodinil, mepanipyrim, nitrapyrin, pyrimethanil;
F.V-2) Protein synthesis inhibitors (anilino-pyrimidines)
antibiotics: blasticidin-S, kasugamycin, kasugamycin hydrochloride-hydrate, mildiomycin, streptomycin, oxytetracyclin, polyoxine, validamycin A;
F.VI) Signal transduction inhibitors
F.VI-1) MAP/Histidine kinase inhibitors (e.g. anilino-pyrimidines)
dicarboximides: fluoroimid, iprodione, procymidone, vinclozolin;
phenylpyrroles: fenpiclonil, fludioxonil;
F.VI-2) G protein inhibitors: quinolines: quinoxyfen;
F.VII) Lipid and membrane synthesis inhibitors
F.VII-1) Phospholipid biosynthesis inhibitors
organophosphorus compounds: edifenphos, iprobenfos, pyrazophos;
dithiolanes: isoprothiolane;
F.VII-2) Lipid peroxidation: aromatic hydrocarbons: dicloran, quintozene, tecnazene, tolclofosmethyl, biphenyl, chloroneb, etridiazole;
F.VII-3) Carboxyl acid amides (CAA fungicides)
cinnamic or mandelic acid amides: dimethomorph, flumorph, mandiproamid, pyrimorph; valinamide carbamates: benthiavalicarb, iprovalicarb, pyribencarb, valifenalate and N-(1-(1-(4-cyano-phenyl)ethanesulfonyl)-but-2-yl) carbamic acid-(4-fluorophenyl) ester;
F.VII-4) Compounds affecting cell membrane permeability and fatty acids:
1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, carbamates: propamocarb, propamocarbhydrochlorid,
   F.VII-5) fatty acid amide hydrolase inhibitors: 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone;
   F.VIII) Inhibitors with Multi Site Action
   F.VIII-1) Inorganic active substances: Bordeaux mixture, copper acetate, copper hydroxide, copper oxychloride, basic copper sulfate, sulfur;
   F.VIII-2) Thio- and dithiocarbamates: ferbam, mancozeb, maneb, metam, methasulphocarb, metiram, propineb, thiram, zineb, ziram;
   F.VIII-3) Organochlorine compounds (e.g. phthalimides, sulfamides, chloronitriles): anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitrophenyl)-N-ethyl-4-methyl-benzenesulfonamide;
   F.VIII-4) Guanidines and other: guanidine, dodine, dodine free base, guazatine, guazatineacetate, iminoctadine, iminoctadine-triacetate, iminoctadine-tris(albesilate), 2,6-dimethyl-1H,5H-[1,4]dithiino[2,3-c:5,6-c′]dipyrrole-1,3,5,7(2H,6H)-tetraone;
   F.VIII-5) Ahtraquinones: dithianon;
   F.IX) Cell wall synthesis inhibitors
   F.IX-1) Inhibitors of glucan synthesis: validamycin, polyoxin B;
   F.IX-2) Melanin synthesis inhibitors: pyroquilon, tricyclazole, carpropamide, dicyclomet, fenoxanil;
   F.X) Plant defence inducers
   F.X-1) Salicylic acid pathway: acibenzolar-5-methyl;
   F.X-2) Others: probenazole, isotianil, tiadinil, prohexadione-calcium;
   phosphonates: fosetyl, fosetyl-aluminum, phosphorous acid and its salts;
   F.XI) Unknown mode of action: bronopol, chinomethionat, cyflufenamid, cymoxanil, dazomet, debacarb, diclomezine, difenzoquat, difenzoquat-methylsulfate, diphenylamin, fenpyrazamine, flumetover, flusulfamide, flutianil, methasulfocarb, nitrapyrin, nitrothal-isopropyl, oxathiapiprolin, oxin-copper, proquinazid, tebufloquin, tecloftalam, triazoxide, 2-butoxy-6-iodo-3-propylchromen-4-one, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N methyl formamidine, N′ (4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2 methyl-4-(3-trimethylsilanyl-propoxy)-phenyl)-Nethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl(h)-1,2,3,4-tetrahydro-naphthalen-1-yl-amide, methoxy-acetic acid 6-tert-butyl-8-fluoro-2,3-dimethyl-quinolin-4-yl ester and N-Methyl-2-{1-[(5-methyl-3-trifluoromethyl-1H-pyrazol-1-yl)acetyl]-piperidin-4-yl}-N-[(1R)-1,2,3,4-tetrahydronaphthalen-1-yl]-4-thiazolecarboxamide, 3-[5-(4-chloro-phenyl)-2,3-dimethyl-isoxazolidin-3 yl]-pyridine, pyrisoxazole, 5-amino-2-isopropyl-3-oxo-4-ortho-tolyl-2,3-dihydro-pyrazole-1 carbothioic acid S-allyl ester, N-(6-methoxy-pyridin-3-yl)cyclopropanecarboxylic acid amide, 5-chloro-1 (4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide.
   F.XII) Growth regulators: abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N 6-benzyladenine, paclobutrazol, prohexadione (prohexadione-calcium), prohydrojasmon, thidiazuron, triapenthenol, tributyl phosphorotrithioate, 2,3,5 tri iodobenzoic acid, trinexapac-ethyl and uniconazole;
   F.XIII) Biological control agents
   Ampelomyces quisqualis (e.g. AQ 10® from Intrachem Bio GmbH & Co. KG, Germany), Aspergillus flavus (e.g. AFLAGUARD® from Syngenta, CH), Aureobasidium pullulans (e.g. BOTECTOR® from bio-ferm GmbH, Germany), Bacillus pumilus (e.g. NRRL Accession No. B-30087 in SONATA® and BALLAD® Plus from AgraQuest Inc., USA), Bacillus subtilis (e.g. isolate NRRL-Nr. B-21661 in RHAPSODY®, SERENADE® MAX and SERENADE® ASO from AgraQuest Inc., USA), Bacillus subtilis var. amyloliquefaciens FZB24 (e.g. TAEGRO® from Novozyme Biologicals, Inc., USA), Candida oleophila 1-82 (e.g. ASPIRE® from Ecogen Inc., USA), Candida saitoana (e.g. BIOCURE® (in mixture with lysozyme) and BIOCOAT® from Micro Flo Company, USA (BASF SE) and Arysta), Chitosan (e.g. ARMOUR-ZEN from BotriZen Ltd., NZ), Clonostachys rosea f. catenulata, also named Gliocladium catenulatum (e.g. isolate J1446: PRESTOP® from Verdera, Finland), Coniothyrium minitans (e.g. CONTANS® from Prophyta, Germany), Cryphonectria parasitica (e.g. Endothia parasitica from CNICM, France), Cryptococcus albidus (e.g. YIELD PLUS® from Anchor Bio-Technologies, South Africa), Fusarium oxysporum (e.g. BIOFOX® from S.I.A.P.A., Italy, FUSACLEAN® from Natural Plant Protection, France), Metschnikowia fructicola (e.g. SHEMER® from Agrogreen, Israel), Microdochium dimerum (e.g. ANTIBOT® from Agrauxine, France), Phlebiopsis gigantea (e.g. ROTSOP® from Verdera, Finland), Pseudozyma flocculosa (e.g. SPORODEX® from Plant Products Co. Ltd., Canada), Pythium oligandrum DV74 (e.g. POLYVERSUM® from Remeslo SSRO, Biopreparaty, Czech Rep.), Reynoutria sachlinensis (e.g. REGALIA® from Marrone Biolnnovations, USA), Talaromyces flavus V117b (e.g. PROTUS® from Prophyta, Germany), Trichoderma asperellum SKT-1 (e.g. ECO—HOPE® from Kumiai Chemical Industry Co., Ltd., Japan), T. atroviride LC52 (e.g. SENTINEL® from Agrimm Technologies Ltd, NZ), T. harzianum T-22 (e.g. PLANTSHIELD® der Firma BioWorks Inc., USA), T. harzianum TH 35 (e.g. ROOT PRO® from Mycontrol Ltd., Israel), T. harzianum T-39 (e.g. TRICHODEX® and TRICHODERMA 2000® from Mycontrol Ltd., Israel and Makhteshim Ltd., Israel), T. harzianum and T. viride (e.g. TRICHOPEL from Agrimm Technologies Ltd, NZ), T. harzianum ICC012 and T. viride ICC080 (e.g. REMEDIER® WP from Is agro Ricerca, Italy), T. polysporum and T. harzianum (e.g. BINAB® from BINAB Bio-Innovation AB, Sweden), T. stromaticum (e.g. TRICOVAB® from C.E.P.L.A.C., Brazil), T. virens GL-21 (e.g. SOILGARD® from Certis LLC, USA), T. viride (e.g. TRIECO® from Ecosense Labs. (India) Pvt. Ltd., Indien, BIO-CURE® F from T. Stanes & Co. Ltd., Indien), T. viride TV1 (e.g. T. viride TV1 from Agribiotec srl, Italy), Ulocladium oudemansii HRU3 (e.g. BOTRY-ZEN® from Botry-Zen Ltd, NZ).

The commercially available compounds II of the group F listed above may be found in The Pesticide Manual, 15th Edition, C. D. S. Tomlin, British Crop Protection Council (2011) among other publications. Their preparation and their activity against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EPA 141 317; EP-A 152 031; EP-A 226 917; EPA 243 970; EPA 256 503; EP-A 428 941; EP-A 532 022; EP-A 1 028 125; EP-A 1 035 122; EPA 1 201 648; EPA 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028,657).

### Applications

The animal pest, i.e. the insects, arachnids and nematodes, the plant, soil or water in which the plant is growing can be contacted with the present compounds of formula I or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

The compounds of formula I or the pesticidal compositions comprising them may be used to protect growing plants and crops from attack or infestation by animal pests, especially insects, acaridae or arachnids by contacting the plant/crop with a pesticidally effective amount of compounds of formula I. The term “crop” refers both to growing and harvested crops.

The compounds of the present invention and the compositions comprising them are particularly important in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The compounds of the present invention are employed as such or in form of compositions by treating the insects or the plants, plant propagation materials, such as seeds, soil, surfaces, materials or rooms to be protected from insecticidal attack with a insecticidally effective amount of the active compounds. The application can be carried out both before and after the infection of the plants, plant propagation materials, such as seeds, soil, surfaces, materials or rooms by the insects.

The present invention also includes a method of combating animal pests which comprises contacting the animal pests, their habit, breeding ground, food supply, cultivated plants, seed, soil, area, material or environment in which the animal pests are growing or may grow, or the materials, plants, seeds, soils, surfaces or spaces to be protected from animal attack or infestation with a pesticidally effective amount of a mixture of at least one active compound I.

Moreover, animal pests may be controlled by contacting the target pest, its food supply, habitat, breeding ground or its locus with a pesticidally effective amount of compounds of formula I. As such, the application may be carried out before or after the infection of the locus, growing crops, or harvested crops by the pest.

The compounds of the invention can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “cultivated plants” is to be understood as including plants which have been modified by breeding, mutagenesis or genetic engineering. Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transtional modification of protein(s) (oligo- or polypeptides) poly for example by glycosylation or polymer additions such as prenylated, acetylated or farnesylated moieties or PEG moieties (e.g. as disclosed in Biotechnol Prog. 2001 July-August; 17(4):720-8, Protein Eng Des Sel. 2004 January; 17(1):57-66, Nat. Protoc. 2007; 2(5):1225-35, Curr Opin Chem. Biol. 2006 October; 10(5):487-91. Epub 2006 Aug. 28, Biomaterials. 2001 March; 22(5):405-17, Bioconjug Chem. 2005 January-February; 16(1):113-21).

The term “cultivated plants” is to be understood also including plants that have been rendered tolerant to applications of specific classes of herbicides, such as hydroxy-phenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A-0242236, EP-A-242246) or oxynil herbicides (see e.g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e.g. imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” is to be understood also including plants that are by the use of recombinant DNA techniques capable to synthesize one or more insecticidal proteins, especially those known from the bacterial genus Bacillus, particularly from Bacillus thuringiensis, such as ä-endotoxins, e.g. CryIA(b), CryIA(c), CryIF, CryIF(a2), CryIIA(b), CryIIIA, CrylIIB(b1) or Cry9c; vegetative insecticidal proteins (VIP), e.g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insectspecific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are dis-closed, for example, in EP-A 374 753, WO 93/007278, WO 95/34656, EP-A 427 529, EP-A 451 878, WO 03/018810 and WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxonomic groups of arthropods, particularly to beetles (Coleoptera), flies (Diptera), and butterflies and moths (Lepidoptera) and to plant parasitic nematodes (Nematoda).

The term “cultivated plants” is to be understood also including plants that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, for example EP-A 0 392 225), plant disease resistance genes (for example potato cultivars, which express resistance genes acting against Phytophthora infestans derived from the mexican wild potato Solanum bulbocastanum) or T4-lyso-zym (e.g. potato cultivars capable of synthesizing these proteins with increased resistance against bacteria such as Erwinia amylvora). The methods for producing such genetically modified plants are generally known to the person skilled in the art and are described, for example, in the publications mentioned above.

The term “cultivated plants” is to be understood also including plants that are by the use of recombinant DNA techniques capable to synthesize one or more proteins to increase the productivity (e.g. bio mass production, grain yield, starch content, oil content or protein content), tolerance to drought, salinity or other growth-limiting environ-mental factors or tolerance to pests and fungal, bacterial or viral pathogens of those plants.

The term “cultivated plants” is to be understood also including plants that contain by the use of recombinant DNA techniques a modified amount of substances of content or new substances of content, specifically to improve human or animal nutrition, for example oil crops that produce health-promoting long-chain omega-3 fatty acids or unsaturated omega-9 fatty acids (e.g. Nexera® rape).

The term “cultivated plants” is to be understood also including plants that contain by the use of recombinant DNA techniques a modified amount of substances of content or new substances of content, specifically to improve raw material production, for example potatoes that produce increased amounts of amylopectin (e.g. Amflora® potato).

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in treating crop plants, the rate of application of the active ingredients of this invention may be in the range of 0.1 g to 4000 g per hectare, desirably from 25 g to 600 g per hectare, more desirably from 50 g to 500 g per hectare.

The compounds of formula I are effective through both contact (via soil, glass, wall, bed net, carpet, plant parts or animal parts), and ingestion (bait, or plant part).

The compounds of the invention may also be applied against non-crop insect pests, such as ants, termites, wasps, flies, mosquitos, crickets, or cockroaches. For use against said non-crop pests, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active compound.

Formulations of compounds of formula I as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250® C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of formula I and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula I and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of formula I and its compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

### Seed Treatment

The compounds of formula I are also suitable for the treatment of seeds in order to protect the seed from insect pest, in particular from soil-living insect pests and the resulting plant's roots and shoots against soil pests and foliar insects.

The compounds of formula I are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedling's roots and shoots from insects, in particular from soil and foliar insects, said method comprising contacting the seeds before sowing and/or after pregermination with a compound of the general formula I or a salt thereof. Particularly preferred is a method, wherein the plant's roots and shoots are protected, more preferably a method, wherein the plants shoots are protected form piercing and sucking insects, most preferably aa method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A-0242236, EP-A-242246) (WO 92/00377) (EP-A-0257993, U.S. Pat. No. 5,013,659) or in transgenic crop plants, for example cotton, with the capability of producing Bacillus thuringiensis toxins (Bt toxins) which make the plants resistant to certain pests (EP-A-0142924, EP-A-0193259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)

### H Gel-Formulations (GF)

I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typcially, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of compounds of formula I for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, polyesters, polystyrene homo and copolymers

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®)

In the treatment of seed, the application rates of the compounds I are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed.

The invention therefore also relates to seed comprising a compound of the formula I, or an agriculturally useful salt of I, as defined herein. The amount of the compound I or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

### Animal Health

The compounds of formula I or the enantiomers or veterinarily acceptable salts thereof are in particular also suitable for being used for combating parasites in and on animals.

An object of the present invention is therfore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of compounds of formula I or the enantiomers or veterinarily acceptable salts thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula I or the enantiomers or veterinarily acceptable salts thereof or a composition comprising it.

The invention also provides a process for the preparation of a composition for treating, controlling, preventing or protecting animals against infestation or infection by parasites which comprises a parasiticidally effective amount of a compound of formula I or the enantiomers or veterinarily acceptable salts thereof or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula I are suitable for combating endo- and ectoparasites in and on animals.

Compounds of formula I or the enantiomers or veterinarily acceptable salts thereof and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula I or the enantiomers or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I or the enantiomers or veterinarily acceptable salts thereof and compositions comprising them are suitable for systemic and/or non-systemic control of ectoand/or endoparasites. They are active against all or some stages of development.

The compounds of formula I are especially useful for combating ectoparasites.

The compounds of formula I are especially useful for combating parasites of the following orders and species, respectively:

fleas (Siphonaptera), e.g. Ctenocephalides fells, Ctenocephalides canis, Xenopsylla cheopis, Pulex irritans, Tunga penetrans, and Nosopsyllus fasciatus,
cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
flies, mosquitoes (Diptera), e.g. Aedes aegypti, Aedes albopictus, Aedes vexans, Anastrepha ludens, Anopheles maculipennis, Anopheles crucians, Anopheles albirnanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliompa hominivorax, Cordylobia anthropophaga, Culicoides furens, Culex pipiens, Culex nigripalpus, Culex quinquefasciatus, Culex tarsalis, Culiseta inornata, Culliseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosirnulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Amblyomma maculatum, Ornithodorus hermsi, Ornithodorus turicata and parasitic mites (Mesostigmata), e.g. Ornithonyssus bacoti and Dermanyssus gallinae,
Actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,
Bugs (Heteropterida): Cimex lectularius, Cimex hemipterus, Reduvius sendis, Triatoma spp., Rhodnius ssp., Panstrongylus ssp. and Arilus critatus,
Anoplurida, e.g. Haematopinus spp., Linognathus spp., Pediculus spp., Phitirus spp., and Solenopotes spp,
Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Fellcola spp,

### Roundworms Nematoda:

Wipeworms and Trichinosis (Trichosyringida), e.g. Trichinellidae (Trichinella spp.), (Trichuridae) Trichuris spp., Capillaria spp,
Rhabditida, e.g. Rhabditis spp, Strongyloides spp., Hekephalobus spp,
Strongylida, e.g. Strongylus spp., Ancylostoma spp., Necator americanus, Bunostomum spp. (Hookworm), Trichostrongylus spp., Haemonchus contortus, Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinana spp., Globocephalus spp., Necator spp., Metastrongylus spp., Muellerius capiliaris, Protostrongylus spp., Angiostrongylus spp., Parelaphostrongylus spp. Aleurostrongylus abstrusus, and Dioctophyma resale,
Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,
Camallanida, e.g. Dracunculus medinensis (guinea worm)
Spirurida, e.g. Thelazia spp. Wuchereria spp., Brugia spp., Onchocerca spp., Dirofilari spp. a, Dipetalonema spp., Setaria spp., Elaeophora spp., Spirocerca lupi, and Habronema spp.,
Thorny headed worms (Acanthocephala), e.g. Acanthocephalus spp., Macracanthorhynchus hirudinaceus and Oncicola spp,

### Planarians (Plathelminthes):

Flukes (Trematoda), e.g. Faciola spp., Fascioloides magna, Paragonimus spp., Dicrocoelium spp., Fasciolopsis busk, Clonorchis sinensis, Schistosoma spp., Trichobilharzia spp., Alaria alata, Paragonimus spp., and Nanocyetes spp,
Cercomeromorpha, in particular Cestoda (Tapeworms), e.g. Diphyllobothrium spp., Tema spp., Echinococcus spp., Dipyldium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula I and compositions containing them are particularly useful for the control of pests from the orders Diptera, Siphonaptera and Ixodida.

Moreover, the use of the compounds of formula I and compositions containing them for combating mosquitoes is especially preferred.

The use of the compounds of formula I and compositions containing them for combating flies is a further preferred embodiment of the present invention.

Furthermore, the use of the compounds of formula I and compositions containing them for combating fleas is especially preferred.

The use of the compounds of formula I and compositions containing them for combating ticks is a further preferred embodiment of the present invention.

The compounds of formula I also are especially useful for combating endoparasites (roundworms nematoda, thorny headed worms and planarians).

Administration can be carried out both prophylactically and therapeutically.

Administration of the active compounds is carried out directly or in the form of suitable preparations, orally, topically/dermally or parenterally.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-inwater or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methyl-pyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and nbutanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions which can be used in the invention can comprise generally from about 0.001 to 95% of the compound of formula I.

Generally it is favorable to apply the compounds of formula I in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

In a preferred embodiment of the present invention, the compositions comprising the compounds of formula I them are applied dermally/topically.

In a further preferred embodiment, the topical application is conducted in the form of compound-containing shaped articles such as collars, medallions, ear tags, bands for fixing at body parts, and adhesive strips and foils.

Generally it is favorable to apply solid formulations which release compounds of formula I in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula I. A detailed list of plastics and elastomers as well as preparation procedures for the shaped articles is given e.g. in WO 03/086075.

### EXAMPLES

The present invention is now illustrated in further details by the following examples, without imposing any limitation thereto.

Compounds can in general be characterized e.g. by coupled High Performance Liquid Chromatography/mass spectrometry (HPLC/MS), by ¹H-NMR and/or by their melting points.

Analytical HPLC column 1: RP-18 column Chromolith Speed ROD from Merck KgaA, Germany).

Elution: acetonitrile+0.1% trifluoroacetic acid (TFA)/water+0.1% trifluoroacetic acid (TFA) in a ratio of from 5:95 to 95:5 in 5 minutes at 40° C.

RT or r.t. ═HPLC retention time; m/z of the [M+H]⁺, [M+Na]+ or [M+K]+ peaks.

Analytical HPLC column 2: Phenomenex Kinetex 1.7 μm XB-C18 100A; 50×2.1 mm

Elution: A: acetonitrile+0.1% trifluoroacetic acid (TFA)/water+0.1% trifluoroacetic acid (TFA) in a ratio of from 5:95 to 95:5 in 1.5 minutes at 50® C.

RT or r.t. ═HPLC retention time; m/z of the [M+H]⁺, [M+Na]+ or [M+K]+ peaks.

¹H-NMR, respectively ¹³C-NMR: The signals are characterized by chemical shift (ppm) vs. tetramethylsilane, respectively CDCl₃ for ¹³C-NMR, by their multiplicity and by their integral (relative number of hydrogen atoms given). The following abbreviations are used to characterize the multiplicity of the signals: m=multiplett, q=quartett, t=triplett, d=doublet and s=singulett.

### S. Synthesis Example

### S.1. Synthesis of N-(4,5-dihydrothiazol-2-yl)-N-ethyl-2-(3-pyridyl)thiazol-5-amine (Compound C.1)

### Step 1:

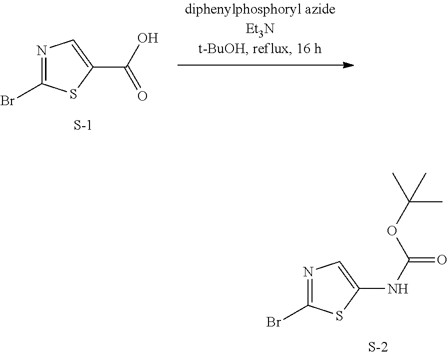

To a stirred solution of S-1 (15 g, 0.072 mol) in t-butanol (80 mL) was added Et₃N (9.9 mL, 0.08 mol). After 10 min, diphenylphosphoryl azide (17 mL, 0.072 mol) was added and the reaction was heated at 80° C. for 16 h. The reaction mixture was then concentrated, diluted with water (200 mL) and extracted with ethyl acetate (2×200 mL). The combined organic layers were dried over anhydrous sodium sulphate, filtered and concentrated to afford 16 g of crude product. The crude product was purified by flash column chromatography (Silica gel: 100-200 mesh) eluting with 20% ethyl acetate in hexane to afford 12 g of S-2 in 60% yield as yellow colored solid.

¹H NMR (400 MHz, DMSO-d6): δ 1.4 (s, 9H), 7.1 (s, 1H), 10.9 (s, 1H) ppm.

### Step 2:

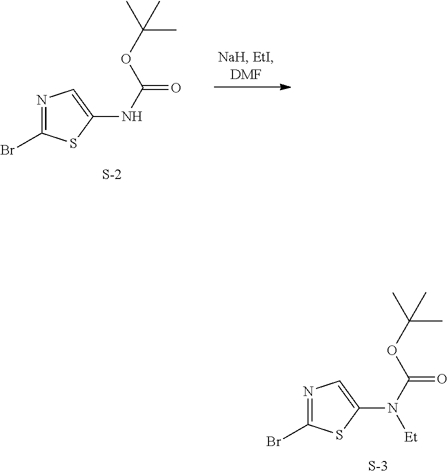

To a stirred solution of S-2 (12 g, 0.043 mol) in DMF (100 mL) was added 60% NaH (2.5 g, 0.0647 mol) at 0° C. stirred for 50 min. After 50 min, ethyl iodide (10.1 g, 0.0647 mol) was slowly added and the reaction was allowed to warm to rt and stirred for 4 h. The reaction mixture was then diluted with ice water (500 mL) and extracted with ethyl acetate (100 mL). The organic layer and dried over anhydrous sodium sulphate, filtered and concentrated to afford 12 g of crude product. The crude product was purified by flash column chromatography (Silica gel: 100-200 mesh) eluting with 20% ethyl acetate in hexane to afford 10 g of S-3 in 77% yield as yellow colored solid.

¹H NMR (400 MHz, DMSO-d6): δ 1.16 (t, J=6.8 Hz, 3H), 1.5 (s, 9H), 3.80 (q, J=6.8 Hz, 2H), 7.4 (s, 1H) ppm.

### Step 3:

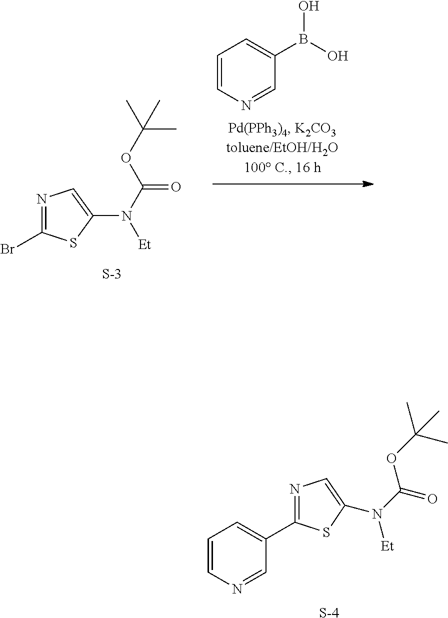

A stirred solution of S-3 (4.5 g, 0.0147 mol), pyridine-3-boronic acid (2.3 g, 0.016 mol) and aq. K₂CO₃ (5 g in 5 mL of H₂O, 0.036 mol) in a mixture of toluene (30 mL) and ethanol (30 mL) was degassed with argon for 30 min. After 30 min, Pd(PPh₃)₄ (160 mg, 0.00014 mol) was added at rt and the reaction mixture was heated at 110° C. for 16 h. The reaction mixture was then filtered through a Celite pad and the filtrate was concentrated under reduced pressure. The residue was then diluted with water (50 mL) and extracted with ethyl acetate (100 mL). The organic layer was dried over anhydrous sodium sulphate, filtered and concentrated to afford 4.5 g of crude product. The crude product was purified by flash column chromatography (Silica gel: 100-200 mesh) eluting with 20% ethyl acetate in petroleum ether to afford 2.5 g of S-4 in 56% yield as yellow coloured solid.

¹H NMR (400 MHz, DMSO-d6): δ 1.22 (t, J=7.2 Hz, 3H), 3.87-3.85 (q, 2H), 7.51-7.46 (m, 1H), 7.70 (s, 1H), 8.22 (d, J=8 Hz, 1H), 8.60 (d, J=5.6 Hz, 1H), 9.05 (d, J=1.2 Hz, 1H).

### Step 4:

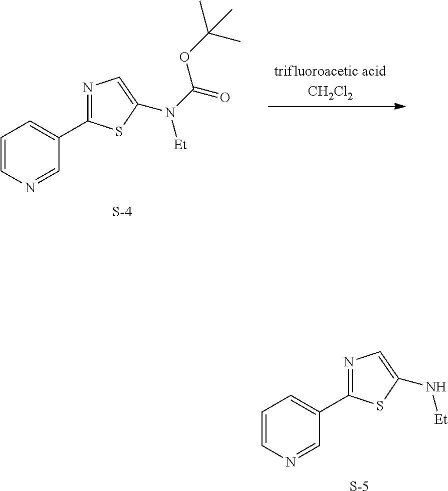

To a stirred solution of S-4 (2.5 g, 0.081 mol) in CH₂Cl₂ (10 mL) was added trifluoroacetic acid (5 mL) at 0° C. and the reaction mixture was warmed to rt and stirred for 2 h. The reaction mixture was then concentrated, the pH was adjusted to 9-10 with aq. Na₂CO₃ (20 mL) and extracted with ethyl acetate (100 mL). The organic layer was separated, dried over anhydrous

Na₂SO₄ and concentrated under reduced pressure to afford 1.5 g of S-5 in 94% yield as black coloured liquid.

¹H NMR (400 MHz, DMSO-d6): δ 1.19 (t, J=7.2 Hz, 3H), 3.50-3.46 (q, J=7.6 Hz, 2H), 6.68 (t, J=5.2 Hz, 1H), 6.86 (s, 1H), 7.43-7.40 (m, 1H), 8.02 (d, J=8 Hz, 1H), 8.48 (d, J=3.6 Hz, 1H), 8.89 (d, J=1.2 Hz, 1H).

### Step 5:

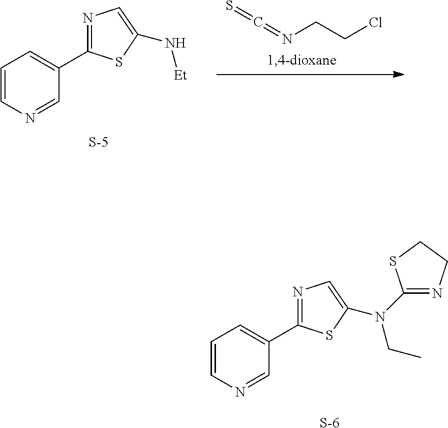

A stirred solution of S-5 (500 mg, 2.44 mmol), 1-chloro-2-isothiocyanatoethane (442 mg, 3.66 mmol) in 1,4-dioxane in a sealed tube was heated to 110° C. for 24 h. 1,4-dioxane was evaporated under reduced pressure to afford 500 mg of crude product. The crude product was then purified by flash column chromatography (Silica gel: 100-200 mesh) eluting the product with 100% EtOAc to afford 85 mg of S-6 (Compound C.1) in 12% yield as pale yellow coloured solid.

¹H NMR (400 MHz, DMSO-d6): δ 1.27 (t, J=7.2 Hz, 3H), 3.48 (t, J=7.6 Hz, 2H), 3.92 (q, J=7.2 Hz, 2H), 4.12 (t, J=8 Hz, 2H), 7.50-7.47 (m, 1H), 7.75 (s, 1H), 8.22-8.19 (m, 1H), 8.6-8.59 (dd, J=4.0, 1.6 Hz, 1H), 9.04 (d, J=1.6 Hz, 1H).

### S.2. Synthesis of 2-(3-Pyridyl)-5-(2-pyridylsulfanyl)thiazole (Compound C.5)

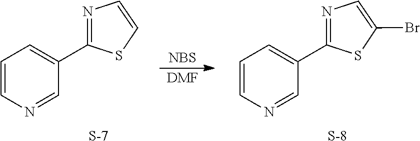

Step 1:

To a stirred solution of S-7 (2.5 g, 0.015 mol) in DMF (25 mL) was added NBS (4.1 g, 0.023 mol) at room temperature. Then the reaction mixture was heated to 60° C. and stirred for 6 h at 60° C. After cooling to room temperature the reaction mixture was diluted with ice water (100 mL), the formed solids were filtered through celite and the filtrate was extracted with ethyl acetate (3×100 mL). The combined organic layers were washed with water (2×100 mL), brine (100 mL), dried over anhydrous sodium sulfate, filtered and concentrated to afford 3 g of crude product. The crude product was purified by flash column chromatography (Silica gel: 100-200 mesh) eluting with 30% ethyl acetate in hexane to afford 1.4 g (38%) of S-8 as off white solid.

1H NMR (400 MHz, CDCl3): δ 9.09 (d, J=2.5 Hz, 1H), 8.68 (dd, J=4.8, 1.8 Hz, 1H), 8.17 (dt, J=7.9, 2.0 Hz, 1H), 7.80 (d, J=1.4 Hz, 1H), 7.40 (dd, J=8.1, 4.9 Hz, 1H).

### Step 2:

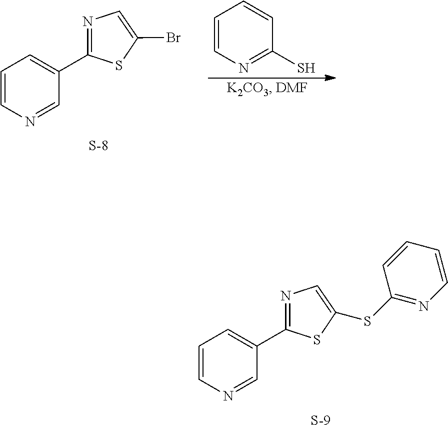

To a stirred solution of S-8 (1.0 g, 0.004 mol) in DMF (10 mL) was added pyridine-2-thiol (0.55 g, 0.005 mol) followed by K₂CO₃ (1.15 g, 0.0083 mol) at room temperature. Then the reaction mixture was heated to 100° C. and stirred for 16 h at 100° C. After cooling to room temperature the reaction mixture was diluted with ice water (100 mL) and extracted with ethyl acetate (3×100 mL). The combined organic layers were washed with water (2×100 mL), brine (100 mL), dried over anhydrous sodium sulfate, filtered and concentrated to afford 1 g of crude product. The crude product was purified by flash column chromatography (Silica gel: 100-200 mesh) eluting with 40% ethyl acetate in hexane to afford 0.45 g (40%) of S-9 (Compound C.5) as pale yellow solid.

1H NMR (400 MHz, CDCl3): δ 9.18 (d, J=2.3 Hz, 1H), 8.69 (dd, J=4.9, 1.7 Hz, 1H), 8.51-8.42 (m, 1H), 8.25 (dt, J=8.1, 2.0 Hz, 1H), 8.03 (s, 1H), 7.56 (td, J=7.8, 1.9 Hz, 1H), 7.41 (dd, J=8.0, 4.8 Hz, 1H), 7.17-7.04 (m, 2H).

### S.3 Synthesis of 4-Bromo-2-(3-pyridyl)-5-(2-pyridylsulfanyl)thiazole (Compound C.17)

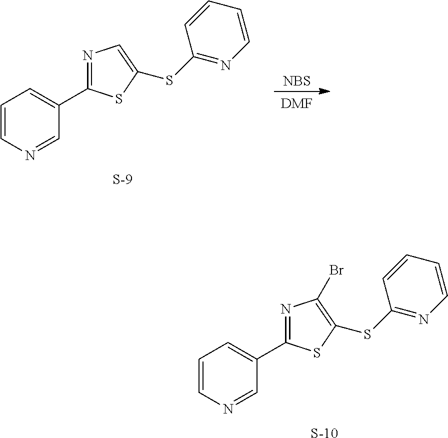

To a stirred solution of S-9 (0.4 g, 0.001 mol) in DMF (10 mL) was added NBS (0.39 g, 0.002 mol) at room temperature and stirred for 16 h at room temperature. Then the reaction mixture was diluted with ice water (100 mL), the precipitated solid was filtered and dried to afford 0.3 g of crude product. The crude product was purified by flash column chromatography (Silica gel: 100-200 mesh) eluting with 40% ethyl acetate in hexane to afford 0.11 g (24%) of S-10 (Compound C.17) as light brown solid.

1H NMR (400 MHz, CDCl3): δ 9.16 (s, 1H), 8.71 (s, 1H), 8.52-8.42 (m, 1H), 8.25 (dt, J=8.0, 1.9 Hz, 1H), 7.59 (td, J=7.8, 1.8 Hz, 1H), 7.42 (dd, J=8.0, 4.8 Hz, 1H), 7.18-7.06 (m, 2H).

### S.4 Synthesis of 4-Chloro-2-(3-pyridyl)-5-(2-pyridylsulfanyl)thiazole (Compound C.15)

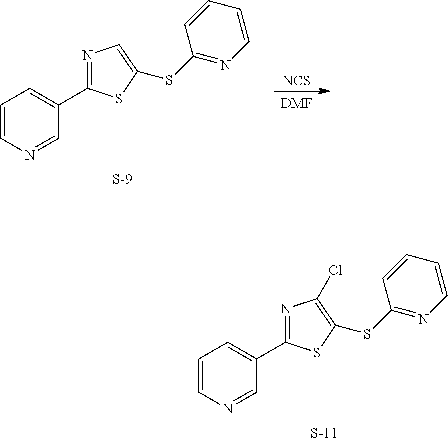

To a stirred solution of S-9 (0.33 g, 0.001 mol) in DMF (5 mL) was added NCS (0.24 g, 0.001 mol) at room temperature and stirred for 16 h at room temperature. Then the reaction mixture was diluted with ice water (50 mL), the precipitated solid was filtered and dried to afford 0.3 g of crude product. The crude product was purified by triturating with diethyl ether. The residue was dried to afford 0.17 g (45%) of S-11 (Compound C.15) as a light brown solid.

1H NMR (400 MHz, CDCl3): δ 9.16 (s, 1H), 8.71 (d, J=5.0 Hz, 1H), 8.50-8.42 (m, 1H), 8.26 (dt, J=8.1, 1.9 Hz, 1H), 7.59 (td, J=7.8, 1.9 Hz, 1H), 7.42 (dd, J=7.9, 4.8 Hz, 1H), 7.17-7.06 (m, 2H).

### S.5 Synthesis of 2-(3-Pyridyl)-5-(2-pyridylsulfinyl)thiazole (Compound C.6)

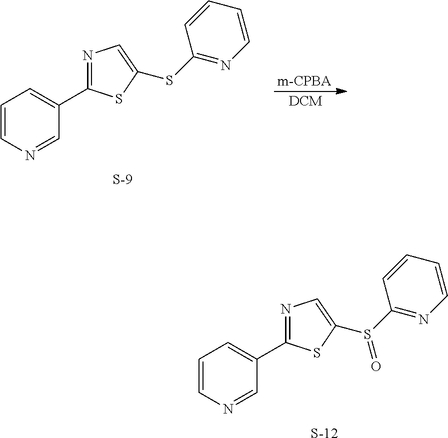

To a stirred solution of S-9 (0.5 g, 0.0018 mol) in DCM (10 mL) was added metachloroperoxybenzoic acid (0.27 g, 0.002 mol) at 0° C. and stirred for 2 h at room temperature. Then the reaction mixture was washed with saturated NaHCO₃ solution (25 mL), with water (25 mL), brine (25 mL), dried over anhydrous sodium sulfate, filtered and concentrated to afford 0.3 g of crude product. The crude product was purified by flash column chromatography (Silica gel: 100-200 mesh) eluting with 5% MeOH in DCM to afford 0.11 g (22%) of S-12 (Compound C.6) as a off white solid.

1H NMR (400 MHz, CDCl3): δ 9.13-9.08 (m, 1H), 8.71-8.60 (m, 2H), 8.34 (s, 1H), 8.18 (ddd, J=8.9, 3.3, 1.5 Hz, 2H), 8.01 (td, J=7.8, 1.7 Hz, 1H), 7.47-7.34 (m, 2H).

### S.6 Synthesis of 2-(3-Pyridyl)-5-(2-pyridylsulfonyl)thiazole (Compound C.7)

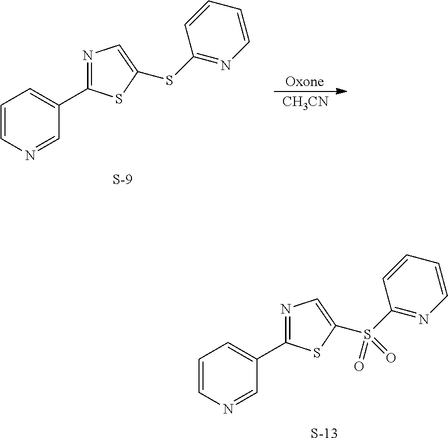

To a stirred solution of S-9 (0.5 g, 0.0018 mol) in acetonitrile (10 mL) and water (2 mL) was added Oxone (2.27 g, 0.0036 mol) followed by NaHCO₃ (0.07 g, 0.0009 mol) at 0° C. and stirred for 24 h at room temperature. Then the reaction mixture was filtered through celite and the filtrate was dried over anhydrous sodium sulphate, filtered and concentrated to afford 0.5 g of crude product. The crude product was purified by flash column chromatography (Silica gel: 100-200 mesh) eluting with 5% MeOH in DCM to afford 0.3 g of as off white solid. The solid was further purified by preparative HPLC to afford 0.17 g (30%) of S-13 (Compound C.7) as off white solid.

1H NMR (400 MHz, CDCl3): δ 9.13-9.08 (m, 1H), 8.71-8.60 (m, 2H), 8.34 (s, 1H), 8.18 (ddd, J=8.9, 3.3, 1.5 Hz, 2H), 8.01 (td, J=7.8, 1.7 Hz, 1H), 7.47-7.34 (m, 2H).

### S.7 Synthesis of 2-(3-Pyridyl)-5-(2-pyridylmethyl)thiazole (Compound C.19)

### Step 1:

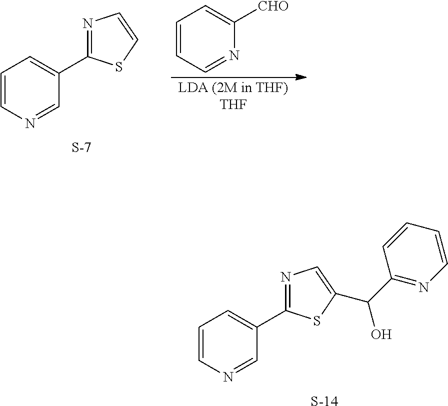

To a stirred solution of S-7 (6.0 g, 0.0370 mol) in dry THF (100 mL) at −78° C. was added LDA (36 mL, 0.0744 mol, 2M in THF) and the resulting reaction mixture was stirred for 30 min. Then Picolinaldehyde (6 g, 0.0555 moles) was added in one portion, the mixture was warmed to room temperature and then stirred for 2 h. Reaction progress was monitored by TLC. Reaction mixture was quenched with aq. NH₄Cl solution (100 mL) and extracted with ethyl acetate (200 mL). The organic layer was dried over anhydrous sodium sulfate, filtered and concentrated to afford 7.0 g of crude compound. The crude product was purified by flash column chromatography (Silica gel: 100-200 mesh) eluting with 2% Methanol in DCM to afford 4.0 g (40%) of S-14 as a brown liquid.

1H NMR (400 MHz, DMSO-d6): δ 9.07 (d, J=2.3 Hz, 1H), 8.73-8.58 (m, 1H), 8.54 (dd, J=4.6, 1.8 Hz, 1H), 8.24 (dt, J=7.9, 2.0 Hz, 1H), 7.96-7.76 (m, 2H), 7.65 (d, J=7.8 Hz, 1H), 7.50 (dd, J=8.0, 4.8 Hz, 1H), 7.32 (dd, J=7.5, 4.8 Hz, 1H), 6.68 (d, J=4.7 Hz, 1H), 6.07 (d, J=4.6 Hz, 1H)

### Step 2:

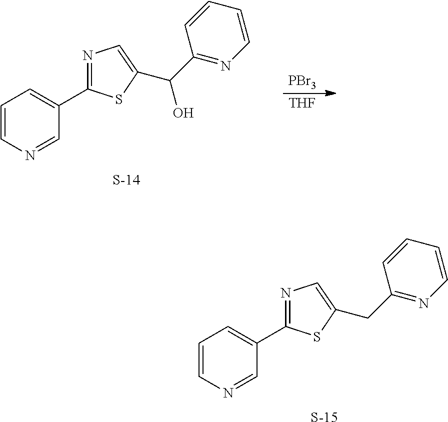

To a stirred solution of S-14 (500 mg, 0.00185 mol) in dry THF (30 mL) was added PBr₃ (10.9 mL, 0.00743 mol) at room temperature, then the reaction mixture was heated to refluxe for 16 h. The reaction mixture cooled and diluted with ice water (3 mL) and then pH was adjusted to ˜9-10 using aq. NaOH solution. The resulting mixture was extracted with ethyl acetate (50 mL). The organic layer was dried over anhydrous sodium sulfate, filtered and concentrated to afford 500 mg of crude compound. The crude product was purified by flash column chromatography (Silica gel: 100-200 mesh) eluting with 2% Methanol in DCM to afford 180 mg (38%) of S-15 (Compound C.19) as brown liquid.

1H NMR (400 MHz, CDCl3) δ 9.11 (d, J=2.4 Hz, 1H), 8.67-8.56 (m, 2H), 8.18 (dd, J=7.7, 2.0 Hz, 1H), 7.76 (s, 1H), 7.66 (td, J=7.6, 1.8 Hz, 1H), 7.35 (dd, J=7.9, 4.8 Hz, 1H), 7.29-7.23 (m, 1H), 7.19 (dd, J=7.5, 4.9 Hz, 1H), 4.38 (s, 2H).

### S.8 Synthesis of (E)-N-Methoxy-1-(2-pyridyl)-1-[2-(3-pyridyl)thiazol-5-yl]methanimine and (Z)—N-methoxy-1-(2-pyridyl)-1-[2-(3-pyridyl)thiazol-5-yl]methanimine (Compound C.20)

### Step 1:

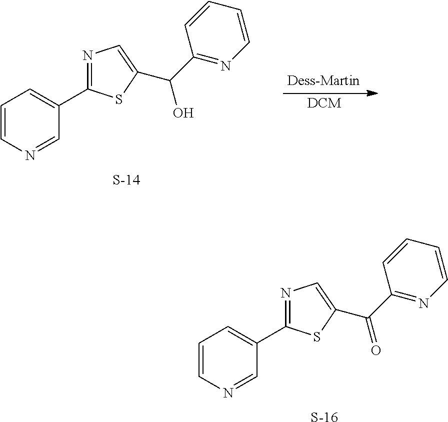

To a stirred solution of S-14 (2 g, 0.0074 mol) in DCM (50 mL) was added Dess-Martin periodinane (4.7 g, 0.011 mol) at 0° C. and then the reaction mixture was stirred at room temperature for 3 h. The reaction mixture was diluted with sat. NaHCO₃ solution (20 mL) and extracted with DCM (60 mL). The organic layer was dried over anhydrous sodium sulfate, filtered and concentrated to afford 1.5 g of crude material. The crude product was purified by flash column chromatography (Silica gel: 100-200 mesh) eluting with 1% Methanol in DCM to afford 1.0 g (54%) of S-16 as brown solid.

1H NMR (400 MHz, CDCl3) δ 9.32 (d, J=2.2 Hz, 1H), 9.08 (d, J=0.9 Hz, 1H), 8.81 (dd, J=4.7, 1.2 Hz, 1H), 8.73 (dd, J=4.8, 1.5 Hz, 1H), 8.39 (dt, J=8.2, 2.0 Hz, 1H), 8.26 (dt, J=7.9, 1.1 Hz, 1H), 7.95 (td, J=7.7, 1.6 Hz, 1H), 7.58 (ddd, J=7.4, 4.7, 1.2 Hz, 1H), 7.44 (dd, J=8.0, 4.8 Hz, 1H).

### Step 2:

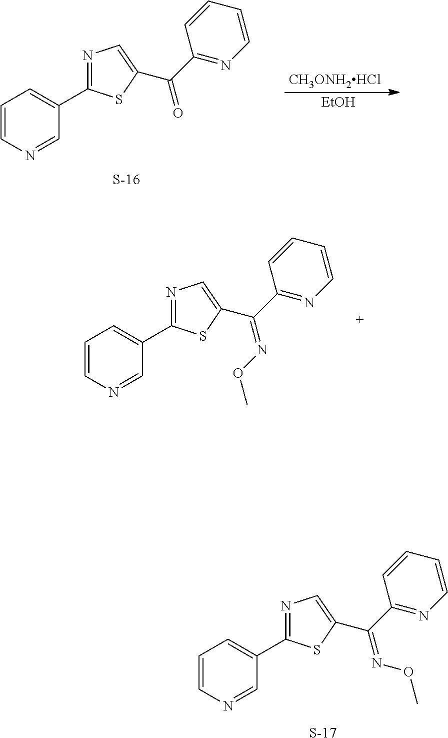

To a stirred solution of S-16 (300 mg, 0.0011 mol) in EtOH (10 mL) was added CH₃₀NH₂.HCl (217 mg, 0.00262 mol) at room temperature, then the reaction mixture was refluxed for 3 h. Reaction progress was monitored by TLC. Reaction mixture was concentrated under reduced pressure and the residue was diluted with DCM (30 mL) and washed with sat. NaHCO₃ (10 mL) solution. The organic layer was dried over anhydrous sodium sulfate, filtered and concentrated to afford 250 mg of crude compound. The solid was washed with diethyl ether (15 mL), filtered and dried to afford 130 mg of S-17 (Compound C.20) as brown solid (mixture of E&Z-isomers, in a ratio of about 1:3).

1H NMR (400 MHz, DMSO-d6): δ 9.19 (dd, J=13.7, 2.2 Hz, 1H), 8.80-8.61 (m, 2H), 8.51 (s, 1H), 8.43-8.28 (m, 1H), 8.05-7.93 (m, 1H), 7.82 (d, J=7.9 Hz, 1H), 7.63-7.51 (m, 2H), 4.19 (s, 3H), 3.93 (s, 1H).

### C. Compound Examples

Compound examples of the present invention are characterized with their ¹H NMR below:

### C.1 N-(4,5-dihydrothiazol-2-yl)-N-ethyl-2-(3-pyridyl)thiazol-5-amine

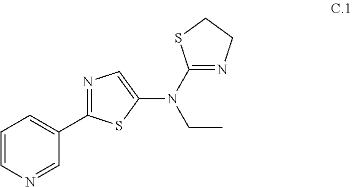 [SMILES: CCN(C1=NCCS1)c1cnc(-c2cccnc2)s1]

¹H NMR (400 MHz, DMSO-d6): δ 1.27 (t, J=7.2 Hz, 3H), 3.48 (t, J=7.6 Hz, 2H), 3.92 (q, J=7.2 Hz, 2H), 4.12 (t, J=8 Hz, 2H), 7.50-7.47 (m, 1H), 7.75 (s, 1H), 8.22-8.19 (m, 1H), 8.6-8.59 (dd, J=4.0, 1.6 Hz, 1H), 9.04 (d, J=1.6 Hz, 1H).

### C.2 N-ethyl-N-[2-(3-pyridyl)thiazol-5-yl]-4,5-dihydrooxazol-2-amine

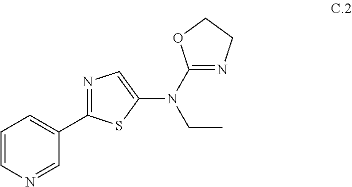 [SMILES: CCN(C1=NCCO1)c1cnc(-c2cccnc2)s1]

1H NMR (400 MHz, DMSO-d6): δ 1.27 (t, J=7.1 Hz, 3H), 3.85 (t, J=8.8 Hz, 2H), 3.90 (q, J=6.6 Hz, 2H), 4.46 (t, J=8.9 Hz, 2H), 7.48 (dd, J=8.0, 4.9 Hz, 1H), 7.68 (s, 1H), 8.22-8.19 (d, J=4.4 Hz, 1H), 8.59-8.58 (dd, J=4.9, 1.4 Hz, 1H), 9.03 (d, J=1.7 Hz, 1H).

### C.3 4-chloro-N-(4,5-dihydrothiazol-2-yl)-N-ethyl-2-(3-pyridyl)thiazol-5-amine

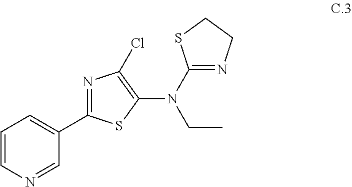 [SMILES: CCN(C1=NCCS1)c1sc(-c2cccnc2)nc1Cl]

1H NMR (400 MHz, DMSO-d6): δ 1.16 (t, J=7.1 Hz, 3H), 3.39 (t, J=7.7 Hz, 2H), 3.66 (q, J=7.3 Hz, 2H), 4.04 (t, J=7.7 Hz, 2H), 7.57 (dd, J=7.5, 2.7 Hz, 1H), 8.32-8.29 (d, J=8.4 Hz, 1H), 8.74-8.72 (dd, J=4.6, 1.6 Hz, 1H), 9.10 (d, J=2.0 Hz, 1H).

### C.4 N-[4-chloro-2-(3-pyridyl)thiazol-5-yl]-N-ethyl-4,5-dihydrooxazol-2-amine

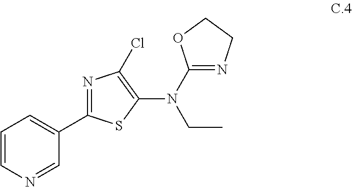 [SMILES: CCN(C1=NCCO1)c1sc(-c2cccnc2)nc1Cl]

1H NMR (400 MHz, DMSO-d6): δ 1.16 (t, J=7.0 Hz, 3H), 3.68 (q, J=7.1 Hz, 2H), 3.73 (t, J=8.6 Hz, 2H), 4.34 (t, J=8.8 Hz, 2H), 7.56 (ddd, J=5.5, 4.8, 0.6 Hz, 1H), 8.29-8.26 (ddd, J=4.0, 2.2, 1.8 Hz, 1H), 8.72-8.70 (dd, J=4.6, 1.5 Hz, 1H), 9.08 (d, J=1.7 Hz, 1H).

### C.5 2-(3-Pyridyl)-5-(2-pyridylsulfanyl)thiazole

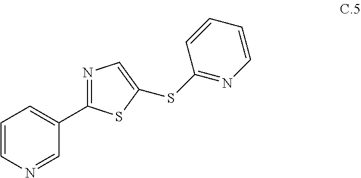 [SMILES: c1ccc(Sc2cnc(-c3cccnc3)s2)nc1]

1H NMR (400 MHz, CDCl3): δ 9.18 (d, J=2.3 Hz, 1H), 8.69 (dd, J=4.9, 1.7 Hz, 1H), 8.51-8.42 (m, 1H), 8.25 (dt, J=8.1, 2.0 Hz, 1H), 8.03 (s, 1H), 7.56 (td, J=7.8, 1.9 Hz, 1H), 7.41 (dd, J=8.0, 4.8 Hz, 1H), 7.17-7.04 (m, 2H).

### C.6 2-(3-Pyridyl)-5-(2-pyridylsulfinyl)thiazole

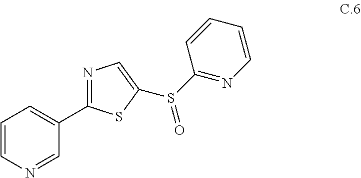 [SMILES: O=S(c1ccccn1)c1cnc(-c2cccnc2)s1]

1H NMR (400 MHz, CDCl3): δ 9.13-9.08 (m, 1H), 8.71-8.60 (m, 2H), 8.34 (s, 1H), 8.18 (ddd, J=8.9, 3.3, 1.5 Hz, 2H), 8.01 (td, J=7.8, 1.7 Hz, 1H), 7.47-7.34 (m, 2H).

### C.7 2-(3-Pyridyl)-5-(2-pyridylsulfonyl)thiazole

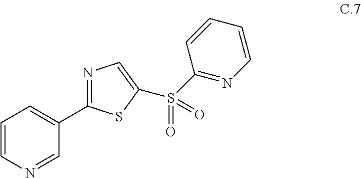 [SMILES: O=S(=O)(c1ccccn1)c1cnc(-c2cccnc2)s1]

1H NMR (400 MHz, CDCl3): δ 9.18 (d, J=2.3 Hz, 1H), 8.79-8.70 (m, 2H), 8.48 (s, 1H), 8.30-8.18 (m, 2H), 7.99 (td, J=7.8, 1.7 Hz, 1H), 7.54 (ddd, J=7.6, 4.7, 1.2 Hz, 1H), 7.43 (dd, J=8.0, 4.8 Hz, 1H).

### C.8 2-(3-Pyridyl)-5-(3-pyridylsulfanyl)thiazole

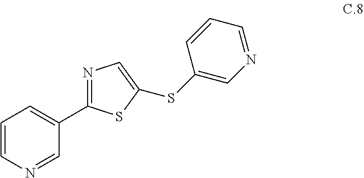 [SMILES: c1cncc(Sc2cnc(-c3cccnc3)s2)c1]

1H NMR (400 MHz, CDCl3) δ 9.13 (s, 1H), 8.69 (d, J=4.9 Hz, 1H), 8.61 (s, 1H), 8.49 (s, 1H), 8.20 (dq, J=7.7, 1.8 Hz, 1H), 8.00 (d, J=1.4 Hz, 1H), 7.62 (dq, J=7.7, 1.7 Hz, 1H), 7.40 (dd, J=8.1, 4.9 Hz, 1H), 7.26 (d, J=1.3 Hz, 1H).

### C.9 2-(3-Pyridyl)-5-(4-pyridylsulfanyl)thiazole

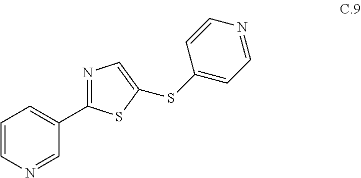 [SMILES: c1cncc(-c2ncc(Sc3ccncc3)s2)c1]

1H NMR (400 MHz, CDCl3) δ 9.18 (d, J=2.4 Hz, 1H), 8.72 (dd, J=4.8, 1.7 Hz, 1H), 8.45 (d, J=5.4 Hz, 2H), 8.25 (dt, J=8.0, 2.0 Hz, 1H), 8.04 (s, 1H), 7.43 (dd, J=8.0, 4.8 Hz, 1H), 7.11-7.03 (m, 2H).

### C.10 2-(3-Pyridyl)-5-pyrimidin-2-ylsulfanyl-thiazole

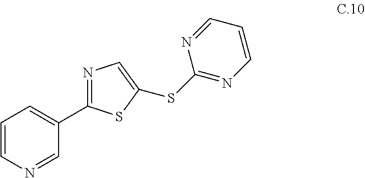 [SMILES: c1cnc(Sc2cnc(-c3cccnc3)s2)nc1]

1H NMR (400 MHz, CDCl3): δ 9.22 (s, 1H), 8.71 (s, 1H), 8.55 (dd, J=4.9, 1.0 Hz, 2H), 8.27 (d, J=7.9 Hz, 1H), 8.01 (d, J=1.0 Hz, 1H), 7.42 (d, J=6.7 Hz, 1H), 7.07 (t, J=4.8 Hz, 1H).

### C.11 2-(3-Pyridyl)-5-pyrimidin-2-ylsulfinyl-thiazole

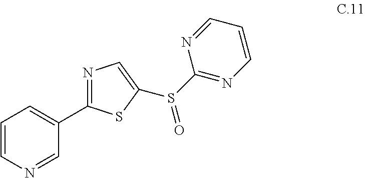 [SMILES: O=S(c1ncccn1)c1cnc(-c2cccnc2)s1]

1H NMR (400 MHz, DMSO): δ 9.14 (d, J=2.3 Hz, 1H), 9.04 (d, J=4.8 Hz, 2H), 8.71 (dd, J=4.9, 1.6 Hz, 1H), 8.64 (s, 1H), 8.33 (dt, J=7.8, 2.2 Hz, 1H), 7.72 (t, J=4.8 Hz, 1H), 7.55 (dd, J=8.0, 4.8 Hz, 1H).

C.12 2-(3-Pyridyl)-5-pyrimidin-2-ylsulfonyl-thiazole

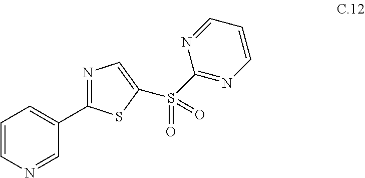 [SMILES: O=S(=O)(c1ncccn1)c1cnc(-c2cccnc2)s1]

1H NMR (400 MHz, CDCl3) δ 8.92 (d, J=4.7 Hz, 1H), 8.78 (s, 1H), 8.38 (d, J=1.1 Hz, 1H), 8.25 (d, J=6.4 Hz, 1H), 7.72 (d, J=8.0 Hz, 1H), 7.47 (t, J=4.8 Hz, 1H), 7.35 (t, J=7.3 Hz, 1H).

### C.13 2-(3-Pyridyl)-5-(2-thienylsulfanyl)thiazole

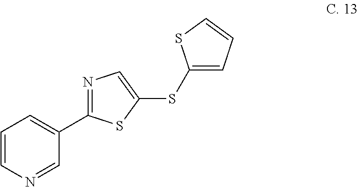 [SMILES: c1cncc(-c2ncc(Sc3cccs3)s2)c1]

1H NMR (400 MHz, DMSO-d6) δ 9.08 (d, J=2.3 Hz, 1H), 8.67 (dd, J=4.9, 1.6 Hz, 1H), 8.26 (dt, J=8.0, 2.0 Hz, 1H), 8.12 (s, 1H), 7.76 (dd, J=5.4, 1.3 Hz, 1H), 7.53 (dd, J=8.0, 4.8 Hz, 1H), 7.45 (dd, J=3.5, 1.3 Hz, 1H), 7.11 (dd, J=5.3, 3.6 Hz, 1H).

### C.14 2-(3-Pyridyl)-5-thiazol-2-ylsulfanyl-thiazole

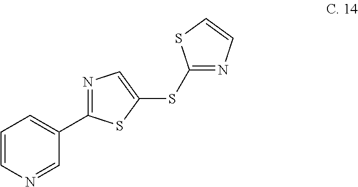 [SMILES: c1cncc(-c2ncc(Sc3nccs3)s2)c1]

1H NMR (400 MHz, CDCl3) δ 9.20 (s, 1H), 8.72 (s, 1H), 8.24 (d, J=7.8 Hz, 1H), 8.10 (s, 1H), 7.72 (d, J=3.3 Hz, 1H), 7.42 (s, 1H), 7.30 (d, J=3.4 Hz, 1H).

### C.15 4-Chloro-2-(3-pyridyl)-5-(2-pyridylsulfanyl)thiazole

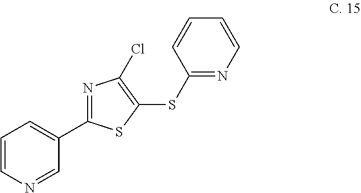 [SMILES: Clc1nc(-c2cccnc2)sc1Sc1ccccn1]

1H NMR (400 MHz, CDCl3): δ 9.16 (s, 1H), 8.71 (d, J=5.0 Hz, 1H), 8.50-8.42 (m, 1H), 8.26 (dt, J=8.1, 1.9 Hz, 1H), 7.59 (td, J=7.8, 1.9 Hz, 1H), 7.42 (dd, J=7.9, 4.8 Hz, 1H), 7.17-7.06 (m, 2H).

### C.16 4-Chloro-2-(3-pyridyl)-5-pyrimidin-2-ylsulfanyl-thiazole

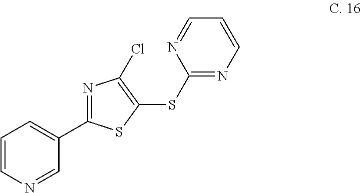 [SMILES: Clc1nc(-c2cccnc2)sc1Sc1ncccn1]

1H NMR (400 MHz, CDCl3) δ 9.17 (d, J=2.4 Hz, 1H), 8.71 (dd, J=4.9, 1.7 Hz, 1H), 8.56 (d, J=4.8 Hz, 2H), 8.26 (dt, J=8.1, 1.9 Hz, 1H), 7.42 (dd, J=8.0, 4.8 Hz, 1H), 7.09 (t, J=4.8 Hz, 1H).

### C.17 4-Bromo-2-(3-pyridyl)-5-(2-pyridylsulfanyl)thiazole

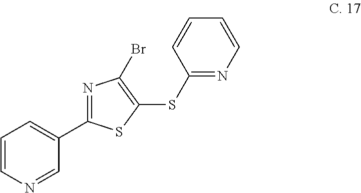 [SMILES: Brc1nc(-c2cccnc2)sc1Sc1ccccn1]

1H NMR (400 MHz, CDCl3) δ 9.16 (s, 1H), 8.71 (s, 1H), 8.52-8.42 (m, 1H), 8.25 (dt, J=8.0, 1.9 Hz, 1H), 7.59 (td, J=7.8, 1.8 Hz, 1H), 7.42 (dd, J=8.0, 4.8 Hz, 1H), 7.18-7.06 (m, 2H).

### C.18 4-Bromo-2-(3-pyridyl)-5-pyrimidin-2-ylsulfanyl-thiazole

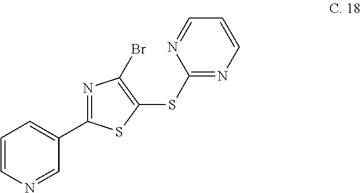 [SMILES: Brc1nc(-c2cccnc2)sc1Sc1ncccn1]

1H NMR (400 MHz, CDCl3) δ 9.17 (s, 1H), 8.71 (d, J=4.7 Hz, 1H), 8.56 (d, J=4.8 Hz, 2H), 8.27 (dt, J=8.0, 2.0 Hz, 1H), 7.42 (dd, J=7.9, 4.8 Hz, 1H), 7.09 (t, J=4.8 Hz, 1H).

### C.19 2-(3-Pyridyl)-5-(2-pyridylmethyl)thiazole

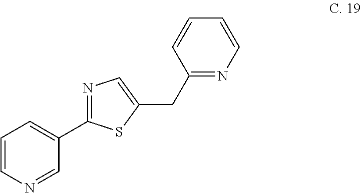 [SMILES: c1ccc(Cc2cnc(-c3cccnc3)s2)nc1]

1H NMR (400 MHz, CDCl3) δ 9.11 (d, J=2.4 Hz, 1H), 8.67-8.56 (m, 2H), 8.18 (dd, J=7.7, 2.0 Hz, 1H), 7.76 (s, 1H), 7.66 (td, J=7.6, 1.8 Hz, 1H), 7.35 (dd, J=7.9, 4.8 Hz, 1H), 7.29-7.23 (m, 1H), 7.19 (dd, J=7.5, 4.9 Hz, 1H), 4.38 (s, 2H).

### C.20 Compound mixture of Z-isomer (Z)-Akmethoxy-1-(2-pyridyl)-1-[2-(3-pyridyhthiazol-5-yl]methanimine and E-isomer (E)-AAMethoxy-1-(2-pyridyl)-1-[2-(3-pyridyhthiazol-5-yl]methanimine

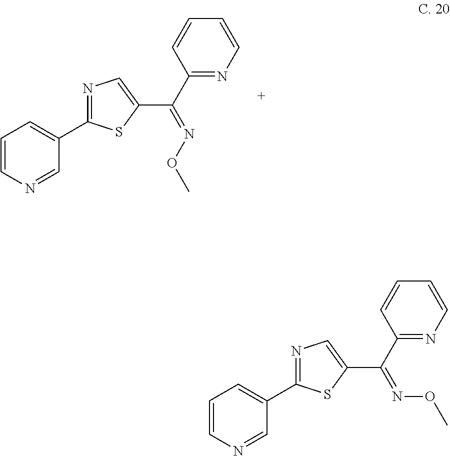 [SMILES: CO/N=C(/c1ccccn1)c1cnc(-c2cccnc2)s1 ; CO/N=C(\c1ccccn1)c1cnc(-c2cccnc2)s1]

(Z)-Akmethoxy-1-(2-pyridyl)-1-[2-(3-pyridyhthiazol-5-yl]methanimine+(E)-AAMethoxy-1-(2-pyridyl)-1-[2-(3-pyridyhthiazol-5-yl]methanimine in a ratio of approximately 4:1

1H NMR (400 MHz, DMSO-d6): δ 9.19 (dd, J=13.7, 2.2 Hz, 1H), 8.80-8.61 (m, 2H), 8.51 (s, 1H), 8.43-8.28 (m, 1H), 8.05-7.93 (m, 1H), 7.82 (d, J=7.9 Hz, 1H), 7.63-7.51 (m, 2H), 4.19 (s, 3H), 3.93 (s, 1H).

### C.21 Compound mixture of E-isomer [(E)-[2-Pyridyl-[2-(3-pyridyl)thiazol-5-yl]methylene]amino]urea and Z-isomer [(Z)-[2-pyridyl-[2-(3-pyridyl)thiazol-5-yl]methylene]amino]urea

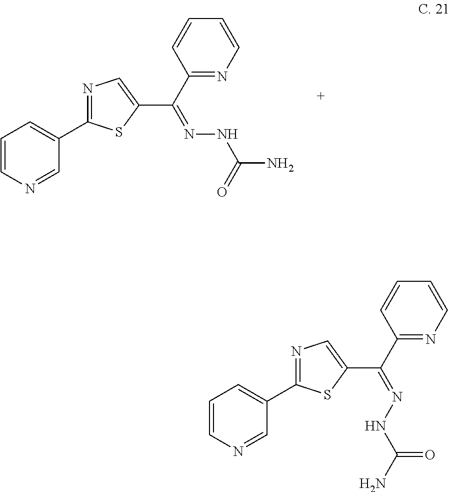 [SMILES: NC(=O)N/N=C(\c1ccccn1)c1cnc(-c2cccnc2)s1 ; NC(=O)N/N=C(/c1ccccn1)c1cnc(-c2cccnc2)s1]

[(E)-[2-Pyridyl-[2-(3-pyridyl)thiazol-5-yl]methylene]amino]urea+[(Z)-[2-pyridyl-[2-(3-pyridyl)thiazol-5-yl]methylene]amino]urea in a ratio of approximately 1:3

1H NMR (400 MHz, DMSO-d6): δ 10.99 (s, 1H), 9.16 (d, J=2.2 Hz, 1H), 8.84 (dt, J=4.6, 1.5 Hz, 1H), 8.70 (td, J=4.8, 1.6 Hz, 1H), 8.35 (ddt, J=14.5, 8.1, 1.9 Hz, 2H), 8.08 (td, J=7.8, 1.8 Hz, 1H), 7.90 (s, 1H), 7.77 (dt, J=7.9, 1.1 Hz, 1H), 7.65-7.51 (m, 2H), 6.78 (s, 1H), 6.64 (s, 2H).

### B. Biological Examples

The biological activity of the compounds of formula I of the present invention can be evaluated in biological tests as described in the following.

### General Conditions

If not otherwise specified, most test solutions are to be prepared as follows:

The active compound is dissolved at the desired concentration in a mixture of 1:1 (vol:vol) distilled water:acteon. The test solutions are prepared at the day of use (and, if not otherwised specified, in general at concentrations wt/vol).

### B.1 Vetch Aphid (Megoura Viciae)

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications.

After application, the leaf disks were air-dried and 5-8 adult aphids were placed on the leaf disks inside the microtiter plate wells. The aphids were then allowed to suck on the treated leaf disks and were incubated at about 23±1° C. and about 50±5% relative humidity for 5 days. Aphid mortality and fecundity were then visually assessed.

In this test, the compound examples of structure C.1, C.3 and C.4 at 800 ppm showed a mortality of at least 75% in comparison with untreated controls.

B.2 Green Peach Aphid (Myzus persicae)

For evaluating control of green peach aphid (Myzus persicae) through systemic means the test unit consisted of 96-well-microtiter plates containing liquid artificial diet under an artificial membrane.

The compounds were formulated using a solution containing 75% v/v water and 25% v/v DMSO. Different concentrations of formulated compounds were pipetted into the aphid diet, using a custom built pipetter, at two replications.

After application, 5-8 adult aphids were placed on the artificial membrane inside the microtiter plate wells. The aphids were then allowed to suck on the treated aphid diet and were incubated at about 23±1° C. and about 50±5% relative humidity for 3 days. Aphid mortality and fecundity was then visually assessed.

In this test, the compound examples of structure C.1 and C.4 at 800 ppm showed a mortality of at least 75% in comparison with untreated controls.

### B.3 Cowpea Aphid (Aphis Craccivora)

The active compound was dissolved at the desired concentration in a mixture of 1:1 (vol:vol) distilled water:acetone. The test solution was prepared at the day of use.

Potted cowpea plants colonized with approximately 100-150 aphids of various stages were sprayed after the pest population has been recorded. Population reduction was assessed after 24, 72, and 120 hours.

In this test, the compound examples of structure C.1, C.3 and C.4 at 100 ppm showed a mortality of at least 75% in comparison with untreated controls.

### B.4 Silverleaf Whitefly (Bemisia Argentifolii)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and about 10 to 12 whitefly adults (approximately 3-5 days old) were introduced. The insects were collected using an aspirator and a nontoxic Tygon® tubing connected to a barrier pipette tip. The tip, containing the collected insects, were gently inserted into the soil containing the treated plant, allowing insects to crawl out of the tip to reach the foliage for feeding. Cups were covered with a reusable screened lid. Test plants were maintained in a growth room at about 25° C. and about 20-40% relative humidity for 3 days, avoiding direct exposure to fluorescent light (24 hour photoperiod) to prevent trapping of heat inside the cup. Mortality was assessed 3 days after treatment, compared to untreated control plants.

In this test, the compound examples of structure C.1 and C.4 at 300 ppm showed a mortality of at least 75% in comparison with untreated controls.

B.5 Tobacco Budworm (Heliothis virescens)

For evaluating control of tobacco budworm (Heliothis virescens) the test unit consists of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom built micro atomizer, at two replications.

After application, microtiter plates were incubated at about 28±1° C. and about 80±5% relative humidity for 5 days. Egg and larval mortality was then visually assessed.

In this test, the compound examples of structure C.11 at 800 ppm showed a mortality of at least 75% in comparison with untreated controls

1-32. (canceled)

33. A method for combating or controlling invertebrate pests comprising contacting the invertebrate pests, or their food supply, habitat or breeding grounds with a compound of general formula (I) or a composition comprising at least one compound of formula (I) 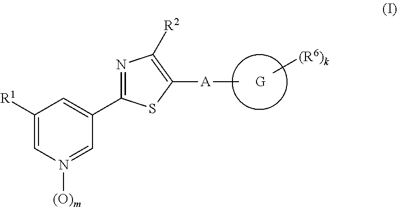
wherein
k is an integer selected from 0, 1, 2, 3, 4, 5 or 6;
m is an integer selected from 0 or 1;
A is selected from O, S(O)ₙ, NR³ or CR^{4a}R^{4b};
G is a group representing
a 3, 4-, 5-, 6- or 7-membered heterocyclic or heteroaromatic monocyclic ring system or
a 7-11-membered heterobiaromatic or heterobicyclic or heterospirobicyclic ring system,
wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized, and wherein the ring system may be optionally substituted with k substituents R⁶, wherein said substituents R⁶ are selected independently from one another, if k is more than one;
R¹ is selected from the group consisting of hydrogen, cyano and halogen;
R² is selected from the group consisting of hydrogen, halogen, cyano, C₁-C₆-alkyl and C₁-C₆-cycloalkyl, wherein the latter two may be partially or fully halogenated and/or may be substituted by 1, 2, 3 or 4 radicals R⁵;
R³ is selected from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷, said substituents R⁷ being identical or different from one another if more than one substituent R⁷ is present,
OR⁸, NR^{9a}R^{9b}, S(O)ₙR⁸, S(O)ₙNR^{9a}R^{9b}, Si(R¹¹)₂R¹²;
phenyl which may be substituted 1, 2, 3, 4 or 5 substituents R¹⁰, said substituents R¹⁰ being identical or different from one another if more than one substituent R¹⁰ is present, and
a 3-, 4-, 5-, 6- or 7-membered saturated, partially unsaturated or fully unsaturated heterocyclic ring wherein said heterocyclic ring comprises 1, 2 or 3 heteroatoms selected from the group consisting of oxygen, nitrogen and sulfur atoms, wherein said nitrogen and sulfur atom(s), independently of one another, may be oxidized, and wherein the heterocyclic ring may be substituted with 1, 2, 3, 4 or 5 substituents R¹⁰, said substituents R¹⁰ being identical or different from one another if more than one substituent R¹⁰ is present;
R^{4a}, R^{4b} are selected independently of one another from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷ and wherein said substituents R⁷ are selected, independently from one another, from the group consisting of OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶, C(═S)R¹⁵, C(═S)NR^{17a}R^{17b}, C(═S)OR¹⁶, C(═S)SR¹⁶, C(═NR^{17a})R¹⁵, C(═NR^{17a})NR^{17a}R^{17b}, and Si(R¹¹)₂R¹²,
or
R^{4a} and R^{ob} together may form a ═CHR¹³, ═CR⁷R¹³, ═NR^{9a} or ═NOR⁸ radical;
R⁵ is each independently from one another selected from the group consisting of cyano, —SCN, C₁-C₆-alkyl, C₁-C₆-haloalkyl, C₁-C₆-alkoxy, C₁-C₆-haloalkox_{Y}, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶, phenyl, optionally substituted with 1, 2, 3, 4 or 5 substituents R¹⁸, which are independently selected from one another,
and a 3-, 4-, 5-, or 6-membered saturated, partly saturated or unsaturated heterocyclic ring comprising 1, 2 or 3 heteroatoms selected from the group consisting of oxygen, nitrogen and/or sulfur, wherein said nitrogen and sulfur atoms, independently of one another, may be oxidized, and wherein the heterocyclic ring may be substituted with 1, 2, 3, 4 or 5 substituents R¹⁸, said substituents R¹⁸ being identical or different from one another if more than one substituent R¹⁸ is present;
R⁶ is each independently from one another selected from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, and wherein the carbon atoms of the aforementioned aliphatic and cyclo-aliphatic radicals may optionally be further substituted independently from one another with one or more R⁷, OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
phenyl, optionally substituted with 1, 2, 3, 4 or 5 substituents R¹⁶ selected independently from one another from,
and a 3-, 4-, 5-, 6- or 7-membered saturated, partly saturated or unsaturated aromatic heterocyclic ring comprising 1, 2, or 3 heteroatoms selected from the group consisting of oxygen, nitrogen and/or sulfur, optionally substituted with 1, 2, 3, 4 or 5 substituents R¹⁶, selected independently from one another, and wherein the nitrogen and/or the sulfur atom(s) of the heterocyclic ring may optionally be oxidized;
R⁷ is each independently from one another selected from the group consisting of hydrogen, halogen, cyano, azido, nitro, —SCN, SF₅, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
phenyl, optionally substituted with 1, 2, 3, 4 or 5 sub-stituents R¹⁸, selected independently from one another,
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized,
or
two R⁷ present on one carbon atom may together form ═O, ═CR¹³R¹⁴; ═S, ═NR^{17a}, ═NOR¹⁶; ═NNR^{17a};
or
two R⁷ may form a 3-, 4-, 5-, 6-, 7- or 8-membered saturated or partly unsaturated carbocyclic or heterocyclic ring together with the carbon atoms to which the two R⁷ are bonded to;
R⁸ is each independently from one another selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₄-C₈-alkylcycloalkyl, C₃-C₈-halocycloalkyl, C₂-C₆-alkenyl, C₂-C₆-haloalkenyl, C₂-C₆-alkinyl, C₂-C₆ haloalkinyl, —Si(R¹¹)₂R¹², S(O)ₙR¹⁶, S(O)ₙNR^{17a}R^{17b}, NR^{17a}R^{17b}, —N═CR¹³R¹⁴, —C(═O)R¹⁵, C(═O)NR^{17a}R^{17b}, C(═S)NR^{17a}R^{17b}, C(═O)OR¹⁶,
phenyl, optionally substituted with one or more substituents R¹⁸; which are selected independently from one another,
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized;
R^{9a}, R^{9b} are each independently from one another selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
S(O)ₙR¹⁶, —S(O)ₙNR^{17a}R^{17b}, C(═O)R¹⁵, C(═O)OR¹⁶, C(═O)NR^{17a}R^{17b}, C(═S)R¹⁵, C(═S)SR¹⁶, C(═S)NR^{17a}R^{17b}, C(═NR^{17a})R¹⁵;
phenyl, optionally substituted with 1, 2, 3 or 4 substituents R¹⁸, selected independently from one another;
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4, substituents R¹⁸, selected independently from one another, and wherein the nitrogen and/or the sulfur atom(s) of the heterocyclic ring may optionally be oxidized;
or,
R^{9a} and R^{9b} are together a C₂-C₇ alkylene chain and form a 3-, 4-, 5-, 6-, 7- or 8-membered saturated, partly saturated or unsaturated aromatic ring together with the nitrogen atom they are bonded to, wherein the alkylene chain may contain one or two heteratoms selected from the group consisting of oxygen, sulfur and nitrogen, and may optionally be substituted with halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
phenyl, optionally substituted with one or more substituents R¹⁸, which are selected independently from one another;
or a 3-, 4-, 5-, 6-, or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents R¹⁸, selected independently from one another, and wherein the nitrogen and/or the sulfur atom(s) of the heterocyclic ring may optionally be oxidized;
or
R^{9a} and R^{9b} together may form a ═CR¹³R¹⁴, ═NR¹⁷ or ═NOR¹⁶ radical;
R¹⁰ is each independently from one another selected from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-allcinyl, wherein the carbon atoms of the aforementioned aliphatic and cyclo-aliphatic radicals may optionally be substituted with one or more R¹⁵, which are selected independently from one another, from the group consisting of Si(R¹¹)₂R¹², OR¹⁶, OS(O)₆R¹⁶, —S(O)ₙR¹⁶, S(O)₆NR^{17a}R^{17b}, NR^{17a}R^{17b}, C(═O)R¹⁵, C(═O)OR¹⁶, —C(═NR^{17a})R¹⁵, C(═O)NR^{17a}R^{17b}, C(═S)NR^{17a}R^{17b},
phenyl, optionally substituted with halogen, cyano, nitro, C₁-C₆-alkyl, C₁-C₆-haloalkyl, C₁-C₆-alkoxy and C₁-C₆-haloalkoxy,
or a 3-, 4-, 5-, 6- or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents selected independently from one another from the group consisting of halogen, cyano, NO₂, C₁-C₆-alkyl, C₁-C₆-haloalkyl, C₁-C₆-alkoxy and C₁-C₆-haloalkoxy, and wherein the nitrogen and/or the sulfur atom(s) of the heterocyclic ring may optionally be oxidized;
or
two R¹⁶ present together on one atom of a partly saturated heterocyclic may be ═O, ═CR¹³R¹⁴; ═S, ═NR^{17a}, ═NOR¹⁶ or ═NNR^{17a};
or,
two R¹⁰ on adjacent carbon atoms may be a bridge selected from the group consisting of CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17a}, CH₂CH═N, CH═CH—NR^{17a}, OCH═N, and SCH═N and form together with the carbon atoms to which the two R¹⁰ are bonded to a 5-membered or 6-membered partly saturated or unsaturated, aromatic carbocyclic or heteocyclic ring, wherein the ring may optionally be substituted with one or two substituents selected from the group consisting of =), OH, CH₃, OCH₃, halogen, cyano, halomethyl and halomethoxy;
R¹¹, R¹² are each independently from one another selected from the group consisting of hydrogen, halogen, C₁-C₆ alkyl, C₁-C₆ haloalkyl, C₁-C₆ alkoxy, C₁-C₆ alkoxyalkyl, C₂-C₆ alkenyl, C₂-C₆ haloalkenyl, C₂-C₆ alkinyl, C₂-C₆ haloalkinyl, C₃-C₈ cycloalkyl, C₃-C₈ halocycloalkyl, C₁-C₆ alkoxyalkyl, C₁-C₆ haloalkoxyalkyl and
phenyl, optionally substituted with one or more substituents R¹⁸; which are selected independently from one another;
R¹³, R¹⁴ are each independently from one another selected from the group consisting of hydrogen, C₁-C₄ alkyl, C₁-C₆ cycloalkyl, C₁-C₄ alkoxyalkyl, phenyl and benzyl;
R¹⁵ is each independently from one another selected from the group consisting of hydrogen, halogen, cyano, nitro, OH, SH, SCN, SF₅, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄ alkoxy;
phenyl, benzyl, pyridyl, and phenoxy, wherein the last four radicals may be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino and di-(C₁-C₆-alkyl)amino,
or
two R¹⁵ present on the same carbon atom may together be ═O, ═CH(C₁-C₄), ═C(C₁-C₄-alkyl)C₁-C₄-alkyl, ═N(C₁-C₆-alkyl) or ═NO(C₁-C₆-alkyl);
R¹⁶ is each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned radicals may be unsubstituted, partially or fully halogenated and/or oxygenated and/or may carry 1 or 2 radicals selected from C₁-C₄ alkoxy, phenyl, benzyl, pyridyl, and phenoxy, wherein the last four radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl;
R^{17a}, R^{17b} are each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
phenyl, benzyl, pyridyl, and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl, or,
R^{17a} and R^{17b} may together be a C₂-C₆ alkylene chain forming a 3- to 7-membered saturated, partly saturated or unsaturated ring together with the nitrogen atom R^{17a} and R^{17b} are bonded to, wherein the alkylene chain may contain 1 or 2 heteroatoms selected from the group consisting of oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
R¹⁸ is each independently from one another selected from the group consisting of hydrogen, halogen, nitro, cyano, OH, SH, C₁-C₆-alkoxy, C₁-C₆-haloalkoxy, C₁-C₆-alkylthio, C₁-C₆-alkylsulfinyl, C₁-C₆-allylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
phenyl, benzyl, pyridyl, and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy), and (C₁-C₆-alkoxy)carbonyl;
or
two R¹⁸ present together on one atom of a partly saturated atom may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
or,
two R¹⁸ on two adjacent carbon atoms may be together a C₂-C₆ alkylene chain, which form together with the carbon atom they are bonded to a 3-, 4-, 5-, 6- or 7-membered saturated, partly saturated or unsaturated aromatic, wherein the alkylene chain may contain 1 or 2 heteroatoms selected from the group consisting of oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
n is an integer selected from 0, 1 or 2;
or an enantiomer, a diastereomer or an agriculturally or a veterinarily acceptable salt thereof.

34. A method for protecting crops, plants, plant propagation material and/or growing plants from attack or infestation by invertebrate pests comprising contacting or treating the crops, plants, plant propagation material and growing plants, or soil, material, surface, space, area or water in which the crops, plants, plant propagation material is stored or the plant is growing, with a compound of general formula (I) or a composition comprising at least one compound of formula (I) 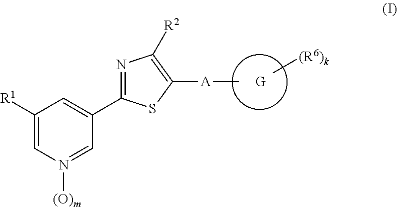
wherein
k is an integer selected from 0, 1, 2, 3, 4, 5 or 6;
m is an integer selected from 0 or 1;
A is selected from O, S(O)ₙ, NR³ or CR^{4a}R^{4b};
G is a group representing
a 3, 4-, 5-, 6- or 7-membered heterocyclic or heteroaromatic monocyclic ring system or
a 7-11-membered heterobiaromatic or heterobicyclic or heterospirobicyclic ring system,
wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized, and wherein the ring system may be optionally substituted with k substituents R⁶, wherein said substituents R⁶ are selected independently from one another, if k is more than one;
R¹ is selected from the group consisting of hydrogen, cyano and halogen;
R² is selected from the group consisting of hydrogen, halogen, cyano, C₁-C₆-alkyl and C₁-C₆-cycloalkyl, wherein the latter two may be partially or fully halogenated and/or may be substituted by 1, 2, 3 or 4 radicals R⁵;
R³ is selected from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷, said substituents R⁷ being identical or different from one another if more than one substituent R⁷ is present,
OR⁸, NR^{9a}R^{9b}, S(O)ₙR⁸, S(O)ₙNR^{9a}R^{9b}, Si(R¹¹)₂R¹²;
phenyl which may be substituted 1, 2, 3, 4 or 5 substituents R¹⁰, said substituents R¹⁰ being identical or different from one another if more than one substituent R¹⁰ is present, and
a 3-, 4-, 5-, 6- or 7-membered saturated, partially unsaturated or fully unsaturated heterocyclic ring wherein said heterocyclic ring comprises 1, 2 or 3 heteroatoms selected from the group consisting of oxygen, nitrogen and sulfur atoms, wherein said nitrogen and sulfur atom(s), independently of one another, may be oxidized, and wherein the heterocyclic ring may be substituted with 1, 2, 3, 4 or 5 substituents R¹⁰, said substituents R¹⁰ being identical or different from one another if more than one substituent R¹⁰ is present;
R^{4a}R^{4b} are selected independently of one another from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷ and wherein said substituents R⁷ are selected, independently from one another, from the group consisting of OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶, C(═S)R¹⁵, C(═S)NR^{17a}R^{17b}, C(═S)OR¹⁶, C(═S)SR¹⁶, C(═NR^{17a})R¹⁵, C(═NR^{17a})NR^{17a}R^{17b}, and Si(R¹¹)₂R¹²,
or
R^{4a} and R^{4b} together may form a ═CHR¹³, ═CR⁷R¹³, ═NR^{9a} or ═NOR⁸ radical;
R⁵ is each independently from one another selected from the group consisting of cyano, —SCN, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)₆R¹⁶, S(O)ₙNR^{17a}R^{17b}, NR^{17a}R^{17b}, C(O)NR^{17a}R^{17b}, C(═S)NR^{17a}R^{17b}, C(═O)OR¹⁶,
phenyl, optionally substituted with 1, 2, 3, 4 or 5 substituents R¹⁸, which are independently selected from one another,
and a 3-, 4-, 5-, or 6-membered saturated, partly saturated or unsaturated heterocyclic ring comprising 1, 2 or 3 heteroatoms selected from the group consisting of oxygen, nitrogen and/or sulfur, wherein said nitrogen and sulfur atoms, independently of one another, may be oxidized, and wherein the heterocyclic ring may be substituted with 1, 2, 3, 4 or 5 substituents R¹⁸, said substituents R¹⁸ being identical or different from one another if more than one substituent R¹⁸ is present;
R⁶ is each independently from one another selected from the group consisting of hydrogen, halogen, cyano, azido, nitro, SCN, SF₅, C₁-C₁₀-alkyl, C₃-C₈-cyeloalkyl, C₂-C₁₀-alkenyl, C₂-C₁₀-alkinyl, and wherein the carbon atoms of the aforementioned aliphatic and cyclo-aliphatic radicals may optionally be further substituted independently from one another with one or more R⁷,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
phenyl, optionally substituted with 1, 2, 3, 4 or 5 substituents R¹⁰ selected independently from one another from,
and a 3-, 4-, 5-, 6- or 7-membered saturated, partly saturated or unsaturated aromatic heterocyclic ring comprising 1, 2, or 3 heteroatoms selected from the group consisting of oxygen, nitrogen and/or sulfur, optionally substituted with 1, 2, 3, 4 or 5 substituents R¹⁶, selected independently from one another, and wherein the nitrogen and/or the sulfur atom(s) of the heterocyclic ring may optionally be oxidized;
R⁷ is each independently from one another selected from the group consisting of hydrogen, halogen, cyano, azido, nitro, —SCN, SF₅, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
phenyl, optionally substituted with 1, 2, 3, 4 or 5 sub-stituents R¹⁸, selected independently from one another,
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized,
or
two R⁷ present on one carbon atom may together form ═O, ═CR¹³R¹⁴; ═S, ═NR^{17a}, ═NOR¹⁶; ═NNR^{17a};
or
two R⁷ may form a 3-, 4-, 5-, 6-, 7- or 8-membered saturated or partly unsaturated carbocyclic or heterocyclic ring together with the carbon atoms to which the two R⁷ are bonded to;
R⁸ is each independently from one another selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloallylthio, C₃-C₈-cycloalkyl, C₄-C₈-alkylcycloalkyl, C₃-C₈-haloeyeloalkyl, C₂-C₆-alkenyl, C₂-C₆-haloalkenyl, C₂-C₆-alkinyl, C₂-C₆ haloalkinyl, —Si(R¹¹)₂R¹², S(O)ₙR¹⁶, S(O)ₙNR^{17a}R^{17b}, NR^{17a}R^{17b}, —N═CR¹³R¹⁴, —C(═O)R¹⁵, C(═O)NR^{17a}R^{17b}, C(═S)NR^{17a}R^{17b}, C(═O)OR¹⁶,
phenyl, optionally substituted with one or more substituents R¹⁸; which are selected independently from one another,
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized;
R^{9a}, R^{9b} are each independently from one another selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
S(O)ₙR¹⁶, —S(O)ₙNR^{17a}R^{17b}, C(═O)R¹⁵, C(═O)OR¹⁶, C(═O)NR^{17a}R^{17b}, C(═S)R¹⁵, C(═S)SR¹⁶, C(═S)NR^{17a}R^{17b}, C(═NR^{17a})R¹⁵;
phenyl, optionally substituted with 1, 2, 3 or 4 substituents R¹⁸, selected independently from one another;
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4, substituents R¹⁸, selected independently from one another, and wherein the nitrogen and/or the sulfur atom(s) of the heterocyclic ring may optionally be oxidized;
or,
R^{9a} and R^{9b} are together a C₂-C₇ alkylene chain and form a 3-, 4-, 5-, 6-, 7- or 8-membered saturated, partly saturated or unsaturated aromatic ring together with the nitrogen atom they are bonded to, wherein the alkylene chain may contain one or two heteratoms selected from the group consisting of oxygen, sulfur and nitrogen, and may optionally be substituted with halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
phenyl, optionally substituted with one or more substituents R¹⁸, which are selected independently from one another;
or a 3-, 4-, 5-, 6-, or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents R¹⁸, selected independently from one another, and wherein the nitrogen and/or the sulfur atom(s) of the heterocyclic ring may optionally be oxidized;
or
R^{9a} and R^{9b} together may form a ═CR¹³R¹⁴, ═NR¹⁷ or ═NOR¹⁶ radical;
R¹⁰ is each independently from one another selected from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, wherein the carbon atoms of the aforementioned aliphatic and cyclo-aliphatic radicals may optionally be substituted with one or more R¹⁵, which are selected independently from one another, from the group consisting of Si(R¹¹)₂R¹², OR¹⁶, OS(O)ₙR¹⁶, —S(O)ₙR¹⁶, S(O)ₙNR^{17a}R^{17b}, NR^{17a}R^{17b}, C(═O)R¹⁵, C(═O)OR¹⁶, —C(═NR^{17a})R¹⁵, C(═O)NR^{17a}R^{17b}, C(═S)NR^{17a}R^{17b},
phenyl, optionally substituted with halogen, cyano, nitro, C₁-C₆-alkyl, C₁-C₆-haloalkyl, C₁-C₆-alkoxy and C₁-C₆-haloalkoxy,
or a 3-, 4-, 5-, 6- or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents selected independently from one another from the group consisting of halogen, cyano, NO₂, C₁-C₆-alkyl, C₁-C₆-haloalkyl, C₁-C₆-alkoxy and C₁-C₆-haloalkoxy, and wherein the nitrogen and/or the sulfur atom(s) of the heterocyclic ring may optionally be oxidized;
or
two R¹⁰ present together on one atom of a partly saturated heterocyclic may be ═O, ═CR¹³R¹⁴; ═S, ═NR^{17a}, ═NOR¹⁶ or ═NNR^{17a};
or,
two R¹⁰ on adjacent carbon atoms may be a bridge selected from the group consisting of CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17a}, CH₂CH═N, CH═CH—NR^{17a}, OCH═N, and SCH═N and form together with the carbon atoms to which the two R¹⁰ are bonded to a 5-membered or 6-membered partly saturated or unsaturated, aromatic carbocyclic or heteocyclic ring, wherein the ring may optionally be substituted with one or two substituents selected from the group consisting of ═O, OH, CH₃, OCH₃, halogen, cyano, halomethyl and halomethoxy;
R¹¹, R¹² are each independently from one another selected from the group consisting of hydrogen, halogen, C₁-C₆ alkyl, C₁-C₆ haloalkyl, C₁-C₆ alkoxy, C₁-C₆ alkoxyalkyl, C₂-C₆ alkenyl, C₂-C₆ haloalkenyl, C₂-C₆ alkinyl, C₂-C₆ haloalkinyl, C₃-C₈ cycloalkyl, C₃-C₈ halocycloalkyl, C₁-C₆ alkoxyalkyl, C₁-C₆ haloalkoxyalkyl and
phenyl, optionally substituted with one or more substituents R¹⁸; which are selected independently from one another;
R¹³, R¹⁴ are each independently from one another selected from the group consisting of hydrogen, C₁-C₄ alkyl, C₁-C₆ cycloalkyl, C₁-C₄ alkoxyalkyl, phenyl and benzyl;
R¹⁵ is each independently from one another selected from the group consisting of hydrogen, halogen, cyano, nitro, OH, SH, SCN, SF₅, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄ alkoxy;
phenyl, benzyl, pyridyl, and phenoxy, wherein the last four radicals may be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino and di-(C₁-C₆-alkyl)amino,
or
two R¹⁵ present on the same carbon atom may together be ═O, ═CH(C₁-C₄), ═C(C₁-C₄-alkyl)C₁-C₄-alkyl, ═N(C₁-C₆-alkyl)C₁-C₆-alkyl or ═NO(C₁-C₆-alkyl);
R¹⁶ is each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₃-C₈-cycloalkyl, wherein the four last mentioned radicals may be unsubstituted, partially or fully halogenated and/or oxygenated and/or may carry 1 or 2 radicals selected from C₁-C₄ alkoxy, phenyl, benzyl, pyridyl, and phenoxy, wherein the last four radicals may be unsubstituted, partially or fully halogenated and/or carry 1, 2 or 3 substituents selected from the group consisting of C₁-C₆-alkyl, C₁-C₆-haloallyl, C₁-C₆-alkoxy, C₁-C₆ haloalkoxy and (C₁-C₆-alkoxy)carbonyl;
R^{17a}, R^{17b} are each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
phenyl, benzyl, pyridyl, and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl,
or,
R^{17a} and R^{17b} may together be a C₂-C₆ alkylene chain forming a 3- to 7-membered saturated, partly saturated or unsaturated ring together with the nitrogen atom R^{17a} and R^{17b} are bonded to, wherein the alkylene chain may contain 1 or 2 heteroatoms selected from the group consisting of oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
R¹⁸ is each independently from one another selected from the group consisting of hydrogen, halogen, nitro, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
phenyl, benzyl, pyridyl, and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy), and (C₁-C₆-alkoxy)carbonyl;
or
two R¹⁸ present together on one atom of a partly saturated atom may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
or,
two R¹⁸ on two adjacent carbon atoms may be together a C₂-C₆ alkylene chain, which form together with the carbon atom they are bonded to a 3-, 4-, 5-, 6- or 7-membered saturated, partly saturated or unsaturated aromatic, wherein the alkylene chain may contain 1 or 2 heteroatoms selected from the group consisting of oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
n is an integer selected from 0, 1 or 2;
or an enantiomer, a diastereomer or an agriculturally or a veterinarily acceptable salt thereof.

35. A method for treating, controlling, preventing or protecting animals against infestation or infection by parasites by administering or applying orally, topically or parenterally to the animals a compound of general formula (I) or a composition comprising at least one compound of formula (I) 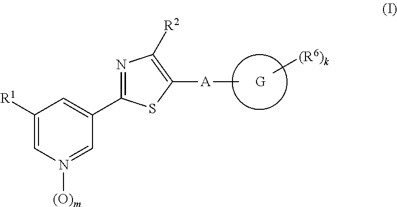
wherein
k is an integer selected from 0, 1, 2, 3, 4, 5 or 6;
m is an integer selected from 0 or 1;
A is selected from O, S(O)ₙ, NR³ or CR^{4a}R^{4b};
G is a group representing
a 3, 4-, 5-, 6- or 7-membered heterocyclic or heteroaromatic monocyclic ring system or
a 7-11-membered heterobiaromatic or heterobicyclic or heterospirobicyclic ring system,
wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized, and
wherein the ring system may be optionally substituted with k substituents R⁶, wherein said substituents R⁶ are selected independently from one another, if k is more than one;
R¹ is selected from the group consisting of hydrogen, cyano and halogen;
R² is selected from the group consisting of hydrogen, halogen, cyano, C₁-C₆-alkyl and C₁-C₆-cycloalkyl, wherein the latter two may be partially or fully halogenated and/or may be substituted by 1, 2, 3 or 4 radicals R⁵;
R³ is selected from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷, said substituents R⁷ being identical or different from one another if more than one substituent R⁷ is present, OR⁸, NR^{9a}R^{9b}, S(O)R⁸, S(O)ₙNR^{9a}R^{9b}, Si(R¹¹)₂R¹²;
phenyl which may be substituted 1, 2, 3, 4 or 5 substituents R¹⁰, said substituents R¹⁰ being identical or different from one another if more than one substituent R¹⁰ is present, and
a 3-, 4-, 5-, 6- or 7-membered saturated, partially unsaturated or fully unsaturated heterocyclic ring wherein said heterocyclic ring comprises 1, 2 or 3 heteroatoms selected from the group consisting of oxygen, nitrogen and sulfur atoms, wherein said nitrogen and sulfur atom(s), independently of one another, may be oxidized, and wherein the heterocyclic ring may be substituted with 1, 2, 3, 4 or 5 substituents R¹⁰, said substituents R¹⁰ being identical or different from one another if more than one substituent R¹⁰ is present;
R^{4a}, R^{4b} are selected independently of one another from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷ and wherein said substituents R⁷ are selected, independently from one another, from the group consisting of OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶, C(═S)R¹⁵, C(═S)NR^{17a}R^{17b}, C(═S)OR¹⁶, C(═S)SR¹⁶, C(═NR^{17a})R¹⁵, C(═NR^{17a})NR^{17a}R^{17b}, and Si(R¹¹)₂R¹²,
or
R^{4a} and R^{4b} together may form a ═CHR¹³, ═CR⁷R¹³, ═NR^{9a} or ═NOR⁸ radical;
R⁵ is each independently from one another selected from the group consisting of cyano, —SCN, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR¹⁶, S(O)NR^{17a}R^{17b}, NR^{17a}R^{17b}, C(═O)NR^{17a}R^{17b}, C(═S)NR^{17a}R^{17b}, C(═O)OR¹⁶,
phenyl, optionally substituted with 1, 2, 3, 4 or 5 substituents R¹⁸, which are independently selected from one another,
and a 3-, 4-, 5-, or 6-membered saturated, partly saturated or unsaturated heterocyclic ring comprising 1, 2 or 3 heteroatoms selected from the group consisting of oxygen, nitrogen and/or sulfur, wherein said nitrogen and sulfur atoms, independently of one another, may be oxidized, and wherein the heterocyclic ring may be substituted with 1, 2, 3, 4 or 5 substituents R¹⁸, said substituents R¹⁸ being identical or different from one another if more than one substituent R¹⁸ is present;
R⁶ is each independently from one another selected from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-allcinyl, and wherein the carbon atoms of the aforementioned aliphatic and cyclo-aliphatic radicals may optionally be further substituted independently from one another with one or more R⁷,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
phenyl, optionally substituted with 1, 2, 3, 4 or 5 substituents R¹⁹ selected independently from one another from,
and a 3-, 4-, 5-, 6- or 7-membered saturated, partly saturated or unsaturated aromatic heterocyclic ring comprising 1, 2, or 3 heteroatoms selected from the group consisting of oxygen, nitrogen and/or sulfur, optionally substituted with 1, 2, 3, 4 or 5 substituents R¹⁰, selected independently from one another, and wherein the nitrogen and/or the sulfur atom(s) of the heterocyclic ring may optionally be oxidized;
R⁷ is each independently from one another selected from the group consisting of hydrogen, halogen, cyano, azido, nitro, —SCN, SF₅, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-allylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
phenyl, optionally substituted with 1, 2, 3, 4 or 5 sub-stituents R¹⁸, selected independently from one another,
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized,
or
two R⁷ present on one carbon atom may together form ═O, ═CR¹³R¹⁴; ═S, NR^{17a}, ═NOR¹⁶; ═NNR^{17a};
or
two R⁷ may form a 3-, 4-, 5-, 6-, 7- or 8-membered saturated or partly unsaturated carbocyclic or heterocyclic ring together with the carbon atoms to which the two R⁷ are bonded to;
R⁸ is each independently from one another selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₄-C₈-alkylcycloalkyl, C₃-C₈-halocycloalkyl, C₂-C₆-alkenyl, C₂-C₆-haloalkenyl, C₂-C₆-alkinyl, C₂-C₆ haloalkinyl, —Si(R¹¹)₂R¹², S(Oₙ)R¹⁶, S(O)ₙNR^{17a}R^{17b}, NR^{17a}R^{17b}, —N═CR¹³R¹⁴, —C(═O)R¹⁵, C(═O)NR^{17a}R^{17b}, C(═S)NR^{17a}R^{17b}, C(═O)OR¹⁶,
phenyl, optionally substituted with one or more substituents R¹⁸; which are selected independently from one another,
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized;
R^{9a}, R^{9b} are each independently from one another selected from the group consisting of hydrogen, C₁-C₆-alkyl, C₁-C₆-halo alkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
S(O)R¹⁶, —S(O)NR^{17a}R^{17b}, C(═O)R¹⁵, C(═O)OR¹⁶, C(═O)NR^{17a}R^{17b}, C(═S)R¹⁵, C(═S)SR¹⁶, C(═S)NR^{17a}R^{17b}, C(═NR^{17a})R¹⁵;
phenyl, optionally substituted with 1, 2, 3 or 4 substituents R¹⁸, selected independently from one another;
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4, substituents R¹⁸, selected independently from one another, and wherein the nitrogen and/or the sulfur atom(s) of the heterocyclic ring may optionally be oxidized;
or,
R^{9a} and R^{9b} are together a C₂-C₇ alkylene chain and form a 3-, 4-, 5-, 6-, 7- or 8-membered saturated, partly saturated or unsaturated aromatic ring together with the nitrogen atom they are bonded to, wherein the alkylene chain may contain one or two heteratoms selected from the group consisting of oxygen, sulfur and nitrogen, and may optionally be substituted with halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
phenyl, optionally substituted with one or more substituents R¹⁸, which are selected independently from one another;
or a 3-, 4-, 5-, 6-, or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents R¹⁸, selected independently from one another, and wherein the nitrogen and/or the sulfur atom(s) of the heterocyclic ring may optionally be oxidized;
or
R^{9a} and R^{9b} together may form a ═CR¹³R¹⁴, ═NR¹⁷ or ═NOR¹⁶ radical;
R¹⁰ is each independently from one another selected from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, wherein the carbon atoms of the aforementioned aliphatic and cycloaliphatic radicals may optionally be substituted with one or more R¹⁵, which are selected independently from one another, from the group consisting of Si(R¹¹)₂R¹², OR¹⁶, OS(O)ₙR¹⁶, —S(O)ₙR¹⁶, S(O)ₙNR^{17a}R^{17b}, NR^{17a}R^{17b}, C(═O)R¹⁵, C(═O)OR¹⁶, —C(═NR^{17a})R¹⁵, C(═O)NR^{17a}R^{17b}, C(═S)NR^{17a}R^{17b},
phenyl, optionally substituted with halogen, cyano, nitro, C₁-C₆-alkyl, C₁-C₆-haloalkyl, C₁-C₆-alkoxy and C₁-C₆-haloalkoxy,
or a 3-, 4-, 5-, 6- or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents selected independently from one another from the group consisting of halogen, cyano, NO₂, C₁-C₆-alkyl, C₁-C₆-haloalkyl, C₁-C₆-alkoxy and C₁-C₆-haloalkoxy, and wherein the nitrogen and/or the sulfur atom(s) of the heterocyclic ring may optionally be oxidized;
or
two R¹⁰ present together on one atom of a partly saturated heterocyclic may be ═O, ═CR¹³R¹⁴; ═S, ═NR^{17a}, ═NOR¹⁶ or ═NNR^{17a};
or,
two R¹⁰ on adjacent carbon atoms may be a bridge selected from the group consisting of CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17b}, CH₂CH═N, CH═CH—NR^{17a}, OCH═N, and SCH═N and form together with the carbon atoms to which the two R¹⁶ are bonded to a 5-membered or 6-membered partly saturated or unsaturated, aromatic carbocyclic or heteocyclic ring, wherein the ring may optionally be substituted with one or two substituents selected from the group consisting of ═O, OH, CH₃, OCH₃, halogen, cyano, halomethyl and halomethoxy;
R¹¹, R¹² are each independently from one another selected from the group consisting of hydrogen, halogen, C₁-C₆ alkyl, C₁-C₆ haloalkyl, C₁-C₆ alkoxy, C₁-C₆ alkoxyalkyl, C₂-C₆ alkenyl, C₂-C₆ haloalkenyl, C₂-C₆ alkinyl, C₂-C₆ haloalkinyl, C₃-C₈ cycloalkyl, C₃-C₈ halocycloalkyl, C₁-C₆ alkoxyalkyl, C₁-C₆ haloalkoxyalkyl and
phenyl, optionally substituted with one or more substituents R¹⁸; which are selected independently from one another;
R¹³, R¹⁴ are each independently from one another selected from the group consisting of hydrogen, C₁-C₄ alkyl, C₁-C₆ cycloalkyl, C₁-C₄ alkoxyalkyl, phenyl and benzyl;
R¹⁵ is each independently from one another selected from the group consisting of hydrogen, halogen, cyano, nitro, OH, SH, SCN, SF₅, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄ alkoxy;
phenyl, benzyl, pyridyl, and phenoxy, wherein the last four radicals may be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino and di-(C₁-C₆-alkyl)amino,
or
two R¹⁵ present on the same carbon atom may together be ═O, ═CH(C₁-C₄), ═C(C₁-C₄-alkyl)C₁-C₄-alkyl, ═N(C₁-C₆-alkyl) or ═NO(C₁-C₆-alkyl);
R¹⁶ is each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-Cₒ-alkylsulfonyl, C₁-C₆-haloalkylthio, trimethylsilyl, triethylsilyl, tertbutyldimethylsilyl, C₁-C₆-alkyl, C₂-C₆-alkenyl, C₂-C₆-alkinyl, C₃-C₈-cycloalkyl, wherein the four last mentioned radicals may be unsubstituted, partially or fully halogenated and/or oxygenated and/or may carry 1 or 2 radicals selected from C₁-C₄ alkoxy,
phenyl, benzyl, pyridyl, and phenoxy, wherein the last four radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl;
R^{17a}, R^{17b} are each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl, C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
phenyl, benzyl, pyridyl, and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl,
or,
R^{17a} and R^{17b} may together be a C₂-C₆ alkylene chain forming a 3- to 7-membered saturated, partly saturated or unsaturated ring together with the nitrogen atom R^{17a} and R^{17b} are bonded to, wherein the alkylene chain may contain 1 or 2 heteroatoms selected from the group consisting of oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
R¹⁸ is each independently from one another selected from the group consisting of hydrogen, halogen, nitro, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy, phenyl, benzyl, pyridyl, and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy), and (C₁-C₆-alkoxy)carbonyl;
or
two R¹⁸ present together on one atom of a partly saturated atom may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
or,
two R¹⁸ on two adjacent carbon atoms may be together a C₂-C₆ alkylene chain, which form together with the carbon atom they are bonded to a 3-, 4-, 5-, 6- or 7-membered saturated, partly saturated or unsaturated aromatic, wherein the alkylene chain may contain 1 or 2 heteroatoms selected from the group consisting of oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
n is an integer selected from 0, 1 or 2;
or an enantiomer, a diastereomer or an agriculturally or a veterinarily acceptable salt thereof.

36. The method according to claim 33, wherein the method is using a substituted 3-pyridyl thiazole compound of the general formula (I), wherein
R¹ is hydrogen or fluoro.

37. The method according to claim 33, wherein the method is using a substituted 3-pyridyl thiazole compound of the general formula (I), wherein
R² is selected from the group consisting of hydrogen, fluoro, chloro, bromo, cyano, methyl, ethyl, propyl, isopropyl, tertbutyl, cyclopropyl, difluoromethyl, trifluoromethyl, pentafluoroethyl, 2,2-difluoroethyl or 2,2,2-trifluoroethyl.

38. The method according to claim 33, wherein the method is using a substituted 3-pyridyl thiazole compound of the general formula (I), wherein
A is O.

39. The method according to claim 33, wherein the method is using a substituted 3-pyridyl thiazole compound of the general formula (I), wherein
A is S(O), and
n is 0, 1 or 2.

40. The method according to claim 33, wherein the method is using a substituted 3-pyridyl thiazole compound of the general formula (I), wherein
A is NR³, and
R³ is selected from the group consisting of hydrogen, C₁-C₆-alkyl, C₃-C₆-cycloalkyl, C₂-C₆-alkenyl, C₂-C₆-alkynyl, wherein the aforementioned aliphatic and cycloaliphatic radicals each independently may be substituted with 1 to 5 substituents R¹⁵, said substituents R¹⁵ being identical or different from one another if more than one substituent R¹⁵ is present.

41. The method according to claim 33, wherein the method is using a substituted 3-pyridyl thiazole compounds is of the general formula (I), wherein
A is CR^{4a}R^{4b};
R^{4a}, R^{4b} are selected independently of each other from the group consisting of hydrogen, C₁-C₆-alkyl, C₃-C₇-cycloalkyl, C₂-C₆-alkenyl, C₂-C₆-alkynyl, wherein the aforementioned aliphatic and cycloaliphatic radicals each independently may be substituted with 1 to 10 substituents R⁷, said substituents R⁷ being identical or different from one another if more than one substituent R⁷ is present,
OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶, C(═S)R¹⁵, C(═S)NR^{17a}R^{17b},
or
R^{4a} and R^{4b} together may form a ═CHR¹³, ═CR⁷R¹³, ═NR^{9a} or ═NOR⁸ radical.

42. The method according to claim 33, wherein the method is using a substituted 3-pyridyl thiazole compounds is of the general formula (I), wherein
G is a nitrogen containing 4-, 5-, or 6-membered heterocyclic or hetero-aromatic ring system selected from any of the following ring systems G-1k to G-135k: 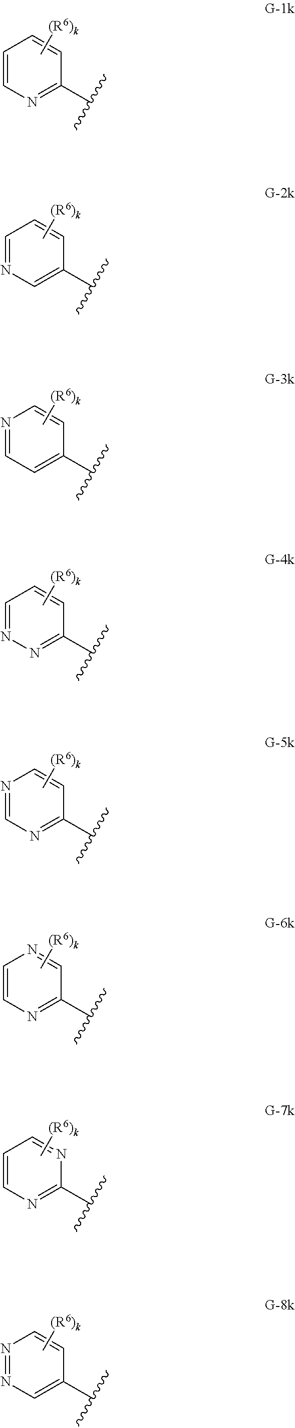 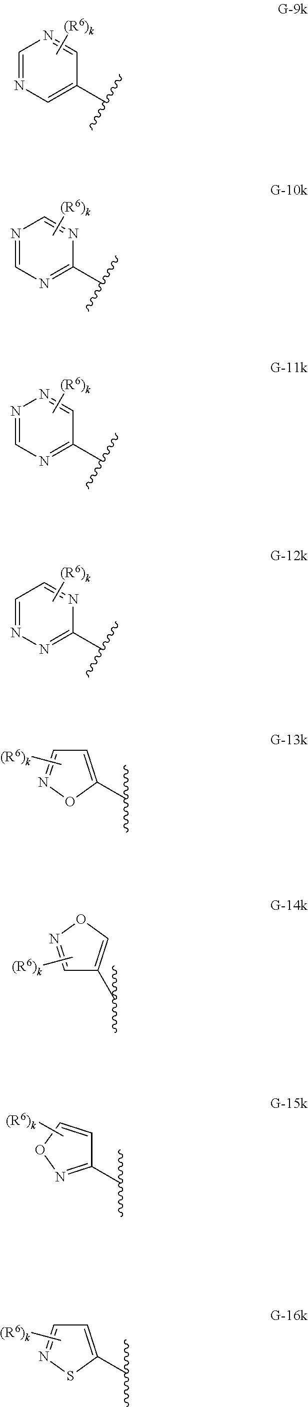 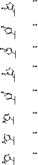 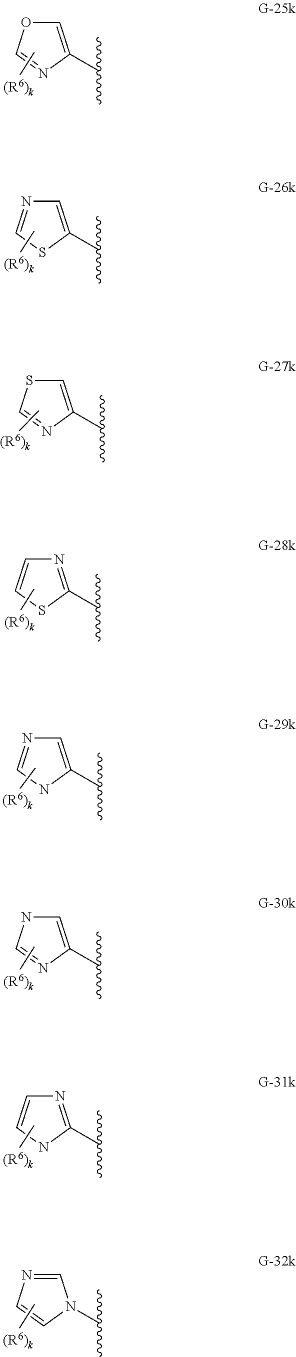 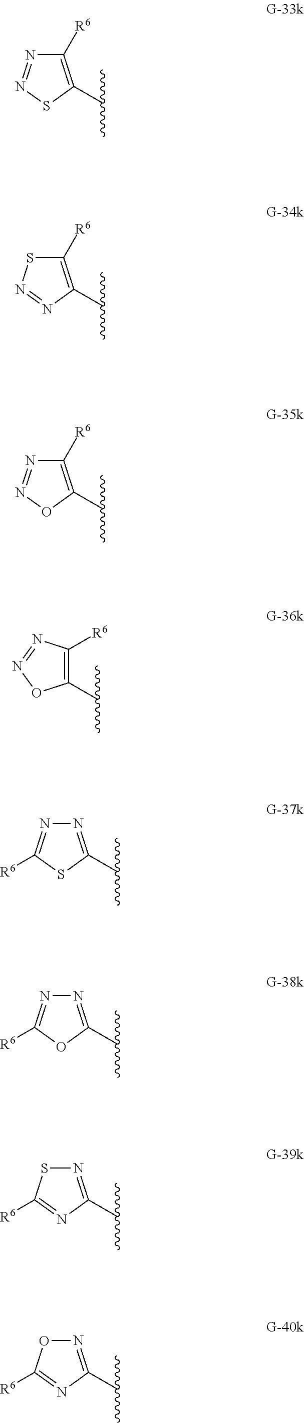 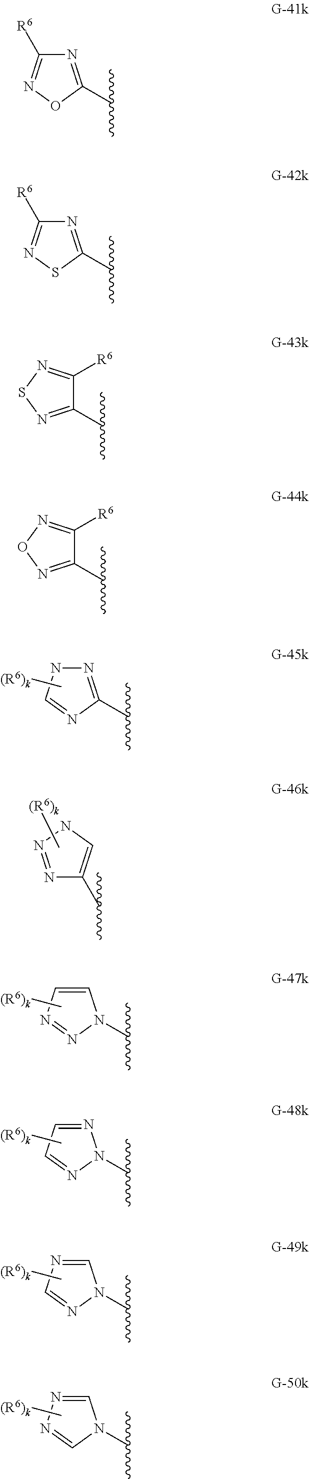 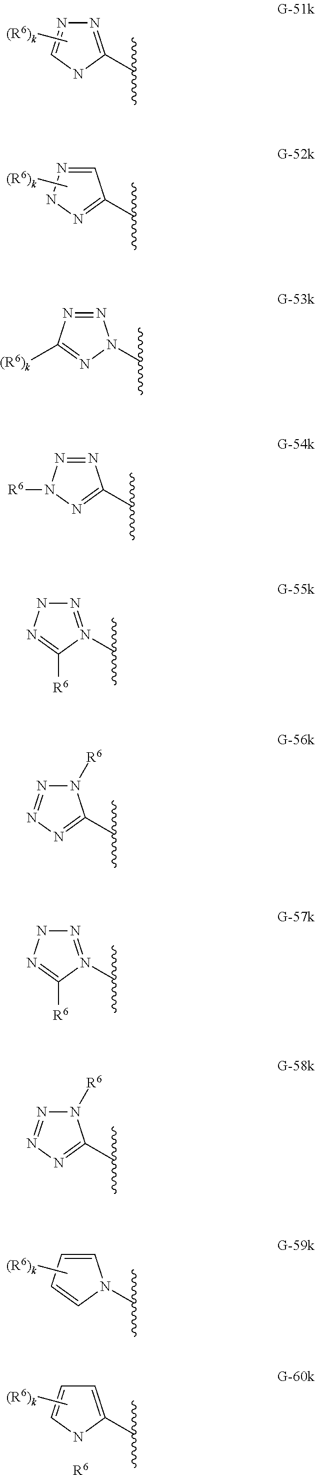 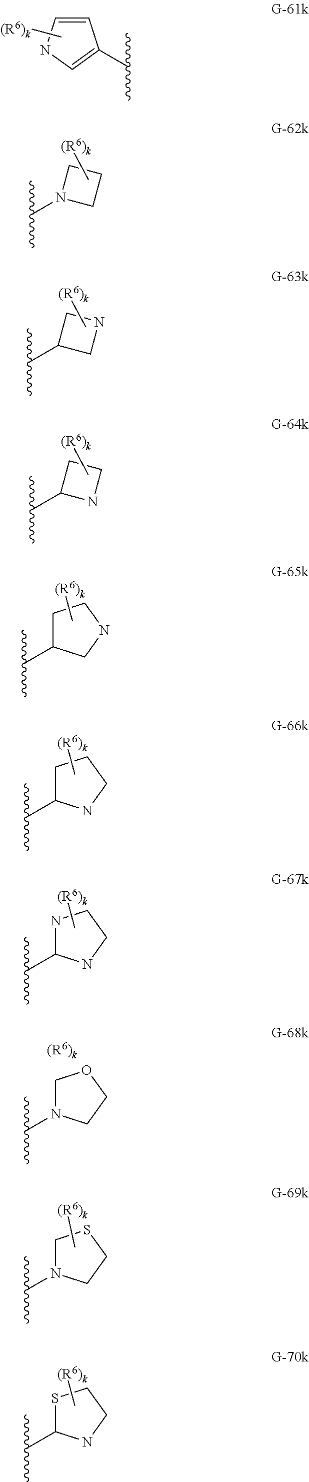 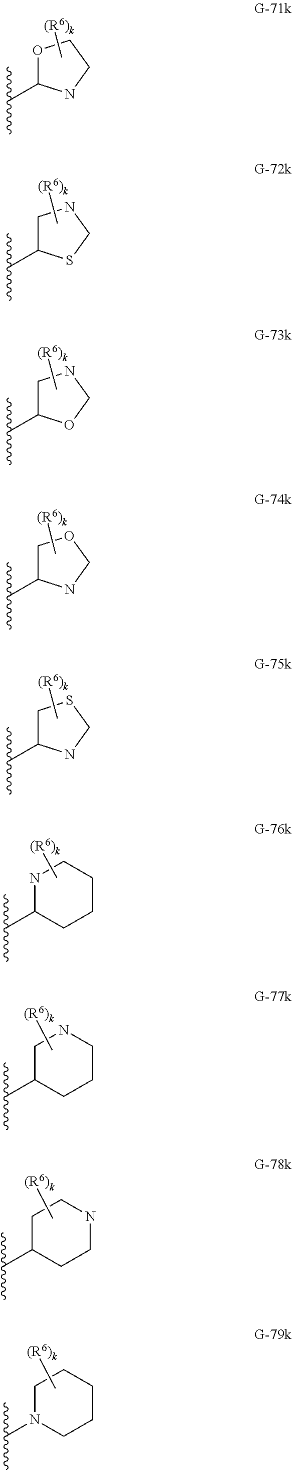 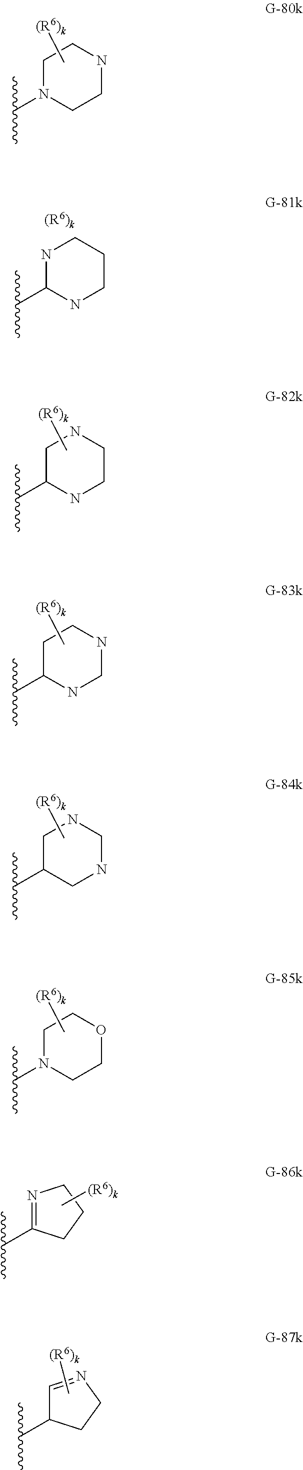 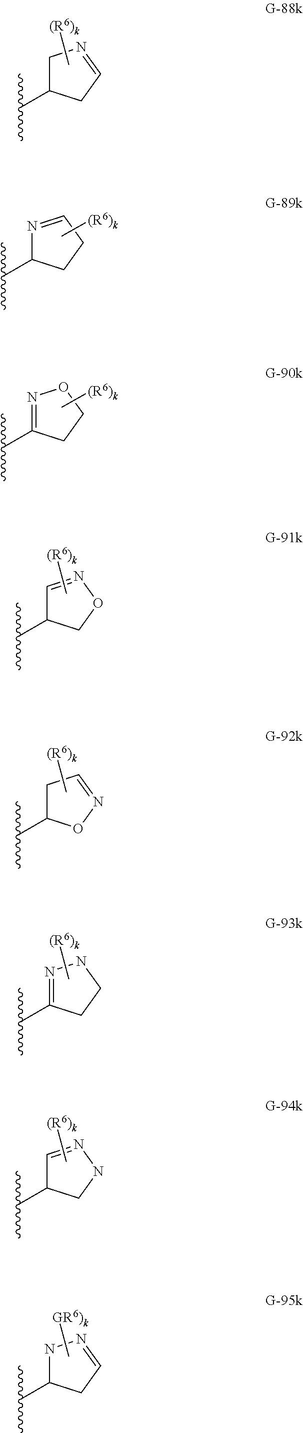 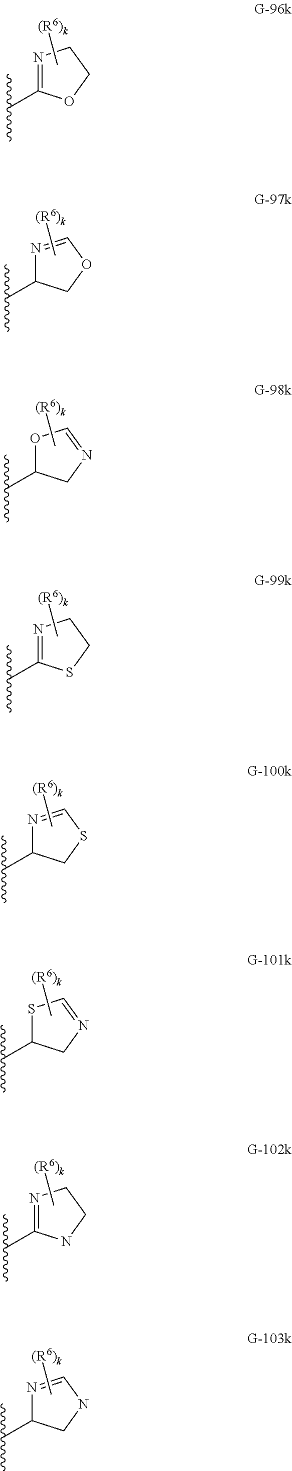 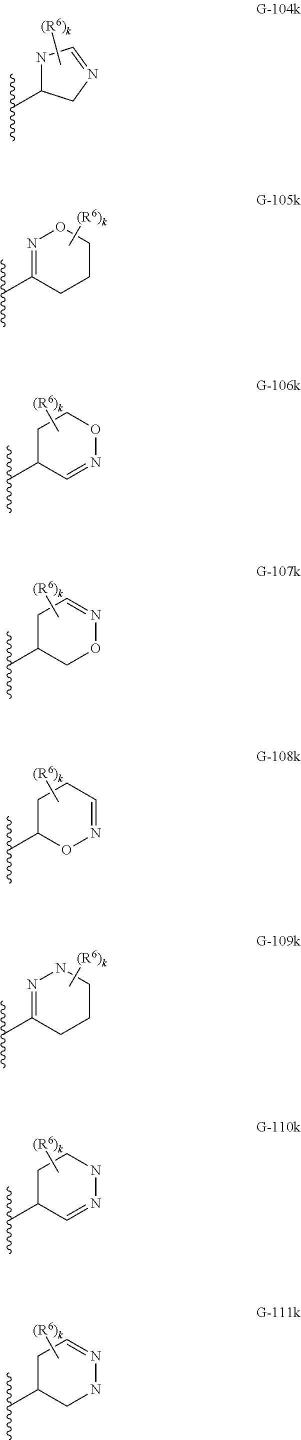 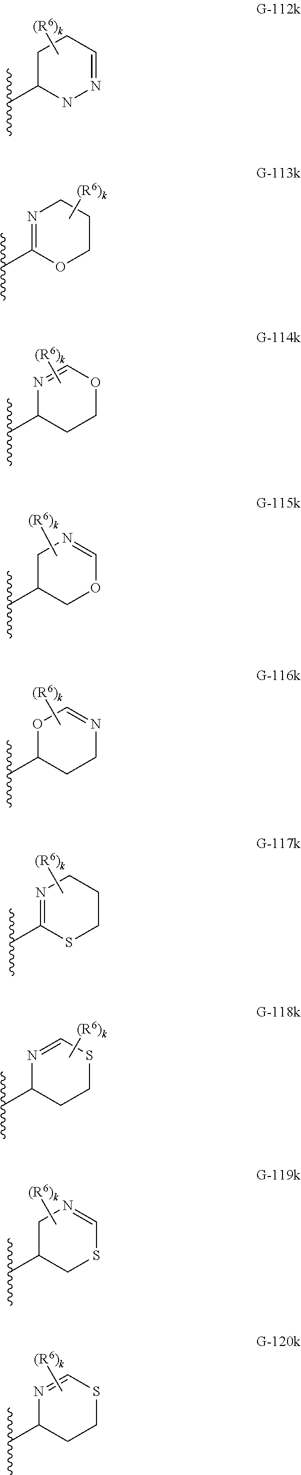 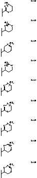 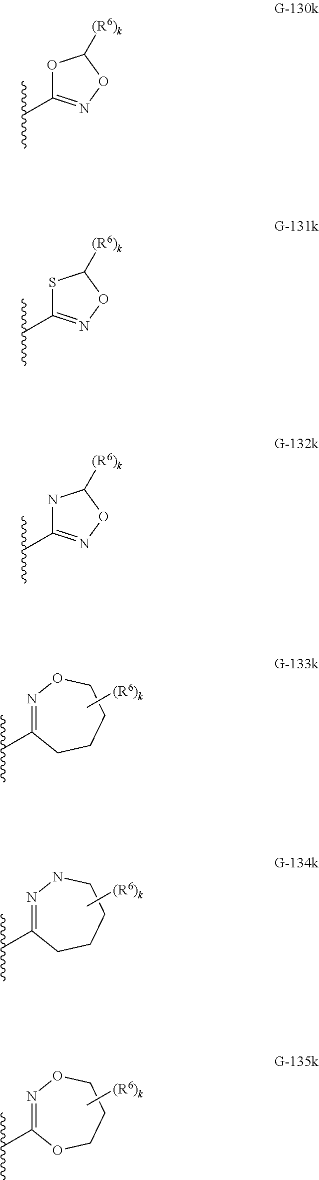
wherein the zigzag line of the moiety G denotes the bond to the group A of formula (I);
k is an integer selected from 0, 1, 2, 3, 4, 5 or 6,
and,
R⁶ is independently from one another selected from the group consisting of hydrogen, halogen, cyano, nitro, SCN, C₁-C₆-alkyl, C₃-C₆-cycloalkyl, C₂-C₆-alkenyl, C₂-C₆-alkinyl, wherein the aforementioned aliphatic and cyclo-aliphatic radicals may optionally be substituted with one or more R⁷, which are selected independently from one another,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b};
phenyl, optionally substituted with 1, 2, 3, 4, or 5 substituents R¹⁰ selected independently from one another;
a 3-, 4-, 5-, 6- or 7-membered saturated, partly saturated or unsaturated aromatic heterocyclic ring comprising 1, 2, 3 or 4 heteroatoms selected from oxygen, nitrogen and/or sulfur, optionally substituted with one or more, e.g. 1, 2, 3, 4, or 5 substituents selected independently from R¹⁰, and wherein the nitrogen and/or the sulfur atom(s) of the heterocyclic ring may optionally be oxidized;
or
two R⁶ present on one ring carbon or sulfur atom may together form ═O, ═S, ═NR^{17a}.

43. The method according to claim 33, wherein
A is selected from O, NR³ or CR^{4a}R^{4b}, and
G is the nitrogen containing 4-, 5-, or 6-membered heterocyclic or hetero-aromatic ring system selected from any of the following ring systems:
G-1k, G-2k, G-3k, G-5k, G-7k, G-9k, G-19k, G-21k, G-22k, G-26k, G-27k, G-28k, G-29k, G-30k, G-31k, G-32k, G-37k, G-45k, G-49k, G-50k, G-51k, G-90k, G-93k, G-96k, G-99k, G-102k, G-113k, G-117k, G-121k, G-125k, G-126k, G-127k, G-130k, G-131k, G-132k, G-135k as defined in claim 10, and wherein each R⁶ is independently from one another selected from the group consisting of hydrogen, halogen, cyano, nitro, C₁-C₆-alkyl, C₃-C₆-cycloalkyl, wherein the carbon atoms of the aforementioned aliphatic and cyclo-aliphatic radicals may optionally be partially or fully halogenated and/or may carry 1 or 2 radicals R¹⁵, which are selected independently from one another,
OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶;
or a ring system selected from phenyl, pyridyl, pyrimidyl, pyrazolyl, thiazolyl, oxazolyl or thiophenyl, thiazolinyl, oxazolinyl, isoxazolinyl, pyrazolinyl, imidazolinyl, each optionally substituted with one or more, e.g. 1, 2, 3, 4, or 5 substituents selected independently from R¹⁸;
or two R⁶ present on one ring carbon or sulfur atom may together form ═O, ═S, ═NR^{17a};
and wherein
k is an integer selected from 0, 1, 2 or 3.

44. The method according to claim 33, wherein
A is selected from S(O)ₙ, and
G is the nitrogen containing 4-, 5-, or 6-membered heterocyclic or hetero-aromatic ring system is selected from any of the following ring systems:
G-1k to G-21k, G-23k to G-31k, G-33k to G-46k, G-51k, G-52k, G-54k, G-56k, G-58k, G-60k, G-61k, G-63k to G-67k, G-70k to G-78k, G-81k to G-84k, G-86k to G-104k, G-113k, G-117k and G-121k,
and
R⁶ is independently selected from the group consisting of hydrogen, halogen, cyano, nitro, C₁-C₆-alkyl, C₃-C₆-cycloalkyl, wherein the carbon atoms of the aforementioned aliphatic and cyclo-aliphatic radicals may optionally be partially or fully halogenated and/or may carry 1 or 2 radicals R¹⁵, which are selected independently from one another,
OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶;
or a ring system selected from phenyl, pyridyl, pyrimidyl, pyrazolyl, thiazolyl, oxazolyl or thiophenyl, thiazolinyl, oxazolinyl, isoxazolinyl, pyrazolinyl, imidazolinyl, each optionally substituted with one or more, e.g. 1, 2, 3, 4, or 5 substituents selected independently from R¹⁸;
or two R⁶ present on one ring carbon or sulfur atom may together form ═O, ═S, ═NR^{17a};
and wherein
k is 0, 1, 2 or 3.

45. A compound of t formula (I)-1 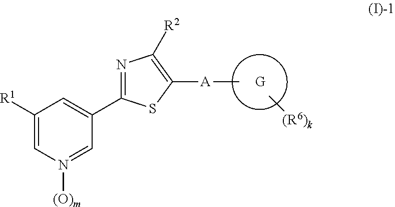
wherein
A is O or NR³, and wherein
k is an integer selected from 0, 1, 2, 3, 4, 5 or 6;
n′ is an integer selected from 0 or 1;
A is selected from O, S(O)ₙ, NR³ or CR^{4a}R^{4b};
G is a group representing
a 3, 4-, 5-, 6- or 7-membered heterocyclic or heteroaromatic monocyclic ring system or
a 7-11-membered heterobiaromatic or heterobicyclic or heterospirobicyclic ring system,
wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized, and wherein the ring system may be optionally substituted with k substituents R⁶, wherein said substituents R⁶ are selected independently from one another, if k is more than one;
R¹ is selected from the group consisting of hydrogen, cyano and halogen;
R² is selected from the group consisting of hydrogen, halogen, cyano, C₁-C₆-alkyl and C₁-C₆-cycloalkyl, wherein the latter two may be partially or fully halogenated and/or may be substituted by 1, 2, 3 or 4 radicals R⁵;
R³ is selected from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷, said substituents R⁷ being identical or different from one another if more than one substituent R⁷ is present,
OR⁸, NR^{9a}R^{9b}, S(O)ₙR⁸, S(O)ₙNR^{9a}R^{9b}, Si(R¹¹)₂R¹²;
phenyl which may be substituted 1, 2, 3, 4 or 5 substituents R¹⁰, said substituents R¹⁰ being identical or different from one another if more than one substituent R¹⁰ is present, and
a 3-, 4-, 5-, 6- or 7-membered saturated, partially unsaturated or fully unsaturated heterocyclic ring wherein said heterocyclic ring comprises 1, 2 or 3 heteroatoms selected from the group consisting of oxygen, nitrogen and sulfur atoms, wherein said nitrogen and sulfur atom(s), independently of one another, may be oxidized, and wherein the heterocyclic ring may be substituted with 1, 2, 3, 4 or 5 substituents R¹⁰, said substituents R¹⁰ being identical or different from one another if more than one substituent R¹⁰ is present;
R^{4a}, R^{4b} are selected independently of one another from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷ and wherein said substituents R⁷ are selected, independently from one another, from the group consisting of OR¹⁶, NR¹⁷R^{17b}, S(O)ₙR¹⁶, S(O)ₙNR^{17a}R^{17b}, C(═O)R¹⁵, C(═O)NR^{17a}R^{17b}, C(═O)OR¹⁶, C(═S)R¹⁵, C(═S)NR^{17a}R^{17b}, C(═S)OR¹⁶, C(═S)SR¹⁶, C(—NR^{17a})R¹⁵, C(NR^{17a})NR^{17a}R^{17b}, and Si(R¹¹)₂R¹²,
or
R^{4a} and R^{4b} together may form a ═CHR¹³, ═CR⁷R¹³, ═NR^{9a} or ═NOR⁸ radical;
R⁵ is each independently from one another selected from the group consisting of cyano, —SCN, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR₁₆, S(O)ₙNR^{17a}R^{17b}, NR^{17a}R^{17b}, C(═O)NR^{17a}R^{17b}, C(═S)NR^{17a}R^{17b}, C(═O)OR¹⁶,
phenyl, optionally substituted with 1, 2, 3, 4 or 5 substituents R¹⁸, which are independently selected from one another,
and a 3-, 4-, 5-, or 6-membered saturated, partly saturated or unsaturated heterocyclic ring comprising 1, 2 or 3 heteroatoms selected from the group consisting of oxygen, nitrogen and/or sulfur, wherein said nitrogen and sulfur atoms, independently of one another, may be oxidized, and wherein the heterocyclic ring may be substituted with 1, 2, 3, 4 or 5 substituents R¹⁸, said substituents R¹⁸ being identical or different from one another if more than one substituent R¹⁸ is present;
R⁶ is each independently from one another selected from the group consisting of hydrogen, halogen, cyano, azido, nitro, SCN, SF₅, C₃-C₈-cycloalkyl, C₂-C₁₀-alkenyl, C₂-C₁₀-alkinyl, and wherein the carbon atoms of the aforementioned aliphatic and cyclo-aliphatic radicals may optionally be further substituted independently from one another with one or more R⁷,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
phenyl, optionally substituted with 1, 2, 3, 4 or 5 substituents R¹⁰ selected independently from one another from,
and a 3-, 4-, 5-, 6- or 7-membered saturated, partly saturated or unsaturated aromatic heterocyclic ring comprising 1, 2, or 3 heteroatoms selected from the group consisting of oxygen, nitrogen and/or sulfur, optionally substituted with 1, 2, 3, 4 or 5 substituents R¹⁰, selected independently from one another, and wherein the nitrogen and/or the sulfur atom(s) of the heterocyclic ring may optionally be oxidized;
R⁷ is each independently from one another selected from the group consisting of hydrogen, halogen, cyano, azido, nitro, —SCN, SF₅, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)R¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
phenyl, optionally substituted with 1, 2, 3, 4 or 5 sub-stituents R¹⁸, selected independently from one another,
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized,
or
two R⁷ present on one carbon atom may together form ═O, ═CR¹³R¹⁴; ═S, ═NR^{17a}, ═NOR¹⁶; ═NNR^{17a};
or
two R⁷ may form a 3-, 4-, 5-, 6-, 7- or 8-membered saturated or partly unsaturated carbocyclic or heterocyclic ring together with the carbon atoms to which the two R⁷ are bonded to;
R⁸ is each independently from one another selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₄-C₈-alkylcycloalkyl, C₃-C₈-halocycloalkyl, C₂-C₆-alkenyl, C₂-C₆-haloalkenyl, C₂-C₆-alkinyl, C₂-C₆ haloalkinyl, —Si(R¹¹)₂R¹², S(O)ₙR¹⁶, S(O)ₙNR^{17a}R^{17b}, NR^{17a}R^{17b}, —N═CR¹³R¹⁴, —C(═O)R¹⁵, C(═O)NR^{17a}R^{17b}, C(═S)NR^{17a}R^{17b}, C(═O)OR¹⁶,
phenyl, optionally substituted with one or more substituents R¹⁸; which are selected independently from one another,
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized;
R^{9a}, R^{9b} are each independently from one another selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆-haloalkinyl,
S(O)ₙR¹⁶, —S(O)ₙNR^{17a}R^{17b}, C(═O)R¹⁵, C(═O)OR¹⁶, C(═O)NR^{17a}R^{17b}, C(═S)R¹⁵, C(═S)SR¹⁶, C(═S)NR^{17a}R^{17b}, C(═NR^{17a})R¹⁵;
phenyl, optionally substituted with 1, 2, 3 or 4 substituents R¹⁸, selected independently from one another;
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4, substituents R¹⁸, selected independently from one another, and wherein the nitrogen and/or the sulfur atom(s) of the heterocyclic ring may optionally be oxidized;
or,
R^{9a} and R^{9b} are together a C₂-C₇ alkylene chain and form a 3-, 4-, 5-, 6-, 7- or 8-membered saturated, partly saturated or unsaturated aromatic ring together with the nitrogen atom they are bonded to, wherein the alkylene chain may contain one or two heteratoms selected from the group consisting of oxygen, sulfur and nitrogen, and may optionally be substituted with halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
phenyl, optionally substituted with one or more substituents R¹⁸, which are selected independently from one another;
or a 3-, 4-, 5-, 6-, or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents R¹⁸, selected independently from one another, and wherein the nitrogen and/or the sulfur atom(s) of the heterocyclic ring may optionally be oxidized;
or
R^{9a} and R^{9b} together may form a ═CR¹³R¹⁴, ═NR¹⁷ or ═NOR¹⁶ radical;
R¹⁰ is each independently from one another selected from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, wherein the carbon atoms of the aforementioned aliphatic and cyclo-aliphatic radicals may optionally be substituted with one or more R¹⁵, which are selected independently from one another, from the group consisting of Si(R¹¹)₂R¹², OR¹⁶, OS(O)ₙR¹⁶, —S(O)ₙR¹⁶, S(O)ₙNR^{17a}R^{17b}, NR^{17a}R^{17b}, C(═O)R¹⁵, C(═O)OR¹⁶, —C(═NR^{17a})R¹⁵, C(═O)NR^{17a}R^{17b}, C(═S)NR^{17a}R^{17b},
phenyl, optionally substituted with halogen, cyano, nitro, C₁-C₆-alkyl, C₁-C₆-haloalkyl, C₁-C₆-alkoxy and C₁-C₆-haloalkoxy,
or a 3-, 4-, 5-, 6- or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents selected independently from one another from the group consisting of halogen, cyano, NO₂, C₁-C₆-alkyl, C₁-C₆-haloalkyl, C₁-C₆-alkoxy and C₁-C₆-haloalkoxy, and wherein the nitrogen and/or the sulfur atom(s) of the heterocyclic ring may optionally be oxidized;
or
two R¹⁰ present together on one atom of a partly saturated heterocyclic may be ═O, ═CR¹³R¹⁴; ═S, ═NR^{17a}, ═NOR¹⁶ or ═NNR^{17a};
or,
two R¹⁰ on adjacent carbon atoms may be a bridge selected from the group consisting of CH₂CH_{Z}CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17a}, CH₂CH═N, CH═CH—NR^{17a}, OCH═N, and SCH═N and form together with the carbon atoms to which the two R¹⁰ are bonded to a 5-membered or 6-membered partly saturated or unsaturated, aromatic carbocyclic or heteocyclic ring, wherein the ring may optionally be substituted with one or two substituents selected from the group consisting of ═O, OH, CH₃, OCH₃, halogen, cyano, halomethyl and halomethoxy;
R¹¹, R¹² are each independently from one another selected from the group consisting of hydrogen, halogen, C₁-C₆ alkyl, C₁-C₆ haloalkyl, C₁-C₆ alkoxy, C₁-C₆ alkoxyalkyl, C₂-C₆ alkenyl, C₂-C₆ haloalkenyl, C₂-C₆ alkinyl, C₂-C₆ haloalkinyl, C₃-C₈ cycloalkyl, C₃-C₈ halocycloalkyl, C₁-C₆ alkoxyalkyl, C₁-C₆ haloalkoxyalkyl and
phenyl, optionally substituted with one or more substituents R¹⁸; which are selected independently from one another;
R¹³, R¹⁴ are each independently from one another selected from the group consisting of hydrogen, C₁-C₄ alkyl, C₁-C₆ cycloalkyl, C₁-C₄ alkoxyalkyl, phenyl and benzyl;
R¹⁵ is each independently from one another selected from the group consisting of hydrogen, halogen, cyano, nitro, OH, SH, SCN, SF₅, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄ alkoxy;
phenyl, benzyl, pyridyl, and phenoxy, wherein the last four radicals may be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino and di-(C₁-C₆-alkyl)amino,
or
two R¹⁵ present on the same carbon atom may together be ═O, ═CH(C₁-C₄), ═C(C₁-C₄-alkyl)C₁-C₄-alkyl, ═N(C₁-C₆-alkyl) or ═NO(C₁-C₆-alkyl);
R¹⁶ is each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-eycloalkyl, wherein the four last mentioned radicals may be unsubstituted, partially or fully halogenated and/or oxygenated and/or may carry 1 or 2 radicals selected from C₁-C₄ alkoxy, phenyl, benzyl, pyridyl, and phenoxy, wherein the last four radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl;
R^{17a}, R^{17b} are each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
phenyl, benzyl, pyridyl, and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl,
or,
R^{17a} and R^{17b} may together be a C₂-C₆ alkylene chain forming a 3- to 7-membered saturated, partly saturated or unsaturated ring together with the nitrogen atom R^{17a} and R^{17b} are bonded to, wherein the alkylene chain may contain 1 or 2 heteroatoms selected from the group consisting of oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
R¹⁸ is each independently from one another selected from the group consisting of hydrogen, halogen, nitro, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
phenyl, benzyl, pyridyl, and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy), and (C₁-C₆-alkoxy)carbonyl;
or
two R¹⁸ present together on one atom of a partly saturated atom may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
or,
two R¹⁸ on two adjacent carbon atoms may be together a C₂-C₆ alkylene chain, which form together with the carbon atom they are bonded to a 3-, 4-, 5-, 6- or 7-membered saturated, partly saturated or unsaturated aromatic, wherein the alkylene chain may contain 1 or 2 heteroatoms selected from the group consisting of oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
n is an integer selected from 0, 1 or 2;
or an enantiomer, diastereomer or agriculturally or veterinarily acceptable salt thereof.

46. A substituted 3-pyridyl thiazole compound of the general formula (I)-2 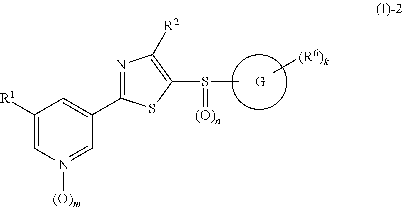
wherein
G is a molecular group representing a ring system selected from
a 3, 4-, 5-, 6- or 7-membered heterocyclic or heteroaromatic monocyclic ring system or
a 7-11-membered heterobiaromatic or heterobicyclic or heterospirobicyclic ring system,
wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized, and wherein the ring system is attached via a carbon ring atom to the remainder of the molecule, and
wherein the ring system may be optionally substituted with k substituents R⁶, wherein said substituents R⁶ are selected independently from one another, if k is more than one,
and wherein
k is an integer selected from 0, 1, 2, 3, 4, 5 or 6;
m is an integer selected from 0 or 1;
R¹ is selected from the group consisting of hydrogen, cyano and halogen;
R² is selected from the group consisting of hydrogen, halogen, cyano, C₁-C₆-alkyl and C₁-C₆-cycloalkyl, wherein the latter two may be partially or fully halogenated and/or may be substituted by 1, 2, 3 or 4 radicals R⁵;
R³ is selected from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷, said substituents R⁷ being identical or different from one another if more than one substituent R⁷ is present,
OR⁸, NR^{9a}R^{9b}, S(O)ₙR⁸, S(O)ₙNR^{9a}R^{9b}, Si(R¹¹)₂R¹²;
phenyl which may be substituted 1, 2, 3, 4 or 5 substituents R¹⁰, said substituents R¹⁰ being identical or different from one another if more than one substituent R¹⁰ is present, and
a 3-, 4-, 5-, 6- or 7-membered saturated, partially unsaturated or fully unsaturated heterocyclic ring wherein said heterocyclic ring comprises 1, 2 or 3 heteroatoms selected from the group consisting of oxygen, nitrogen and sulfur atoms, wherein said nitrogen and sulfur atom(s), independently of one another, may be oxidized, and wherein the heterocyclic ring may be substituted with 1, 2, 3, 4 or 5 substituents R¹⁰, said substituents R¹⁰ being identical or different from one another if more than one substituent R¹⁰ is present;
R^{4a}, R^{4b} are selected independently of one another from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷ and wherein said substituents R⁷ are selected, independently from one another, from the group consisting of OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶, C(═S)R¹⁵, C(═S)NR^{17a}R^{17b}, C(═S)OR¹⁶, C(═S)SR¹⁶, C(═NR^{17a})R¹⁵, C(═NR^{17a})NR^{17a}R^{17b}, and Si(R¹¹)₂R¹²,
or
R^{4a} and R^{4b} together may form a ═CHR¹³, ═CR⁷R¹³, ═NR^{9a} or ═NOR⁸ radical;
R⁵ is each independently from one another selected from the group consisting of cyano, —SCN, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
phenyl, optionally substituted with 1, 2, 3, 4 or 5 substituents R¹⁸, which are independently selected from one another,
and a 3-, 4-, 5-, or 6-membered saturated, partly saturated or unsaturated heterocyclic ring comprising 1, 2 or 3 heteroatoms selected from the group consisting of oxygen, nitrogen and/or sulfur, wherein said nitrogen and sulfur atoms, independently of one another, may be oxidized, and wherein the heterocyclic ring may be substituted with 1, 2, 3, 4 or 5 substituents R¹⁸, said substituents R¹⁸ being identical or different from one another if more than one substituent R¹⁸ is present;
R⁶ is each independently from one another selected from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, and wherein the carbon atoms of the aforementioned aliphatic and cyclo-aliphatic radicals may optionally be further substituted independently from one another with one or more R⁷,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
phenyl, optionally substituted with 1, 2, 3, 4 or 5 substituents R¹⁰ selected independently from one another from,
and a 3-, 4-, 5-, 6- or 7-membered saturated, partly saturated or unsaturated aromatic heterocyclic ring comprising 1, 2, or 3 heteroatoms selected from the group consisting of oxygen, nitrogen and/or sulfur, optionally substituted with 1, 2, 3, 4 or 5 substituents R¹⁰, selected independently from one another, and wherein the nitrogen and/or the sulfur atom(s) of the heterocyclic ring may optionally be oxidized;
R⁷ is each independently from one another selected from the group consisting of hydrogen, halogen, cyano, azido, nitro, —SCN, SF₅, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
phenyl, optionally substituted with 1, 3, 4 or 5 sub-stituents R¹⁸, selected independently from one another,
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized,
or
two R⁷ present on one carbon atom may together form ═O, ═CR¹³R¹⁴; ═S, ═NR^{17a}, ═NOR¹⁶; ═NNR^{17a};
or
two R⁷ may form a 3-, 4-, 5-, 6-, 7- or 8-membered saturated or partly unsaturated carbocyclic or heterocyclic ring together with the carbon atoms to which the two R⁷ are bonded to;
R⁸ is each independently from one another selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₄-C₈-alkylcycloalkyl, C₃-C₈-halocycloalkyl, C₂-C₆-alkenyl, C₂-C₆-haloalkenyl, C₂-C₆-alkinyl, C₂-C₆ haloalkinyl, —Si(R¹¹)₂R¹², S(O)ₙR¹⁶, S(O)ₙNR^{17a}R^{17b}, NR^{17a}R^{17b}, —N—CR¹³R¹⁴, —C(═O)R¹⁵, C(═O)NR^{17a}R^{17b}, C(═S)NR^{17a}R^{17b}, C(═O)OR¹⁶,
phenyl, optionally substituted with one or more substituents R¹⁸; which are selected independently from one another,
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized;
R^{9a}, R^{9b} are each independently from one another selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
S(O)ₙR¹⁶, —S(O)ₙNR^{17a}R^{17b}, C(═O)R¹⁵, C(═O)OR¹⁶, C(═O)NR^{17a}R^{17b}, C(═S)R¹⁵, C(═S)SR¹⁶, C(═S)NR^{17a}R^{17b}, C(═NR^{17a})R¹⁵;
phenyl, optionally substituted with 1, 2, 3 or 4 substituents R¹⁸, selected independently from one another;
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4, substituents R¹⁸, selected independently from one another, and wherein the nitrogen and/or the sulfur atom(s) of the heterocyclic ring may optionally be oxidized;
or,
R^{9a} and R^{9b} are together a C₂-C₇ alkylene chain and form a 3-, 4-, 5-, 6-, 7- or 8-membered saturated, partly saturated or unsaturated aromatic ring together with the nitrogen atom they are bonded to, wherein the alkylene chain may contain one or two heteratoms selected from the group consisting of oxygen, sulfur and nitrogen, and may optionally be substituted with halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
phenyl, optionally substituted with one or more substituents R¹⁸, which are selected independently from one another;
or a 3-, 4-, 5-, 6-, or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents R¹⁸, selected independently from one another, and wherein the nitrogen and/or the sulfur atom(s) of the heterocyclic ring may optionally be oxidized;
or
R^{9a} and R^{9b} together may form a ═CR¹³R¹⁴, ═NR¹⁷ or ═NOR¹⁶ radical;
R¹⁰ is each independently from one another selected from the group consisting of hydrogen, halogen, cyano, azido, nitro, SCN, SF₅, C₃-C₈-cycloalkyl, C₂-C₁₀-alkenyl, C₂-C₁₀-alkinyl, wherein the carbon atoms of the aforementioned aliphatic and cyclo-aliphatic radicals may optionally be substituted with one or more R¹⁵, which are selected independently from one another, from the group consisting of Si(R¹¹)₂R¹², OR¹⁶, OS(O)ₙR¹⁶, —S(O)ₙR¹⁶, S(O)ₙNR^{17a}R^{17b}, NR^{17a}R^{17b}, C(═O)R¹⁵, C(═O)OR¹⁶, —C(═NR^{17a})R¹⁵, C(═O)NR^{17a}R^{17b}, C(═S)NR^{17a}R^{17b},
phenyl, optionally substituted with halogen, cyano, nitro, C₁-C₆-alkyl, C₁-C₆-alkoxy and C₁-C₆-haloalkoxy,
or a 3-, 4-, 5-, 6- or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents selected independently from one another from the group consisting of halogen, cyano, NO₂, C₁-C₆-haloalkyl, C₁-C₆-alkoxy and C₁-C₆-haloalkoxy, and wherein the nitrogen and/or the sulfur atom(s) of the heterocyclic ring may optionally be oxidized;
or
two R¹⁶ present together on one atom of a partly saturated heterocyclic may be ═O, ═CR¹³R¹⁴; ═S, ═NR^{17a}, ═NOR¹⁶ or ═NNR^{17a};
or,
two R¹⁰ on adjacent carbon atoms may be a bridge selected from the group consisting of CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17a}, CH₂CH═N, CH═CH—NR^{17a}, OCH═N, and SCH═N and form together with the carbon atoms to which the two Ri® are bonded to a 5-membered or 6-membered partly saturated or unsaturated, aromatic carbocyclic or heteocyclic ring, wherein the ring may optionally be substituted with one or two substituents selected from the group consisting of ═O, OH, CH₃, OCH₃, halogen, cyano, halomethyl and halomethoxy;
R¹¹, R¹² are each independently from one another selected from the group consisting of hydrogen, halogen, C₁-C₆ alkyl, C₁-C₆ haloalkyl, C₁-C₆ alkoxy, C₁-C₆ alkoxyalkyl, C₂-C₆ alkenyl, C₂-C₆ haloalkenyl, C₂-C₆ alkinyl, C₂-C₆ haloalkinyl, C₃-C₈ cycloalkyl, C₃-C₈ halocycloalkyl, C₁-C₆ alkoxyalkyl, C₁-C₆ haloalkoxyalkyl and
phenyl, optionally substituted with one or more substituents R¹⁸; which are selected independently from one another;
R¹³, R¹⁴ are each independently from one another selected from the group consisting of hydrogen, C₁-C₄ alkyl, C₁-C₆ cycloalkyl, C₁-C₄ alkoxyalkyl, phenyl and benzyl;
R¹⁵ is each independently from one another selected from the group consisting of hydrogen, halogen, cyano, nitro, OH, SH, SCN, SF₅, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄ alkoxy;
phenyl, benzyl, pyridyl, and phenoxy, wherein the last four radicals may be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino and di-(C₁-C₆-alkyl)amino,
or
two R¹⁵ present on the same carbon atom may together be ═O, ═CH(C₁-C₄), ═C(C₁-C₄-alkyl)C₁-C₄-alkyl, ═N(C₁-C₆-alkyl) or ═NO(C₁-C₆-alkyl);
R¹⁶ is each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned radicals may be unsubstituted, partially or fully halogenated and/or oxygenated and/or may carry 1 or 2 radicals selected from C₁-C₄ alkoxy, phenyl, benzyl, pyridyl, and phenoxy, wherein the last four radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl;
R^{17a}, R^{17b} are each independently from one another selected from the group consisting of hydrogen, cyano, C₁-C₆-alkoxy, C₁-C₆-haloalkoxy, C₁-C₆-allylthio, C₁-Cₒ-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
phenyl, benzyl, pyridyl, and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl,
or,
R^{17a} and R^{17b} may together be a C₂-C₆ alkylene chain forming a 3- to 7-membered saturated, partly saturated or unsaturated ring together with the nitrogen atom R^{17a} and R^{17b} are bonded to, wherein the alkylene chain may contain 1 or 2 heteroatoms selected from the group consisting of oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
R¹⁸ is each independently from one another selected from the group consisting of hydrogen, halogen, nitro, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
phenyl, benzyl, pyridyl, and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy), and (C₁-C₆-alkoxy)carbonyl;
or
two R¹⁸ present together on one atom of a partly saturated atom may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
or,
two R¹⁸ on two adjacent carbon atoms may be together a C₂-C₆ alkylene chain, which form together with the carbon atom they are bonded to a 3-, 4-, 5-, 6- or 7-membered saturated, partly saturated or unsaturated aromatic, wherein the alkylene chain may contain 1 or 2 heteroatoms selected from the group consisting of oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
n is an integer selected from 0, 1 or 2;
and/or an enantiomer, diastereomer or agriculturally or veterinarily acceptable salt thereof.

47. A substituted 3-pyridyl thiazole compound of the general formula (I)-3 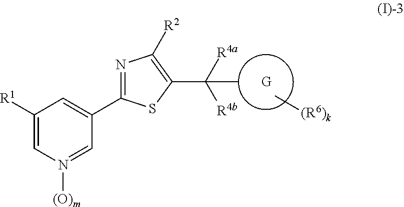
wherein
k is an integer selected from 0, 1, 2, 3, 4, 5 or 6;
m is an integer selected from 0 or 1;
R¹ is selected from the group consisting of hydrogen, cyano and halogen;
R² is selected from the group consisting of hydrogen, halogen, cyano, C₃-C₆-alkyl, C₁-C₆-cycloalkyl, the latter two may be partially or fully halogenated and/or may be substituted by one or more radicals R⁵;
and wherein
R^{4a}, R^{4b} are selected independently of one another from the group consisting of hydrogen, cyano, C₁-C₁₀-alkyl, C₃-C₈-cycloalkyl, C₂-C₁₀-alkenyl, C₂-C₁₀-alkynyl, wherein the aforementioned aliphatic and cycloaliphatic radicals may be substituted with 1 to 10 substituents R⁷ and wherein said substituents R⁷ are selected, independently from one another, from the group consisting of OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶, C(═S)R¹⁵, C(═S)NR^{17a}R^{17b}, C(═S)OR¹⁶, C(═S)SR¹⁶, C(═NR^{17a})R¹⁵, C(═NR^{17a})NR^{17a}R^{17b}, and Si(R¹¹)₂R¹²,
or
R^{4a} and R^{4b} together may form a ═CHR¹³, ═CR⁷R¹³, ═NR^{9a} or ═NOR⁸ radical;
R⁵ is each independently from one another selected from the group consisting of cyano, —SCN, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(S)NR^{17a}R^{17b}, C(═O)OR¹⁶,
phenyl, optionally substituted with 1, 2, 3, 4 or 5 substituents R¹⁸, which are independently selected from one another,
and a 3-, 4-, 5-, or 6-membered saturated, partly saturated or unsaturated heterocyclic ring comprising 1, 2 or 3 heteroatoms selected from the group consisting of oxygen, nitrogen and/or sulfur, wherein said nitrogen and sulfur atoms, independently of one another, may be oxidized, and wherein the heterocyclic ring may be substituted with 1, 2, 3, 4 or 5 substituents R¹⁸, said substituents R¹⁸ being identical or different from one another if more than one substituent R¹⁸ is present;
R⁶ is each independently from one another selected from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, and wherein the carbon atoms of the aforementioned aliphatic and cyclo-aliphatic radicals may optionally be further substituted independently from one another with one or more R⁷,
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
phenyl, optionally substituted with 1, 2, 3, 4 or 5 substituents R¹⁰ selected independently from one another from,
and a 3-, 4-, 5-, 6- or 7-membered saturated, partly saturated or unsaturated aromatic heterocyclic ring comprising 1, 2, or 3 heteroatoms selected from the group consisting of oxygen, nitrogen and/or sulfur, optionally substituted with 1, 2, 3, 4 or 5 substituents R¹⁰, selected independently from one another, and wherein the nitrogen and/or the sulfur atom(s) of the heterocyclic ring may optionally be oxidized;
R⁷ is each independently from one another selected from the group consisting of hydrogen, halogen, cyano, azido, nitro, —SCN, SF₅, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
phenyl, optionally substituted with 1, 2, 3, 4 or 5 sub-stituents R¹⁸, selected independently from one another,
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized,
or
two R⁷ present on one carbon atom may together form ═O, ═CR¹³R¹⁴; ═S, ═NR^{17a}, ═NOR¹⁶; ═NNR^{17a};
or
two R⁷ may form a 3-, 4-, 5-, 6-, 7- or 8-membered saturated or partly unsaturated carbocyclic or heterocyclic ring together with the carbon atoms to which the two R⁷ are bonded to;
R⁸ is each independently from one another selected from the group consisting of hydrogen, cyano, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, C₃-C₈-cycloalkyl, C₄-C₈-alkylcycloalkyl, C₃-C₈-halocycloalkyl, C₂-C₆-alkenyl, C₂-C₆-haloalkenyl, C₂-C₆-alkinyl, C₂-C₆ haloalkinyl, —Si(R¹¹)₂R¹², S(O)ₙR¹⁶, S(O)NR^{17a}R^{17b}, NR^{17a}R^{17b}, —N═CR¹³R¹⁴, —C(═O)R¹⁵, C(═O)NR^{17a}R^{17b}, C(═S)NR^{17a}R^{17b}, —C(═O)OR¹⁶,
phenyl, optionally substituted with one or more substituents R¹⁸; which are selected independently from one another,
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4 substituents R¹⁸, selected independently from one another, and wherein the nitrogen and/or the sulfur atom(s) of the heterocyclic ring may optionally be oxidized;
R^{9a}, R^{9b} are each independently from one another selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
S(O)ₙR¹⁶, —S(O)ₙNR^{17a}R^{17b}, C(═O)R¹⁵, C(═O)OR¹⁶, C(═O)NR^{17a}R^{17b}, C(═S)R¹⁵, C(═S)SR¹⁶, C(═S)NR^{17a}R^{17b}, C(═NR^{17a})R¹⁵;
phenyl, optionally substituted with 1, 2, 3 or 4 substituents R¹⁸, selected independently from one another;
and a 3-, 4-, 5-, 6- or 7-membered saturated, partly saturated or unsaturated aromatic heterocyclic ring comprising 1, 2 or 3 heteroatoms selected from the group consisting of oxygen, nitrogen and/or sulfur, optionally substituted with 1, 2, 3 or 4, substituents R¹⁸, selected independently from one another, and wherein the nitrogen and/or the sulfur atom(s) of the heterocyclic ring may optionally be oxidized;
or,
R^{9a} and R^{9b} are together a C₂-C₇ alkylene chain and form a 3-, 4-, 5-, 6-, 7- or 8-membered saturated, partly saturated or unsaturated aromatic ring together with the nitrogen atom they are bonded to, wherein the alkylene chain may contain one or two heteratoms selected from the group consisting of oxygen, sulfur and nitrogen, and may optionally be substituted with halogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkinyl, C₂-C₆ haloalkinyl,
phenyl, optionally substituted with one or more substituents R¹⁸, which are selected independently from one another;
or a 3-, 4-, 5-, 6-, or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents R¹⁸, selected independently from one another, and wherein the nitrogen and/or the sulfur atom(s) of the heterocyclic ring may optionally be oxidized;
or
R^{9a} and R^{9b} together may form a ═CR¹³R¹⁴, ═NR¹⁷ or ═NOR¹⁶ radical;
R¹⁰ is each independently from one another selected from the group consisting of hydrogen, halogen, cyano, azido, nitro, SCN, SF₅, C₁-C₁₀-alkyl, C₃-C₈-cycloalkyl, C₂-C₁₀-alkenyl, C₂-C₁₀-alkinyl, wherein the carbon atoms of the aforementioned aliphatic and cyclo-aliphatic radicals may optionally be substituted with one or more R¹⁵, which are selected independently from one another, from the group consisting of Si(R¹¹)₂R¹², OR¹⁶, OS(O)ₙR¹⁶, —S(O)ₙR¹⁶, S(O)ₙNR^{17a}R^{17b}, NR^{17a}R^{17b}, C(═O)R¹⁵, C(═O)OR¹⁶, —C(═NR^{17a})R¹⁵, C(═O)NR^{17a}R^{17b}, C(═S)NR^{17a}R^{17b},
phenyl, optionally substituted with halogen, cyano, nitro, C₁-C₆-alkyl, C₁-C₆-haloalkyl, C₁-C₆-alkoxy and C₁-C₆-haloalkox_{Y},
or a 3-, 4-, 5-, 6- or 7-membered saturated, partly saturated or unsaturated aromatic heterocyclic ring comprising 1, 2 or 3 heteroatoms selected from the group consisting of oxygen, nitrogen and sulfur, optionally substituted with one or more substituents selected independently from one another from the group consisting of halogen, cyano, NO₂, C₁-C₆-alkyl, C₁-C₆-haloalkyl, C₁-C₆-alkoxy and C₁-C₆-haloalkoxy, and wherein the nitrogen and/or the sulfur atom(s) of the heterocyclic ring may optionally be oxidized;
or
two R¹⁰ present together on one atom of a partly saturated heterocyclic may be ═O, ═CR¹³R¹⁴; ═S, ═NR^{17a}, ═NOR¹⁶ or ═NNR^{17a};
or,
two R¹⁰ on adjacent carbon atoms may be a bridge selected from the group consisting of CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17a}, CH₂CH═N, CH═CH—NR^{17a}, OCH═N, and SCH═N and form together with the carbon atoms to which the two R¹⁰ are bonded to a 5-membered or 6-membered partly saturated or unsaturated, aromatic carbocyclic or heteocyclic ring, wherein the ring may optionally be substituted with one or two substituents selected from the group consisting of ═O, OH, CH₃, OCH₃, halogen, cyano, halomethyl and halomethoxy;
R¹¹, R¹² are each independently from one another selected from the group consisting of hydrogen, halogen, C₁-C₆ alkyl, C₁-C₆ haloalkyl, C₁-C₆ alkoxy, C₁-C₆ alkoxyalkyl, C₂-C₆ alkenyl, C₂-C₆ haloalkenyl, C₂-C₆ alkinyl, C₂-C₆ haloalkinyl, C₃-C₈ cycloalkyl, C₃-C₈ halocycloalkyl, C₁-C₆ alkoxyalkyl, C₁-C₆ haloalkoxyalkyl and
phenyl, optionally substituted with one or more substituents R¹⁸; which are selected independently from one another;
R¹³, R¹⁴ are each independently from one another selected from the group consisting of hydrogen, C₁-C₄ alkyl, C₁-C₆ cycloalkyl, C₁-C₄ alkoxyalkyl, phenyl and benzyl;
R¹⁵ is each independently from one another selected from the group consisting of hydrogen, halogen, cyano, nitro, OH, SH, SCN, SF₅, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄ alkoxy;
phenyl, benzyl, pyridyl, and phenoxy, wherein the last four radicals may be unsubstituted, partially or fully halogenated and/or to carry 1, 2 or 3 substituents selected from the group consisting of C₁-C₆-alkyl, C₁-C₆-haloalkyl, C₁-C₆-alkoxy, C₁-C₆ haloalkoxy, (C₁-C₆-alkoxy)carbonyl, (C₁-C₆-alkyl)amino and di-(C₁-C₆-alkyl)amino,
or
two R¹⁵ present on the same carbon atom may together be ═O, ═CH(C₁-C₄), ═C(C₁-C₄-alkyl)C₁-C₄-alkyl, ═N(C₁-C₆-alkyl) or ═NO(C₁-C₆-alkyl);
R¹⁶ is each independently from one another selected from the group consisting of hydrogen, cyano, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned radicals may be unsubstituted, partially or fully halogenated and/or oxygenated and/or may carry 1 or 2 radicals selected from C₁-C₄ alkoxy, phenyl, benzyl, pyridyl, and phenoxy, wherein the last four radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl;
R^{17a}, R^{17b} are each independently from one another selected from the group consisting of hydrogen, cyano, C₁-C₆-alkoxy, C₁-C₆-haloalkoxy, C₁-C₆-allylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
phenyl, benzyl, pyridyl, and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy and (C₁-C₆-alkoxy)carbonyl,
or,
R^{17a} and R^{17b} may together be a C₂-C₆ alkylene chain forming a 3- to 7-membered saturated, partly saturated or unsaturated ring together with the nitrogen atom R^{17a} and R^{17b} are bonded to, wherein the alkylene chain may contain 1 or 2 heteroatoms selected from the group consisting of oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
R¹⁸ is each independently from one another selected from the group consisting of hydrogen, halogen, nitro, cyano, OH, SH, C₁-C₆-alkoxy, C₁-C₆-haloalkoxy, C₁-C₆-alkylthio, C₁-C₆-alkylsulfinyl, C₁-C₆-alkylsulfonyl, C₁-C₆-haloalkylthio, trimethylsilyl, triethylsilyl, tertbutyldimethylsilyl,
C₁-C₆-alkyl, C₂-C₆-alkenyl, C₂-C₆-alkinyl, C₃-C₈-cycloalkyl, wherein the four last mentioned aliphatic and cyclo-aliphatic radicals may be unsubstituted, partially or fully halogenated and/or oxygenated and/or may carry 1 or 2 radicals selected from C₁-C₄-alkoxy,
phenyl, benzyl, pyridyl, and phenoxy, wherein the four last mentioned radicals may be unsubstituted, partially or fully halogenated and/or carry 1, 2 or 3 substituents selected from the group consisting of C₁-C₆-alkyl, C₁-C₆-haloalkyl, C₁-C₆-alkoxy, C₁-C₆ haloalkoxy), and (C₁-C₆-alkoxy)carbonyl;
or
two R¹⁸ present together on one atom of a partly saturated atom may be ═O, ═S, ═N(C₁-C₆-alkyl), ═NO(C₁-C₆-alkyl), ═CH(C₁-C₄-alkyl) or ═C(C₁-C₄-alkyl)C₁-C₄-alkyl;
or,
two R¹⁸ on two adjacent carbon atoms may be together a C₂-C₆ alkylene chain, which form together with the carbon atom they are bonded to a 3-, 4-, 5-, 6- or 7-membered saturated, partly saturated or unsaturated aromatic, wherein the alkylene chain may contain 1 or 2 heteroatoms selected from the group consisting of oxygen, sulfur or nitrogen, and may optionally be substituted with halogen, C₁-C₄-haloalkyl, C₁-C₄-alkoxy or C₁-C₄-haloalkoxy, and wherein the nitrogen and/or the sulfur atom(s) of the heterocyclic ring may optionally be oxidized;
n is an integer selected from 0, 1 or 2;
and/or an enantiomer, diastereomer or agriculturally or veterinarily acceptable salt thereof.

48. The compound of claim 47,
wherein
R² is methyl, ethyl, difluoromethyl, trifluoromethyl, pentafluoroethyl, 2,2-difluoroethyl or 2,2,2-trifluoroethyl.
G is a molecular group representing a ring system selected from
a 3, 4-, 5-, 6- or 7-membered heterocyclic or heteroaromatic monocyclic ring system or
a 7-11-membered heterobiaromatic or heterobicyclic or heterospirobicyclic ring system,
wherein each ring of the ring system comprises 1, 2 or 3 heteroatoms selected from the group consisting of oxygen, nitrogen and sulfur atoms, and wherein said nitrogen and sulfur atom(s), independently of one another, may be oxidized, and wherein the ring system is attached via a carbon ring atom to the remainder of the molecule, and
wherein the ring system may be optionally substituted with k substituents R⁶, wherein said substituents R⁶ are selected independently from one another, if k is more than one,
and/or an enantiomer, diastereomer or agriculturally or veterinarily acceptable salt thereof.

49. A substituted 3-pyridyl thiazole compound according to claim 45, wherein
R¹ is selected from the group consisting of hydrogen or fluoro.

50. A substituted 3-pyridyl thiazole compoundaccording to claim 45, wherein
R¹ is selected from the group consisting of hydrogen or fluoro;
R² is selected from the group consisting of fluoro, chloro, bromo, cyano, methyl, ethyl, propyl, isopropyl, tertbutyl, cyclopropyl, difluoromethyl, trifluoromethyl, 2,2-difluoroethyl or 2,2,2-trifluoroethyl.

51. A substituted 3-pyridyl thiazole compound according to claim 47, wherein
R² is selected from the group consisting of hydrogen, fluoro, chloro, bromo, cyclopropyl, propyl, tertbutyl.

52. A substituted 3-pyridyl thiazole compoundaccording to claim 45, wherein
G is a nitrogen containing 4-, 5-, or 6-membered heterocyclic or hetero-aromatic ring system selected from any of the ring systems G-1k to G-135k defined in claim 42,
and wherein each
R⁶ is independently selected from the group consisting of hydrogen, halogen, cyano, nitro, C₁-C₆-alkyl, C₃-C₆-cycloalkyl, wherein the carbon atoms of the aforementioned aliphatic and cyclo-aliphatic radicals may optionally be partially or fully halogenated and/or may carry 1 or 2 radicals R¹⁵, which are selected independently from one another,
OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶;
or a ring system selected from phenyl, pyridyl, pyrimidyl, pyrazolyl, thiazolyl, oxazolyl or thiophenyl, thiazolinyl, oxazolinyl, isoxazolinyl, pyrazolinyl, imidazolinyl, each optionally substituted with one or more, e.g. 1, 2, 3, 4, or 5 substituents selected independently from R¹⁸;
or two R⁶ present on one ring carbon or sulfur atom may together form ═O, ═S, ═NR^{17a};
and wherein
k is an integer of 0, 1, 2 or 3.

53. A substituted 3-pyridyl thiazole compound according to claim 46,
wherein
G is a nitrogen containing 4-, 5-, or 6-membered heterocyclic or hetero-aromatic ring system selected from any of the following ring systems:
G-1k to G-21k, G-23k to G-31k, G-33k to G-46k, G-51k, G-52k, G-54k, G-56k, G-58k, G-60k, G-61k, G-63k to G-67k, G-70k to G-78k, G-81k to G-84k, G-86k to G-135k as defined in claim 42,
and wherein each
R⁶ is independently selected from the group consisting of hydrogen, halogen, cyano, nitro, C₁-C₆-alkyl, C₃-C₆-cycloalkyl, wherein the carbon atoms of the aforementioned aliphatic and cyclo-aliphatic radicals may optionally be partially or fully halogenated and/or may carry 1 or 2 radicals R¹⁵, which are selected independently from one another,
OR¹⁶, NR^{17a}R^{17b}, S(O)ₙR¹⁶, S(O)ₙNR^{17a}R^{17b}, C(═O)R¹⁵, C(═O)NR^{17a}R^{17b}, C(═O)OR¹⁶;
or a ring system selected from phenyl, pyridyl, pyrimidyl, pyrazolyl, thiazolyl, oxazolyl or thiophenyl, thiazolinyl, oxazolinyl, isoxazolinyl, pyrazolinyl, imidazolinyl, each optionally substituted with one or more, e.g. 1, 2, 3, 4, or 5 substituents selected independently from R¹⁸;
or two R⁶ present on one ring carbon or sulfur atom may together form ═O, ═S, ═NR^{17a};
and wherein
k is an integer of 0, 1, 2 or 3.

54. An agricultural or veterinary composition comprising a compound of formula (I) as defined in claim 45.

55. The method according to claim 34, wherein the plant propagation material are seeds.

56. Seed treated with a compound of formula (I) as defined in claim 45.
